(19) United States
(12) Patent Application Publication
(10) Pub. No.: US 2025/0019332 A1
(43) Pub. Date: Jan. 16, 2025

(54) (HET)ARYL SUBSTITUTED BISPHENOL COMPOUNDS AND THERMOPLASTIC RESINS

(71) Applicant: REUTER CHEMISCHE APPARATEBAU e.K., Freiburg (DE)

(72) Inventors: Vasyl ANDRUSHKO, Freiburg (DE); Karl REUTER, Freiburg (DE); Philipp KOSCHKER, Freiburg (DE); Florian STOLZ, Freiburg (DE); Mark KANTOR, Freiburg (DE); Noriyuki KATO, Tokyo (JP); Katsushi NISHIMORI, Tokyo (JP); Atsushi MOTEGI, Tokyo (JP); Kentaro ISHIHARA, Tokyo (JP); Shinya IKEDA, Niigata (JP); Takafumi WATANABE, Tokyo (JP); Tatsunobu OGATA, Tokyo (JP); Kazutaka TAKAMATSU, Tokyo (JP); Yutaro HARADA, Tokyo (JP)

(21) Appl. No.: 18/687,253

(22) PCT Filed: Sep. 8, 2022
(86) PCT No.: PCT/EP2022/074976

§ 371 Date: Feb. 27, 2024

(30) Foreign Application Priority Data
Sep. 10, 2021 (EP) 21195940.8
Apr. 26, 2022 (EP) 22169991.1

(51) Int. Cl. C07C 43/23; C07C 317/22; C07C 323/20; C07D 333/76; C07D 339/08; C08G 64/08; G02B 1/04

(52) U.S. Cl. CPC C07C 43/23; C07C 317/22; C07C 323/20; C07D 333/76; C07D 339/08; C08G 64/08; C08G 64/081; G02B 1/041

(57) ABSTRACT

The present invention relates to compounds of the formula (I) where X is selected from the group consisting of a single bond, O, N—(C₁-C₄)-alkyl, N—Ar¹, CR⁵R⁶, S, S(O) and SO₂; Z¹ and Z² are independently selected from hydrogen, -Alk-OH, —CH₂—Ar²—CH₂—OH, -Alk′-C(O)OR^{x}, —CH₂—Ar²—C(O)ORx and —C(O)—Ar²—C(O)OR^{x}, where R^{x} is selected from the group consisting of hydrogen, phenyl, benzyl and C₁-C₄-alkyl; R¹ and R² are independently selected from the group consisting of optionally substituted mono- or polycyclic aryl having from 6 to 26 carbon atoms as ring members and optionally substituted mono- or polycyclic hetaryl having a total of 5 to 26 atoms as ring members, R⁵ is selected from the group consisting of hydrogen, C₁-C₄-alkyl and a radical Ar¹; R⁶ is selected from the group consisting of hydrogen and C₁-C₄-alkyl; Alk is C₂-C₄-alkandiyl; provided that R¹ and R² are not both phenyl, if R³ and R⁴ are both hydrogen. The present invention also relates to use of the compounds of the formula (I) as defined herein as a monomer for producing thermoplastic resins comprising a structural unit represented by formulae (II) where # represents a connection point to a neighboring structural unit; and where X, Z^{1a}, Z^{2a}, R¹, R², R³ and R⁴ are as defined herein, and to such thermoplastic resins. The present invention also relates optical devices made of a thermoplastic resin comprising a structural unit represented by formulae (II) as defined herein.

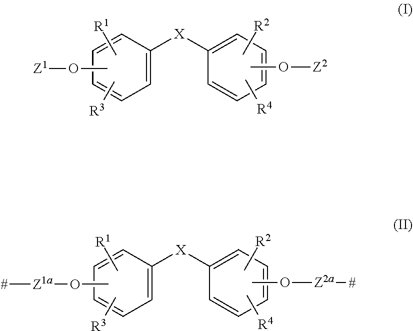

The present invention relates to (het)aryl substituted bisphenol compounds that are suitable as monomers for preparing thermoplastic resins, such as polycarbonate resins, which have beneficial optical and mechanical properties and can be used for producing optical devices.

### BACKGROUND OF INVENTION

Optical devices, such as optical lenses made of optical resin instead of optical glass are advantageous in that they can be produced in large numbers by injection molding. Nowadays, optical resins, in particular, transparent polycarbonate resins, are frequently used for producing camera lenses. In this regard, resins with a higher refractive index are highly desirable, as they allow for reducing the size and weight of final products. In general, when using an optical material with a higher refractive index, a lens element of the same refractive power can be achieved with a surface having less curvature, so that the amount of aberration generated on this surface can be reduced. As a result, it is possible to reduce the number of lenses, to reduce the eccentric sensitivity of lenses and/or to reduce the lens thickness to thereby achieve weight reduction.

EP2034337 describes a copolycarbonate resin which comprises 99 to 51 mol % of a repeating unit derived from 9,9-bis(4-(2-hydroxyethoxy)phenyl)fluorene and 1 to 49 mol % of a repeating unit derived from bisphenol A. The resin is suitable for preparing an optical lens having a low Abbe number of 23 to 26 and a refractive index from 1.62 to 1.64.

U.S. Pat. No. 9,360,593 describes polycarbonate resins having repeating units derived from 2,2′-Bis(2-hydroxyethoxy)-1,1-binaphthyl. It is said that the polycarbonate resins have beneficial optical properties in terms of a high refractive index, a low Abbe's number, a high degree of transparency, low birefringence, and a glass transition temperature suitable for injection molding. Co-Polycarbonates of bis(2-hydroxyethoxy)-1,1-binaphthyl with 10,10-bis(4-hydroxyphenyl)anthrone monomers and their use for preparing optical lenses are described in US 2016/0319069. The copolymers have been reported to have a good moisture resistance, and have refractive indices ranging from about 1.662 to 1.667.

WO 2019/043060 describes thermoplastic resins for producing optical materials, where the thermoplastic resins comprise a polymerized compound of formula (2)

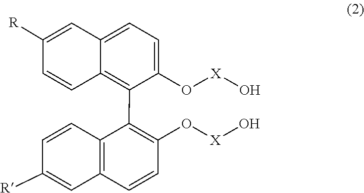

where
X is e.g. C₂-C₄-alkandiyl;
R and R′ are identical or different and selected from optionally substituted mono or polycyclic aryl having from 6 to 36 carbon atoms and optionally substituted mono- or polycyclic hetaryl having a total of 5 to 36 atoms.

WO 2019/154727 describes thermoplastic resins for producing optical materials, where the thermoplastic resins comprise a polymerized compound of formula (3)

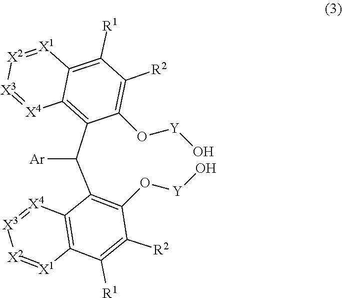

where
R¹, R² are e.g. hydrogen;
Y is an alkylene group having 2, 3 or 4 carbon atoms,
Ar is selected from mono- or polycyclic aryl and mono- or polycyclic hetaryl;
X¹, X², X³, X⁴ are CH, C—R^{x} or N, provided that in each ring at most two of X¹, X², X³, X⁴ are N;
R^{x} is e.g. halogen, CN or CH═CH₂.

WO 2020/079225 describes thermoplastic resins for producing optical materials, where the thermoplastic resins comprise a polymerized compound of formula (4)

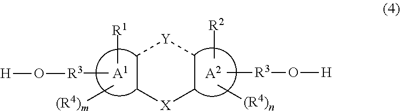

where
A¹, A² are selected from mono- or bicyclic aromatic radicals and mono- or bicyclic heteroaromatic radicals,
X represents e.g. a single bond, O, NH, or an optionally substituted carbon atom
Y is e.g. absent or represents a single bond or has the meaning given for X;
R¹, R² are hydrogen, a radical Ar′ or a radical R^{a};
R³ is in particular O-alkylene;
m, n are 0, 1 or 2;
R⁴, R⁵ are e.g. selected from CN and a radical R^{a};
R^{a} is selected from the group consisting of C≡C—R¹¹ and Ar—C≡C—R¹¹ where R¹¹ and Ar are aromatic radicals,
where at least one of the radicals attached to A¹ or A² is a radical R^{a}.

S. R. Turner et al., High Performance Polymers 17 (2005) pp. 361-376 describe amorphous copolyesters derived from bisphenols, such as bis(2-hydroxyethoxy)-2,2′-diphenyl]-bisphenol S (=di-[4-(2-hydroxyethoxy)-2-phenyl]-phenylsulfon) and bis[(2-hydroxyethoxy)-2,2′phenyl]-,4,4′-biphenol.

Monomers for producing thermoplastic resins having a high refraction index generally also lead to a positive birefringence value of the resins. For optical devices, birefringence is an undesirable property. To date, the positive birefringence is compensated by using co-monomers having a negative birefringence, such as 9,9-bis(4-(2-hydroxyethoxy)phenyl)fluorene. However, these co-monomers reduce the refraction index of the resulting polymer. Presently, hardly any monomers are known that provide for high refractive index and low birefringence.

Despite the advances made in the field of optical resins, there is still an ongoing need for monomers for preparing optical resins, in particular polycarbonate resins and polyester resins, which monomers result in a high refractive index and which are therefore useful for making optical devices, in particular lenses. Apart from that, the monomers should not impair the other optical properties of the optical resins, such as low Abbe's number, a high degree of transparency and low birefringence. Moreover, the monomers should be easy to prepare. In addition, the resins, in particular polyesters and polycarbonates, obtained from these monomers should have good moisture and heat resistance and they should have a sufficiently high glass transition temperature suitable for injection molding.

### PRIOR ART DOCUMENTS

### Patent Documents

Patent Document 1: EP2034337
Patent Document 2: U.S. Pat. No. 9,360,593
Patent Document 3: US 2016/0319069
Patent Document 4: WO 2019/043060
Patent Document 5: WO 2019/154727
Patent Document 6: WO 2020/079225

### Non-Patent Document

Non-patent document 1: S. R. Turner et al., High Performance Polymers 17 (2005) pp. 361-376

### SUMMARY OF INVENTION

It was surprisingly found that the compounds of the formula (I) as described herein are useful monomers for preparing thermoplastic resins, in particular for polycarbonates and polyesters, having high transparency and high refractive index and also impart an appropriate glass transition temperature to the polycarbonates and polyesters. Such thermoplastic resins are therefore suitable for producing optical resins where high transparency and high refractive index are required. Some of the monomers of the formula (I) described herein provide for both a high refractive index and a low or even negative birefringence. Moreover, the compounds of the formula (I) can be easily incorporated into polyesters and polycarbonates and are thermally stable under the polymerization conditions. Therefore, the resulting polyesters and polycarbonates have low yellowness. Thus, thermoplastic resins containing the monomers of the formula (I) in polymerized form can advantageously be used for preparing optical devices made of resins.

Therefore, the present invention relates to compounds of the formula (I)

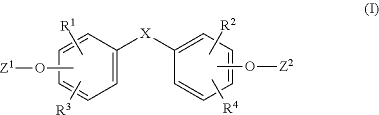

where
X is selected from the group consisting of a single bond, O, N—(C₁-C₄)-alkyl, N—Ar¹, CR⁵R⁶, S, S(O) and SO₂;
Z¹ and Z² are independently selected from hydrogen, -Alk¹-OH, —CH₂—Ar²—CH₂—OH, -Alk²-C(O)OR^{x}, —CH₂—Ar²—C(O)OR^{x} and —C(O)—Ar²—C(O)OR^{x},
   where R^{x} is selected from the group consisting of hydrogen, phenyl, benzyl and C₁-C₄-alkyl;
R¹ and R² are independently selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms,
   where mono- or polycyclic aryl and mono- or polycyclic hetaryl are unsubstituted or carry 1, 2, 3 or 4 radicals R^{Ar};
R³ and R⁴ are independently selected from the group consisting of hydrogen, mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms,
   where mono- or polycyclic aryl and mono- or polycyclic hetaryl are unsubstituted or carry 1, 2, 3 or 4 radicals R^{Ar};
R⁵ is selected from the group consisting of hydrogen and C₁-C₄-alkyl;
R⁶ is selected from the group consisting of hydrogen and C₁-C₄-alkyl;
Ar¹ is selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms,
   where mono- or polycyclic aryl and mono- or polycyclic hetaryl are unsubstituted or carry 1, 2, 3 or 4 radicals R^{Ar};
Ar² is selected from the group consisting of phenylene, naphthylene and biphenylylene;
Alk is C₂-C₄-alkandiyl;
Alk′ is C₁-C₄-alkandiyl;
R^{Ar} is selected from the group consisting of R, OR, CHₙR₃₋ₙ, NR₂ and CH═CHR′, where R^{Ar} may identical or different if more than one is present on the same (het)aryl group;
R is selected from the group consisting of methyl, ethyl, phenyl, naphthyl, phenanthrenyl and triphenylenyl, where phenyl, naphthyl, phenanthrenyl and triphenylenyl are unsubstituted or substituted by 1, 2, 3 or 4 identical or different radicals R″;
R′ is selected from hydrogen, methyl, phenyl and naphthyl, where phenyl and naphthyl are unsubstituted or substituted by 1, 2, 3 or 4 identical or different radicals R″;
R″ is selected from the group consisting of phenyl, OCH₃, CH₃, N(CH₃)₂ and C(O)CH₃; and
n is 0, 1 or 2;
provided that R¹ and R² are not both phenyl if R³ and R⁴ are both hydrogen.

The above compounds are particularly useful in the preparation of thermoplastic resins, in particular for optical resins as defined herein, especially for polycarbonate resins.

When used as monomers for the preparation of optical resins, in particular polycarbonate resins and polyester resins, the compounds of the formula (I) provide for resins with high refractive indices. Moreover, compounds of formula (I) provide for high transparency of the resins and they do not significantly impair other optical properties and the mechanical properties of the resins. In particular, these resins fulfil the other requirements of optical resins, such as low Abbe's number, a high degree of transparency and low birefringence. Moreover, the monomers provide sufficiently high glass transition temperatures to the optical resins produced therefrom. Apart from that, the monomers of formula (I) can be easily prepared and obtained in high yields and high purity. In particular, the compounds of formula (I) can be obtained in crystalline form, which allows for an efficient purification to the degree required in the preparation of optical resins. In particular, the compounds of formula (I) can be obtained in a purity which provides for low haze, which is in particular important for the use in the preparation of optical resins. Compounds of formula (I), which do not bear color-imparting radicals, such as some of the radicals R¹, R², R³, R⁴ and Ar¹, can also be obtained in a purity, which provides for a low yellowness index Y.I. and low APHA color number, as determined in accordance with ASTM E313, which may also be important for the use in the preparation of optical resins.

The invention also relates to a thermoplastic resin comprising a polymerized unit of the compounds of formula (I), i.e. a thermoplastic resin comprising a structural unit represented by formula (II) below;

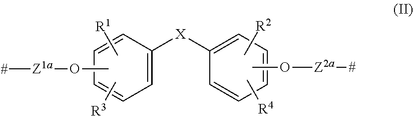

where
# represents a connection point to a neighboring structural unit;
and where Z^{1a} and Z^{2a}, respectively, is derived from Z¹ or Z² in formula (I), if Z¹ or Z² is hydrogen, by replacing hydrogen with a single bond, or, if Z¹ or Z² is not hydrogen, by replacing the —OH or —OR^{x} group of Z¹ or Z² with an oxo (—O—) unit, and where Z¹, Z², X, R¹, R², R³ and R⁴ are as defined herein above.

The invention further relates to a thermoplastic resin selected from copolycarbonate resins, copolyestercarbonate resins and copolyester resins, where the thermoplastic resin in addition to the structural units of formula (II) also comprises structural units of the formula (V),

#-O—R^{z}-A¹-R^{z}—O-#- (V)
where
# represents a connection point to a neighboring structural unit;
A¹ is a polycyclic radical bearing at least 2 benzene rings, wherein the benzene rings may be connected by A and/or directly fused to each other and/or fused by a non-benzene carbocycle, where A¹ is unsubstituted or substituted by 1, 2 or 3 radicals R^{aa}, which are selected from the group consisting of halogen, C₁-C₆-alkyl, C₅-C₆-cycloalkyl and phenyl;
A is selected from the group consisting of a single bond, O, C═O, S, SO₂, CH₂, CH—Ar, CAr₂, CH(CH₃), C(CH₃)₂ and a radical of the formula (A′)

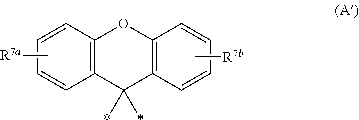

where
Q represents a single bond, O, NH, C═O, CH₂ or CH═CH;
R^{7a}, R^{7b}, independently of each other are selected from the group consisting of hydrogen, fluorine, CN, R, OR, CHₖR₃₋ₖ, NR₂, C(O)R and C(O)NH₂, where R is as defined herein and k is 0, 1, 2 or 3; and
* represents the connection point to a benzene ring;
Ar is selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk¹, O-Alk²-, O-Alk²-[O-Alk²-]ₚ- or O-Alk³-C(O)— where O is bound to A¹, and where
   p is an integer from 1 to 10;
   Alk¹ is C₁-C₄-alkandiyl;
   Alk² is C₂-C₄-alkandiyl; and
   Alk³ is C₁-C₄-alkandiyl.

The invention further relates to an optical device made of a thermoplastic resin as defined above, in particular from a polyester and especially from a polycarbonate.

### DETAILED DESCRIPTION OF INVENTION

If X is a single bond the compounds of formula (I) may, depending on the types and positions the substituents —O—Z¹, —O—Z², R¹, R², R³ and R⁴, have axial chirality due to a possibly limited rotation along the bond between the two phenylene moieties. In that case the compounds of the formula (I) can therefore exist in the form of their (S)-enantiomer and their (R)-enantiomer. Consequently, the compounds of formula (I) may exist as a racemic mixture or as non-racemic mixtures or in the form of their pure (S)- and (R)-enantiomers, respectively. The present invention relates to both the racemic and the non-racemic mixtures of the enantiomers of the compounds of formula (I), where X is a single bond, and also to their pure (S)- and (R)-enantiomers, as far as these enantiomers exist.

In terms of the present invention, the term “C₁-C₄-alkandiyl group” may alternatively also be designated “alkylene group having 1, 2, 3 or 4 carbon atoms” and refers to a bivalent, saturated, aliphatic hydrocarbon radical having 1, 2, 3 or 4 carbon atoms. Examples of C₂-C₄-alkandiyl are in particular the methylene group (CH₂), linear alkandiyl such as 1,2-ethandiyl (CH₂CH₂), 1,3-propandiyl (CH₂CH₂CH₂) and 1,4-butdandiyl (CH₂CH₂CH₂CH₂), but also branched alkandiyl such as 1-methyl-1,2-ethandiyl, 1-methyl-1,2-propandiyl, 2-methyl-1,2-propandiyl, 2-methyl-1,3-propandiyl and 1,3-butandiyl.

In terms of the present invention, the term “monocyclic aryl” refers to a monovalent aromatic monocyclic radical, such as in particular phenyl.

In terms of the present invention, the term “monocyclic hetaryl” refers to a monovalent heteroaromatic monocyclic radical, i.e. a heteroaromatic monocycle linked by a single covalent bond to the remainder of the molecule, where the ring member atoms are part of a conjugate π-electron system, where the heteroaromatic monocycle has 5 or 6 ring atoms, which comprise as heterocyclic ring members 1, 2, 3 or 4 nitrogen atoms or 1 oxygen atom and 0, 1, 2 or 3 nitrogen atoms, or 1 sulphur atom and 0, 1, 2 or 3 nitrogen atoms, where the remaining ring atoms are carbon atoms. Examples include furyl (=furanyl), pyrrolyl (=1H-pyrrolyl), thienyl (=thiophenyl), imidazolyl (=1H-imidazolyl), pyrazolyl (=1H-pyrazolyl), 1,2,3-triazolyl, 1,2,4-triazolyl, tetrazolyl, oxazolyl, thiazolyl, isoxazolyl, isothiazolyl, 1,3,4-oxadiazolyl, 1,3,4-thiadiazolyl, pyridyl (=pyridinyl), pyrazinyl, pyridazinyl, pyrimidinyl and triazinyl.

In terms of the present invention, the term “mono- or polycyclic aryl” refers to a monovalent aromatic monocyclic radical as defined herein or to a monovalent aromatic polycyclic radical, i.e. a polycyclic arene linked by a single covalent bond to the remainder of the molecule, where the polycyclic arene is
(i) an aromatic polycyclic hydrocarbon, i.e. a completely unsaturated polycyclic hydrocarbon, where each of the carbon atoms is part of a conjugate π-electron system,
(ii) a polycyclic hydrocarbon which bears at least 1 phenyl ring which is fused to a saturated or unsaturated 4 to 10-membered mono- or bicyclic hydrocarbon ring,
(iii) a polycyclic hydrocarbon which bears at least 2 phenyl rings which are linked to each other by a covalent bond or which are fused to each other directly and/or which are fused to a saturated or unsaturated 4 to 10-membered mono- or bicyclic hydrocarbon ring.

Mono- or polycyclic aryl has from 6 to 26, often from 6 to 24 carbon atoms, e.g. 6, 9, 10, 12, 13, 14, 16, 17, 18, 19, 20, 22 or 24 carbon atoms as ring atoms, in particular from 6 to 20 carbon atoms, especially 6, 10, 12, 13, 14, 16, 17 or 18 carbon atoms. Polycyclic aryl typically has 10 to 26 carbon atoms as ring atoms, in particular from 10 to 20 carbon atoms, especially 10, 12, 13, 14, 16, 17 or 18 carbon atoms.

In this context, polycyclic aryl bearing 2, 3 or 4 phenyl rings which are linked to each other via a single bond include e.g. biphenylyl and terphenylyl. Polycyclic aryl bearing 2, 3 or 4 phenyl rings which are directly fused to each other include e.g. naphthyl, anthracenyl, phenanthrenyl, pyrenyl, triphenylenyl, chrysenyl and benzo[c]phenanthrenyl. Polycyclic aryl bearing 2, 3 or 4 phenyl rings which are fused to a saturated or unsaturated 4- to 10-membered mono- or bicyclic hydrocarbon ring include e.g. 9H-fluorenyl, biphenylenyl, tetraphenylenyl, acenaphthenyl (1,2-dihydroacenaphthylenyl), acenaphthylenyl, 9,10-dihydroanthracen-1-yl, 1,2,3,4-tetrahydrophenanthrenyl, 5,6,7,8-tetrahydrophenanthrenyl, cyclopent[fg]acenaphthylenyl, phenalenyl, fluoranthenyl, benzo[k]fluoranthenyl, perylenyl, 9,10-dihydro-9,10[1′,2′]-benzenoanthracenyl, dibenzo[a,e][8]annulenyl, 9,9′-spirobi[9H-fluoren]yl and spiro[1H-cyclobuta[de]naphthalene-1,9′-[9H]fluoren]yl.

Mono- or polycylic aryl includes, by way of example phenyl, naphthyl, 9H-fluorenyl, phenanthryl, anthracenyl, pyrenyl, chrysenyl, benzo[c]phenanthrenyl, acenaphthenyl, acenaphthylenyl, 2,3-dihydro-1H-indenyl, 5,6,7,8-tetrahydro-naphthalenyl, cyclopent[fg]acenaphthylenyl, 2,3-dihydrophenalenyl, 9,10-dihydroanthracen-1-yl, 1,2,3,4-tetrahydrophenanthrenyl, 5,6,7,8-tetrahydrophenanthrenyl, fluoranthenyl, benzo[k]fluoranthenyl, biphenylenyl, triphenylenyl, tetraphenylenyl, 1,2-dihydroacenaphthylenyl, dibenzo[a,e][8]annulenyl, perylenyl, biphenylyl, terphenylyl, naphthylenphenyl, phenanthrylphenyl, anthracenylphenyl, pyrenylphenyl, 9H-fluorenylphenyl, di(naphthylen)phenyl, naphthylenbiphenyl, tri(phenyl)phenyl, tetra(phenyl)phenyl, pentaphenyl(phenyl), phenylnaphthyl, binaphthyl, phenanthrylnaphthyl, pyrenylnaphthyl, phenylanthracenyl, biphenylanthracenyl, naphthalenylanthracenyl, phenanthrylanthracenyl, dibenzo[a,e][8]annulenyl, 9,10-dihydro-9,10[1′,2′]benzoanthracenyl, 9,9′-spirobi-9H-fluorenyl and spiro[1H-cyclobuta[de]naphthalene-1,9′-[9H]fluoren]yl.

In terms of the present invention, the term “mono- or polycyclic hetaryl” refers to a monovalent heteroaromatic monocyclic radical as defined herein or to a monovalent heteroaromatic polycyclic radical, i.e. a polycyclic hetarene linked by a single covalent bond to the remainder of the molecule, where
(i) the polycyclic hetarene bears a heteroaromatic monocycle as defined above and at least one, e.g. 1, 2, 3, 4 or 5, further aromatic rings selected from phenyl and heteroaromatic monocycles as defined above, where the aromatic rings of the polycyclic hetarene are linked to each other by a covalent bond and/or fused to each other directly and/or fused to a saturated or unsaturated 4 to 10-membered mono- or bicyclic hydrocarbon ring, or
(ii) the polycyclic hetarene bears at least one saturated or partially or fully unsaturated 5-, 6-, 7- or 8-membered heterocyclic ring bearing 1, 2 or 3 heteroatoms selected from oxygen, sulphur and nitrogen as ring atoms, such as 2H-pyran, 4H-pyran, thiopyran, 1,4-dihydropyridin, 4H-1,4-oxazin, 4H-1,4-thiazin, 1,4-dioxin, oxepin, thiepin, dioxin, dithiin, dioxepin, dithiepin, dioxocine, dithiocine and at least one, e.g. 1, 2, 3, 4 or 5, aromatic rings selected from phenyl and heteroaromatic monocycles as defined above, where at least one of the aromatic rings is directly fused to the saturated or partially unsaturated 5- to 8-membered heterocyclic ring and where the aromatic rings of the polycyclic hetarene are linked to each other by a covalent bond or fused to each other directly and/or fused to a saturated or unsaturated 4 to 10-membered mono- or bicyclic hydrocarbon ring.

Mono- or polycyclic hetaryl has from 5 to 26, often from 5 to 24 ring atoms, in particular 5 to 20 ring atoms, which comprise 1, 2, 3 or 4 atoms selected from nitrogen atoms, sulphur atoms and oxygen atoms, where the remainder of the ring atoms are carbon atoms. Polycyclic hetaryl generally has from 9 to 26, often from 9 to 24 ring atoms, in particular 9 to 20 ring atoms, which comprise 1, 2, 3 or 4 atoms selected from nitrogen atoms, sulphur atoms and oxygen atoms, where the remainder of the ring atoms are carbon atoms.

Examples of polycyclic hetaryl include, but are not limited to, benzofuryl, benzothienyl, dibenzofuranyl (=dibenzo[b,d]furanyl), dibenzothienyl (=dibenzo[b,d]thienyl), naphthofuryl, naphthothienyl, furo[3,2-b]furanyl, furo[2,3-b]furanyl, furo[3,4-b]furanyl, thieno[3,2-b]thienyl, thieno[2,3-b]thienyl, thieno[3,4-b]thienyl, oxanthrenyl, thianthrenyl, indolyl (=1H-indolyl), isoindolyl (=2H-isoindolyl), carbazolyl, indolizinyl, benzopyrazolyl, benzimidazolyl, benzoxazolyl, benzothiazolyl, benzo[c,d]indolyl, 1H-benzo[g]indolyl, quinolinyl, isoquinolinyl, acridinyl, phenazinyl, quinazolinyl, quinoxalinyl, phenoxazinyl, phenthiazinyl, benzo[b][1,5]naphthyridinyl, cinnolinyl, 1,5-naphthyridinyl, 1,8-naphthyridinyl, phenylpyrrolyl, naphthylpyrrolyl, dipyridyl, phenylpyridyl, naphthylpyridyl, pyrido[4,3-b]indolyl, pyrido[3,2-b]indolyl, pyrido[3,2-g]quinolinyl, pyrido[2,3-b][1,8]naphthyridinyl, pyrrolo[3,2-b]pyridinyl, pteridinyl, puryl, 9H-xanthenyl, 9H-thioxanthenyl, 2H-chromenyl, 2H-thiochromenyl, phenanthridinyl, phenanthrolinyl, benzo[1,2-b:4,3-b′]difuranyl, benzo[1,2-b:6,5-b′]difuranyl, benzo[1,2-b:5,4-b′]difuranyl, benzo[1,2-b:4,5-b′]difuranyl, naphthofuranyl, benzo[b]naphtho[1,2-d]furanyl, benzo[b]naphtho[2,3-d]furanyl, benzo[b]naphtho[2,1-d]furanyl, tribenzo[b,d,f]oxepinyl, dibenzo[b,d]thienyl, naphtho[1,2-b]thienyl, naphtho[2,3-b]thienyl, naphtho[2,1-b]thienyl, benzo[b]naphtho[1,2-d]thienyl, benzo[b]naphtho[2,3-d]thienyl, benzo[b]naphtho[2,1-d]thienyl, 6H-dibenzo[b,d]thiopyranyl, 5H,9H-[1]benzothiopyrano[5,4,3-c,d,e][2]benzothiopyranyl, 5H,10H-[1]benzothiopyrano[5,4,3-c,d,e][2]benzothiopyranyl, benzo[1,2-b:4,3-b′]bisthienyl, benzo[1,2-b:6,5-b′]bisthienyl, benzo[1,2-b:5,4-b′]bisthienyl, benzo[1,2-b:4,5-b′]bisthienyl, 1,4-benzodithiinyl, naphtho[1,2-b][1,4]dithiinyl, naphtho[2,3-b][1,4]dithiinyl, thianthrenyl, benzo[a]thianthrenyl, benzo[b]thianthrenyl, dibenzo[a,c]thianthrenyl, dibenzo[a,h]thianthrenyl, dibenzo[a,i]thianthrenyl, dibenzo[a,j]thianthrenyl, dibenzo[b,i]thianthrenyl, 2H-naphtho[1,8-b,c]thienyl, 5H-phenanthro[4,5-b,c,d]thiopyranyl, 10,11-dihydrodibenzo[b,f]thiepinyl, 6,7-dihydrodibenzo[b,d]thiepinyl, dibenzo[b,f]thiepinyl, dibenzo[b,d]thiepinyl, 6H-dibenzo[d,f][1,3]dithiepinyl, tribenzo[b,d,f]thiepinyl, benzothieno[3,4-c,d]thieno[2,3,4-j,k][2]benzothiepinyl, dinaphtho[1,8-bc:1′,8′-f,g][1,5]dithiocinyl, furo[3,2-g]quinolinyl, furo[2,3-g]quinolinyl, furo[2,3-g]quinoxalinyl, benzo[g]chromenyl, thieno[3,2-f][1]benzothienyl, thieno[2,3-f][1]benzothienyl, thieno[3,2-g]quinolinyl, thieno[2,3-g]quinolinyl, thieno[2,3-g]quinoxalinyl, benzo[g]thiochromenyl, pyrrolo[3,2,1-h,i]indolyl, benzo[g]quinoxalinyl, benzo[f]quinoxalinyl, and benzo[h]isoquinolinyl.

In terms of the present invention, the terms “phenylene”, “naphthylene” and “biphenylylene” refer, as customary in the art, to diradikals of benzene, naphthalene and biphenyl, respectiviely. Accordingly, the terms “phenylene”, “naphthylene” and “biphenylylene” are used herein synonymously with the terms phendiyl, naphthalendiyl and biphenyldiyl, respectively.

In terms of the present invention, a “structural unit” is a structural element which is present repeatedly in the polymer backbone of the thermoplastic resin. Therefore, the terms “structural unit” and “repeating unit” are used synonymously.

In terms of the present invention, the term “optical device” refers to a device that is transparent for visible light and manipulates light beams, in particular by refraction.

Optical devices include but are not limited to prisms, lenses, optical films and combinations thereof, especially lenses for cameras and lenses for glasses.

The remarks made below as to preferred embodiments of the variables (substituents) of the compounds of formula (I) and of the structural units of formula (II) are valid on their own as well as preferably in combination with each other.

The remarks made below concerning preferred embodiments of the variables further are valid on their own as well as preferably in combination with each other concerning the compounds of formula (I) and the structural units of formula (II), where applicable, as well as concerning the uses according to the invention.

If the both R³ and R⁴ in formula (I) are hydrogen, the radicals R¹ and R² are preferably selected from the group consisting of polycyclic aryl having from 10 to 26 carbon atoms as ring atoms and polycyclic hetaryl having a total of 9 to 26 atoms, which are ring member atoms, where 1, 2, 3 or 4 of the ring member atoms of polycyclic hetaryl are selected from the group consisting of nitrogen, sulfur and oxygen, while the remainder of these ring member atoms of hetaryl are carbon atoms, where polycyclic aryl and polycyclic hetaryl are unsubstituted or carry 1, 2, 3 or 4 radicals R^{Ar}.

In formula (I) and likewise in formula (II), the variables X, R¹, R², R³, R⁴, Z¹ and Z² on their own or preferably in any combination preferably have the following meanings:

Preference is given to those variables Z¹ and Z² in formula (I) that are independently selected from hydrogen, -Alk-OH, —CH₂—Ar²—CH₂—OH, -Alk′-C(O)OR^{x} and —CH₂—Ar²—C(O)OR^{x}, and accordingly to those variables Z^{1a} and Z^{2a} in formula (II) that are independently selected from -Alk-O—, —CH₂—Ar²—CH₂—O—, -Alk′-C(O)O— and —CH₂—Ar²—C(O)O—, where Alk, -Alk′, Ar² and R^{x} have the meanings defined herein, in particular the preferred meanings.

In a preferred group (1) of embodiments, the variables Z¹ and Z² in formula (I) are independently selected from -Alk-OH and —CH₂—Ar²—CH₂—OH and accordingly the variables Z^{1a} and Z^{2a} in formula (II) are independently selected from -Alk-O— and —CH₂—Ar²—CH₂—O—, wherein Alk is preferably a linear C₂-C₄-alkandiyl, such as 1,2-ethandiyl (CH₂—CH₂), 1,3-propandiyl or 1,4-butandiyl, and in particular is 1,2-ethandiyl, and Ar² is preferably selected from 1,4-phenylene, 1,3-phenylene, 2,6-naphthylene, 1,4-naphthylene, 1,5-naphthylene and 4,4′-biphenylylene. It is also preferred in this context that the variables Z¹ and Z² in formula (I) or the variables Z^{1a} and Z^{2a} in formula (II) are identical to each other.

Accordingly, in a particularly preferred subgroup (1.1) of embodiments the variables Z¹ and Z² in formula (I) are selected from 2-hydroxyethyl (i.e. 2-(HO)-ethyl), hydroxymethyl-phenyl-methyl (i.e. HO-methyl-phenyl-methyl), hydroxymethyl-naphthyl-methyl and hydroxymethyl-biphenylyl-methyl, especially from 2-hydroxyethyl, 4-(hydroxymethyl)phenyl)methyl, (3-(hydroxymethyl)phenyl)methyl, (4-(hydroxymethyl)-1-naphthyl)methyl, (5-(hydroxymethyl)-1-naphthyl)methyl, (6-(hydroxymethyl)-2-naphthyl)methyl and 4′-(hydroxymethyl)-1,1′-biphenylyl-4-methyl, and specifically from 2-hydroxyethyl, 4-(hydroxymethyl)phenyl)methyl and (3-(hydroxymethyl)phenyl)methyl. Correspondingly, in this particularly preferred group (1.1) of embodiments the variables Z^{1a} and Z^{2a} in formula (II) are selected from 2(-O)-ethyl, —O-methyl-phenyl-methyl and —O-methyl-naphthyl-methyl, especially from 2(-O)-ethyl, (4(-O-methyl)phenyl)methyl, (3(O-methyl)phenyl)methyl, (4(-O-methyl)-1-naphthyl)methyl, (5(-O-methyl)-1-naphthyl)methyl, (6(-O-methyl)-2-naphthyl)methyl and 4′(-O-methyl)-1,1′-biphenylyl-4-methyl, and specifically from 2(-O)-ethyl, (4(-O-methyl)phenyl)methyl and (3(-Omethyl)phenyl)methyl, (4(-O-methyl)-1-naphthyl)methyl.

In a particular subgroup (1′) of embodiments the variables Z¹ and Z² in formula (I) have identical meanings and, likewise, the variables Z^{1a} and Z^{2a} in formula (II) have identical meanings, which are selected from the meanings defined in groups (1) and (1.1), of embodiments.

In another group (2) of embodiments the variables Z¹ and Z² in formulae (I) and (II) are both hydrogen and accordingly the variables Z^{1a} and Z^{2a} in formula (II) are both a single bond.

In a preferred group (3) of embodiments, the variables Z¹ and Z² in formula (I) are independently selected from -Alk′-C(O)OR^{x} and —CH₂—Ar²—C(O)OR^{x} and accordingly the variables Z^{1a} and Z^{2a} in formula (II) are independently selected from -Alk′-C(O)O— and —CH₂—Ar²—C(O)O—, wherein Alk′ is preferably a linear C₁-C₄-alkandiyl, such as methylene or 1,2-ethandiyl (CH₂—CH₂), and in particular is methylene, Ar² is preferably selected from 1,4-phenylene, 1,3-phenylene, 2,6-naphthylene, 1,5-naphthylene and 1,4-naphthylene, and R^{x} is preferably hydrogen or C₁-C₄-alkyl, and in particular is methyl. It is also preferred in this context that the variables Z¹ and Z² or the variables Z^{1a} and Z^{2a} are identical to each other.

Accordingly, in a particularly preferred subgroup (3.1) of embodiments the variables Z¹ and Z² in formula (I) are selected from methoxycarbonyl-methyl (i.e. CH₃O—C(O)-methyl), methoxycarbonyl-phenyl-methyl (i.e. CH₃O—C(O)-phenyl-methyl) and methoxycarbonyl-naphthyl-methyl, especially from methoxycarbonyl-methyl, (4-(methoxycarbonyl)phenyl)methyl, (3-(methoxycarbonyl)phenyl)methyl, (4-(methoxycarbonyl)-1-naphthyl)methyl, (5-(methoxycarbonyl)-1-naphthyl)methyl and (6-(methoxycarbonyl)-2-naphthyl)methyl, and specifically from methoxycarbonyl-methyl, (4-(methoxycarbonyl)phenyl)methyl and (3-(methoxycarbonyl)phenyl)methyl. Correspondingly, in this particularly preferred group (3.1) of embodiments the variables Z^{1a} and Z^{2a} in formula (II) are selected from —O—C(O)-methyl, —O—C(O)-phenyl-methyl and —O—C(O)-naphthyl-methyl, especially from —O—C(O)-methyl, (4(-O—C(O)-phenyl)methyl, (3(-O—C(O)-phenyl)methyl, (4-(-O—C(O)-)-1-naphthyl)methyl, (5-(-O—C(O)-)-1-naphthyl)methyl and (6-(-O—C(O)-)-2-naphthyl)methyl, and specifically from —O—C(O)— methyl, (4(-O—C(O)-phenyl)methyl and (3(-O—C(O)-phenyl)methyl.

In a particular subgroup (3′) of embodiments the variables Z¹ and Z² in formula (I) have identical meanings and, likewise, the variables Z^{1a} and Z^{2a} in formula (II) have identical meanings, which are selected from the meanings defined in groups (3) and (3.1), of embodiments.

In preferred group (4) of embodiments, which is a combination of groups (1.1), (2) and (3.1) of embodiments, the variables Z¹ and Z² in formula (I) are selected from hydrogen, 2-hydroxyethyl, methoxycarbonyl-methyl, hydroxymethyl-phenyl-methyl, hydroxymethyl-naphthyl-methyl, hydroxymethyl-biphenylyl-methyl, methoxycarbonylphenyl-methyl and methoxycarbonyl-naphthyl-methyl, in particular selected from hydrogen, 2-hydroxyethyl, methoxycarbonyl-methyl, (4-(hydroxymethyl)phenyl)methyl, (3-(hydroxymethyl)phenyl)methyl, (4-(hydroxymethyl)-1-naphthyl)methyl, (5-(hydroxymethyl)-1-naphthyl)methyl, (6-(hydroxymethyl)-2-naphthyl)methyl, 4′-(hydroxymethyl)-1,1′-biphenylyl-4-methyl, (4-(methoxycarbonyl)phenyl)methyl, (3-(methoxycarbonyl)phenyl)methyl, (4-(methoxycarbonyl)-1-naphthyl)methyl, (5-(methoxycarbonyl)-1-naphthyl)methyl and (6-(methoxycarbonyl)-2-naphthyl)methyl, especially selected from hydrogen, 2-hydroxyethyl, methoxycarbonyl-methyl, (4-(hydroxymethyl)phenyl)methyl, (3-(hydroxymethyl)phenyl)methyl, (4-(methoxycarbonyl)phenyl)methyl and (3-(methoxycarbonyl)phenyl)methyl, and specifically selected from hydrogen, 2-hydroxyethyl, (4-(hydroxymethyl)phenyl)methyl and (3-(hydroxymethyl)phenyl)methyl. Correspondingly, in this preferred group (4) of embodiments the variables Z^{1a} and Z^{2a} in formula (II) are selected from a single bond, 2(-O)ethyl, —O—C(O)-methyl, —O-methyl-phenyl-methyl, —O-methyl-naphthyl-methyl, —O—C(O)— phenyl-methyl and —O—C(O)-naphthyl-methyl, in particular selected from a single bond, 2(-O)-ethyl, —O—C(O)-methyl, (4(-O-methyl)phenyl)methyl, (3(-O-methyl)phenyl)methyl, (4(-O-methyl)-1-naphthyl)methyl, (5(-O-methyl)-1-naphthyl)methyl, (6(-O-methyl)-2-naphthyl)methyl, (4(-O—C(O)-phenyl)methyl, (3-(-O—C(O)-phenyl)methyl, (4-(-O—C(O)-)-1-naphthyl)methyl, (5-(-O—C(O)-)-1-naphthyl)methyl and (6-(methoxycarbonyl)-2-naphthyl)methyl, especially selected from a single bond, 2(-O)-ethyl, —O—C(O)-methyl, (4(-O-methyl)phenyl)methyl, (3(-O-methyl)phenyl)methyl, 4(-O—C(O)-phenyl)methyl and (3-(-O—C(O)-phenyl)methyl, and specifically selected from a single bond, 2(-O)-ethyl, (4(-O-methyl)phenyl)methyl and (3(-O-methyl)phenyl)methyl.

In a particular subgroup (4′) of embodiments the variables Z¹ and Z² in formula (I) have identical meanings and, likewise, the variables Z^{1a} and Z^{2a} in formula (II) have identical meanings, which are selected from the meanings defined in group (4) of embodiments.

The variable X is preferably selected from the group consisting of a single bond, O, N-methyl, N-ethyl, N-n-propyl, N-isopropyl, N-sec-butyl, N-iso-butyl, N-tert-butyl, N—Ar¹, CH₂, C(CH₃)₂, CH(CH₃), C(CH₃)(CH₂CH₃), S, SO and SO₂, where Ar¹ in N—Ar¹ is as defined herein and wherein Ar¹ is in particular selected from the group consisting of phenyl, naphthyl, phenanthryl, biphenylyl, fluorenyl, pyrenyl, chrysenyl, benzo[b]furanyl, dibenzo[b,d]furanyl, naphtho[1,2-b]furanyl, naphtho[2,3-b]furanyl, naphtho[2,1-b]furanyl, oxanthrenyl, benzo[b]thienyl, dibenzo[b,d]thienyl, naphtho[1,2-b]thienyl, naphtho[2,3-b]thienyl, naphtho[2,1-b]thienyl and thianthrenyl, and in particular selected from the group consisting of phenyl, naphthyl, phenanthryl, biphenylyl, fluorenyl, benzo[b]furanyl, dibenzo[b,d]furanyl, benzo[b]thienyl and dibenzo[b,d]thienyl.

In particular, the variable X is selected from the group consisting of a single bond, O, N-methyl, N-ethyl, N-n-propyl, N-isopropyl, N-tert-butyl, N—Ar¹, CH₂, C(CH₃)₂, CH(CH₃), C(CH₃)(CH₂CH₃), S and SO₂, where Ar¹ is selected from the group consisting of phenyl, naphthyl, such as naphth-1-yl or naphth-2-yl, phenanthryl, such as phenanthren-1-yl, phenanthren-2-yl, phenanthren-3-yl, phenanthren-4-yl or phenanthren-9-yl, biphenylyl, such as biphenyl-2-yl, biphenyl-3-yl or biphenyl-4-yl, fluorenyl, such as fluoren-1-yl, fluoren-2-yl, fluoren-3-yl or fluoren-4-yl, benzo[b]furanyl, such as benzo[b]furan-2-yl, benzo[b]furan-3-yl, benzo[b]furan-4-yl, benzo[b]furan-5-yl, benzo[b]furan-6-yl or benzo[b]furan-7-yl, dibenzo[b,d]furanyl, such as dibenzo[b,d]furan-1-yl, dibenzo[b,d]furan-2-yl, dibenzo[b,d]furan-3-yl or dibenzo[b,d]furan-4-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, and dibenzo[b,d]thienyl, such as dibenzo[b,d]thien-1-yl, dibenzo[b,d]thien-2-yl, dibenzo[b,d]thien-3-yl or dibenzo[b,d]thien-4-yl.

In a preferred group (5) of embodiments the variable X is selected from the group consisting of a single bond, O, N-phenyl, N-naphthyl, N-phenanthryl, CH₂, C(CH₃)₂, CH(CH₃), S, S(O), and SO₂, in particular from the group consisting of a single bond, O, N-phenyl, N-naphth-1-yl, N-naphth-2-yl, N-phenanthren-9-yl, CH₂, C(CH₃)₂, S, S(O) and SO₂, especially from the group consisting of a single bond, O, CH₂, C(CH₃)₂, S, S(O) and SO₂ and specifically from the group consisting of a single bond, C(CH₃)₂, S and SO₂.

In a particular subgroup (5′) of embodiments the variable X is CH₂, C(CH₃)₂ or CH(CH₃), and specifically is C(CH₃)₂. In another particular subgroup (5″) of embodiments the variable X is S or SO₂. In yet anyother particular subgroup (5′″) of embodiments the variable X is a single bond.

Preferably, the variables R¹ and R² are independently selected from the group of mono- or polycyclic aryl having from 6 to 18 carbon atoms as ring atoms and polycyclic hetaryl having a total of 9 to 26 atoms, in particular 9 to 18 atoms, which are ring members, where 1 or 2 of these ring member atoms of hetaryl are oxygen or sulfur atoms, while the remainder of these ring member atoms of hetaryl are carbon atoms, where mono- or polycyclic aryl and polycyclic hetaryl are unsubstituted or carry 1 or 2 radicals R^{Ar}, where R^{Ar} has one of the meanings defined herein, especially one of the meanings mentioned as preferred (group 6 of embodiments). More preferably, at least one of R¹ and R², in particular both R¹ and R² are selected from polycyclic aryl having from 10 to 18 carbon atoms as ring member atoms and polycyclic hetaryl having a total of 9 to 18 ring member atoms.

According to a more preferred group (6.1) of embodiments, R¹ and R² are independently selected from phenyl, naphthyl, such as naphth-1-yl or naphth-2-yl, 1,2-dihydroacenaphthylenyl, such as 1,2-dihydroacenaphthylen-3-yl or 1,2-dihydroacenaphthylen-5-yl, biphenylyl, such as biphenyl-4-yl, biphenyl-3-yl or biphenyl-2-yl, fluorenyl, such as fluoren-1-yl, fluoren-2-yl, fluoren-3-yl or fluoren-4-yl, 11H-benzo[a]fluorenyl, such as 11H-benzo[a]fluoren-7-yl, 11H-benzo[b]fluorenyl, such as 11H-benzo[b]fluoren-1-yl, 7H-benzo[c]fluorenyl, such as 7H-benzo[c]fluoren-5-yl or 7H-benzo[c]fluoren-10-yl, phenanthrenyl, such as phenanthren-1-yl, phenanthren-2-yl, phenanthren-3-yl, phenanthren-4-yl or phenanthren-9-yl, benzo[c]phenanthrenyl, such as benzo[c]phenanthren-1-yl, benzo[c]phenanthren-2-yl, benzo[c]phenanthren-3-yl, benzo[c]phenanthren-4-yl, benzo[c]phenanthren-5-yl or benzo[c]phenanthren-6-yl, pyrenyl, such as pyren-1-yl, pyren-2-yl or pyren-4-yl, chrysenyl, such as chrysen-1-yl, chrysen-2-yl, chrysen-3-yl, chrysen-4-yl, chrysen-5-yl or chrysen-6-yl, picenyl, such as picen-3-yl, triphenylenyl, such as triphenylen-1-yl or triphenylen-2-yl, benzo[b]furanyl, such as benzo[b]furan-2-yl, benzo[b]furan-3-yl, benzo[b]furan-4-yl, benzo[b]furan-5-yl, benzo[b]furan-6-yl or benzo[b]furan-7-yl, dibenzo[b,d]furanyl, such as dibenzo[b,d]furan-1-yl, dibenzo[b,d]furan-2-yl, dibenzo[b,d]furan-3-yl or dibenzo[b,d]furan-4-yl, naphtho[1,2-b]furanyl, such as naphtho[1,2-b]furan-5-yl, naphtho[2,3-b]furanyl, such as naphtho[2,3-b]furan-3-yl, naphtho[2,3-b]furan-4-yl or naphtho[2,3-b]furan-9-yl, naphtho[2,1-b]furanyl, such as naphtho[2,1-b]furan-2-yl or naphtho[2,1-b]furan-5-yl, benzo[b]naphtho[1,2-d]furanyl, such as benzo[b]naphtho[1,2-d]furan-1-yl or benzo[b]naphtho[1,2-d]furan-4-yl, benzo[b]naphtho[2,3-d]furanyl, such as benzo[b]naphtho[2,3-d]furan-2-yl, benzo[b]naphtho[2,3-d]furan-4-yl or benzo[b]naphtho[2,3-d]furan-6-yl, benzo[b]naphtho[2,1-d]furanyl, such as benzo[b]naphtho[2,1-d]furan-6-yl or benzo[b]naphtho[2,1-d]furan-7-yl, benzo[1,2-b:4,3-b′]difuranyl, such as benzo[1,2-b:4,3-b′]difuran-7-yl, benzo[1,2-b:6,5-b′]difuranyl, such as benzo[1,2-b:6,5-b′]difuran-4-yl, benzo[1,2-b:5,4-b′]difuranyl, such as benzo[1,2-b:5,4-b′]difuran-4-yl or benzo[1,2-b:5,4-b′]difuran-8-yl, benzo[1,2-b:4,5-b′]difuranyl, such as benzo[1,2-b:4,5-b′]difuran-4-yl, tribenzo[b,d,f]oxepinyl, such as tribenzo[b,d,f]oxepin-6-yl or tribenzo[b,d,f]oxepin-8-yl, 2H-naphtho[1,8-d,e][1,3]dioxinyl, such as 2H-naphtho[1,8-d,e][1,3]dioxin-2-yl or 2H-naphtho[1,8-d,e][1,3]dioxin-6-yl, dinaphtho[2,3-b:2′,3′-d]furanyl, such as dinaphtho[2,3-b:2′,3′-d]furan-3-yl or dinaphtho[2,3-b:2′,3′-d]furan-5-yl, oxanthrenyl, such as oxanthren-1-yl or oxanthren-2-yl, benzo[a]oxanthrenyl, such as benzo[a]oxanthren-1-yl, benzo[a]oxanthren-2yl, benzo[a]oxanthren-6-yl or benzo[a]oxanthren-7-yl, benzo[b]oxanthrenyl, such as benzo[b]oxanthren-1-yl, benzo[b]oxanthren-2-yl or benzo[b]oxanthren-6-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, dibenzo[b,d]thienyl, such as dibenzo[b,d]thien-1-yl, dibenzo[b,d]thien-2-yl, dibenzo[b,d]thien-3-yl or dibenzo[b,d]thien-4-yl, naphtho[1,2-b]thienyl, such as naphtho[1,2-b]thien-5-yl, naphtho[2,3-b]thienyl, such as naphtho[2,3-b]thien-3-yl, naphtho[2,3-b]thien-4-yl or naphtho[2,3-b]thien-9-yl, naphtho[2,1-b]thienyl, such as naphtho[2,1-b]thien-2-yl or naphtho[2,1-b]thien-5-yl, benzo[b]naphtho[1,2-d]thienyl, such as benzo[b]naphtho[1,2-d]thien-1-yl or benzo[b]naphtho[1,2-d]thien-4-yl, benzo[b]naphtho[2,3-d]thienyl, such as benzo[b]naphtho[2,3-d]thien-2-yl, benzo[b]naphtho[2,3-d]thien-4-yl or benzo[b]naphtho[2,3-d]thien-6-yl, benzo[b]naphtho[2,1-d]thienyl, such as benzo[b]naphtho[2,1-d]thien-7-yl, benzo[1,2-b:4,3-b′]dithienyl, such as benzo[1,2-b:4,3-b′]dithien-7-yl, benzo[1,2-b:6,5-b′]dithienyl, such as benzo[1,2-b:6,5-b′]dithien-4-yl, benzo[1,2-b:5,4-b′]dithienyl, such as benzo[1,2-b:5,4-b′]dithien-4-yl or benzo[1,2-b:5,4-b′]dithien-8-yl, benzo[1,2-b:4,5-b′]dithienyl, such as benzo[1,2-b:4,5-b′]dithien-4-yl, 9H-thioxanthenyl, such as 9H-thioxanthen-4-yl, 6H-dibenzo[b,d]thiopyranyl, such as 6H-dibenzo[b,d]thiopyran-2-yl or 6H-dibenzo[b,d]thiopyran-4-yl, 1,4-benzodithiinyl, such as 1,4-benzodithiin-2-yl, 1,4-benzodithiin-5-yl or 1,4-benzodithiin-6-yl, naphtho[1,2-b][1,4]dithiinyl, such as naphtho[1,2-b][1,4]dithiin-2-yl or naphtho[1,2-b][1,4]dithiin-6-yl, naphtho[2,3-b][1,4]dithiinyl, such as naphtho[2,3-b][1,4]dithiin-5-yl, thianthrenyl, such as thianthren-1-yl or thianthren-2-yl, benzo[a]thianthrenyl, such as benzo[a]thianthren-1-yl, benzo[a]thianthren-2-yl, benzo[a]thianthren-6-yl or benzo[a]thianthren-7-yl, benzo[b]thianthrenyl, such as benzo[b]thianthren-1-yl, benzo[b]thianthren-2-yl or benzo[b]thianthren-6-yl, dibenzo[a,c]thianthrenyl, such as dibenzo[a,c]thianthren-10-yl or dibenzo[a,c]thianthren-11-yl, dibenzo[a,h]thianthrenyl, such as dibenzo[a,h]thianthren-6-yl, dibenzo[a,i]thianthrenyl, such as dibenzo[a,i]thianthren-6-yl, dibenzo[a,j]thianthrenyl, such as dibenzo[a,j]thianthren-6-yl, dibenzo[b,i]thianthrenyl, such as dibenzo[b,i]thianthren-5-yl, 2H-naphtho[1,8-b,c]thienyl, such as 2H-naphtho[1,8-b,c]thien-6-yl or 2H-naphtho[1,8-b,c]thien-8-yl, dibenzo[b,d]thiepinyl, such as dibenzo[b,d]thiepin-2-yl, dibenzo[b,f]thiepinyl, such as dibenzo[b,f]thiepin-2-yl or dibenzo[b,f]thiepin-4-yl, 5H-phenanthro[4,5-b,c,d]thiopyranyl, such as 5H-phenanthro[4,5-b,c,d]thiopyran-1-yl, 5H-phenanthro[4,5-b,c,d]thiopyran-2-yl, 5H-phenanthro[4,5-b,c,d]thiopyran-3-yl, 5H-phenanthro[4,5-b,c,d]thiopyran-7-yl, 5H-phenanthro[4,5-b,c,d]thiopyran-9-yl or 5H-phenanthro[4,5-b,c,d]thiopyran-10-yl, tribenzo[b,d,f]thiepinyl, such as tribenzo[b,d,f]thiepin-6-yl or tribenzo[b,d,f]thiepin-8-yl, 2,5-dihydronaphtho[1,8-b,c:4,5-b′c′]dithienyl, such as 2,5-dihydronaphtho[1,8-b,c:4,5-b′c′]dithien-3-yl or 2,5-dihydronaphtho[1,8-b,c:4,5-b′c′]dithien-7-yl, 2,6-dihydronaphtho[1,8-b,c:5,4-b′c′]dithienyl, such as 2,6-dihydronaphtho[1,8-b,c:5,4-b′c′]dithien-4-yl, tetrabenzo[a,c,h,j]thianthrenyl, such as tetrabenzo[a,c,h,j]thianthren-3-yl, benzo[b]naphtho[1,8-e,f][1,4]dithiepinyl, such as benzo[b]naphtho[1,8-e,f][1,4]dithiepin-2-yl, dinaphtho[2,3-b:2′,3′-d]thienyl, such as dinaphtho[2,3-b:2′,3′-d]thien-3-yl or dinaphtho[2,3-b:2′,3′-d]thien-5-yl, 5H-phenanthro[1,10-b,c]thienyl, such as 5H-phenanthro[1,10-b,c]thien-1-yl or 5H-phenanthro[1,10-b,c]thien-3-yl, 7H-phenanthro[1,10-c,b]thienyl, such as 7H-phenanthro[1,10-c,b]thien-1-yl or 7H-phenanthro[1,10-c,b]thien-9-yl, dibenzo[d,d′]benzo[1,2-b:4,5-b′]dithienyl, such as dibenzo[d,d′]benzo[1,2-b:4,5-b′]dithien-4-yl or dibenzo[d,d′]benzo[1,2-b:4,5-b′]dithien-6-yl, and dibenzo[d,d′]benzo[1,2-b:5,4-b′]dithienyl, such as dibenzo[d,d′]benzo[1,2-b:5,4-b′]dithien-4-yl or dibenzo[d,d′]benzo[1,2-b:5,4-b′]dithien-6-yl, which may be unsubstituted or may carry 1 or 2 radicals R^{Ar}.

According to a particularly preferred group (6.2) of embodiments, R¹ and R² are independently selected from phenyl, naphthyl, such as naphth-1-yl or naphth-2-yl, 1,2-dihydroacenaphthylenyl, such as 1,2-dihydroacenaphthylen-3-yl or 1,2-dihydroacenaphthylen-5-yl, biphenylyl, such as biphenyl-4-yl, biphenyl-3-yl or biphenyl-2-yl, fluorenyl, such as fluoren-1-yl, fluoren-2-yl, fluoren-3-yl or fluoren-4-yl, phenanthrenyl, such as phenanthren-1-yl, phenanthren-2-yl, phenanthren-3-yl, phenanthren-4-yl or phenanthren-9-yl, benzo[c]phenanthrenyl, such as benzo[c]phenanthren-1-yl, benzo[c]phenanthren-2-yl, benzo[c]phenanthren-3-yl, benzo[c]phenanthren-4-yl, benzo[c]phenanthren-5-yl or benzo[c]phenanthren-6-yl, pyrenyl, such as pyren-1-yl, pyren-2-yl or pyren-4-yl, chrysenyl, such as chrysen-1-yl, chrysen-2-yl, chrysen-3-yl, chrysen-4-yl, chrysen-5-yl or chrysen-6-yl, triphenylenyl, such as triphenylen-1-yl or triphenylen-2-yl, benzo[b]furanyl, such as benzo[b]furan-2-yl, benzo[b]furan-3-yl, benzo[b]furan-4-yl, benzo[b]furan-5-yl, benzo[b]furan-6-yl or benzo[b]furan-7-yl, dibenzo[b,d]furanyl, such as dibenzo[b,d]furan-1-yl, dibenzo[b,d]furan-2-yl, dibenzo[b,d]furan-3-yl or dibenzo[b,d]furan-4-yl, naphtho[1,2-b]furanyl, such as naphtho[1,2-b]furan-5-yl, naphtho[2,3-b]furanyl, such as naphtho[2,3-b]furan-3-yl, naphtho[2,3-b]furan-4-yl or naphtho[2,3-b]furan-9-yl, naphtho[2,1-b]furanyl, such as naphtho[2,1-b]furan-2-yl or naphtho[2,1-b]furan-5-yl, benzo[b]naphtho[1,2-d]furanyl, such as benzo[b]naphtho[1,2-d]furan-1-yl or benzo[b]naphtho[1,2-d]furan-4-yl, benzo[b]naphtho[2,3-d]furanyl, such as benzo[b]naphtho[2,3-d]furan-2-yl, benzo[b]naphtho[2,3-d]furan-4-yl or benzo[b]naphtho[2,3-d]furan-6-yl, benzo[b]naphtho[2,1-d]furanyl, such as benzo[b]naphtho[2,1-d]furan-6-yl or benzo[b]naphtho[2,1-d]furan-7-yl, oxanthrenyl, such as oxanthren-1-yl or oxanthren-2-yl, benzo[a]oxanthrenyl, such as benzo[a]oxanthren-1-yl, benzo[a]oxanthren-2-yl, benzo[a]oxanthren-6-yl or benzo[a]oxanthren-7-yl, benzo[b]oxanthrenyl, such as benzo[b]oxanthren-1-yl, benzo[b]oxanthren-2-yl or benzo[b]oxanthren-6-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, dibenzo[b,d]thienyl, such as dibenzo[b,d]thien-1-yl, dibenzo[b,d]thien-2-yl, dibenzo[b,d]thien-3-yl or dibenzo[b,d]thien-4-yl, naphtho[1,2-b]thienyl, such as naphtho[1,2-b]thien-5-yl, naphtho[2,3-b]thienyl, such as naphtho[2,3-b]thien-3-yl, naphtho[2,3-b]thien-4-yl or naphtho[2,3-b]thien-9-yl, naphtho[2,1-b]thienyl, such as naphtho[2,1-b]thien-2-yl or naphtho[2,1-b]thien-5-yl, benzo[b]naphtho[1,2-d]thienyl, such as benzo[b]naphtho[1,2-d]thien-1-yl or benzo[b]naphtho[1,2-d]thien-4-yl, benzo[b]naphtho[2,3-d]thienyl, such as benzo[b]naphtho[2,3-d]thien-2-yl, benzo[b]naphtho[2,3-d]thien-4-yl or benzo[b]naphtho[2,3-d]thien-6-yl, benzo[b]naphtho[2,1-d]thienyl, such as benzo[b]naphtho[2,1-d]thien-7-yl, thianthrenyl, such as thianthren-1-yl or thianthren-2-yl, benzo[a]thianthrenyl, such as benzo[a]thianthren-1-yl, benzo[a]thianthren-2-yl, benzo[a]thianthren-6-yl or benzo[a]thianthren-7-yl, benzo[b]thianthrenyl, such as benzo[b]thianthren-1-yl, benzo[b]thianthren-2-yl or benzo[b]thianthren-6-yl, 2H-naphtho[1,8-b,c]thienyl, such as 2H-naphtho[1,8-b,c]thien-6-yl or 2H-naphtho[1,8-b,c]thien-8-yl, dibenzo[b,d]thiepinyl, such as dibenzo[b,d]thiepin-2-yl, dibenzo[b,f]thiepinyl, such as dibenzo[b,f]thiepin-2-yl or dibenzo[b,f]thiepin-4-yl, and tribenzo[b,d,f]thiepinyl, such as tribenzo[b,d,f]thiepin-6-yl or tribenzo[b,d,f]thiepin-8-yl, which may be unsubstituted or may carry 1 radical R^{Ar}.

In an especially preferred group (6.3) of embodiments, R¹ and R² are independently selected from phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl, and thianthrenyl, and especially selected from phenyl, naphth-1-yl, naphth-2-yl, 1,2-dihydroacenaphthylen-5-yl, phenanthren-9-yl, pyren-1-yl, pyren-2-yl, pyren-4-yl, triphenylen-1-yl, triphenylen-2-yl, dibenzo[b,d]furan-2-yl, dibenzo[b,d]furan-4-yl, benzo[b]thien-3-yl, benzo[b]thien-4-yl, benzo[b]thien-5-yl, benzo[b]thien-6-yl, benzo[b]thien-7-yl, dibenzo[b,d]thien-2-yl, dibenzo[b,d]thien-4-yl, thianthren-1-yl and thianthren-2-yl. In a subgroup (6.3a) of group (6.3) of embodiments, R¹ and R² are independently selected from phenyl, naphthyl, 1,2-dihydroacenaphthylenyl, phenanthrenyl, triphenylenyl, pyrenyl, dibenzo[b,d]furanyl, benzo[b]thienyl, dibenzo[b,d]thienyl and thianthrenyl, and specifically selected from phenyl, naphthyl, phenanthrenyl, dibenzo[b,d]thienyl and thianthrenyl.

In a particular subgroup group (6′) of embodiments, the variables R¹ and R² have the same meaning which is selected from the meanings defined herein for R¹ and R², especially those mentioned as preferred, and in particular selected from the meanings defined in groups (6), (6.1), (6.2), (6.3) or (6.3a) of embodiments.

In a preferred group (7) of embodiments, the variables R³ and R⁴ are different from hydrogen. In other words, the variables R³ and R⁴ are selected from the group consisting of mono- or polycyclic aryl having from 6 to 26 carbon atoms as ring member atoms and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms, where mono- or polycyclic aryl and mono- or polycyclic hetaryl are unsubstituted or carry 1, 2, 3 or 4 radicals R^{Ar}. More preferably, at least one of R³ and R⁴, in particular both R³ and R⁴ are selected from polycyclic aryl having from 10 to 18 carbon atoms and polycyclic hetaryl having a total of 9 to 26 atoms.

Preferably, in this group (7) of embodiments, the variables R³ and R⁴ are independently selected from the group consisting of mono- or polycyclic aryl having from 6 to 18 carbon atoms as ring members and polycyclic hetaryl having a total of 9 to 26 atoms, which are ring members, where 1 or 2 of these atoms are oxygen or sulfur atoms, while the remainder of these atoms are carbon atoms, where mono- or polycyclic aryl and polycyclic hetaryl are unsubstituted or carry 1 or 2 radicals R^{Ar}, where R^{Ar} has one of the meanings defined herein, especially one of the meanings mentioned as preferred (hereinafter group (7.1) of embodiments).

More preferably, in this group (7) of embodiments, R³ and R⁴ are independently selected from phenyl, naphthyl, such as naphth-1-yl or naphth-2-yl, 1,2-dihydroacenaphthylenyl, such as 1,2-dihydroacenaphthylen-3-yl or 1,2-dihydroacenaphthylen-5-yl, biphenylyl, such as biphenyl-4-yl, biphenyl-3-yl or biphenyl-2-yl, fluorenyl, such as fluoren-1-yl, fluoren-2-yl, fluoren-3-yl or fluoren-4-yl, 11H-benzo[a]fluorenyl, such as 11H-benzo[a]fluoren-7-yl, 11H-benzo[b]fluorenyl, such as 11H-benzo[b]fluoren-1-yl, 7H-benzo[c]fluorenyl, such as 7H-benzo[c]fluoren-5-yl or 7H-benzo[c]fluoren-10-yl, phenanthrenyl, such as phenanthren-1-yl, phenanthren-2-yl, phenanthren-3-yl, phenanthren-4-yl or phenanthren-9-yl, benzo[c]phenanthrenyl, such as benzo[c]phenanthren-1-yl, benzo[c]phenanthren-2-yl, benzo[c]phenanthren-3-yl, benzo[c]phenanthren-4-yl, benzo[c]phenanthren-5-yl or benzo[c]phenanthren-6-yl, pyrenyl, such as pyren-1-yl, pyren-2-yl or pyren-4-yl, chrysenyl, such as chrysen-1-yl, chrysen-2-yl, chrysen-3-yl, chrysen-4-yl, chrysen-5-yl or chrysen-6-yl, picenyl, such as picen-3-yl, triphenylenyl, such as triphenylen-1-yl or triphenylen-2-yl, benzo[b]furanyl, such as benzo[b]furan-2-yl, benzo[b]furan-3-yl, benzo[b]furan-4-yl, benzo[b]furan-5-yl, benzo[b]furan-6-yl or benzo[b]furan-7-yl, dibenzo[b,d]furanyl, such as dibenzo[b,d]furan-1-yl, dibenzo[b,d]furan-2-yl, dibenzo[b,d]furan-3-yl or dibenzo[b,d]furan-4-yl, naphtho[1,2-b]furanyl, such as naphtho[1,2-b]furan-5-yl, naphtho[2,3-b]furanyl, such as naphtho[2,3-b]furan-3-yl, naphtho[2,3-b]furan-4-yl or naphtho[2,3-b]furan-9-yl, naphtho[2,1-b]furanyl, such as naphtho[2,1-b]furan-2-yl or naphtho[2,1-b]furan-5-yl, benzo[b]naphtho[1,2-a]furanyl, such as benzo[b]naphtho[1,2-a]furan-1-yl or benzo[b]naphtho[1,2-a]furan-4-yl, benzo[b]naphtho[2,3-a]furanyl, such as benzo[b]naphtho[2,3-a]furan-2-yl, benzo[b]naphtho[2,3-a]furan-4-yl or benzo[b]naphtho[2,3-a]furan-6-yl, benzo[b]naphtho[2,1-a]furanyl, such as benzo[b]naphtho[2,1-a]furan-6-yl or benzo[b]naphtho[2,1-a]furan-7-yl, benzo[1,2-b:4,3-b′]difuranyl, such as benzo[1,2-b:4,3-b′]difuran-7-yl, benzo[1,2-b:6,5-b′]difuranyl, such as benzo[1,2-b:6,5-b′]difuran-4-yl, benzo[1,2-b:5,4-b′]difuranyl, such as benzo[1,2-b:5,4-b′]difuran-4-yl or benzo[1,2-b:5,4-b′]difuran-8-yl, benzo[1,2-b:4,5-b′]difuranyl, such as benzo[1,2-b:4,5-b′]difuran-4-yl, tribenzo[b,d,f]oxepinyl, such as tribenzo[b,d,f]oxepin-6-yl or tribenzo[b,d,f]oxepin-8-yl, 2H-naphtho[1,8-d,e][1,3]dioxinyl, such as 2H-naphtho[1,8-d,e][1,3]dioxin-2-yl or 2H-naphtho[1,8-d,e][1,3]dioxin-6-yl, dinaphtho[2,3-b:2′,3′-d]furanyl, such as dinaphtho[2,3-b:2′,3′-G]furan-3-yl or dinaphtho[2,3-b:2′,3′-d]furan-5-yl, oxanthrenyl, such as oxanthren-1-yl or oxanthren-2-yl, benzo[a]oxanthrenyl, such as benzo[a]oxanthren-1-yl, benzo[a]oxanthren-2yl, benzo[a]oxanthren-6-yl or benzo[a]oxanthren-7-yl, benzo[b]oxanthrenyl, such as benzo[b]oxanthren-1-yl, benzo[b]oxanthren-2-yl or benzo[b]oxanthren-6-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, dibenzo[b,d]thienyl, such as dibenzo[b,d]thien-1-yl, dibenzo[b,d]thien-2-yl, dibenzo[b,d]thien-3-yl or dibenzo[b,d]thien-4-yl, naphtho[1,2-b]thienyl, such as naphtho[1,2-b]thien-5-yl, naphtho[2,3-b]thienyl, such as naphtho[2,3-b]thien-3-yl, naphtho[2,3-b]thien-4-yl or naphtho[2,3-b]thien-9-yl, naphtho[2,1-b]thienyl, such as naphtho[2,1-b]thien-2-yl or naphtho[2,1-b]thien-5-yl, benzo[b]naphtho[1,2-d]thienyl, such as benzo[b]naphtho[1,2-d]thien-1-yl or benzo[b]naphtho[1,2-d]thien-4-yl, benzo[b]naphtho[2,3-d]thienyl, such as benzo[b]naphtho[2,3-d]thien-2-yl, benzo[b]naphtho[2,3-d]thien-4-yl or benzo[b]naphtho[2,3-d]thien-6-yl, benzo[b]naphtho[2,1-d]thienyl, such as benzo[b]naphtho[2,1-d]thien-7-yl, benzo[1,2-b:4,3-b′]dithienyl, such as benzo[1,2-b:4,3-b′]dithien-7-yl, benzo[1,2-b:6,5-b′]dithienyl, such as benzo[1,2-b:6,5-b′]dithien-4-yl, benzo[1,2-b:5,4-b′]dithienyl, such as benzo[1,2-b:5,4-b′]dithien-4-yl or benzo[1,2-b:5,4-b′]dithien-8-yl, benzo[1,2-b:4,5-b′]dithienyl, such as benzo[1,2-b:4,5-b′]dithien-4-yl, 9H-thioxanthenyl, such as 9H-thioxanthen-4-yl, 6H-dibenzo[b,d]thiopyranyl, such as 6H-dibenzo[b,d]thiopyran-2-yl or 6H-dibenzo[b,d]thiopyran-4-yl, 1,4-benzodithiinyl, such as 1,4-benzodithiin-2-yl, 1,4-benzodithiin-5-yl or 1,4-benzodithiin-6-yl, naphtho[1,2-b][1,4]dithiinyl, such as naphtho[1,2-b][1,4]dithiin-2-yl or naphtho[1,2-b][1,4]dithiin-6-yl, naphtho[2,3-b][1,4]dithiinyl, such as naphtho[2,3-b][1,4]dithiin-5-yl, thianthrenyl, such as thianthren-1-yl or thianthren-2-yl, benzo[a]thianthrenyl, such as benzo[a]thianthren-1-yl, benzo[a]thianthren-2-yl, benzo[a]thianthren-6-yl or benzo[a]thianthren-7-yl, benzo[b]thianthrenyl, such as benzo[b]thianthren-1-yl, benzo[b]thianthren-2-yl or benzo[b]thianthren-6-yl, dibenzo[a,c]thianthrenyl, such as dibenzo[a,c]thianthren-10-yl or dibenzo[a,c]thianthren-11-yl, dibenzo[a,h]thianthrenyl, such as dibenzo[a,h]thianthren-6-yl, dibenzo[a,i]thianthrenyl, such as dibenzo[a,i]thianthren-6-yl, dibenzo[a,j]thianthrenyl, such as dibenzo[a,j]thianthren-6-yl, dibenzo[b,i]thianthrenyl, such as dibenzo[b,i]thianthren-5-yl, 2H-naphtho[1,8-b,c]thienyl, such as 2H-naphtho[1,8-b,c]thien-6-yl or 2H-naphtho[1,8-b,c]thien-8-yl, dibenzo[b,d]thiepinyl, such as dibenzo[b,d]thiepin-2-yl, dibenzo[b,f]thiepinyl, such as dibenzo[b,f]thiepin-2-yl or dibenzo[b,f]thiepin-4-yl, 5H-phenanthro[4,5-b,c,d]thiopyranyl, such as 5H-phenanthro[4,5-b,c,d]thiopyran-1-yl, 5H-phenanthro[4,5-b,c,d]thiopyran-2-yl, 5H-phenanthro[4,5-b,c,d]thiopyran-3-yl, 5H-phenanthro[4,5-b,c,d]thiopyran-7-yl, 5H-phenanthro[4,5-b,c,d]thiopyran-9-yl or 5H-phenanthro[4,5-b,c,d]thiopyran-10-yl, tribenzo[b,d,f]thiepinyl, such as tribenzo[b,d,f]thiepin-6-yl or tribenzo[b,d,f]thiepin-8-yl, 2,5-dihydronaphtho[1,8-b,c:4,5-b′c′]dithienyl, such as 2,5-dihydronaphtho[1,8-b,c:4,5-b′c′]dithien-3-yl or 2,5-dihydronaphtho[1,8-b,c:4,5-b′c′]dithien-7-yl, 2,6-dihydronaphtho[1,8-b,c:5,4-b′c′]dithienyl, such as 2,6-dihydronaphtho[1,8-b,c:5,4-b′c′]dithien-4-yl, tetrabenzo[a,c,h,j]thianthrenyl, such as tetrabenzo[a,c,h,j]thianthren-3-yl, benzo[b]naphtho[1,8-e,f][1,4]dithiepinyl, such as benzo[b]naphtho[1,8-e,f][1,4]dithiepin-2-yl, dinaphtho[2,3-b:2′,3′-d]thienyl, such as dinaphtho[2,3-b:2′,3′-d]thien-3-yl or dinaphtho[2,3-b:2′,3′-d]thien-5-yl, 5H-phenanthro[1,10-b,c]thienyl, such as 5H-phenanthro[1,10-b,c]thien-1-yl or 5H-phenanthro[1,10-b,c]thien-3-yl, 7H-phenanthro[1,10-c,b]thienyl, such as 7H-phenanthro[1,10-c,b]thien-1-yl or 7H-phenanthro[1,10-c,b]thien-9-yl, dibenzo[d,d′]benzo[1,2-b:4,5-b′]dithienyl, such as dibenzo[d,d′]benzo[1,2-b:4,5-b′]dithien-4-yl or dibenzo[d,d′]benzo[1,2-b:4,5-b′]dithien-6-yl, and dibenzo[d,d′]benzo[1,2-b:5,4-b′]dithienyl, such as dibenzo[d,d′]benzo[1,2-b:5,4-b′]dithien-4-yl or dibenzo[d,d′]benzo[1,2-b:5,4-b′]dithien-6-yl, where the aforementioned mono- or polycyclic aryl and polycyclic hetaryl are unsubstituted or carry 1 or 2 radicals R^{Ar} (hereinafter group (7.2) of embodiments).

In particular, in this group (7) of embodiments, R³ and R⁴ are independently selected from phenyl, naphthyl, such as naphth-1-yl or naphth-2-yl, 1,2-dihydroacenaphthylenyl, such as 1,2-dihydroacenaphthylen-3-yl or 1,2-dihydroacenaphthylen-5-yl, biphenylyl, such as biphenyl-4-yl, biphenyl-3-yl or biphenyl-2-yl, fluorenyl, such as fluoren-1-yl, fluoren-2-yl, fluoren-3-yl or fluoren-4-yl, phenanthrenyl, such as phenanthren-1-yl, phenanthren-2-yl, phenanthren-3-yl, phenanthren-4-yl or phenanthren-9-yl, benzo[c]phenanthrenyl, such as benzo[c]phenanthren-1-yl, benzo[c]phenanthren-2-yl, benzo[c]phenanthren-3-yl, benzo[c]phenanthren-4-yl, benzo[c]phenanthren-5-yl or benzo[c]phenanthren-6-yl, pyrenyl, such as pyren-1-yl, pyren-2-yl or pyren-4-yl, chrysenyl, such as chrysen-1-yl, chrysen-2-yl, chrysen-3-yl, chrysen-4-yl, chrysen-5-yl or chrysen-6-yl, triphenylenyl, such as triphenylen-1-yl or triphenylen-2-yl, benzo[b]furanyl, such as benzo[b]furan-2-yl, benzo[b]furan-3-yl, benzo[b]furan-4-yl, benzo[b]furan-5-yl, benzo[b]furan-6-yl or benzo[b]furan-7-yl, dibenzo[b,d]furanyl, such as dibenzo[b,d]furan-1-yl, dibenzo[b,d]furan-2-yl, dibenzo[b,d]furan-3-yl or dibenzo[b,d]furan-4-yl, naphtho[1,2-b]furanyl, such as naphtho[1,2-b]furan-5-yl, naphtho[2,3-b]furanyl, such as naphtho[2,3-b]furan-3-yl, naphtho[2,3-b]furan-4-yl or naphtho[2,3-b]furan-9-yl, naphtho[2,1-b]furanyl, such as naphtho[2,1-b]furan-2-yl or naphtho[2,1-b]furan-5-yl, benzo[b]naphtho[1,2-d]furanyl, such as benzo[b]naphtho[1,2-d]furan-1-yl or benzo[b]naphtho[1,2-d]furan-4-yl, benzo[b]naphtho[2,3-d]furanyl, such as benzo[b]naphtho[2,3-d]furan-2-yl, benzo[b]naphtho[2,3-d]furan-4-yl or benzo[b]naphtho[2,3-d]furan-6-yl, benzo[b]naphtho[2,1-d]furanyl, such as benzo[b]naphtho[2,1-d]furan-6-yl or benzo[b]naphtho[2,1-d]furan-7-yl, oxanthrenyl, such as oxanthren-1-yl or oxanthren-2-yl, benzo[a]oxanthrenyl, such as benzo[a]oxanthren-1-yl, benzo[a]oxanthren-2-yl, benzo[a]oxanthren-6-yl or benzo[a]oxanthren-7-yl, benzo[b]oxanthrenyl, such as benzo[b]oxanthren-1-yl, benzo[b]oxanthren-2-yl or benzo[b]oxanthren-6-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, dibenzo[b,d]thienyl, such as dibenzo[b,d]thien-1-yl, dibenzo[b,d]thien-2-yl, dibenzo[b,d]thien-3-yl or dibenzo[b,d]thien-4-yl, naphtho[1,2-b]thienyl, such as naphtho[1,2-b]thien-5-yl, naphtho[2,3-b]thienyl, such as naphtho[2,3-b]thien-3-yl, naphtho[2,3-b]thien-4-yl or naphtho[2,3-b]thien-9-yl, naphtho[2,1-b]thienyl, such as naphtho[2,1-b]thien-2-yl or naphtho[2,1-b]thien-5-yl, benzo[b]naphtho[1,2-d]thienyl, such as benzo[b]naphtho[1,2-d]thien-1-yl or benzo[b]naphtho[1,2-d]thien-4-yl, benzo[b]naphtho[2,3-d]thienyl, such as benzo[b]naphtho[2,3-d]thien-2-yl, benzo[b]naphtho[2,3-d]thien-4-yl or benzo[b]naphtho[2,3-d]thien-6-yl, benzo[b]naphtho[2,1-d]thienyl, such as benzo[b]naphtho[2,1-d]thien-7-yl, thianthrenyl, such as thianthren-1-yl or thianthren-2-yl, benzo[a]thianthrenyl, such as benzo[a]thianthren-1-yl, benzo[a]thianthren-2-yl, benzo[a]thianthren-6-yl or benzo[a]thianthren-7-yl, benzo[b]thianthrenyl, such as benzo[b]thianthren-1-yl, benzo[b]thianthren-2-yl or benzo[b]thianthren-6-yl, 2H-naphtho[1,8-b,c]thienyl, such as 2H-naphtho[1,8-b,c]thien-6-yl or 2H-naphtho[1,8-b,c]thien-8-yl, dibenzo[b,d]thiepinyl, such as dibenzo[b,d]thiepin-2-yl, dibenzo[b,f]thiepinyl, such as dibenzo[b,f]thiepin-2-yl or dibenzo[b,f]thiepin-4-yl, and tribenzo[b,d,f]thiepinyl, such as tribenzo[b,d,f]thiepin-6-yl or tribenzo[b,d,f]thiepin-8-yl, where the aforementioned mono- or polycyclic aryl and polycyclic hetaryl are unsubstituted or carry 1 radical R^{Ar} (hereinafter group (7.3) of embodiments).

In an especially preferred group (7.4) of embodiments, R³ and R⁴ are independently selected from phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl, and thianthrenyl, and especially selected from phenyl, naphth-1-yl, naphth-2-yl, 1,2-dihydroacenaphthylen-5-yl, phenanthren-9-yl, pyren-1-yl, pyren-2-yl, pyren-4-yl, triphenylen-1-yl, triphenylen-2-yl, dibenzo[b,d]furan-2-yl, dibenzo[b,d]furan-4-yl, benzo[b]thien-3-yl, benzo[b]thien-4-yl, benzo[b]thien-5-yl, benzo[b]thien-6-yl, benzo[b]thien-7-yl, dibenzo[b,d]thien-2-yl, dibenzo[b,d]thien-4-yl, thianthren-1-yl and thianthren-2-yl. In a subgroup (7.4a) of group (7.4) of embodiments, R¹ and R² are independently selected from naphthyl, 1,2-dihydroacenaphthylenyl, phenanthrenyl, benzo[b]furanyl, dibenzo[b,d]furanyl, benzo[b]thienyl and dibenzo[b,d]thienyl.

It was found that a particular subgroup (7.5) of group (7) of embodiments provides for a high refractive index and a negative birefringence. These subgroup (7.5) of embodiments relates to compounds of the formula (I), in particular to compounds of the formula (Ia-1), where R³ and R⁴ are different from hydrogen and where at least two and preferably four of the substituents R¹, R², R³ and/or R⁴ are bulky or sterically hindered substituents selected from polycyclic aryl and polycyclic hetaryl as defined herein. In this context bulky substituents R¹, R², R³ and/or R⁴ are in particular substituents from the following groups:
polycyclic aryl and polycyclic hetaryl which bear at least a phenyl ring bound to the phenyl ring in formulae (I) and (Ia-1), where the phenyl ring of R¹, R², R³ and/or R⁴ is fused to at least one of aromatic rings having 6 to 14 carbon atoms as ring member atoms and saturated or partly or fully unsatuarted heterocyclic rings having 5 to 13 ring member atoms, where at least one of the rings fused to said phenyl ring is fused to the bond between the ortho- and the meta-positions of the phenyl ring, and
polycyclic aryl having 14 to 26, in particular 14 to 20 carbon atoms as ring member atoms and from polycyclic hetaryl having 13 to 26, in particular 13 to 20, atoms which are ring members, where 1, 2, 3 or 4 of these ring member atoms are selected from nitrogen, sulfur and oxygen, while the remainder of these ring member atoms are carbon atoms.

Examples of said bulky substiutents include but are not limited to naphthyl, phenanthryl, pyrenyl, triphenylenyl, 1,2-dihydroacenaphthylenyl, dibenzo[b,d]thienyl, thianthrenyl, dibenzo[b,d]furanyl and 9H-fluorene-3-yl, and especially include but are not limited to 1-naphthyl, 9-phenanthryl, pyren-1-yl, pyren-4-yl, 1-triphenylenyl, 1,2-dihydroacenaphthylenyl, dibenzo[b,d]thien-4-yl, dibenzo[b,d]furan-4-yl and thianthren-1-yl.

While the reason for this beneficial effect of bulky substituents R¹, R², R³ and/or R⁴ is not completely clear, it is likely that due to their steric hindrance, these substituents are forced to an orientation within the resin that is perpendicular to the main chain, which reduces the birefringence of the resin.

Accordingly, thermoplastic resins having a low birefringence can be obtained according to the present invention by balancing the positive birefringence imparted to the resin by co-monomers, such as those of formula (IV), with the negative birefringence imparted by monomers of formula (I), in particular of formula (Ia-1) according to the embodiment (7.5).

In a particular subgroup group (7′) of embodiments, the variables R³ and R⁴ have the same meaning which is selected from the meanings defined herein for R³ and R⁴, especially those mentioned as preferred, and in particular selected from the meanings defined in groups (7), (7.1), (7.2), (7.3), (7.4), (7.4a) or (7.5) of embodiments.

In a particular group (8) of embodiments, the variables R¹, R², R³ and R⁴ have the same meaning. In this group (8) of embodiments the identical meaning of variables R¹, R², R³ and R⁴ is preferably selected from the meanings defined herein, especially those mentioned as preferred, and is preferably selected from the meanings defined in groups (6), in particular as defined in group (6.1) of embodiments, more particular as defined in group (6.2) of embodiments, even more preferably as defined in group (6.3) of embodiments and especially as defined in group (6.3a) of embodiments. In this particular group (8) of embodiments, the variables R¹, R², R³ and R⁴ are more preferably as defined in groups (7.1), (7.2), (7.3), (7.4), (7.4a) or (7.5) of embodiments.

In a further particular group (9) of embodiments, the variables R³ and R⁴ are both hydrogen. Additionally, in this group (9) of embodiments the variables R¹ and R² preferably have the same meaning which is selected from the meanings defined herein, especially those mentioned herein as preferred, and preferably selected from the meanings defined in group (6), in particular as defined in group (6.1) of embodiments, more particular as defined in group (6.2) of embodiments, even more preferably as defined in group (6.3) of embodiments and especially as defined in group (6.3a) of embodiments.

In a preferred group (10) of embodiments the substituents R¹, R², R³ and R⁴ of the formulae (I) are all located in meta positions relative to the moiety X, i.e. according to this group of embodiments the compound of the formula (I) is a compound of the formula (Ia),

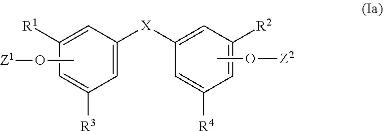

where the variables X, Z¹, Z², R¹, R², R³ and R⁴ have the meanings defined herein, and in particular the meanings mentioned as preferred and where R³ and R⁴ are preferably different from hydrogen and were in particular R¹, R², R³ and R⁴ have the same meaning.

Likewise, according to this preferred group (10) of embodiments the structural unit of formula (II) is a structural unit of formula (IIa),

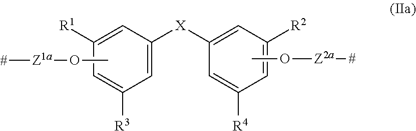

where the variables X, Z¹, Z², R¹, R², R³ and R⁴ have the meanings defined herein, and in particular the meanings mentioned as preferred and where R³ and R⁴ are preferably different from hydrogen and were in particular R¹, R², R³ and R⁴ have the same meaning.

A skilled person will readily appreciate that in the formulae (I), (Ia), (II) and (IIa) the meanings of Z¹ and Z² given in one or more of groups (1), (1.1) and (1′) of embodiments may be combined with the meanings of X according to group (5) or one of groups (5′), (5″) and (5′″) of embodiments, with the meanings of R¹ and R² according to one or more of groups (6), (6.1), (6.2), (6.3), (6.3a) and (6′) of embodiments and also with the meanings of R³ and R⁴ according to one or more of groups (7), (7.1), (7.2), (7.3), (7.4), (7.4a), (7.5) and (7′) of embodiments and also with either group (8) or group (9) of embodiments. A skilled person will also appreciate that in the formulae (I), (Ia), (II) and (IIa) the meanings of Z¹ and Z² given in group (2) of embodiments may be combined with the meanings of X according to group (5) or one of the groups (5′), (5″) or (5′″) of embodiments, with the meanings of R¹ and R² according to one or more of groups (6), (6.1), (6.2), (6.3), (6.3a) and (6′) of embodiments and also with the meanings of R³ and R⁴ according to one or more of groups (7), (7.1), (7.2), (7.3), (7.4), (7.4a), (7.5) and (7′) of embodiments and also with either group (8) or group (9) of embodiments. A skilled person will also appreciate that in the formulae (I), (Ia), (II) and (IIa) the meanings of Z¹ and Z² given in one or more of groups (3), (3.1) and (3′) of embodiments may be combined with the meanings of X according to group (5) or one of groups (5′), (5″) and (5′″) of embodiments, with the meanings of R¹ and R² according to one or more of groups (6), (6.1), (6.2), (6.3), (6.3a) and (6′) of embodiments and also with the meanings of R³ and R⁴ according to one or more of groups (7), (7.1), (7.2), (7.3), (7.4), (7.4a), (7.5) and (7′) of embodiments and also with either group (8) or group (9) of embodiments. A skilled person will also appreciate that in the formulae (I), (Ia), (II) and (IIa) the meanings of Z¹ and Z² given in one of groups (4) and (4.1) of embodiments may be combined with the meanings of X according to group (5) or one of groups (5′), (5″) or (5′″) of embodiments, with the meanings of R¹ and R² according to one or more of groups (6), (6.1), (6.2), (6.3), (6.3a) and (6′) of embodiments and also with the meanings of R³ and R⁴ according to one or more of groups (7), (7.1), (7.2), (7.3), (7.4), (7.4a), (7.5) and (7′) of embodiments and also with either group (8) or group (9) of embodiments.

Apart from that and if not stated otherwise, the variables Ar¹, R⁵, R⁶, R^{Ar}, R, R′, R″ and n either alone or preferably in combination with each other and with the meanings and preferred meanings of the variables X, R¹, R², R³, R⁴, Z¹ and Z² described above, have the following meanings.

Ar¹ is preferably a mono- or polycyclic aryl having from 6 to 18 carbon atoms as ring member atoms and polycyclic hetaryl having a total of 9 to 16 atoms, which are ring member atoms, where 1 or 2 of these ring member atoms of hetaryl are sulfur atoms, while the remainder of these ring member atoms of hetaryl are carbon atoms, where mono- or polycyclic aryl and polycyclic hetaryl are unsubstituted or carry 1 or 2 radicals R^{Ar}, where R^{Ar} has one of the meanings defined herein, especially one of the meanings mentioned as preferred. Preference is given here to unsubstituted radicals Ar¹.

More preferably, Ar¹ is selected from phenyl, naphthyl, such as naphth-1-yl or naphth-2-yl, fluorenyl, such as fluoren-1-yl, fluoren-2-yl, fluoren-3-yl or fluoren-4-yl, 11H-benzo[a]fluorenyl, such as 11H-benzo[a]fluoren-7-yl, 11H-benzo[b]fluorenyl, such as 11H-benzo[b]fluoren-1-yl, 7H-benzo[c]fluorenyl, such as 7H-benzo[c]fluoren-5-yl or 7H-benzo[c]fluoren-10-yl, phenanthrenyl, such as phenanthren-1-yl, phenanthren-2-yl, phenanthren-3-yl, phenanthren-4-yl or phenanthren-9-yl, benzo[c]phenanthrenyl, such as benzo[c]phenanthren-1-yl, benzo[c]phenanthren-2-yl, benzo[c]phenanthren-3-yl, benzo[c]phenanthren-4-yl, benzo[c]phenanthren-5-yl or benzo[c]phenanthren-6-yl, pyrenyl, such as pyren-1-yl, pyren-2-yl or pyren-4-yl, chrysenyl, such as chrysen-1-yl, chrysen-2-yl, chrysen-3-yl, chrysen-4-yl, chrysen-5-yl or chrysen-6-yl, triphenylenyl, such as triphenylen-1-yl or triphenylen-2-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, dibenzo[b,d]thienyl, such as dibenzo[b,d]thien-1-yl, dibenzo[b,d]thien-2-yl, dibenzo[b,d]thien-3-yl or dibenzo[b,d]thien-4-yl, naphtho[1,2-b]thienyl, such as naphtho[1,2-b]thien-5-yl, naphtho[2,3-b]thienyl, such as naphtho[2,3-b]thien-3-yl, naphtho[2,3-b]thien-4-yl or naphtho[2,3-b]thien-9-yl, naphtho[2,1-b]thienyl, such as naphtho[2,1-b]thien-2-yl or naphtho[2,1-b]thien-5-yl, benzo[1,2-b:4,3-b′]dithienyl, such as benzo[1,2-b:4,3-b′]dithien-7-yl, benzo[1,2-b:6,5-b′]dithienyl, such as benzo[1,2-b:6,5-b′]dithien-4-yl, benzo[1,2-b:5,4-b′]dithienyl, such as benzo[1,2-b:5,4-b′]dithien-4-yl or benzo[1,2-b:5,4-b′]dithien-8-yl, benzo[1,2-b:4,5-b′]dithienyl, such as benzo[1,2-b:4,5-b′]dithien-4-yl, and thianthrenyl, such as thianthren-1-yl or thianthren-2-yl.

Even more preferably, Ar¹ is selected from phenyl, naphthyl, fluorenyl, phenanthrenyl, pyrenyl, chrysenyl, triphenylenyl, benzo[b]furanyl, dibenzo[b,d]furanyl, benzo[b]thienyl, dibenzo[b,d]thienyl and thianthrenyl, especially from phenyl, naphthyl, phenanthrenyl, chrysenyl and dibenzo[b,d]thienyl, in particular from phenyl, naphthyl and phenanthrenyl, and specifically from phenyl, naphth-1-yl, naphth-2-yl and phenanthren-9-yl.

R⁵ is preferably selected from the group consisting of hydrogen, methyl, ethyl and a radical Ar¹, where Ar¹ has one of the meanings defined herein, especially a preferred one. More preferably, R⁵ is hydrogen, methyl or ethyl, and in particular is hydrogen or methyl.

R⁶ is preferably selected from the group consisting of hydrogen, methyl and ethyl, and in particular is hydrogen or methyl.

R^{Ar} is preferably selected from the group consisting of R, OR and CHₙR₃₋ₙ, and more preferably from the group of R and OR, where n is 0, 1 or 2, especially 1 or 2, and the variable R has one of the meanings defined herein, especially a preferred one. In particular, the radical R^{Ar} is selected from the group consisting of methyl, methoxy, phenyl, naphthyl, phenanthrenyl and triphenylenyl, and specifically is phenyl, naphthyl or phenanthrenyl.

R is preferably selected from the group consisting of methyl, phenyl, naphthyl, phenanthrenyl and triphenylenyl, which are unsubstituted or substituted by 1, 2 or 3 identical or different radicals R″, where R″, independently of each occurrence, has one of the meanings defined herein, in particular a preferred one. More preferably, R is selected from the group consisting of phenyl, naphthyl and phenanthrenyl, which are unsubstituted.

R′ is preferably selected from the group consisting of hydrogen, methyl, phenyl and naphthyl, where phenyl and naphthyl are unsubstituted or substituted by 1, 2 or 3 identical or different radicals R″, where R″, independently of each occurrence, has one of the meanings defined herein, in particular a preferred one. More preferably, R′ is unsubstituted phenyl or unsubstituted naphthyl.

R″ is preferably selected from the group consisting of phenyl, OCH₃ and CH₃.

The variable n is preferably 1 or 2.

In a particular subgroup (10.1) of group (10) of embodiments, where in formula (Ia) the groups Z¹ and Z² are both Z which has one of the meanings defined herein for Z¹ and Z², in particular one of the preferred meanings, and the groups —O—Z are both in para positions relative to the moiety X, the compound of formula (I) is a compound of the formula (Ia-1),

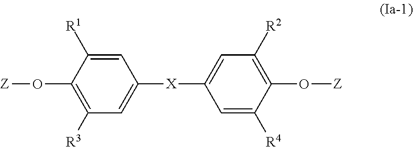

where X, R¹, R², R³ and R⁴ have the meanings defined herein, in particular the meanings mentioned as preferred and where R³ and R⁴ are in particular different from hydrogen.

In this subgroup (10.1) of group (10) of embodiments the structural unit of the formulae (II) or (IIa) is a structural unit of the formula (IIa-1),

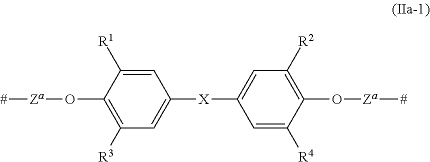

where # represents a connection point to a neighboring structural unit and where Z^{a} has one of the meanings defined herein for Z^{1a} and Z^{2a}, in particular one of the preferred meanings, the variables X, R¹, R², R³ and R⁴ have the meanings defined herein, in particular the meanings mentioned as preferred and where R³ and R⁴ are in particular different from hydrogen.

Preferably, the moiety X in formulae (Ia-1) and (IIa-1) is as defined in group (5), group (5′) or group (5″) of embodiments. Thus, the moiety X is here in particular selected from the group consisting of a single bond, O, N-phenyl, N-naphthyl, N-phenanthryl, CH₂, C(CH₃)₂, CH(CH₃), S, S(O) and SO₂, and more particularly selected from the group consisting of a single bond, O, N-phenyl, N-naphth-1-yl, N-naphth-2-yl, N-phenanthren-9-yl, CH₂, C(CH₃)₂, CH(CH₃), S and SO₂, especially selected from the group consisting of a single bond, O, CH₂, C(CH₃)₂, S and SO₂ and specifically from the group consisting of a single bond, C(CH₃)₂, S and SO₂. Particular preference is given in this context to X being C(CH₃)₂. Particular preference is also given in this context to X being S or SO₂.

Preference is also given to compounds of the formula (Ia-1) and to structural units of the formula (IIa-1), where the substituents R¹ and R², independently of one another, are each as defined in groups (6), (6.1), (6.2), (6.3) and (6.3a) of embodiments and where the substituents R³ and R⁴, independently of one another, are each as defined in groups (7), (7.1), (7.2), (7.3), (7.4), (7.4a) and (7.5) of embodiments.

Even more preference is given to compounds of the formula (Ia-1) and to structural units of the formula (IIa-1), where the substitutents R¹, R², R³ and R⁴ have the same meaning, which is especially one of the meanings mentioned herein as preferred, and in particular one of the meanings defined in groups (6), (6.1), (6.2), (6.3) and (6.3a) and groups (7), (7.1), (7.2), (7.3), (7.4), (7.4a) and (7.5) of embodiments.

Particular preference is given to compounds of the formula (Ia-1) and to structural units of the formula (IIa-1), where the substitutents R¹, R², R³ and R⁴ have the same meaning which is selected from the group consisting of phenyl, naphth-1-yl, naphth-2-yl, 1,2-dihydroacenaphthylen-5-yl, phenanthren-9-yl, pyren-1-yl, pyren-2-yl, pyren-4-yl, triphenylen-1-yl, triphenylen-2-yl, dibenzo[b,d]furan-2-yl, dibenzo[b,d]furan-4-yl, benzo[b]thien-3-yl, benzo[b]thien-4-yl, benzo[b]thien-5-yl, benzo[b]thien-6-yl, benzo[b]thien-7-yl, dibenzo[b,d]thien-2-yl, dibenzo[b,d]thien-4-yl, thianthren-1-yl and thianthren-2-yl. Special preference is given to compounds of the formula (Ia-1) and to structural units of the formula (IIa-1), where the substitutents R¹, R², R³ and R⁴ are independently selected from phenyl, naphthyl, 1,2-dihydroacenaphthylenyl, phenanthrenyl, benzo[b]thienyl, dibenzo[b,d]thienyl and thianthrenyl, and specifically selected from phenyl, naphth-1-yl, naphth-2-yl, phenanthren-9-yl, dibenzo[b,d]thien-2-yl, dibenzo[b,d]thien-4-yl, thianthren-1-yl and thianthren-2-yl.

Examples of the particular subgroup (10.1) are the compounds of the formula (Ia-1) and the structural units of formula (IIa-1), in which the combination of the moiety X, the groups Z and the variable RV is as defined in any one of the lines 1 to 442 in table A below, where the variable R^{y} represents the identical meaning of the substituents R¹, R², R³ and R⁴.

TABLE A

| | X | R^{y} (R¹ = R² = R³ = R⁴) | Z |
|---|---|---|---|
| 1 | C(CH₃)₂ | phenyl | hydrogen |
| 2 | C(CH₃)₂ | naphth-1-yl | hydrogen |
| 3 | C(CH₃)₂ | naphth-2-yl | hydrogen |
| 4 | C(CH₃)₂ | phenanthren-9-yl | hydrogen |
| 5 | C(CH₃)₂ | dibenzo[b,d]thien-2-yl | hydrogen |
| 6 | C(CH₃)₂ | dibenzo[b,d]thien-4-yl | hydrogen |
| 7 | C(CH₃)₂ | thianthren-1-yl | hydrogen |
| 8 | C(CH₃)₂ | thianthren-2-yl | hydrogen |
| 9 | SO₂ | phenyl | hydrogen |
| 10 | SO₂ | naphth-1-yl | hydrogen |
| 11 | SO₂ | naphth-2-yl | hydrogen |
| 12 | SO₂ | phenanthren-9-yl | hydrogen |
| 13 | SO₂ | dibenzo[b,d]thien-2-yl | hydrogen |
| 14 | SO₂ | dibenzo[b,d]thien-4-yl | hydrogen |
| 15 | SO₂ | thianthren-1-yl | hydrogen |
| 16 | SO₂ | thianthren-2-yl | hydrogen |
| 17 | S | phenyl | hydrogen |
| 18 | S | naphth-1-yl | hydrogen |
| 19 | S | naphth-2-yl | hydrogen |
| 20 | S | phenanthren-9-yl | hydrogen |
| 21 | S | dibenzo[b,d]thien-2-yl | hydrogen |
| 22 | S | dibenzo[b,d]thien-4-yl | hydrogen |
| 23 | S | thianthren-1-yl | hydrogen |
| 24 | S | thianthren-2-yl | hydrogen |
| 25 | single bond | phenyl | hydrogen |
| 26 | single bond | naphth-1-yl | hydrogen |
| 27 | single bond | naphth-2-yl | hydrogen |
| 28 | single bond | phenanthren-9-yl | hydrogen |
| 29 | single bond | dibenzo[b,d]thien-2-yl | hydrogen |
| 30 | single bond | dibenzo[b,d]thien-4-yl | hydrogen |
| 31 | single bond | thianthren-1-yl | hydrogen |
| 32 | single bond | thianthren-2-yl | hydrogen |
| 33 | C(CH₃)₂ | phenyl | 2-hydroxyethyl |
| 34 | C(CH₃)₂ | naphth-1-yl | 2-hydroxyethyl |
| 35 | C(CH₃)₂ | naphth-2-yl | 2-hydroxyethyl |
| 36 | C(CH₃)₂ | phenanthren-9-yl | 2-hydroxyethyl |
| 37 | C(CH₃)₂ | triphenylen-1-yl | 2-hydroxyethyl |
| 38 | C(CH₃)₂ | triphenylen-2-yl | 2-hydroxyethyl |
| 39 | C(CH₃)₂ | pyren-1-yl | 2-hydroxyethyl |
| 40 | C(CH₃)₂ | pyren-2-yl | 2-hydroxyethyl |
| 41 | C(CH₃)₂ | pyren-4-yl | 2-hydroxyethyl |
| 42 | C(CH₃)₂ | 1,2-dihydro- | 2-hydroxyethyl |
| | | acenaphthylen-5-yl | |
| 43 | C(CH₃)₂ | dibenzo[b,d]furan-2-yl | 2-hydroxyethyl |
| 44 | C(CH₃)₂ | dibenzo[b,d]furan-4-yl | 2-hydroxyethyl |
| 45 | C(CH₃)₂ | benzo[b]thien-3-yl | 2-hydroxyethyl |
| 46 | C(CH₃)₂ | benzo[b]thien-4-yl | 2-hydroxyethyl |
| 47 | C(CH₃)₂ | benzo[b]thien-5-yl | 2-hydroxyethyl |
| 48 | C(CH₃)₂ | benzo[b]thien-6-yl | 2-hydroxyethyl |
| 49 | C(CH₃)₂ | benzo[b]thien-7-yl | 2-hydroxyethyl |
| 50 | C(CH₃)₂ | dibenzo[b,d]thien-2-yl | 2-hydroxyethyl |
| 51 | C(CH₃)₂ | dibenzo[b,d]thien-4-yl | 2-hydroxyethyl |
| 52 | C(CH₃)₂ | thianthren-1-yl | 2-hydroxyethyl |
| 53 | C(CH₃)₂ | thianthren-2-yl | 2-hydroxyethyl |
| 54 | SO₂ | phenyl | 2-hydroxyethyl |
| 55 | SO₂ | naphth-1-yl | 2-hydroxyethyl |
| 56 | SO₂ | naphth-2-yl | 2-hydroxyethyl |
| 57 | SO₂ | phenanthren-9-yl | 2-hydroxyethyl |
| 58 | SO₂ | triphenylen-1-yl | 2-hydroxyethyl |
| 59 | SO₂ | triphenylen-2-yl | 2-hydroxyethyl |
| 60 | SO₂ | pyren-1-yl | 2-hydroxyethyl |
| 61 | SO₂ | pyren-2-yl | 2-hydroxyethyl |
| 62 | SO₂ | pyren-4-yl | 2-hydroxyethyl |
| 63 | SO₂ | 1,2-dihydro- | 2-hydroxyethyl |
| | | acenaphthylen-5-yl | |
| 64 | SO₂ | dibenzo[b,d]furan-2-yl | 2-hydroxyethyl |
| 65 | SO₂ | dibenzo[b,d]furan-4-yl | 2-hydroxyethyl |
| 66 | SO₂ | benzo[b]thien-3-yl | 2-hydroxyethyl |
| 67 | SO₂ | benzo[b]thien-4-yl | 2-hydroxyethyl |
| 68 | SO₂ | benzo[b]thien-5-yl | 2-hydroxyethyl |
| 69 | SO₂ | benzo[b]thien-6-yl | 2-hydroxyethyl |
| 70 | SO₂ | benzo[b]thien-7-yl | 2-hydroxyethyl |
| 71 | SO₂ | dibenzo[b,d]thien-2-yl | 2-hydroxyethyl |
| 72 | SO₂ | dibenzo[b,d]thien-4-yl | 2-hydroxyethyl |
| 73 | SO₂ | thianthren-1-yl | 2-hydroxyethyl |
| 74 | SO₂ | thianthren-2-yl | 2-hydroxyethyl |
| 75 | S | phenyl | 2-hydroxyethyl |
| 76 | S | naphth-1-yl | 2-hydroxyethyl |
| 77 | S | naphth-2-yl | 2-hydroxyethyl |
| 78 | S | phenanthren-9-yl | 2-hydroxyethyl |
| 79 | S | triphenylen-1-yl | 2-hydroxyethyl |
| 80 | S | triphenylen-2-yl | 2-hydroxyethyl |
| 81 | S | pyren-1-yl | 2-hydroxyethyl |
| 82 | S | pyren-2-yl | 2-hydroxyethyl |
| 83 | S | pyren-4-yl | 2-hydroxyethyl |
| 84 | S | 1,2-dihydro- | 2-hydroxyethyl |
| | | acenaphthylen-5-yl | |
| 85 | S | dibenzo[b,d]furan-2-yl | 2-hydroxyethyl |
| 86 | S | dibenzo[b,d]furan-4-yl | 2-hydroxyethyl |
| 87 | S | benzo[b]thien-3-yl | 2-hydroxyethyl |
| 88 | S | benzo[b]thien-4-yl | 2-hydroxyethyl |
| 89 | S | benzo[b]thien-5-yl | 2-hydroxyethyl |
| 90 | S | benzo[b]thien-6-yl | 2-hydroxyethyl |
| 91 | S | benzo[b]thien-7-yl | 2-hydroxyethyl |
| 92 | S | dibenzo[b,d]thien-2-yl | 2-hydroxyethyl |
| 93 | S | dibenzo[b,d]thien-4-yl | 2-hydroxyethyl |
| 94 | S | thianthren-1-yl | 2-hydroxyethyl |
| 95 | S | thianthren-2-yl | 2-hydroxyethyl |
| 96 | S(O) | phenyl | 2-hydroxyethyl |
| 97 | S(O) | naphth-1-yl | 2-hydroxyethyl |
| 98 | S(O) | naphth-2-yl | 2-hydroxyethyl |
| 99 | S(O) | phenanthren-9-yl | 2-hydroxyethyl |
| 100 | S(O) | triphenylen-1-yl | 2-hydroxyethyl |
| 101 | S(O) | triphenylen-2-yl | 2-hydroxyethyl |
| 102 | S(O) | pyren-1-yl | 2-hydroxyethyl |
| 103 | S(O) | pyren-2-yl | 2-hydroxyethyl |
| 104 | S(O) | pyren-4-yl | 2-hydroxyethyl |
| 105 | S(O) | 1,2-dihydro- | 2-hydroxyethyl |
| | | acenaphthylen-5-yl | |
| 106 | S(O) | dibenzo[b,d]furan-2-yl | 2-hydroxyethyl |
| 107 | S(O) | dibenzo[b,d]furan-4-yl | 2-hydroxyethyl |
| 108 | S(O) | benzo[b]thien-3-yl | 2-hydroxyethyl |
| 109 | S(O) | benzo[b]thien-4-yl | 2-hydroxyethyl |
| 110 | S(O) | benzo[b]thien-5-yl | 2-hydroxyethyl |
| 111 | S(O) | benzo[b]thien-6-yl | 2-hydroxyethyl |
| 112 | S(O) | benzo[b]thien-7-yl | 2-hydroxyethyl |
| 113 | S(O) | dibenzo[b,d]thien-2-yl | 2-hydroxyethyl |
| 114 | S(O) | dibenzo[b,d]thien-4-yl | 2-hydroxyethyl |
| 115 | S(O) | thianthren-1-yl | 2-hydroxyethyl |
| 116 | S(O) | thianthren-2-yl | 2-hydroxyethyl |
| 117 | O | phenyl | 2-hydroxyethyl |
| 118 | O | naphth-1-yl | 2-hydroxyethyl |
| 119 | O | naphth-2-yl | 2-hydroxyethyl |
| 120 | O | phenanthren-9-yl | 2-hydroxyethyl |
| 121 | O | triphenylen-1-yl | 2-hydroxyethyl |
| 122 | O | triphenylen-2-yl | 2-hydroxyethyl |
| 123 | O | pyren-1-yl | 2-hydroxyethyl |
| 124 | O | pyren-2-yl | 2-hydroxyethyl |
| 125 | O | pyren-4-yl | 2-hydroxyethyl |
| 126 | O | 1,2-dihydro- | 2-hydroxyethyl |
| | | acenaphthylen-5-yl | |
| 127 | O | dibenzo[b,d]furan-2-yl | 2-hydroxyethyl |
| 128 | O | dibenzo[b,d]furan-4-yl | 2-hydroxyethyl |
| 129 | O | benzo[b]thien-3-yl | 2-hydroxyethyl |
| 130 | O | benzo[b]thien-4-yl | 2-hydroxyethyl |
| 131 | O | benzo[b]thien-5-yl | 2-hydroxyethyl |
| 132 | O | benzo[b]thien-6-yl | 2-hydroxyethyl |
| 133 | O | benzo[b]thien-7-yl | 2-hydroxyethyl |
| 134 | O | dibenzo[b,d]thien-2-yl | 2-hydroxyethyl |
| 135 | O | dibenzo[b,d]thien-4-yl | 2-hydroxyethyl |
| 136 | O | thianthren-1-yl | 2-hydroxyethyl |
| 137 | O | thianthren-2-yl | 2-hydroxyethyl |
| 138 | CH₂ | phenyl | 2-hydroxyethyl |
| 139 | CH₂ | naphth-1-yl | 2-hydroxyethyl |
| 140 | CH₂ | naphth-2-yl | 2-hydroxyethyl |
| 141 | CH₂ | phenanthren-9-yl | 2-hydroxyethyl |
| 142 | CH₂ | triphenylen-1-yl | 2-hydroxyethyl |
| 143 | CH₂ | triphenylen-2-yl | 2-hydroxyethyl |
| 144 | CH₂ | pyren-1-yl | 2-hydroxyethyl |
| 145 | CH₂ | pyren-2-yl | 2-hydroxyethyl |
| 146 | CH₂ | pyren-4-yl | 2-hydroxyethyl |
| 147 | CH₂ | 1,2-dihydro- | 2-hydroxyethyl |
| | | acenaphthylen-5-yl | |
| 148 | CH₂ | dibenzo[b,d]furan-2-yl | 2-hydroxyethyl |
| 149 | CH₂ | dibenzo[b,d]furan-4-yl | 2-hydroxyethyl |
| 150 | CH₂ | benzo[b]thien-3-yl | 2-hydroxyethyl |
| 151 | CH₂ | benzo[b]thien-4-yl | 2-hydroxyethyl |
| 152 | CH₂ | benzo[b]thien-5-yl | 2-hydroxyethyl |
| 153 | CH₂ | benzo[b]thien-6-yl | 2-hydroxyethyl |
| 154 | CH₂ | benzo[b]thien-7-yl | 2-hydroxyethyl |
| 155 | CH₂ | dibenzo[b,d]thien-2-yl | 2-hydroxyethyl |
| 156 | CH₂ | dibenzo[b,d]thien-4-yl | 2-hydroxyethyl |
| 157 | CH₂ | thianthren-1-yl | 2-hydroxyethyl |
| 158 | CH₂ | thianthren-2-yl | 2-hydroxyethyl |
| 159 | N-phenyl | phenyl | 2-hydroxyethyl |
| 160 | N-phenyl | naphth-1-yl | 2-hydroxyethyl |
| 161 | N-phenyl | naphth-2-yl | 2-hydroxyethyl |
| 162 | N-phenyl | phenanthren-9-yl | 2-hydroxyethyl |
| 163 | N-phenyl | triphenylen-1-yl | 2-hydroxyethyl |
| 164 | N-phenyl | triphenylen-2-yl | 2-hydroxyethyl |
| 165 | N-phenyl | pyren-1-yl | 2-hydroxyethyl |
| 166 | N-phenyl | pyren-2-yl | 2-hydroxyethyl |
| 167 | N-phenyl | pyren-4-yl | 2-hydroxyethyl |
| 168 | N-phenyl | 1,2-dihydro- | 2-hydroxyethyl |
| | | acenaphthylen-5-yl | |
| 169 | N-phenyl | dibenzo[b,d]furan-2-yl | 2-hydroxyethyl |
| 170 | N-phenyl | dibenzo[b,d]furan-4-yl | 2-hydroxyethyl |
| 171 | N-phenyl | benzo[b]thien-3-yl | 2-hydroxyethyl |
| 172 | N-phenyl | benzo[b]thien-4-yl | 2-hydroxyethyl |
| 173 | N-phenyl | benzo[b]thien-5-yl | 2-hydroxyethyl |
| 174 | N-phenyl | benzo[b]thien-6-yl | 2-hydroxyethyl |
| 175 | N-phenyl | benzo[b]thien-7-yl | 2-hydroxyethyl |
| 176 | N-phenyl | dibenzo[b,d]thien-2-yl | 2-hydroxyethyl |
| 177 | N-phenyl | dibenzo[b,d]thien-4-yl | 2-hydroxyethyl |
| 178 | N-phenyl | thianthren-1-yl | 2-hydroxyethyl |
| 179 | N-phenyl | thianthren-2-yl | 2-hydroxyethyl |
| 180 | N-naphth-1-yl | phenyl | 2-hydroxyethyl |
| 181 | N-naphth-1-yl | naphth-1-yl | 2-hydroxyethyl |
| 182 | N-naphth-1-yl | naphth-2-yl | 2-hydroxyethyl |
| 183 | N-naphth-1-yl | phenanthren-9-yl | 2-hydroxyethyl |
| 184 | N-naphth-1-yl | triphenylen-1-yl | 2-hydroxyethyl |
| 185 | N-naphth-1-yl | triphenylen-2-yl | 2-hydroxyethyl |
| 186 | N-naphth-1-yl | pyren-1-yl | 2-hydroxyethyl |
| 187 | N-naphth-1-yl | pyren-2-yl | 2-hydroxyethyl |
| 188 | N-naphth-1-yl | pyren-4-yl | 2-hydroxyethyl |
| 189 | N-naphth-1-yl | 1,2-dihydro- | 2-hydroxyethyl |
| | | acenaphthylen-5-yl | |
| 190 | N-naphth-1-yl | dibenzo[b,d]furan-2-yl | 2-hydroxyethyl |
| 191 | N-naphth-1-yl | dibenzo[b,d]furan-4-yl | 2-hydroxyethyl |
| 192 | N-naphth-1-yl | benzo[b]thien-3-yl | 2-hydroxyethyl |
| 193 | N-naphth-1-yl | benzo[b]thien-4-yl | 2-hydroxyethyl |
| 194 | N-naphth-1-yl | benzo[b]thien-5-yl | 2-hydroxyethyl |
| 195 | N-naphth-1-yl | benzo[b]thien-6-yl | 2-hydroxyethyl |
| 196 | N-naphth-1-yl | benzo[b]thien-7-yl | 2-hydroxyethyl |
| 197 | N-naphth-1-yl | dibenzo[b,d]thien-2-yl | 2-hydroxyethyl |
| 198 | N-naphth-1-yl | dibenzo[b,d]thien-4-yl | 2-hydroxyethyl |
| 199 | N-naphth-1-yl | thianthren-1-yl | 2-hydroxyethyl |
| 200 | N-naphth-1-yl | thianthren-2-yl | 2-hydroxyethyl |
| 201 | N-naphth-2-yl | phenyl | 2-hydroxyethyl |
| 202 | N-naphth-2-yl | naphth-1-yl | 2-hydroxyethyl |
| 203 | N-naphth-2-yl | naphth-2-yl | 2-hydroxyethyl |
| 204 | N-naphth-2-yl | phenanthren-9-yl | 2-hydroxyethyl |
| 205 | N-naphth-2-yl | triphenylen-1-yl | 2-hydroxyethyl |
| 206 | N-naphth-2-yl | triphenylen-2-yl | 2-hydroxyethyl |
| 207 | N-naphth-2-yl | pyren-1-yl | 2-hydroxyethyl |
| 208 | N-naphth-2-yl | pyren-2-yl | 2-hydroxyethyl |
| 209 | N-naphth-2-yl | pyren-4-yl | 2-hydroxyethyl |
| 210 | N-naphth-2-yl | 1,2-dihydro- | 2-hydroxyethyl |
| | | acenaphthylen-5-yl | |
| 211 | N-naphth-2-yl | dibenzo[b,d]furan-2-yl | 2-hydroxyethyl |
| 212 | N-naphth-2-yl | dibenzo[b,d]furan-4-yl | 2-hydroxyethyl |
| 213 | N-naphth-2-yl | benzo[b]thien-3-yl | 2-hydroxyethyl |
| 214 | N-naphth-2-yl | benzo[b]thien-4-yl | 2-hydroxyethyl |
| 215 | N-naphth-2-yl | benzo[b]thien-5-yl | 2-hydroxyethyl |
| 216 | N-naphth-2-yl | benzo[b]thien-6-yl | 2-hydroxyethyl |
| 217 | N-naphth-2-yl | benzo[b]thien-7-yl | 2-hydroxyethyl |
| 218 | N-naphth-2-yl | dibenzo[b,d]thien-2-yl | 2-hydroxyethyl |
| 219 | N-naphth-2-yl | dibenzo[b,d]thien-4-yl | 2-hydroxyethyl |
| 220 | N-naphth-2-yl | thianthren-1-yl | 2-hydroxyethyl |
| 221 | N-naphth-2-yl | thianthren-2-yl | 2-hydroxyethyl |
| 222 | N-phenanthren- | phenyl | 2-hydroxyethyl |
| | 9-yl | | |
| 223 | N-phenanthren- | naphth-1-yl | 2-hydroxyethyl |
| | 9-yl | | |
| 224 | N-phenanthren- | naphth-2-yl | 2-hydroxyethyl |
| | 9-yl | | |
| 225 | N-phenanthren- | phenanthren-9-yl | 2-hydroxyethyl |
| | 9-yl | | |
| 226 | N-phenanthren- | triphenylen-1-yl | 2-hydroxyethyl |
| | 9-yl | | |
| 227 | N-phenanthren- | triphenylen-2-yl | 2-hydroxyethyl |
| | 9-yl | | |
| 228 | N-phenanthren- | pyren-1-yl | 2-hydroxyethyl |
| | 9-yl | | |
| 229 | N-phenanthren- | pyren-2-yl | 2-hydroxyethyl |
| | 9-yl | | |
| 230 | N-phenanthren- | pyren-4-yl | 2-hydroxyethyl |
| | 9-yl | | |
| 231 | N-phenanthren- | 1,2-dihydro- | 2-hydroxyethyl |
| | 9-yl | acenaphthylen-5-yl | |
| 232 | N-phenanthren- | dibenzo[b,d]furan-2-yl | 2-hydroxyethyl |
| | 9-yl | | |
| 233 | N-phenanthren- | dibenzo[b,d]furan-4-yl | 2-hydroxyethyl |
| | 9-yl | | |
| 234 | N-phenanthren- | benzo[b]thien-3-yl | 2-hydroxyethyl |
| | 9-yl | | |
| 235 | N-phenanthren- | benzo[b]thien-4-yl | 2-hydroxyethyl |
| | 9-yl | | |
| 236 | N-phenanthren- | benzo[b]thien-5-yl | 2-hydroxyethyl |
| | 9-yl | | |
| 237 | N-phenanthren- | benzo[b]thien-6-yl | 2-hydroxyethyl |
| | 9-yl | | |
| 238 | N-phenanthren- | benzo[b]thien-7-yl | 2-hydroxyethyl |
| | 9-yl | | |
| 239 | N-phenanthren- | dibenzo[b,d]thien-2-yl | 2-hydroxyethyl |
| | 9-yl | | |
| 240 | N-phenanthren- | dibenzo[b,d]thien-4-yl | 2-hydroxyethyl |
| | 9-yl | | |
| 241 | N-phenanthren- | thianthren-1-yl | 2-hydroxyethyl |
| | 9-yl | | |
| 242 | N-phenanthren- | thianthren-2-yl | 2-hydroxyethyl |
| | 9-yl | | |
| 243 | single bond | phenyl | 2-hydroxyethyl |
| 244 | single bond | naphth-1-yl | 2-hydroxyethyl |
| 245 | single bond | naphth-2-yl | 2-hydroxyethyl |
| 246 | single bond | phenanthren-9-yl | 2-hydroxyethyl |
| 247 | single bond | dibenzo[b,d]thien-2-yl | 2-hydroxyethyl |
| 248 | single bond | dibenzo[b,d]thien-4-yl | 2-hydroxyethyl |
| 249 | single bond | thianthren-1-yl | 2-hydroxyethyl |
| 250 | single bond | thianthren-2-yl | 2-hydroxyethyl |
| 251 | C(CH₃)₂ | phenyl | methoxycarbonyl-methyl |
| 252 | C(CH₃)₂ | naphth-1-yl | methoxycarbonyl-methyl |
| 253 | C(CH₃)₂ | naphth-2-yl | methoxycarbonyl-methyl |
| 254 | C(CH₃)₂ | phenanthren-9-yl | methoxycarbonyl-methyl |
| 255 | C(CH₃)₂ | dibenzo[b,d]thien-2-yl | methoxycarbonyl-methyl |
| 256 | C(CH₃)₂ | dibenzo[b,d]thien-4-yl | methoxycarbonyl-methyl |
| 257 | C(CH₃)₂ | thianthren-1-yl | methoxycarbonyl-methyl |
| 258 | C(CH₃)₂ | thianthren-2-yl | methoxycarbonyl-methyl |
| 259 | SO₂ | phenyl | methoxycarbonyl-methyl |
| 260 | SO₂ | naphth-1-yl | methoxycarbonyl-methyl |
| 261 | SO₂ | naphth-2-yl | methoxycarbonyl-methyl |
| 262 | SO₂ | phenanthren-9-yl | methoxycarbonyl-methyl |
| 263 | SO₂ | dibenzo[b,d]thien-2-yl | methoxycarbonyl-methyl |
| 264 | SO₂ | dibenzo[b,d]thien-4-yl | methoxycarbonyl-methyl |
| 265 | SO₂ | thianthren-1-yl | methoxycarbonyl-methyl |
| 266 | SO₂ | thianthren-2-yl | methoxycarbonyl-methyl |
| 267 | S | phenyl | methoxycarbonyl-methyl |
| 268 | S | naphth-1-yl | methoxycarbonyl-methyl |
| 269 | S | naphth-2-yl | methoxycarbonyl-methyl |
| 270 | S | phenanthren-9-yl | methoxycarbonyl-methyl |
| 271 | S | dibenzo[b,d]thien-2-yl | methoxycarbonyl-methyl |
| 272 | S | dibenzo[b,d]thien-4-yl | methoxycarbonyl-methyl |
| 273 | S | thianthren-1-yl | methoxycarbonyl-methyl |
| 274 | S | thianthren-2-yl | methoxycarbonyl-methyl |
| 275 | single bond | phenyl | methoxycarbonyl-methyl |
| 276 | single bond | naphth-1-yl | methoxycarbonyl-methyl |
| 277 | single bond | naphth-2-yl | methoxycarbonyl-methyl |
| 278 | single bond | phenanthren-9-yl | methoxycarbonyl-methyl |
| 279 | single bond | dibenzo[b,d]thien-2-yl | methoxycarbonyl-methyl |
| 280 | single bond | dibenzo[b,d]thien-4-yl | methoxycarbonyl-methyl |
| 281 | single bond | thianthren-1-yl | methoxycarbonyl-methyl |
| 282 | single bond | thianthren-2-yl | methoxycarbonyl-methyl |
| 283 | C(CH₃)₂ | phenyl | (4-(hydroxymethyl)phenyl)methyl |
| 284 | C(CH₃)₂ | naphth-1-yl | (4-(hydroxymethyl)phenyl)methyl |
| 285 | C(CH₃)₂ | naphth-2-yl | (4-(hydroxymethyl)phenyl)methyl |
| 286 | C(CH₃)₂ | phenanthren-9-yl | (4-(hydroxymethyl)phenyl)methyl |
| 287 | C(CH₃)₂ | dibenzo[b,a]thien-2-yl | (4-(hydroxymethyl)phenyl)methyl |
| 288 | C(CH₃)₂ | dibenzo[b,d]thien-4-yl | (4-(hydroxymethyl)phenyl)methyl |
| 289 | C(CH₃)₂ | thianthren-1-yl | (4-(hydroxymethyl)phenyl)methyl |
| 290 | C(CH₃)₂ | thianthren-2-yl | (4-(hydroxymethyl)phenyl)methyl |
| 291 | SO₂ | phenyl | (4-(hydroxymethyl)phenyl)methyl |
| 292 | SO₂ | naphth-1-yl | (4-(hydroxymethyl)phenyl)methyl |
| 293 | SO₂ | naphth-2-yl | (4-(hydroxymethyl)phenyl)methyl |
| 294 | SO₂ | phenanthren-9-yl | (4-(hydroxymethyl)phenyl)methyl |
| 295 | SO₂ | dibenzo[b,d]thien-2-yl | (4-(hydroxymethyl)phenyl)methyl |
| 296 | SO₂ | dibenzo[b,d]thien-4-yl | (4-(hydroxymethyl)phenyl)methyl |
| 297 | SO₂ | thianthren-1-yl | (4-(hydroxymethyl)phenyl)methyl |
| 298 | SO₂ | thianthren-2-yl | (4-(hydroxymethyl)phenyl)methyl |
| 299 | S | phenyl | (4-(hydroxymethyl)phenyl)methyl |
| 300 | S | naphth-1-yl | (4-(hydroxymethyl)phenyl)methyl |
| 301 | S | naphth-2-yl | (4-(hydroxymethyl)phenyl)methyl |
| 302 | S | phenanthren-9-yl | (4-(hydroxymethyl)phenyl)methyl |
| 303 | S | dibenzo[b,d]thien-2-yl | (4-(hydroxymethyl)phenyl)methyl |
| 304 | S | dibenzo[b,d]thien-4-yl | (4-(hydroxymethyl)phenyl)methyl |
| 305 | S | thianthren-1-yl | (4-(hydroxymethyl)phenyl)methyl |
| 306 | S | thianthren-2-yl | (4-(hydroxymethyl)phenyl)methyl |
| 307 | single bond | phenyl | (4-(hydroxymethyl)phenyl)methyl |
| 308 | single bond | naphth-1-yl | (4-(hydroxymethyl)phenyl)methyl |
| 309 | single bond | naphth-2-yl | (4-(hydroxymethyl)phenyl)methyl |
| 310 | single bond | phenanthren-9-yl | (4-(hydroxymethyl)phenyl)methyl |
| 311 | single bond | dibenzo[b,d]thien-2-yl | (4-(hydroxymethyl)phenyl)methyl |
| 312 | single bond | dibenzo[b,d]thien-4-yl | (4-(hydroxymethyl)phenyl)methyl |
| 313 | single bond | thianthren-1-yl | (4-(hydroxymethyl)phenyl)methyl |
| 314 | single bond | thianthren-2-yl | (4-(hydroxymethyl)phenyl)methyl |
| 315 | C(CH₃)₂ | phenyl | (3-(hydroxymethyl)phenyl)methyl |
| 316 | C(CH₃)₂ | naphth-1-yl | (3-(hydroxymethyl)phenyl)methyl |
| 317 | C(CH₃)₂ | naphth-2-yl | (3-(hydroxymethyl)phenyl)methyl |
| 318 | C(CH₃)₂ | phenanthren-9-yl | (3-(hydroxymethyl)phenyl)methyl |
| 319 | C(CH₃)₂ | dibenzo[b,d]thien-2-yl | (3-(hydroxymethyl)phenyl)methyl |
| 320 | C(CH₃)₂ | dibenzo[b,d]thien-4-yl | (3-(hydroxymethyl)phenyl)methyl |
| 321 | C(CH₃)₂ | thianthren-1-yl | (3-(hydroxymethyl)phenyl)methyl |
| 322 | C(CH₃)₂ | thianthren-2-yl | (3-(hydroxymethyl)phenyl)methyl |
| 323 | SO₂ | phenyl | (3-(hydroxymethyl)phenyl)methyl |
| 324 | SO₂ | naphth-1-yl | (3-(hydroxymethyl)phenyl)methyl |
| 325 | SO₂ | naphth-2-yl | (3-(hydroxymethyl)phenyl)methyl |
| 326 | SO₂ | phenanthren-9-yl | (3-(hydroxymethyl)phenyl)methyl |
| 327 | SO₂ | dibenzo[b,d]thien-2-yl | (3-(hydroxymethyl)phenyl)methyl |
| 328 | SO₂ | dibenzo[b,d]thien-4-yl | (3-(hydroxymethyl)phenyl)methyl |
| 329 | SO₂ | thianthren-1-yl | (3-(hydroxymethyl)phenyl)methyl |
| 330 | SO₂ | thianthren-2-yl | (3-(hydroxymethyl)phenyl)methyl |
| 331 | S | phenyl | (3-(hydroxymethyl)phenyl)methyl |
| 332 | S | naphth-1-yl | (3-(hydroxymethyl)phenyl)methyl |
| 333 | S | naphth-2-yl | (3-(hydroxymethyl)phenyl)methyl |
| 334 | S | phenanthren-9-yl | (3-(hydroxymethyl)phenyl)methyl |
| 335 | S | dibenzo[b,d]thien-2-yl | (3-(hydroxymethyl)phenyl)methyl |
| 336 | S | dibenzo[b,d]thien-4-yl | (3-(hydroxymethyl)phenyl)methyl |
| 337 | S | thianthren-1-yl | (3-(hydroxymethyl)phenyl)methyl |
| 338 | S | thianthren-2-yl | (3-(hydroxymethyl)phenyl)methyl |
| 339 | single bond | phenyl | (3-(hydroxymethyl)phenyl)methyl |
| 340 | single bond | naphth-1-yl | (3-(hydroxymethyl)phenyl)methyl |
| 341 | single bond | naphth-2-yl | (3-(hydroxymethyl)phenyl)methyl |
| 342 | single bond | phenanthren-9-yl | (3-(hydroxymethyl)phenyl)methyl |
| 343 | single bond | dibenzo[b,d]thien-2-yl | (3-(hydroxymethyl)phenyl)methyl |
| 344 | single bond | dibenzo[b,d]thien-4-yl | (3-(hydroxymethyl)phenyl)methyl |
| 345 | single bond | thianthren-1-yl | (3-(hydroxymethyl)phenyl)methyl |
| 346 | single bond | thianthren-2-yl | (3-(hydroxymethyl)phenyl)methyl |
| 347 | C(CH₃)₂ | phenyl | (4-(hydroxymethyl)-1-naphthyl)methyl |
| 348 | C(CH₃)₂ | naphth-1-yl | (4-(hydroxymethyl)-1-naphthyl)methyl |
| 349 | C(CH₃)₂ | naphth-2-yl | (4-(hydroxymethyl)-1-naphthyl)methyl |
| 350 | C(CH₃)₂ | phenanthren-9-yl | (4-(hydroxymethyl)-1-naphthyl)methyl |
| 351 | C(CH₃)₂ | dibenzo[b,d]thien-2-yl | (4-(hydroxymethyl)-1-naphthyl)methyl |
| 352 | C(CH₃)₂ | dibenzo[b,d]thien-4-yl | (4-(hydroxymethyl)-1-naphthyl)methyl |
| 353 | C(CH₃)₂ | thianthren-1-yl | (4-(hydroxymethyl)-1-naphthyl)methyl |
| 354 | C(CH₃)₂ | thianthren-2-yl | (4-(hydroxymethyl)-1-naphthyl)methyl |
| 355 | SO₂ | phenyl | (4-(hydroxymethyl)-1-naphthyl)methyl |
| 356 | SO₂ | naphth-1-yl | (4-(hydroxymethyl)-1-naphthyl)methyl |
| 357 | SO₂ | naphth-2-yl | (4-(hydroxymethyl)-1-naphthyl)methyl |
| 358 | SO₂ | phenanthren-9-yl | (4-(hydroxymethyl)-1-naphthyl)methyl |
| 359 | SO₂ | dibenzo[b,d]thien-2-yl | (4-(hydroxymethyl)-1-naphthyl)methyl |
| 360 | SO₂ | dibenzo[b,d]thien-4-yl | (4-(hydroxymethyl)-1-naphthyl)methyl |
| 361 | SO₂ | thianthren-1-yl | (4-(hydroxymethyl)-1-naphthyl)methyl |
| 362 | SO₂ | thianthren-2-yl | (4-(hydroxymethyl)-1-naphthyl)methyl |
| 363 | S | phenyl | (4-(hydroxymethyl)-1-naphthyl)methyl |
| 364 | S | naphth-1-yl | (4-(hydroxymethyl)-1-naphthyl)methyl |
| 365 | S | naphth-2-yl | (4-(hydroxymethyl)-1-naphthyl)methyl |
| 366 | S | phenanthren-9-yl | (4-(hydroxymethyl)-1-naphthyl)methyl |
| 367 | S | dibenzo[b,d]thien-2-yl | (4-(hydroxymethyl)-1-naphthyl)methyl |
| 368 | S | dibenzo[b,d]thien-4-yl | (4-(hydroxymethyl)-1-naphthyl)methyl |
| 369 | S | thianthren-1-yl | (4-(hydroxymethyl)-1-naphthyl)methyl |
| 370 | S | thianthren-2-yl | (4-(hydroxymethyl)-1-naphthyl)methyl |
| 371 | single bond | phenyl | (4-(hydroxymethyl)-1-naphthyl)methyl |
| 372 | single bond | naphth-1-yl | (4-(hydroxymethyl)-1-naphthyl)methyl |
| 373 | single bond | naphth-2-yl | (4-(hydroxymethyl)-1-naphthyl)methyl |
| 374 | single bond | phenanthren-9-yl | (4-(hydroxymethyl)-1-naphthyl)methyl |
| 375 | single bond | dibenzo[b,d]thien-2-yl | (4-(hydroxymethyl)-1-naphthyl)methyl |
| 376 | single bond | dibenzo[b,d]thien-4-yl | (4-(hydroxymethyl)-1-naphthyl)methyl |
| 377 | single bond | thianthren-1-yl | (4-(hydroxymethyl)-1-naphthyl)methyl |
| 378 | single bond | thianthren-2-yl | (4-(hydroxymethyl)-1-naphthyl)methyl |
| 379 | C(CH₃)₂ | phenyl | (5-(hydroxymethyl)-1-naphthyl)methyl |
| 380 | C(CH₃)₂ | naphth-1-yl | (5-(hydroxymethyl)-1-naphthyl)methyl |
| 381 | C(CH₃)₂ | naphth-2-yl | (5-(hydroxymethyl)-1-naphthyl)methyl |
| 382 | C(CH₃)₂ | phenanthren-9-yl | (5-(hydroxymethyl)-1-naphthyl)methyl |
| 383 | C(CH₃)₂ | dibenzo[b,d]thien-2-yl | (5-(hydroxymethyl)-1-naphthyl)methyl |
| 384 | C(CH₃)₂ | dibenzo[b,d]thien-4-yl | (5-(hydroxymethyl)-1-naphthyl)methyl |
| 385 | C(CH₃)₂ | thianthren-1-yl | (5-(hydroxymethyl)-1-naphthyl)methyl |
| 386 | C(CH₃)₂ | thianthren-2-yl | (5-(hydroxymethyl)-1-naphthyl)methyl |
| 387 | SO₂ | phenyl | (5-(hydroxymethyl)-1-naphthyl)methyl |
| 388 | SO₂ | naphth-1-yl | (5-(hydroxymethyl)-1-naphthyl)methyl |
| 389 | SO₂ | naphth-2-yl | (5-(hydroxymethyl)-1-naphthyl)methyl |
| 390 | SO₂ | phenanthren-9-yl | (5-(hydroxymethyl)-1-naphthyl)methyl |
| 391 | SO₂ | dibenzo[b,d]thien-2-yl | (5-(hydroxymethyl)-1-naphthyl)methyl |
| 392 | SO₂ | dibenzo[b,d]thien-4-yl | (5-(hydroxymethyl)-1-naphthyl)methyl |
| 393 | SO₂ | thianthren-1-yl | (5-(hydroxymethyl)-1-naphthyl)methyl |
| 394 | SO₂ | thianthren-2-yl | (5-(hydroxymethyl)-1-naphthyl)methyl |
| 395 | S | phenyl | (5-(hydroxymethyl)-1-naphthyl)methyl |
| 396 | S | naphth-1-yl | (5-(hydroxymethyl)-1-naphthyl)methyl |
| 397 | S | naphth-2-yl | (5-(hydroxymethyl)-1-naphthyl)methyl |
| 398 | S | phenanthren-9-yl | (5-(hydroxymethyl)-1-naphthyl)methyl |
| 399 | S | dibenzo[b,d]thien-2-yl | (5-(hydroxymethyl)-1-naphthyl)methyl |
| 400 | S | dibenzo[b,d]thien-4-yl | (5-(hydroxymethyl)-1-naphthyl)methyl |
| 401 | S | thianthren-1-yl | (5-(hydroxymethyl)-1-naphthyl)methyl |
| 402 | S | thianthren-2-yl | (5-(hydroxymethyl)-1-naphthyl)methyl |
| 403 | single bond | phenyl | (5-(hydroxymethyl)-1-naphthyl)methyl |
| 404 | single bond | naphth-1-yl | (5-(hydroxymethyl)-1-naphthyl)methyl |
| 405 | single bond | naphth-2-yl | (5-(hydroxymethyl)-1-naphthyl)methyl |
| 406 | single bond | phenanthren-9-yl | (5-(hydroxymethyl)-1-naphthyl)methyl |
| 407 | single bond | dibenzo[b,d]thien-2-yl | (5-(hydroxymethyl)-1-naphthyl)methyl |
| 408 | single bond | dibenzo[b,d]thien-4-yl | (5-(hydroxymethyl)-1-naphthyl)methyl |
| 409 | single bond | thianthren-1-yl | (5-(hydroxymethyl)-1-naphthyl)methyl |
| 410 | single bond | thianthren-2-yl | (5-(hydroxymethyl)-1-naphthyl)methyl |
| 411 | C(CH₃)₂ | phenyl | (6-(hydroxymethyl)-2-naphthyl)methyl |
| 412 | C(CH₃)₂ | naphth-1-yl | (6-(hydroxymethyl)-2-naphthyl)methyl |
| 413 | C(CH₃)₂ | naphth-2-yl | (6-(hydroxymethyl)-2-naphthyl)methyl |
| 414 | C(CH₃)₂ | phenanthren-9-yl | (6-(hydroxymethyl)-2-naphthyl)methyl |
| 415 | C(CH₃)₂ | dibenzo[b,d]thien-2-yl | (6-(hydroxymethyl)-2-naphthyl)methyl |
| 416 | C(CH₃)₂ | dibenzo[b,d]thien-4-yl | (6-(hydroxymethyl)-2-naphthyl)methyl |
| 417 | C(CH₃)₂ | thianthren-1-yl | (6-(hydroxymethyl)-2-naphthyl)methyl |
| 418 | C(CH₃)₂ | thianthren-2-yl | (6-(hydroxymethyl)-2-naphthyl)methyl |
| 419 | SO₂ | phenyl | (6-(hydroxymethyl)-2-naphthyl)methyl |
| 420 | SO₂ | naphth-1-yl | (6-(hydroxymethyl)-2-naphthyl)methyl |
| 421 | SO₂ | naphth-2-yl | (6-(hydroxymethyl)-2-naphthyl)methyl |
| 422 | SO₂ | phenanthren-9-yl | (6-(hydroxymethyl)-2-naphthyl)methyl |
| 423 | SO₂ | dibenzo[b,d]thien-2-yl | (6-(hydroxymethyl)-2-naphthyl)methyl |
| 424 | SO₂ | dibenzo[b,d]thien-4-yl | (6-(hydroxymethyl)-2-naphthyl)methyl |
| 425 | SO₂ | thianthren-1-yl | (6-(hydroxymethyl)-2-naphthyl)methyl |
| 426 | SO₂ | thianthren-2-yl | (6-(hydroxymethyl)-2-naphthyl)methyl |
| 427 | S | phenyl | (6-(hydroxymethyl)-2-naphthyl)methyl |
| 428 | S | naphth-1-yl | (6-(hydroxymethyl)-2-naphthyl)methyl |
| 429 | S | naphth-2-yl | (6-(hydroxymethyl)-2-naphthyl)methyl |
| 430 | S | phenanthren-9-yl | (6-(hydroxymethyl)-2-naphthyl)methyl |
| 431 | S | dibenzo[b,d]thien-2-yl | (6-(hydroxymethyl)-2-naphthyl)methyl |
| 432 | S | dibenzo[b,d]thien-4-yl | (6-(hydroxymethyl)-2-naphthyl)methyl |
| 433 | S | thianthren-1-yl | (6-(hydroxymethyl)-2-naphthyl)methyl |
| 434 | S | thianthren-2-yl | (6-(hydroxymethyl)-2-naphthyl)methyl |
| 435 | single bond | phenyl | (6-(hydroxymethyl)-2-naphthyl)methyl |
| 436 | single bond | naphth-1-yl | (6-(hydroxymethyl)-2-naphthyl)methyl |
| 437 | single bond | naphth-2-yl | (6-(hydroxymethyl)-2-naphthyl)methyl |
| 438 | single bond | phenanthren-9-yl | (6-(hydroxymethyl)-2-naphthyl)methyl |
| 439 | single bond | dibenzo[b,d]thien-2-yl | (6-(hydroxymethyl)-2-naphthyl)methyl |
| 440 | single bond | dibenzo[b,d]thien-4-yl | (6-(hydroxymethyl)-2-naphthyl)methyl |
| 441 | single bond | thianthren-1-yl | (6-(hydroxymethyl)-2-naphthyl)methyl |
| 442 | single bond | thianthren-2-yl | (6-(hydroxymethyl)-2-naphthyl)methyl |

Amongst the compounds of formula (Ia-1) and the structural units of formula (IIa-1) recited in table A, particular preference is given to those compounds and structural units of formulae (Ia-1) and (IIa-1), where moiety X is C(CH₃)₂, SO₂, S, or a single bond. In other words, particular preference is given to the compounds of the formula (Ia-1) in which the combination of the moiety X, the groups Z and the variable R^{y} is as defined in any one of the lines 1 to 95 and 243 to 442 in table A above, where the variable R^{y} represents the identical meaning of the substituents R¹, R², R³ and R⁴.

In a particular subgroup (10.2) of group (10) of embodiments, where in formula (Ia) the groups Z¹ and Z² are both Z which has one of the meanings defined herein for Z¹ and Z², in particular one of the preferred meanings, and the groups —O—Z are both in ortho positions relative to the moiety X, the compound of formula (I) is a compound of the formula (Ia-2),

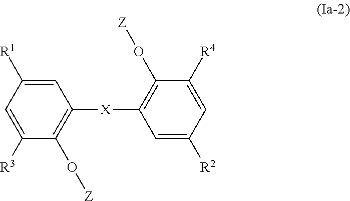

where X, R¹, R², R³ and R⁴ have the meanings defined herein, in particular the meanings mentioned as preferred and where R³ and R⁴ are in particular different from hydrogen.

In this subgroup (10.2) of group (10) of embodiments the structural unit of the formulae (II) or (IIa) is a structural unit of the formula (IIa-2),

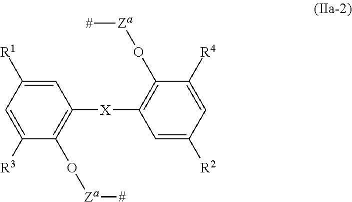

where # represents a connection point to a neighboring structural unit and where Z^{a} has one of the meanings defined herein for Z^{1a} and Z^{2a}, in particular one of the preferred meanings, the variables X, R¹, R², R³ and R⁴ have the meanings defined herein, in particular the meanings mentioned as preferred and where R³ and R⁴ are in particular different from hydrogen.

Preferably, the moiety X in formulae (Ia-2) and (IIa-2) is as defined in group (5) or group (5′″) of embodiments. Thus, the moiety X is here in particular selected from the group consisting of a single bond, O, N-phenyl, N-naphthyl, N-phenanthryl, CH₂, C(CH₃)₂, CH(CH₃), S, S(O) and SO₂, more particularly selected from the group consisting of a single bond, O, N-phenyl, N-naphth-1-yl, N-naphth-2-yl, N-phenanthren-9-yl, CH₂, C(CH₃)₂, CH(CH₃), S and SO₂, especially selected from the group consisting of a single bond, O, CH₂, C(CH₃)₂, S and SO₂ and specifically from the group consisting of a single bond, C(CH₃)₂, S and SO₂. Particular preference is given in this context to X being a single bond.

Preference is also given to compounds of the formula (Ia-2) and to structural units of the formula (IIa-2), where the substituents R¹ and R², independently of one another, are each as defined in groups (6), (6.1), (6.2), (6.3) and (6.3a) of embodiments and where the substituents R³ and R⁴, independently of one another, are each as defined in groups (7), (7.1), (7.2), (7.3), (7.4), (7.4a) and (7.5) of embodiments.

Even more preference is given to compounds of the formula (Ia-2) and to structural units of the formula (IIa-2), where the substitutents R¹, R², R³ and R⁴ have the same meaning, which is especially one of the meanings mentioned herein as preferred, and in particular one of the meanings defined in group (6), (6.1), (6.2), (6.3) and (6.3a) and groups (7), (7.1), (7.2), (7.3), (7.4), (7.4a) and (7.5) of embodiments.

Particular preference is given to compounds of the formula (Ia-2) and to structural units of the formula (IIa-2), where the substitutents R¹, R², R³ and R⁴ have the same meaning which is selected from the group consisting of phenyl, naphth-1-yl, naphth-2-yl, 1,2-dihydroacenaphthylen-5-yl, phenanthren-9-yl, pyren-1-yl, pyren-2-yl, pyren-4-yl, triphenylen-1-yl, triphenylen-2-yl, dibenzo[b,d]furan-2-yl, dibenzo[b,d]furan-4-yl, benzo[b]thien-3-yl, benzo[b]thien-4-yl, benzo[b]thien-5-yl, benzo[b]thien-6-yl, benzo[b]thien-7-yl, dibenzo[b,d]thien-2-yl, dibenzo[b,d]thien-4-yl, thianthren-1-yl and thianthren-2-yl, and specifically selected from phenyl, naphth-1-yl, naphth-2-yl, phenanthren-9-yl, dibenzo[b,d]thien-2-yl, dibenzo[b,d]thien-4-yl, thianthren-1-yl and thianthren-2-yl.

Examples of the particular subgroup (10.2) are the compounds of the formula (Ia-2) and the structural units of formula (IIa-2), in which the combination of the moiety X and the variable R^{x} is as defined in any one of the lines 1 to 64 in table B below, where the variable R^{y} represents the identical meaning of the substituents R¹, R², R³ and R⁴.

TABLE B

| | X | R^{y} (R¹ = R² = R³ = R⁴) | Z |
|---|---|---|---|
| 1 | single bond | phenyl | hydrogen |
| 2 | single bond | naphth-1-yl | hydrogen |
| 3 | single bond | naphth-2-yl | hydrogen |
| 4 | single bond | phenanthren-9-yl | hydrogen |
| 5 | single bond | dibenzo[b,d]thien-2-yl | hydrogen |
| 6 | single bond | dibenzo[b,d]thien-4-yl | hydrogen |
| 7 | single bond | thianthren-1-yl | hydrogen |
| 8 | single bond | thianthren-2-yl | hydrogen |
| 9 | single bond | phenyl | 2-hydroxyethyl |
| 10 | single bond | naphth-1-yl | 2-hydroxyethyl |
| 11 | single bond | naphth-2-yl | 2-hydroxyethyl |
| 12 | single bond | phenanthren-9-yl | 2-hydroxyethyl |
| 13 | single bond | dibenzo[b,d]thien-2-yl | 2-hydroxyethyl |
| 14 | single bond | dibenzo[b,d]thien-4-yl | 2-hydroxyethyl |
| 15 | single bond | thianthren-1-yl | 2-hydroxyethyl |
| 16 | single bond | thianthren-2-yl | 2-hydroxyethyl |
| 17 | single bond | phenyl | methoxycarbony-methyl |
| 18 | single bond | naphth-1-yl | methoxycarbony-methyl |
| 19 | single bond | naphth-2-yl | methoxycarbony-methyl |
| 20 | single bond | phenanthren-9-yl | methoxycarbony-methyl |
| 21 | single bond | dibenzo[b,d]thien-2-yl | methoxycarbony-methyl |
| 22 | single bond | dibenzo[b,d]thien-4-yl | methoxycarbony-methyl |
| 23 | single bond | thianthren-1-yl | methoxycarbony-methyl |
| 24 | single bond | thianthren-2-yl | methoxycarbony-methyl |
| 25 | single bond | phenyl | (4-(hydroxymethyl)phenyl)methyl |
| 26 | single bond | naphth-1-yl | (4-(hydroxymethyl)phenyl)methyl |
| 27 | single bond | naphth-2-yl | (4-(hydroxymethyl)phenyl)methyl |
| 28 | single bond | phenanthren-9-yl | (4-(hydroxymethyl)phenyl)methyl |
| 29 | single bond | dibenzo[b,d]thien-2-yl | (4-(hydroxymethyl)phenyl)methyl |
| 30 | single bond | dibenzo[b,d]thien-4-yl | (4-(hydroxymethyl)phenyl)methyl |
| 31 | single bond | thianthren-1-yl | (4-(hydroxymethyl)phenyl)methyl |
| 32 | single bond | thianthren-2-yl | (4-(hydroxymethyl)phenyl)methyl |
| 33 | single bond | phenyl | (3-(hydroxymethyl)phenyl)methyl |
| 34 | single bond | naphth-1-yl | (3-(hydroxymethyl)phenyl)methyl |
| 35 | single bond | naphth-2-yl | (3-(hydroxymethyl)phenyl)methyl |
| 36 | single bond | phenanthren-9-yl | (3-(hydroxymethyl)phenyl)methyl |
| 37 | single bond | dibenzo[b,d]thien-2-yl | (3-(hydroxymethyl)phenyl)methyl |
| 38 | single bond | dibenzo[b,d]thien-4-yl | (3-(hydroxymethyl)phenyl)methyl |
| 39 | single bond | thianthren-1-yl | (3-(hydroxymethyl)phenyl)methyl |
| 40 | single bond | thianthren-2-yl | (3-(hydroxymethyl)phenyl)methyl |
| 41 | single bond | phenyl | (4-(hydroxymethyl)-1-naphthyl)methyl |
| 42 | single bond | naphth-1-yl | (4-(hydroxymethyl)-1-naphthyl)methyl |
| 43 | single bond | naphth-2-yl | (4-(hydroxymethyl)-1-naphthyl)methyl |
| 44 | single bond | phenanthren-9-yl | (4-(hydroxymethyl)-1-naphthyl)methyl |
| 45 | single bond | dibenzo[b,d]thien-2-yl | (4-(hydroxymethyl)-1-naphthyl)methyl |
| 46 | single bond | dibenzo[b,d]thien-4-yl | (4-(hydroxymethyl)-1-naphthyl)methyl |
| 47 | single bond | thianthren-1-yl | (4-(hydroxymethyl)-1-naphthyl)methyl |
| 48 | single bond | thianthren-2-yl | (4-(hydroxymethyl)-1-naphthyl)methyl |
| 49 | single bond | phenyl | (5-(hydroxymethyl)-1-naphthyl)methyl |
| 50 | single bond | naphth-1-yl | (5-(hydroxymethyl)-1-naphthyl)methyl |
| 51 | single bond | naphth-2-yl | (5-(hydroxymethyl)-1-naphthyl)methyl |
| 52 | single bond | phenanthren-9-yl | (5-(hydroxymethyl)-1-naphthyl)methyl |
| 53 | single bond | dibenzo[b,d]thien-2-yl | (5-(hydroxymethyl)-1-naphthyl)methyl |
| 54 | single bond | dibenzo[b,d]thien-4-yl | (5-(hydroxymethyl)-1-naphthyl)methyl |
| 55 | single bond | thianthren-1-yl | (5-(hydroxymethyl)-1-naphthyl)methyl |
| 56 | single bond | thianthren-2-yl | (5-(hydroxymethyl)-1-naphthyl)methyl |
| 57 | single bond | phenyl | (6-(hydroxymethyl)-2-naphthyl)methyl |
| 58 | single bond | naphth-1-yl | (6-(hydroxymethyl)-2-naphthyl)methyl |
| 59 | single bond | naphth-2-yl | (6-(hydroxymethyl)-2-naphthyl)methyl |
| 60 | single bond | phenanthren-9-yl | (6-(hydroxymethyl)-2-naphthyl)methyl |
| 61 | single bond | dibenzo[b,d]thien-2-yl | (6-(hydroxymethyl)-2-naphthyl)methyl |
| 62 | single bond | dibenzo[b,d]thien-4-yl | (6-(hydroxymethyl)-2-naphthyl)methyl |
| 63 | single bond | thianthren-1-yl | (6-(hydroxymethyl)-2-naphthyl)methyl |
| 64 | single bond | thianthren-2-yl | (6-(hydroxymethyl)-2-naphthyl)methyl |

The compounds of the formula (I), where X, Z¹, Z², R¹, R², R³ and R⁴ each have one of the meanings defined herein, can, for example, be prepared by analogy to the process shown in the following reaction scheme 1, which is especially suitable for compounds (1), wherein R¹, R², R³ and R⁴ have the same meaning and Z¹ and Z² are identical groups selected from -Alk-OH, —CH₂—Ar²—CH₂—OH, Alk-C(O)OR^{x} and —CH₂—Ar²—C(O)OR^{x} as defined herein. The corresponding compounds (1), wherein Z¹ and Z² are both hydrogen, can be obtained e.g. by modifying the process of scheme 1 such that step b) is omitted and compound (2) is subjected directly to reaction step c).

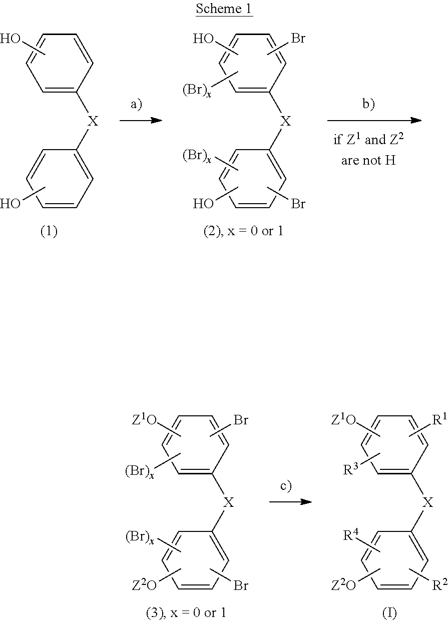

Each one of the conversions in steps a), b) and c) of scheme 1 can be accomplished by employing one or more of the reactions steps of the processes described herein below in connection with schemes 2a, 2b, 2c, 3, 4a, 4b, 5, 6a, 6b, 6c, 7a and 7b, or by apparent variations of these reactions steps, or, alternatively, by procedures well-established in preparative organic chemistry, or combinations thereof.

The compounds of the formula (Ia′), which are compounds of formula (Ia) as defined herein, where R¹, R², R³ and R⁴ have the same meaning, i.e. are identical substituents Ar selected from optionally substituted mono- or polycyclic (het)aryl as defined herein and where Z¹ and Z² are identical groups Z′ selected from -Alk-OH, —CH₂—Ar²—CH₂—OH, -Alk-C(O)OR^{x} and —CH₂—Ar²—C(O)OR^{x} as defined herein, can, for example, be prepared by analogy with the reactions depicted in the following reaction scheme 2a.

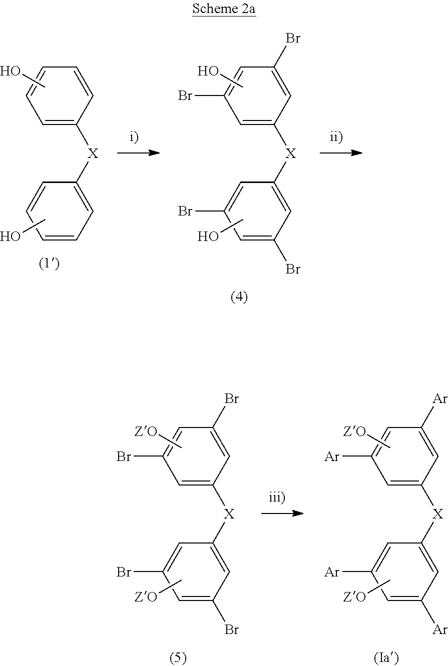

In the initial step i) the bisphenol (1′), whose hydroxyl groups are each located either in ortho position or in the para position relative to the moiety X, is reacted with the suitable brominating agent to afford the corresponding tetrabrominated derivative (4). A suitable brominating agent is in particular elemental bromine, which is typically used in a 3- to 15-fold molar excess in relation to the bisphenol (1′). In step ii) the tetrabromo bisphenol (4) can be converted to the compound (5) by reaction with a reagent Y—Z′, wherein Y is a suitable leaving group, such as a chloride, bromide, iodide, tosylate or mesitylate group and Z′ is -Alk-OH, —CH₂—Ar²—CH₂—OH, -Alk-C(O)OR^{x} or —CH₂—Ar²—C(O)OR^{x}, in the presence of a base, e.g. an oxo base, such as an alkaline carbonate like potassium carbonate. The conversion in step iii) of scheme 2a can be accomplished via a Suzuki coupling reaction by treating the tetrabromide (5) with a boronic acid of the formula Ar—B(OH)₂, where Ar has one of the meanings defined herein for substituents R¹ and R², or with an ester or anhydride of said boronic acid, in particular its C₁-C₄-alkyl ester, in the presence of a transition metal catalyst, in particular a palladium catalyst. Suitable palladium catalysts are in particular those which bear at least one tri-substituted phosphine ligand, such as e.g. tetrakis(triphenylphosphine) palladium and tetrakis(tritolylphosphine) palladium. Frequently, the palladium catalyst is prepared in situ from a suitable palladium precursor, such as e.g. palladium(II) acetate (Pd(OAc)₂, and a suitable phosphine ligand, like in particular triarylphosphines, such as e.g. triphenylphosphine and tritolylphosphine. Usually, the reaction is performed in the presence of a base, in particular an oxo base, such as an alkaline carbonate or an earth alkaline carbonate, such as e.g. potassium carbonate.

In case the group Z′ of the compound (5) is hydroxylethyl, the conversion shown in reactions step ii) of scheme 2a can be conducted using 2-chloro-ethanol as reagent Y—Z′, or alternatively, ethylene carbonate or ethylene oxide, in particular ethylene carbonate, instead of a reagent Y—Z′. Such conversions with 2-chloro-ethanol, ethylene carbonate or ethylene oxide are carried out in the presence of a base, e.g. an oxo base, such as an alkaline carbonate like potassium carbonate.

As a further example, if the group Z′ of the compound (5) is -Alk-C(O)OR^{x}, the conversion shown in reactions step ii) of scheme 2a can be conducted using Hal-Alk-C(O)OR^{x}, as reageant Y—Z′, where Hal is a halogen, such as especially bromine or chlorine, by analogy to the process described for instance in T. Ema, J. Org. Chem., 2010, 75(13), 4492-4500 or T. Ema et al., Org. Lett., 2006, 8, 17, 3773-3775. If desired, the thus introduced ester groups O-Alk-C(O) R^{x} can afterwards be converted into the corresponding acid groups O-Alk-C(O)OH using well known procedures of ester hydrolysis.

Suitable reaction conditions as well as suitable reagents for step i) of scheme 2a can be taken e.g. from U.S. Pat. Nos. 3,363,007, 5,208,389, JP H049346, CN 101100416, U.S. Pat. No. 6,147,264, L. Kumar et al., Organic Process Research & Development, 2010, 14(1), 174-179, S. Dev et al., Polymer, 2017, 133, 20-29, R.-N. Wang et al., Hebei Gongye Daxue Xuebao, 2012, 41(3), 42-45, J. Lu et al., Crystal Growth & Design, 2011, 11(8), 3551-3557, K.-B. Oh et al., Bioorganic & Medicinal Chemistry Letters, 2008, 18(1), 104-108, Y. Xin et al., Huaxue Yanjiu Yu Yingyong, 2006, 18(11), 1346-1348, Q. Yang et al., China, CN 111072529, CN 103992209, CN102898337, and V. A. Orlova et al., Trudy Vsesoyuznogo Instituta Gel'mintologii imeni K. I. Skryabina, 1971, 18, 201-205; for step ii) of scheme 2a e.g. from JP S50105638 (A), JP S5846034 (A), JPS5251351 (A), Imai, Hirokazu; et al. Japan, JP 2013249373, JP 2013249374, JP 2008143854, and JP H0338563 (A); and for step iii) of schemes 2a e.g. from A. Suzuki et al., Chem. Rev., 1995, 95, 2457-2483; N. Zhe et al., J. Med. Chem., 2005, 48 (5), 1569-1609; Young et al., J. Med. Chem., 2004, 47 (6), 1547-1552; C. Slee et al., Bioorg. Med. Chem. Lett., 2001, 9, 3243-3253; T. Zhang et al., Tetrahedron Lett., 2011, 52, 311-313; S. Bourrain et al., Synlett, 2004, 5, 795-798; and B. Li et al., Europ. J. Org. Chem., 2011, 3932-3937.

Alternatively, to prepare the compounds of the formula (Ia′), the sequence of steps i), ii) and iii) shown in scheme 2a may be changed according to schemes 2b and 2c below.

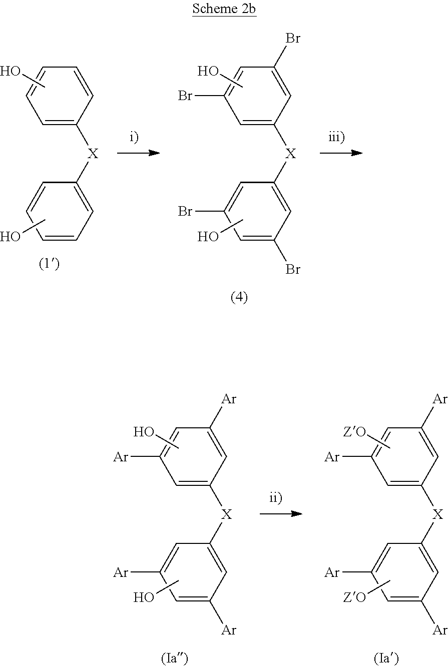

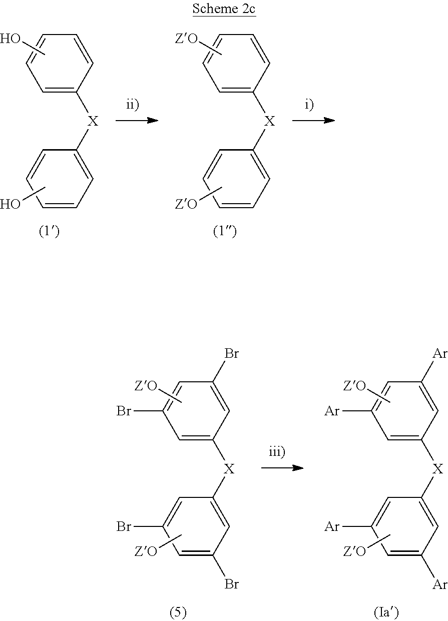

The reactions of steps i), ii) and iii) according to schemes 2b and 2c may be conducted using the same or very similar reaction conditions as those described for steps i), ii) and iii) of scheme 2a. The compound of formula (Ia″) obtained in the second reaction step of scheme 2b is a compound of formula (Ia) as defined herein, where R¹, R², R³ and R⁴ are all identical substituents Ar as defined in the context of scheme 2a and where Z¹ and Z² are both hydrogen. Thus, the sequence of steps i) and iii) according to scheme 2b) is suitable for preparing such compounds (Ia) of the present invention.

As an alternative to step i) in the schemes 2a and 2b, the tetrabrominated bisphenol of formula (4), where X is a moiety CH₂, can also be prepared by condensation of 2,6-dibromophenol or 2,4-dibromophenol with formaldehylde, as depicted in scheme 3 below.

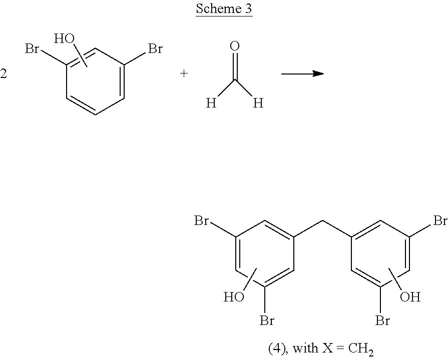

This reaction is described by K.-W. Chi et al., Journal of the Korean Chemical Society, 2003, 47(4), 412-416.

As an alternative to the synthesis according to schemes 2a or 2c, the tetrabromide of formula (5), where X is S(O) and Z′ is -Alk-OH, —CH₂—Ar²—CH₂—OH, -Alk-C(O)OR^{x} or —CH₂—Ar²—C(O)OR^{x} as defined herein, can also be prepared by reducing the corresponding compound (5) with X being SO₂. In turn, the tetrabromide of formula (5), where X is S(O), can be reduced to the corresponding sulfide, thus providing an alternative approach to the compound (5) with X being S. Likewise, the compounds (Ia′), where X is S(O) or S, are also accessible via reduction of the corresponding compounds (Ia′) with a SO₂ or S(O) moiety in position X. These conversions are summarized in the schemes 4a and 4b below.

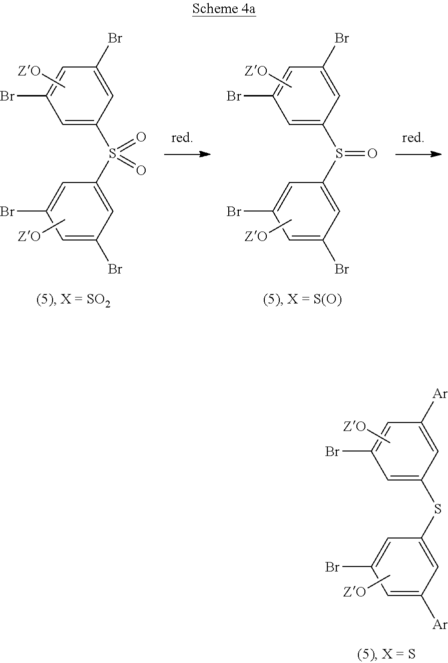

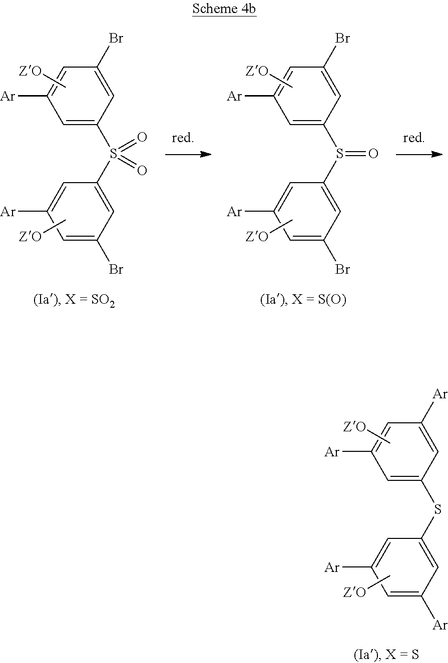

The reductive conversions shown in schemes 4a and 4b can be performed using procedures well established in the art for transforming sulfones into sulfoxides and sulfoxides into sulfides, respectively. For instance, sulfoxides may be converted to the respective sulfoxides by initial reaction with 4-chlorobenzenediazonium tetrafluoroborate followed by reduction with sodium borohydrate, while sulfoxides may be converted to the respective sulfides by reducing with lithium aluminium hydride or elemental sulfur.

An alternative to the processes according to schemes 2a to 2c for the preparation of the compound of the formula (Ia-1), where X is N—Ar¹, is the synthesis shown in scheme 5 below. The 2,6-diaryl phenol or 2,4-diaryl phenol (6) is first brominated and its hydroxyl group is then converted into a protective methoxymethyl (MOM) ether group to yield the intermediate (7), which is afterwards reacted with the arylamine (8) in the presence of a palladium catalyst. Final deprotection affords the compound (Ia″) with X being N—Ar¹, where Ar¹ is as defined herein, which can be transformed into the respective compound of formula (Ia′) according to scheme 2b. An analogous process is described in detail in Y. Matsuta et al., Chemistry—An Asian Journal, 2017, 12(15), 1889-1894.

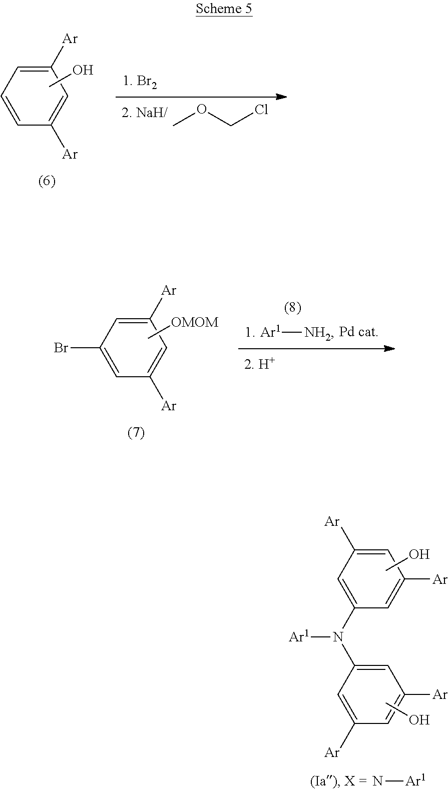

The compounds of the formula (Ia′″), which are compounds of formula (Ia) of the present invention, where X has one of the meanings defined herein. R³ and R⁴ are both hydrogen, R¹ and R² are identical substituents Ar selected from optionally substituted mono- or polycyclic (het)aryl as defined herein, and Z¹ and Z² are identical groups Z′ selected from -Alk-OH, —CH₂—Ar²—CH₂—OH, -Alk-C(O)OR^{x} and —CH₂—Ar²—C(O)OR^{x} as defined herein, can, for example, be prepared by analogy with the processes depicted in the following reaction scheme 6a.

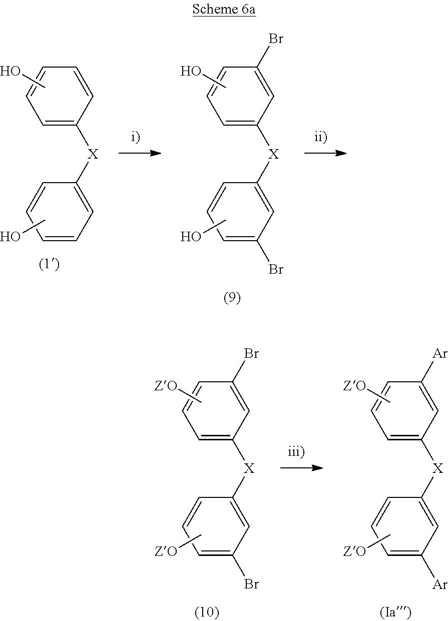

The reactions steps i), ii) and iii) of scheme 6a can in principle be carried out in analogy with the steps i) to iii) described above in connection with the preparation of compounds of the formula (Ia′) depicted in scheme 2a. However, the bromination in present step i), unlike the one in step i) of scheme 2a, is typically conducted using a 1.5- to 5-fold excess of bromine relative to the bisphenol (1′), which is as defined in the context of the process of scheme 2a above.

Suitable reaction conditions as well as suitable reagents for step i) of scheme 6a can be derived from the prior art documents listed above in connection with the process depicted in scheme 2a. In this regard additional specific information on step ii) of scheme 6a can be taken e.g. from CA 663542, U.S. Pat. No. 4,093,555; GB, 1 489 659 A; and on step iii) of scheme 6a from JP H02111743 (A), JP H08208775 (A), and S. R. Turner et al., High Performance Polymers, 2005, 17(3), 361-376.

The compounds of the formula (Ia′″) may alternatively be prepared by rearranging the order of steps i), ii) and iii) shown in scheme 6a in accordance to schemes 6b and 6c below.

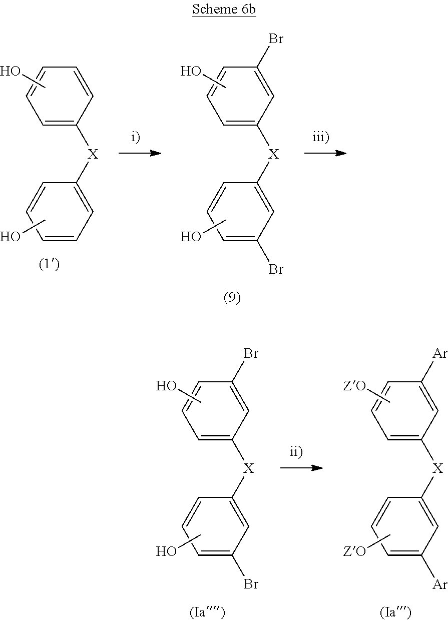

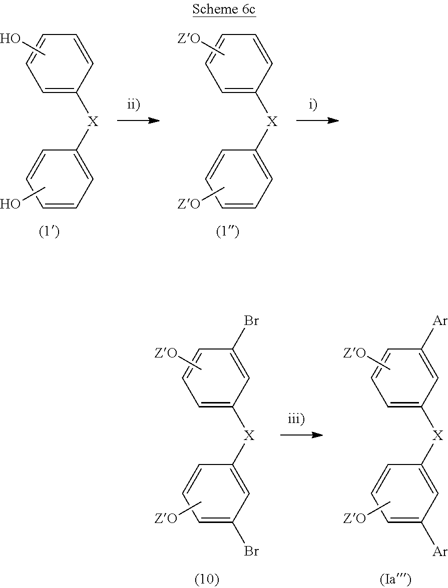

The reactions of steps i), ii) and iii) according to schemes 6b and 6c may be conducted using the same or very similar reaction conditions as those described for steps i), ii) and iii) of scheme 6a. The compound of formula (Ia″″) obtained in the second reaction step of scheme 6b is a compound of formula (Ia) as defined herein, where R³ and R⁴ are both hydrogen and R¹ an R² are identical substituents Ar as defined above, and where Z¹ and Z² are both hydrogen. Thus, the sequence of steps i) and iii) according to scheme 6b) is suitable for preparings such compounds (Ia) of the present invention.

As an alternative to the synthesis according to schemes 6a or 6c, the dibromide of formula (10), where X is S(O) and Z′ is -Alk-OH, —CH₂—Ar²—CH₂—OH, -Alk-C(O)OR^{x} or —CH₂—Ar²—C(O)OR^{x} as defined herein, can also be prepared by reducing the corresponding compound (10) with X being SO₂. In turn, the bisphenol compound of formula (10), where X is S(O), can be reduced to the corresponding sulfide, thus providing an alternative approach to the compound (10) with X being S. Likewise, the compounds (Ia′″), where X is S(O) or S, are also accessible via reduction of the corresponding compounds (Ia′″) with a SO₂ or S(O) moiety in position X. These conversions are summarized in the schemes 7a and 7b below.

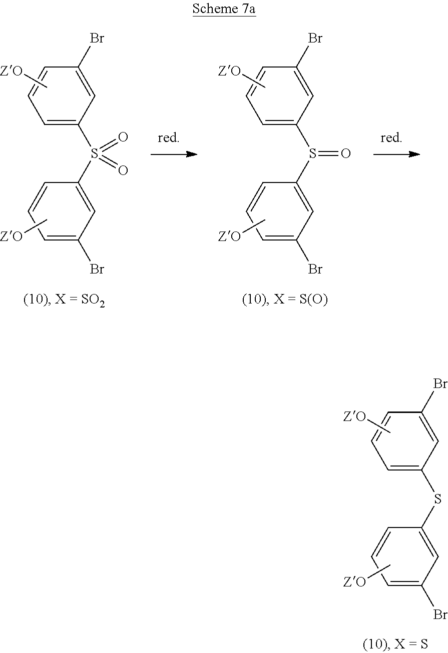

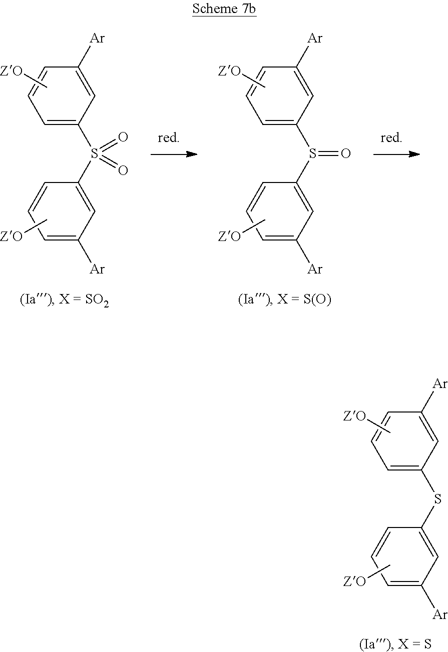

The reductive conversions shown in schemes 7a and 7b can be performed using procedures well established in the art for transforming sulfones into sulfoxides and sulfoxides into sulfides, respectively, such as those described above in connection with the processes of schemes 4a and 4b.

Alternative processes for preparing the dibrominated bisphenol of formula (9), where X is a moiety CH₂, and the compounds of the formulae (Ia′″) and (Ia″″), where X is N—Ar, can be readily derived from the processes described above in connection with schemes 3 and 5 by using as starting compounds 2- or 4-bromophenol instead of 2,6-dibromophenol ad a 2- or 4-aryl phenol instead of the 2,6-diaryl phenol (6), respectively.

Further compounds of formula (I) can be prepared by employing apparent variations of the reactions described above and combinations thereof with procedures well-established in preparative organic chemistry.

The reaction mixtures obtained in the individual steps of the syntheses for preparing the compounds described in reaction schemes 1, 2a, 2b, 2c, 3, 4a, 4b, 5, 6a, 6b, 6c, 7a and 7b above are usually worked up in a conventional way, e.g. by mixing with water, separating the phases and, where appropriate, purifying the crude products by washing, chromatography or crystallization. The intermediates in some cases result in the form of colourless or pale brownish, viscous oils, which are freed of volatiles or purified under reduced pressure and at moderately elevated temperature. If the intermediates are obtained as solids, the purification can be achieved by recrystallization or washing procedures, such as slurry washing.

The starting compounds used in the syntheses shown in schemes 1, 2a, 2b, 2c, 3, 4a, 4b, 5, 6a, 6b, 6c, 7a and 7b above to prepare compounds of formula (I) are commercially available or can be prepared by methods known from the art.

As stated above, the compounds of the present invention can be obtained in high purity, which means that a product is obtained, which does not contain significant amounts of organic impurities different from the compound of formula (I), except for volatiles. Usually, the purity of compounds of formula (I) is at least 95%, in particular at least 98% and especially at least 99%, based on the non-volatile organic matter, i.e. the product contains at most 5%, in particular at most 2% and especially at most 1% of non-volatile impurities different from the compound of formula (I).

The term “volatiles” refers to organic compounds, which have a boiling point of less than 200° C. at standard pressure (10⁵ Pa). Consequently, non-volatile organic matter is understood to mean compounds having a boiling point, which exceeds 200° C. at standard pressure.

It is a particular benefit of the invention that the compounds of formula (I) and likewise their solvates, can often be obtained in crystalline form. In the crystalline form the compound of formula (I) may be present in pure form or in the form of a solvate with water or an organic solvent. Therefore, a particular aspect of the invention relates to the compounds of formula (I), which are essentially present in crystalline form. In particular, the invention relates to crystalline forms, where the compound of formula (I) is present without solvent and to the crystalline solvates of the compounds of formula (I), where the crystals contain a solvent incorporated.

It is a particular benefit of the invention that the compounds of the formula (I) and likewise their solvates, can often be easily crystallized from conventional organic solvents. This allows for an efficient purification of the compounds of formula (I). Suitable organic solvents for crystallizing the compounds of the formula (I) or their solvates, include but are not limited to aromatic hydrocarbons such as toluene or xylene, aliphatic ketones in particular ketones having from 3 to 6 carbon atoms, such as acetone, methyl ethyl ketone, methyl isopropyl ketone or diethyl ketone, aliphatic and alicyclic ethers, such as diethyl ether, dipropyl ether, methyl isobutyl ether, methyl tert-butyl ether, ethyl tertbutyl ether, dioxane or tetrahydrofuran, aliphatic-aromatic ethers, such as anisole, and aliphatic alcohols having 1 to 4 carbon atoms, such as methanol, ethanol or isopropanol, as well as mixtures thereof.

Alternatively, the compounds of the formula (I) and likewise their solvates, can be obtained in purified form by employing other simple and efficient methods for purifying the raw products of these compounds, such as in particular slurry washing the raw solids obtained directly after the conversion to prepare the compounds of formula (I). Slurry washing is typically conducted at ambient temperature or elevated temperatures of usually about 30 to 90° C., in particular 40 to 80° C. Suitable organic solvents here are in principle the same as those listed above as being suitable for crystallizing the compounds of formula (I), such as in particular the mentioned aromatic hydrocarbons, aliphatic ketones and aliphatic ethers, e.g. toluene, methyl ethyl ketone and methyl tertbutyl ether.

Accordingly, the compounds of formula (I) used for the preparation of the thermoplastic polymers, in particular the polycarbonates, as defined herein, can be easily prepared and obtained in high yield and high purity. In particular, compounds of formula (I) can be obtained in crystalline form, which allows for an efficient purification to the degree required in the preparation of optical resins. In particular, these compounds can be obtained in a purity which provides for high refractive indices and also low haze, which is particularly important for the use in the preparation of optical resins of which the optical devise is made of. In conclusion, the compounds of formula (I) are particularly useful as monomers in the preparation of the optical resins.

A skilled person will readily appreciate that the formula (I) of the monomer used corresponds to the formula (II) of the structural unit comprised in the thermoplastic resin. Likewise, the formulae (Ia), (Ia-1) and (Ia-2), respectively, of the monomer used corresponds to the formulae (IIa), (IIa-1) and (IIa-2), respectively, of the structural unit comprised in the thermoplastic resin.

A skilled person will also appreciate that the structural units of the formulae (II), (IIa), (IIa-1) and (IIa-2), are repeating units within the polymer chains of the thermoplastic resin.

In addition to the structural units of the formulae (II), (IIa), (IIa-1) and (IIa-2), respectively, the thermoplastic resin may have structural units different therefrom. In a preferred embodiment, these further structural units are derived from aromatic monomers of the formula (IV) resulting in structural units of the formula (V):

HO—R^{z}-A¹-R^{z}—OH (IV)

#-O—R^{z}-A¹-R^{z}—O—# (V)
where
# represents a connection point to a neighboring structural unit;
A¹ is a polycyclic radical bearing at least 2 benzene rings, wherein the benzene rings may be connected by A and/or directly fused to each other and/or fused by a non-benzene carbocycle, where A¹ is unsubstituted or substituted by 1, 2 or 3 radicals R^{aa}, which are selected from the group consisting of halogen, C₁-C₆-alkyl, C₅-C₆-cycloalkyl and phenyl;
A is selected from the group consisting of a single bond, O, C═O, S, SO₂, CH₂, CH—Ar, CAr₂, CH(CH₃), C(CH₃)₂ and a radical of the formula (A′)

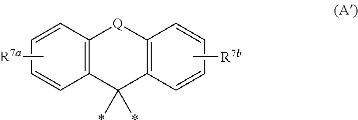

where
Q represents a single bond, O, NH, C═O, CH₂ or CH═CH;
R^{7a}, R^{7b}, independently of each other are selected from the group consisting of hydrogen, fluorine, CN, R, OR, CHₖR₃₋ₖ, NR₂, C(O)R and C(O)NH₂, where R is as defined herein and k is 0, 1, 2 or 3; and
* represents the connection point to a benzene ring;
Ar is selected from the group consisting of mono- or polycyclic aryl having from 6 to 26 carbon atoms as ring member atoms and mono- or polycyclic hetaryl having a total of 5 to 26 atoms, which are ring members, where 1, 2, 3 or 4 of these ring member atoms of hetaryl are selected from nitrogen, sulphur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk¹, O-Alk²-, O-Alk²-[O-Alk²-]ₚ- or O-Alk³-C(O)— where O is bound to A¹, and where
   p is an integer from 1 to 10;
   Alk¹ is C₁-C₄-alkandiyl;
   Alk² is C₂-C₄-alkandiyl; and
   Alk³ is C₁-C₄-alkandiyl.

If R^{z} in formula (IV) is O-Alk³-C(O), the esters, in particular the C₁-C₄-alkyl esters, of the monomers of formula (IV) may be used instead.

In the context of formulae (IV) and (V), A¹ is in particular a polycyclic radical bearing 2 benzene or naphthaline rings, wherein the benzene rings are connected by A. In this context A is in particular selected from the group consisting of a single bond, CH—Ar, CAr₂, and a radical A′.

In the context of formulae (IV) and (V), R^{z} is in particular O-Alk²-, where Alk² is in particular linear alkandiyl having 2 to 4 carbon atoms and especially O—CH₂CH₂.

Amongst the monomers of formula (IV) preference is given to monomers of the general formulae (IV-1) to (IV-6)

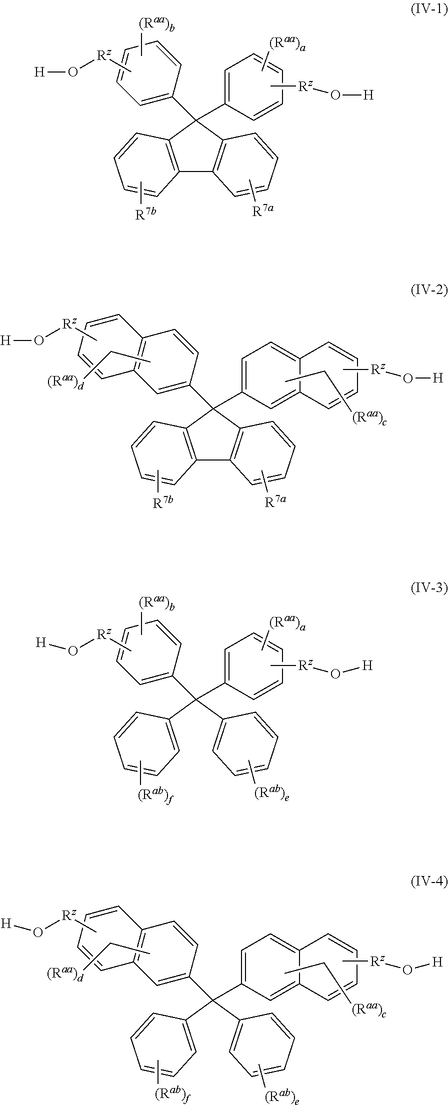

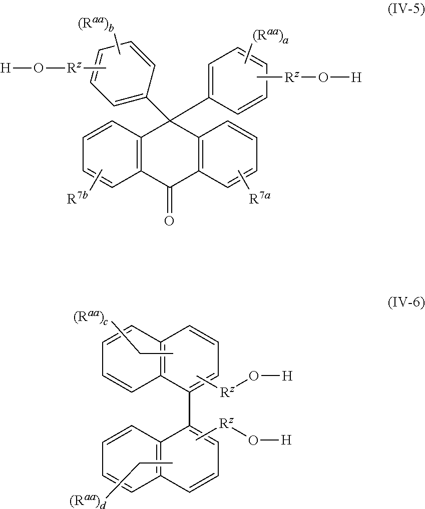

where
a and b are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
and where R^{z}, R^{aa}, R^{ab}, R^{7a} and R^{7b} are as defined for formula (IV) and where R^{z} is in particular selected from a single bond, CH₂ and OCH₂CH₂.

Amongst the monomers of formula (IV) particular preference is given to monomers of the general formulae (IV-11) to (IV-20), where R^{z} and R^{aa} are as defined herein and R^{z} is in particular selected from a single bond, CH₂ and O—CH₂CH₂, and especially is O—CH₂CH₂:

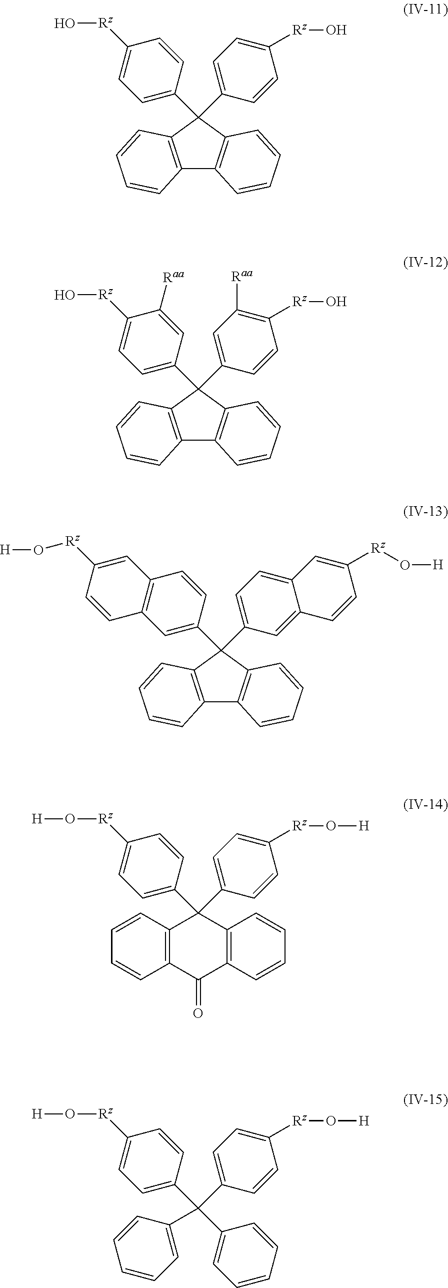

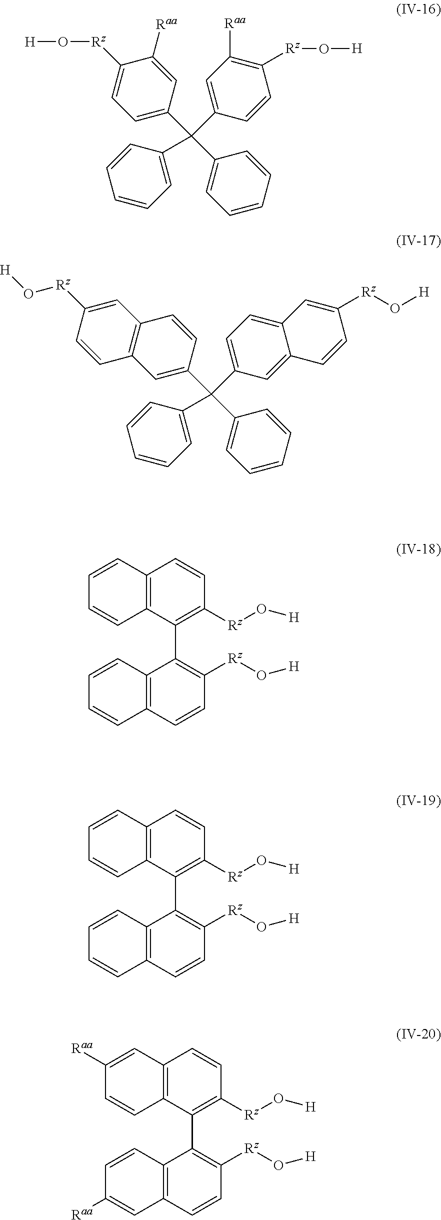

Examples of compounds of the formulae (IV-11) to (IV-20) are 9,9-bis(4-hydroxyphenyl)fluorene, 9,9-bis(4-hydroxy-3-methylphenyl)fluorene, 9,9-bis(4-hydroxy-3-isopropylphenyl)fluorene, 9,9-bis(4-hydroxy-3-tert.-butylphenyl)fluorene, 9,9-bis(4-hydroxy-3-cyclohexylphenyl)fluorene, 9,9-bis(4-hydroxy-3-phenylphenyl)fluorene, 9,9-bis(4-(2-hydroxyethoxy)phenyl)fluorene, 9,9-bis(4-(2-hydroxyethoxy)-3-methylphenyl)fluorene, 9,9-bis(4-(2-hydroxyethoxy)-3-isopropylphenyl)fluorene, 9,9-bis(4-(2-hydroxyethoxy)-3-tert.-butylphenyl)fluorene, 9,9-bis(4-(2-hydroxyethoxy)-3-cyclohexylphenyl)fluorene, 9,9-bis(4-(2-hydroxyethoxy)-3-phenylphenyl)fluorene also termed BPPEF, 9,9-bis(6-hydroxy-2-naphthyl)fluorene, 9,9-bis(6-(2-hydroxyethoxy)-2-naphthyl)fluorene, also termed 9,9-bis(6-(2-hydroxyethoxy)naphthalene-2-yl)fluorene (BNEF), 10,10-bis(4-hydroxyphenyl)anthracen-9-on, 10,10-bis(4-(2-hydroxyethoxy)phenyl)anthracen-9-on, 4,4′-dihydroxy-tetraphenylmethane, 4,4′-di-(2-hydroxyethoxy)-tetraphenylmethane, 3,3′-diphenyl-4,4′-dihydroxy-tetraphenylmethane, di-(6-hydroxy-2-naphthyl)-diphenylmethane, 2,2′-[1,1′-binaphthalene-2,2′-diylbis(oxy)]diethanol also termed 2,2′-bis(2-hydroxyethoxy)-1,1′-binaphtyl or 2,2′-bis(2-hydroxyethoxy)-1,1′-binaphthalene (BNE), 2,2′-bis(1-hydroxymethoxy)-1,1′-binaphtyl, 2,2′-bis(3-hydroxypropyloxy)-1,1′-binaphtyl, 2,2′-bis(4-hydroxybutoxy)-1,1′-binaphtyl, 2,2′-bis(2-hydroxyethoxy)-6,6′-diphenyl-1,1′-binaphthalene, 2,2′-bis(2-hydroxyethoxy)-6,6′-di(naphthalene-1-yl)-1,1′-binaphthalene, 2,2′-bis(2-hydroxymethoxy)-6,6′-diphenyl-1,1′-binaphthalene, 2,2′-bis(2-hydroxymethoxy)-6,6′-di(naphthalene-1-yl)-1,1′-binaphthalene, 2,2′-bis(2-hydroxypropoxy)-6,6′-diphenyl-1,1′-binaphthalene, 2,2′-bis(2-hydroxypropoxy)-6,6′-di(naphthalene-1-yl)-1,1′-binaphthalene, 2,2′-bis(2-hydroxyethoxy)-6,6′-di(naphthalene-2-yl)-1,1′-binaphthalene, 2,2′-bis(2-hydroxyethoxy)-6,6′-di(9-phenanthryl)-1,1′-binaphthalene and the like. Among the monomers of the general formula (IV) or of formulae (IV-1) to (IV-6), particular preference is given to the monomers of formulae (IV-1), (IV-2), (IV-3) and (IV-6) with more preference given to monomers of formulae (IV-2), (IV-3) and (IV-6) and special preference given to 2,2′-bis(2-hydroxyethoxy)-1,1′-binaphtyl (BNE or BHBNA), 2,2′-bis(2-hydroxyethoxy)-6,6′-diphenyl-1,1′-binaphtyl (DPBHBNA), 9,9-bis(4-(2-hydroxyethoxy)phenyl)fluorene (BPEF), 9,9-bis(6-(2-hydroxyethoxy)-2-naphthyl)fluorene (BNEF) and 9,9-bis(4-(2-hydroxyethoxy)-3-phenylphenyl)fluorene (BPPEF).

Accordingly, amongst the structural units of formula (V) that may be comprised in the thermoplastic resin preference is given to structural units of the general formulae (V-1) to (V-6),

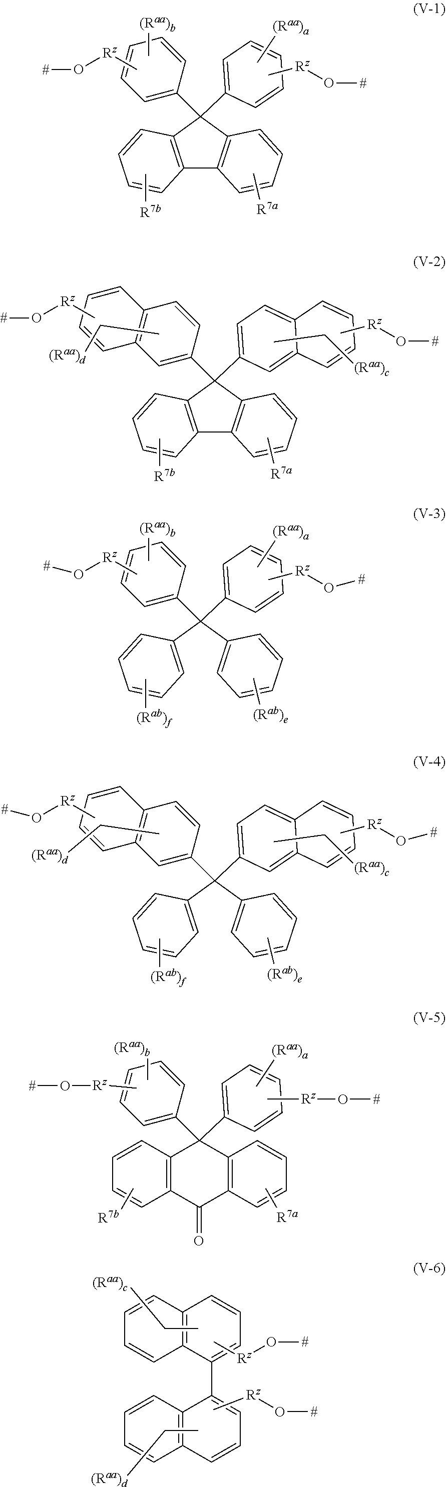

where
a and b are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
and where R^{z}, R^{aa}, R^{ab}, R^{7a} and R^{7b} are as defined for formula (V) and where R^{z} is in particular selected from a single bond, CH₂ and OCH₂CH₂.

Particular preference is given to structural units of the general formulae (V-11) to (V-20), where R^{z} and R^{aa} are as defined herein and where R^{z} is in particular selected from a single bond, CH₂ and O—CH₂CH₂, and especially is O—CH₂CH₂:

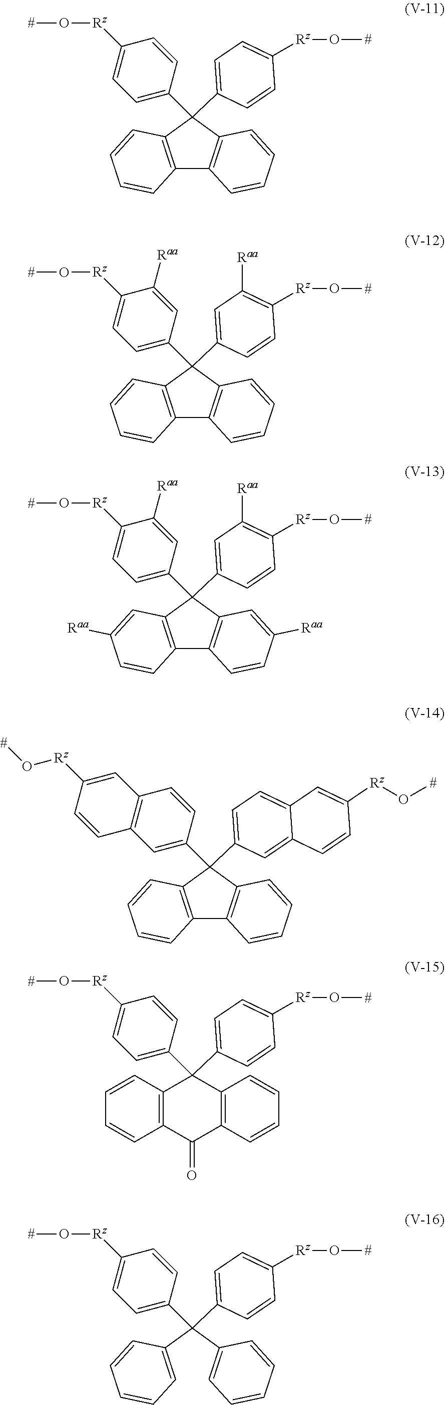

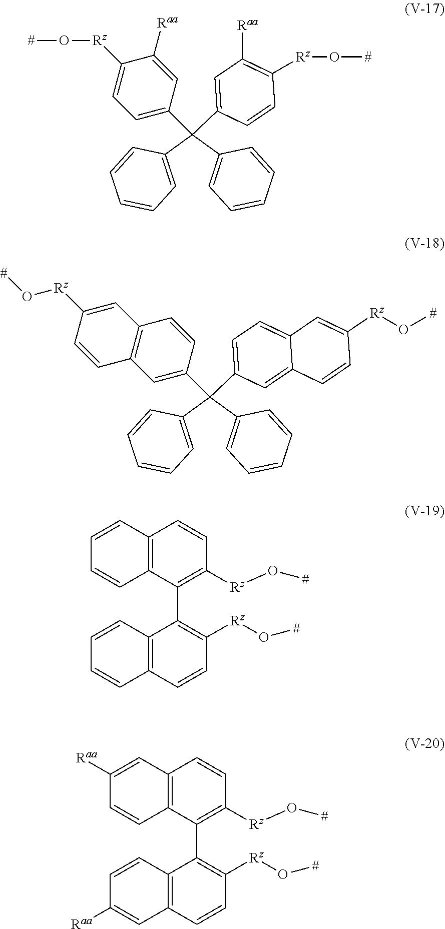

Among the structural units of the formulae (V-1) to (V-6), particular preference is given to the structural units of formulae (V-1), (V-2) and (V-6). Among the structural units of the formulae (V-11) to (V-20), particular preference is given to the structural units of formulae (V-11), (V-12), (V-14), (V-19) and (V-20) with more preference given to structural units of formulae (V-11), (V-19) and (V-20) and special preference given to structural units derived from 2,2′-bis(2-hydroxyethoxy)-1,1′-binaphtyl (BNE or BHBNA), 2,2′-bis(2-hydroxyethoxy)-6,6′-diphenyl-1,1′-binaphtyl (DPBHBNA) and 9,9-bis(4-(2-hydroxyethoxy)phenyl)fluorene (BPEF).

In a particular preferred group of embodiments, the thermoplastic resin of the present invention comprises at least one structural unit of the formulae (IIa-1) or (IIa-2) and at least one structural unit selected from the group consisting of structural units of the formula (V-11), structural units of the formula (V-19) and structural units of the formula (V-20). In this particular group of embodiments, those thermoplastic resins are preferred, where in the structural unit of the formulae (IIa-1) or (IIa-2) the substituents R¹, R², R³ and R⁴ or R¹ and R² are identical and have one of the meanings defined herein, especially one of the meanings mentioned as preferred. In this particular group of embodiments, those thermoplastic resins are preferred, where in the structural units of the formulae (V-11), (V-19) and (V-20) the radicals R^{z} are O—CH₂CH₂.

In the thermoplastic resins of this particular preferred group of embodiments, it is preferred that the total molar ratio of the structural units of the formulae (IIa-1) or (IIa-2) is in the range from 1 to 70 mol-%, preferably in the range from 5 to 60 mol-%, further preferably in the range from 8 to 45 mol-%, and even further preferably in the range from 10 to 30 mol-% of the total amount of structural units of the formulae (II) and (V).

A further particular group (10) of embodiments of the present invention relates to thermoplastic resins having only low, almost no or no birefringence. The resins of this group (10) of embodiments are characterized by having structural units of formula (II), such as in particular formula (IIa-1), wherein R¹, R², R³ and R⁴ are as defined for group 5.5 of embodiments, and additionally one or more structural units different from the structural units of formula (II) which are preferably selected from structural units of the formula (V), in particularly from structural units of formulae (V-11), (V-12), (V-14), (V-19) and (V-20) and specifically from structural units of the formulae (V-11), (V-19) and (V-20). In the thermoplastic resins of this particular preferred group (10) of embodiments, it is preferred that the total molar ratio of the structural units of the formulae (IIa-1) or (IIa-2) is in the range from 0.5 to 70 mol-%, preferably in the range from 1 to 60 mol-%, further preferably in the range from 2 to 45 mol-%, and even further preferably in the range from 3 to 30 mol-% of the total amount of structural units of the formulae (II) and (V).

The compounds of the formulae (IV), (IV-1), (IV-2), (IV-3), (IV-4), (IV-5), (IV-6), (IV-11), (IV-12), (IV-13), (IV-14), (IV-15), (IV-16), (IV-17), (IV-18), (IV-19) and (IV-20) are known or can be prepared by analogy to known methods.

For example, the compounds of the formula (IV-6) can be prepared by various synthesis methods, as disclosed e.g. in JP Publication No. 2014-227387, JP Publication No. 2014-227388, JP Publication No. 2015-168658, and JP Publication No. 2015-187098. For example, 1,1′-binaphthols may be reacted with ethylene glycol monotosylates; alternatively, 1,1′-binaphthols may be reacted with alkylene oxides, halogenoalkanols, or alkylene carbonates; and alternatively, 1,1′-binaphthols may be reacted with ethylene carbonates. Thereby, the compounds of the formula (IV-6) are obtained, where R^{z}—OH is O-Alk²- or O-Alk²-[O-Alk²-]ₚ-.

For example, the compounds of the formula (IV-2) can be prepared by various synthesis methods, as disclosed e.g. in JP Patent Publication No. 5442800, and JP Publication No. 2014-028806. Examples include:
(a) reacting fluorenes with hydroxy naphthalenes in the presence of hydrochloride gas and mercapto-carboxylic acid;
(b) reacting 9-fluorene with hydroxy naphthalenes in the presence of acid catalyst (and alkyl mercaptan);
(c) reacting fluorenes with hydroxy naphthalenes in the presence of hydrochloride and thiols (such as, mercapto-carboxylic acid);
(d) reacting fluorenes with hydroxy naphthalenes in the presence of sulfuric acid and thiols (such as, mercapto-carboxylic acid) and thereafter to crystallize the product from a crystallization solvent which consists of hydrocarbons and a polar solvent(s) to form bisnaphthol fluorene; and the like.

Thereby, compounds of the formula (IV-2) can be obtained, where R^{z} is a single bond.

The compounds of formulae (IV), where R^{z} is O-Alk²- or O-Alk²-[O-Alk²-]ₚ- can be prepared from compounds of formulae (IV), where R^{z} is a single bond, by reaction with alkylene oxides or haloalkanols. For example, reacting 9,9-bis(hydroxynaphthyl)fluorenes of the formula (IV-2) where R^{z} is a single bond with alkylene oxides or haloalkanols results in the compounds of the formula (IV-2) where R^{z} is O-Alk²- or O-Alk²-[O—Alk²-]ₚ-. For example, 9,9-bis[6-(2-hydroxyethoxy)naphthyl] fluorene can be prepared by reacting 9,9-bis[6-(2-hydroxynaphthyl] fluorene with 2-chloroethanol under alkaline conditions.

The monomers of formula (I) and likewise the co-monomers of formula (IV) used for producing the thermoplastic resin may contain certain impurities resulting from their preparation, e.g. hydroxy compounds, which bear an OH group instead of a group O—Z¹—OH or O—Z²—OH, or it may contain a group O-Alk′-[O-Alk′]ₒ instead of a group O-Alk′-, or it may contain a halogen atom instead of a radical R¹, R², R³ or R⁴. The total amount of such impurity compounds is preferably 1000 ppm or lower, more preferably 500 ppm or lower, still more preferably 200 ppm or lower, and especially preferably 100 ppm or lower. The total content of the impurities in the monomers used for preparing the thermoplastic resin is preferably 100 ppm or lower in particular 50 ppm or lower, and more preferably 20 ppm or lower. In particular, the total amount of dihydroxy compounds in which a carbon number of at least one of the radicals Z¹ or Z² differs from the formula (I), is preferably 1000 ppm or lower, more preferably 500 ppm or lower, still more preferably 200 ppm or lower, and especially preferably 100 ppm or lower; in the monomer(s) of which the main component is the dihydroxy compound(s) represented by the formula (I). The total content of the dihydroxy compounds in which a carbon number of at least one of the radicals Z¹ or Z² differs from the formula (I) is further preferably 50 ppm or lower, and more preferably 20 ppm or lower. Likewise, the amount of impurities in the co-monomers of formula (IV) will be in the range given for the monomers of formula (I).

Suitable thermoplastic resins for the preparation of optical devices, such as lenses, are in particular polycarbonates, polyestercarbonates and polyesters. Preferred thermoplastic resins for the preparation of optical devices, such as lenses, are in particular polycarbonates.

Said polycarbonates are structurally characterized by having structural units of at least one of the formulae (II), (IIa), (IIa-1) and (IIa-2), respectively, optionally structural units derived from diol monomers, which are different from the monomer compound of the formula (I), e.g. structural units of the formula (V),

#-O—R^{z}-A¹-R^{z}—O—# (V)
where
#, R^{z} and A¹ are as defined herein above;
   and a structural unit of formula (III-1) stemming from the carbonate forming component:

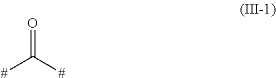

where each # represents a connection point to a neighboring structural unit, i.e. to O at the connection point of the structural unit of the formula (II) and, if present, to O at the connection point of the structural unit of the formula (V).

Said polyesters are structurally characterized by having structural units of at least one of the formulae (II), (IIa), (IIa-1) and (IIa-2), respectively, optionally structural units derived from diol monomers which are different from the monomer compound of the formula (I), e.g. structural units of the formula V, and structural units derived from dicarboxylic acid, e.g. of formula (III-2) in case of a benzene dicarboxylic acid, of formula (III-3) in case of a naphthalene carboxylic acid, of formula (III-4) in case of oxalic acid and of formula (III-5) in case of malonic acid:

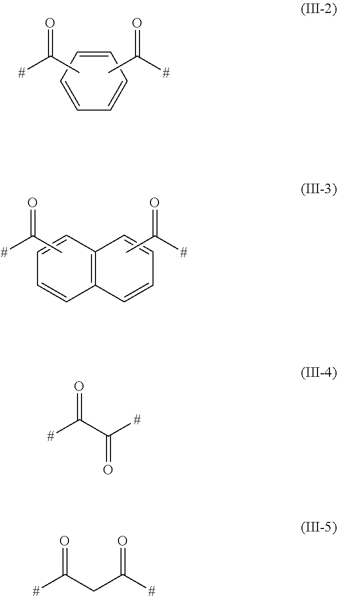

In formula (III-2) to (III-5) each variable # represents a connection point to a neighboring structural unit, i.e. to O of the connection point of the structural unit of the formula (II) and, if present, to O of the connection point of the structural unit of the formula (V).

Said polyestercarbonates are structurally characterized by having structural units of at least one of the formulae (II), (IIa), (IIa-1) and (IIa-2), respectively, optionally structural units derived from diol monomers which are different from the monomer compound of the formula (I), e.g. structural units of the formula (V), a structural unit of formula (III-1) stemming from the carbonate forming component and structural units derived from dicarboxylic acid, e.g. of formula (III-2) in case of a benzene dicarboxylic acid, of formula (III-3) in case of a naphthalene carboxylic acid, of formula (III-4) in case of oxalic acid and of formula (III-5) in case of malonic acid.

A particular group of embodiments relates to thermoplastic copolymer resins, in particular polycarbonates, polyestercarbonates and polyesters, which have both structural units of formula (II) and one or more structural units of formula (V), i.e. resins, in particular polycarbonates, polyestercarbonates and polyesters, which are obtainable by reacting at least one monomer of formula (I) with one or more monomers of formula (IV). In this case the molar ratio of monomers of formula (I) to monomers of formula (IV) and likewise the molar ratio of the structural units of formula (II) to structural units of formula (V) are in the range from 5:95 to 80:20, in particular in the range from 10:90 to 70:30 and especially in the range from 15:85 to 60:40 or in the range from 1:99 to 70:30, in particular in the range from 5:95 to 60:40, more preferably in the range from 8:92 to 45:55 or in the range from 10:90 to 40:60 and especially in the range from 12:88 to 30:70 or in the range from 12:88 to 20:80. Accordingly, the molar ratio of the structural units of the formula (II) is usually from 1 to 70 mol-%, in particular from 5 to 60 mol-%, more preferably in the range from 8 to 45 mol-% or in the range from 10 to 40 mol-%, especially in the range from 12 to 30 mol-% or in the range from 15 to 30 mol-%, and specifically in the range from 12 to 20 mol-% or in the range from 15 to 20 mol-%, based on the total molar amount of structural units of the formulae (II) and (V). Accordingly, the molar ratio of the structural units of the formula (V) is usually from 30 to 99 mol-% in particular from 40 to 95 mol-%, more preferably in the range from 55 to 92 mol-% or in the range from 60 to 90 mol-%, especially in the range from 70 to 88 mol-% or in the range from 70 to 85 mol-%, and specifically in the range from 80 to 88 mol-% or in the range from 80 to 85 mol-%, based on the total molar amount of structural units of the formulae (II) and (V).

The thermoplastic copolymer resins of the present invention, such as a polycarbonate resin may include either one of a random copolymer structure, a block copolymer structure, and an alternating copolymer structure. The thermoplastic resin according to the present invention does not need to include all of structural units (II) and one or more different structural units (V) in one, same polymer molecule. Namely, the thermoplastic copolymer resin according to the present invention may be a blend resin as long as the above-described structures are each included in any of a plurality of polymer molecules. For example, the thermoplastic resin including all of structural units (II) and structural units (V) described above may be a copolymer including all of structural units (II) and structural units (V), it may be a mixture of a homopolymer or a copolymer including at least one structural unit (II) and a homopolymer or a copolymer including at least one structural unit (V) or it may be a blend resin of a copolymer including at least one structural unit (II) and a first structural unit (V) and a copolymer including at least one structural unit (II) and at least one other structural unit (V) different from the first structural units (V); etc.

Thermoplastic polycarbonates are obtainable by polycondensation of a diol component and a carbonate forming component. Similarly, thermoplastic polyesters and polyestercarbonates are obtainable by polycondensation of a diol component and a dicarboxylic acid, or an ester forming derivative thereof, and optionally a carbonate forming component.

Specifically, thermoplastic resins (polycarbonate resins) can be prepared by the following methods.

A method for preparing the thermoplastic resin of the present invention, such as a polycarbonate resin, includes a process of melt polycondensation of a dihydroxy component corresponding to the above-mentioned structural units and a diester carbonate. According to the present invention the dihydroxy compound comprises at least one dihydroxy compound represented by the formula (I), in particular by the formulae (Ia), (Ia-1) or (Ia-2), respectively, as defined herein. In addition to the compound of formula (I), the dihydroxy compound may also comprise one or more dihydroxy compounds represented by the formula (IV), preferably by the formulae (IV-1) to (IV-6), in particular by the formulae (IV-11) to (IV-20), more particularly by the formulae (IV-11), (IV-12), (IV-14), (IV-19 or (IV-20) and especially by the formulae (IV-11), (IV-19) or (IV-20).

As is clear from the above, the polycarbonate resin can be formed by reacting a dihydroxy component with a carbonate precursor, such as a diester carbonate, where the dihydroxy component comprises at least one compound represented by the formulae (I), (Ia), (Ia-1) and (Ia-2), respectively, or a combination of at least one compound represented by the formulae (I), (Ia), (Ia-1) and (Ia-2), respectively, and at least one compound represented by the formulae (IV), (IV-1), (IV-2), (IV-3), (IV-4), (IV-5), (IV-6), (IV-11), (IV-12), (IV-13), (IV-14), (IV-15), (IV-16), (IV-17), (IV-18), (IV-19) or (IV-20). Specifically, a polycarbonate resin can be formed by a melt polycondensation process in which the compound represented by the formulae (I), (Ia), (Ia-1) and (Ia-2), respectively, or a combination thereof with at least one compound of the formulae (IV), (IV-1), (IV-2), (IV-3), (IV-4), (IV-5), (IV-6), (IV-11), (IV-12), (IV-13), (IV-14), (IV-15), (IV-16), (IV-17), (IV-18), (IV-19) or (IV-20) and a carbonate precursor, such as a diester carbonate, are reacted in the presence of a basic compound catalyst, a transesterification catalyst, or a mixed catalyst thereof, or in the absence of a catalyst.

A thermoplastic resin (or a polymer) other than a polycarbonate resin, such as polyestercarbonates and polyesters is obtained by using the dihydroxy compound represented by the formulae (I), (Ia), (Ia-1) and (Ia-2), respectively, or a combination thereof with at least one compound represented by the formulae (IV), (IV-1), (IV-2), (IV-3), (IV-4-), (IV-5), (IV-6), (IV-11), (IV-12), (IV-13), (IV-14), (IV-15), (IV-16), (IV-17), (IV-18), (IV-19) or (IV-20) as a material (or a monomer).

As mentioned before, the monomers of formula (I) and likewise the co-monomers of formula (IV) used for producing the thermoplastic resin may contain impurities resulting from their preparation.

For example, the compound of the formula (Ia-2), where X is C(CH₃)₂, and R¹ and R² are both naphthalen-2-yl, i.e. the compound 2,2′-(propane-2,2-diylbis{[2-(naphthalen-2-yl)-4,1-phenylene]oxy})di(ethan-1-ol) represented by the formula (Ia-2.3)

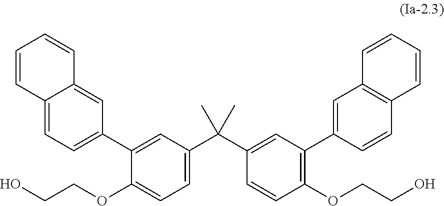 [SMILES: CC(C)(c1ccc(OCCO)c(-c2ccc3ccccc3c2)c1)c1ccc(OCCO)c(-c2ccc3ccccc3c2)c1]

may include e.g. one or more of the following compounds as impurities which are presented in the scheme below:

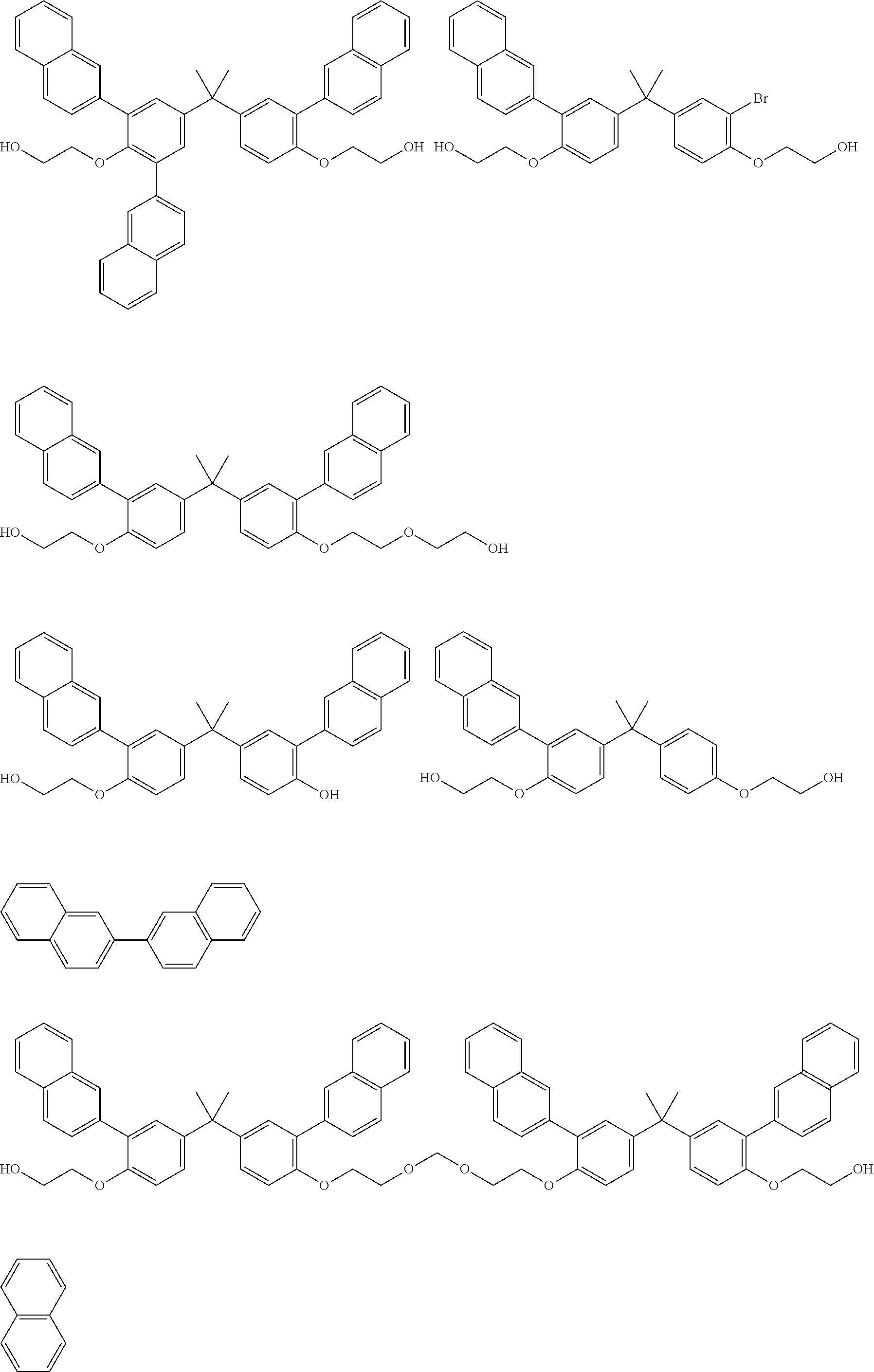

In particular, the total amount of impurities in the compound of formula (Ia-2.3) is preferably 1000 ppm or lower, more preferably 500 ppm or lower, still more preferably 200 ppm or lower, and especially preferably 100 ppm or lower. The total content of dihydroxy compounds in which a carbon number of at least one of the radicals Z¹ or Z² differs from the formula (Ia-2.3) is further preferably 50 ppm or lower, and more preferably 20 ppm or lower.

For example, the monomers of the formulae (IV-2) and (IV-3), where R^{z} is O-Alk²- or O-Alk²-[O-Alk²-]ₚ-, may include a dihydroxy compound in which both R^{z} are a single bond, or a dihydroxy compound in which one of R^{z} is a single bond, instead of O-Alk²- or O-Alk²-[O-Alk²-]ₚ-.

The total amount of such dihydroxy compounds of the formulae (IV-2) or (IV-3) in which at least one of R^{z} differs from O-Alk²- or O-Alk²-[O-Alk²-]ₚ-, is preferably 1000 ppm or lower, more preferably 500 ppm or lower, still more preferably 200 ppm or lower, and especially preferably 100 ppm or lower; in the monomer(s) of which main component is the dihydroxy compound(s) represented by the formulae (IV-2) or (IV-3). The total content of the dihydroxy compounds in which at least one of the values of c and d differs from the formula (IV-2) or (IV-3) is still preferably 50 ppm or lower, and more preferably 20 ppm or lower.

The polycarbonate resins can be obtained by reacting the monomer compounds of the formula (I) or by reacting combination of at least one monomer compound of the formula (I), in particular at least one monomer (I) mentioned herein as preferred, and one or more monomer compounds of the formula (IV), in particular of the formulae (V-11), (V-12), (V-14), (V-19) or (V-20) and especially of the formulae (IV-11), (IV-19) or (IV-20), and the like, as dihydroxy components; with carbonate precursors, such as diester carbonates.

However, in a polymerization process for manufacturing the polycarbonate resins, some compounds of the formulae (I) and (IV) may be converted into impurities, where one of or both of the terminal —Z¹OH, —Z²OH or —R^{z}OH radicals are replaced with a different radical, such as a vinyl terminal radical represented by —OCH═CH₂. Because the amount of such impurities is generally small, the products of the formed polymers can be used as polycarbonate resins without a purification process.

The thermoplastic resin of the present invention may also contain minor amount of impurities, for example, as extra contents of thermoplastic resin composition or a part of the polymer skeleton of the thermoplastic resin. The examples of such impurities include phenols formed by a process for forming the thermoplastic resin, unreacted diester carbonates and monomers. The total amount of impurities in the thermoplastic resin may be 5000 ppm or lower, or 2000 ppm or lower. The total amount of impurities in the thermoplastic resin is preferably 1000 ppm or lower, more preferably 500 ppm or lower, still more preferably 200 ppm or lower, and especially preferably 100 ppm or lower.

The total amount of phenols as impurities in the thermoplastic resin may be 3000 ppm or lower, or 2000 ppm or lower. The total amount of phenols as impurities is preferably 1000 ppm or lower, more preferably 800 ppm or lower, still more preferably 500 ppm or lower, and especially preferably 300 ppm or lower.

The total amount of diester carbonates as impurities in the thermoplastic resin is preferably 1000 ppm or lower, more preferably 500 ppm or lower, still more preferably 100 ppm or lower, and especially preferably 50 ppm or lower.

The total amount of unreacted monomers as impurities in the thermoplastic resin is preferably 3000 ppm or lower, more preferably 2000 ppm or lower, still more preferably 1000 ppm or lower, and especially preferably 500 ppm or lower.

The lower limit of the total amount of these impurities is not important, but may be 0.1 ppm, or 1.0 ppm.

The total amount of residual palladium as impurity in the thermoplastic resin is preferably 50 ppm or lower, more preferably 10 ppm or lower. The amount of residual palladium can be reduced by standard procedures like treatment with an adsorbent, e.g. active charcoal.

Resins having targeted characteristics can be formed by adjusting the amounts of phenols and diester carbonates. The amounts of phenols, diester carbonates, and monomers can be suitably adjusted by arranging the conditions for polycondensation, the working conditions of devices used for polymerization, or the conditions for extrusion molding after the polycondensation process.

The weight-average molecular weight (Mw), as determined by GPC (gel permeation chromatography), of the thermoplastic resin according to the present invention is preferably in the range from 5000 to 100000 Dalton, more preferably 10000 to 80000 Dalton, still more preferably 10000 to 50000 Dalton, and in particular in the range from 15000 to 50000 Dalton. The GPC measurements may be calibrated by using polystyrene standards. The Mw of a thermoplastic resin of the present invention determined this way may also denoted herein as “polystyrene conversion Mw”, “polystyrene converted Mw” or “Mw determined by GPC against a polystyrene standard”. The number-average molecular weight (Mn) of the thermoplastic resin according to the present invention is preferably 3000 to 20000, more preferably 5000 to 15000, and still more preferably 7000 to 14000. The Mn may be determined analogously to the Mw by GPC measurement calibrated against a polystyrene standard, as described herein below. The viscosity-average molecular weight (Mv) of the thermoplastic resin according to the present invention is preferably in the range from 8000 to 20000, more preferably 9000 to 15000, and still more preferably 10000 to 14000.

The value of the molecular weight distribution (Mw/Mn) of the thermoplastic resin according to the present invention is preferably 1.5 to 9.0, more preferably 1.8 to 7.0, and still more preferably 2.0 to 4.0.

When a thermoplastic resin has the value of the weight-average molecular weight (Mw) within the above-mentioned suitable range, a molded article made from the thermoplastic resin has high strength. In addition, such a thermoplastic resin with the suitable Mw value is advantageous for molding because of its excellent fluidity.

In a particular group of embodiments, the thermoplastic resin of the present invention comprises at least 0.3% by weight, preferably at least 0.5% by weight, more preferably at least 0.8% by weight and in particular at least 1.0% by weight of low molecular weight compounds having a molecular weight Mw of less than 1000, based on the total weight of the thermoplastic resin. The upper limit of said content of low molecular weight compounds having a Mw of less than 1000 is typically 7.0% by weight, preferably 5.0% by weight, more preferably 3.0% by weight, even more preferably 2.0% by weight, in particular 1.8% by weight and specifically 1.7% by weight. Accordingly, in this particular group of embodiments the content of low molecular weight compounds having a molecular weight Mw of less than 1000 in the thermoplastic resin is typically in the range of 0.3 to 7.0% by weight, preferably in the range of 0.5 to 5.0% by weight, more preferably 0.8 to 3.0% by weight, even more preferably in the range of 1.0 to 2.0% by weight, in particular in the range of 1.0 to 1.8% by weight and specifically in the range of 1.0 to 1.7% by weight, based in each case on the total weight of the thermoplastic resin.

Thermoplastic resins of the present invention comprising low molecular weight compounds with Mw-values of less than 1000 in an amount within the above ranges form molded bodies that have high mechanical strength. Such thermoplastic resins are in particular not or barely prone to separation or precipitation of said low molecular weight compounds, also known as bleed-out, in the course of molding processes, such as injection molding. In addition, the thermoplastic resins of the present invention, which contain the low molecular weight compounds in the amounts defined above, have the advantageous properties of high molding speed and reduced energy requirements for molding processes due to their high plasticity.

The content of the low-molecular-weight compounds in the thermoplastic resin is determined based on the diagram of the GPC analysis described above. In particular, said content is calculated as the ratio of the total area of the peaks of the low-molecular-weight compounds to the total area of all peaks of the diagram obtained by GPC analysis of a thermoplastic resin. Thus, the content of the low molecular weight compounds in the thermoplastic resin (CLWC) is represented by following formula:

$$CLWC ( \% ) = \frac{\begin{matrix}the total area of peaks of compounds with \\ Mw lower than 1. on GPC analysis\end{matrix}}{( the total area of all peaks of compounds on GPC analysis )} \times 100$$

The above-mentioned polycarbonate resin has a high refractive index (n_{D} or n_{d}) and thus is suitable to an optical lens. The values of the refractive index as referred herein are values of a film having a thickness of 0.1 mm may be measured by use of an Abbe refractive index meter by a method of JIS-K-7142. The refractive index of the polycarbonate resin according to the present invention at 23° C. at a wavelength of 589 nm is, in case the resin includes the structural unit (2), usually 1.640 or higher, preferably 1.650 or higher, more preferably 1.660 or higher, still more preferably 1.670 or higher, and in particular 1.680 or higher. For example, the refractive index of the copolycarbonate resin including the structural unit (2) and a structural unit (V) according to the present invention is preferably 1.660 to 1.720, more preferably 1.670 to 1.720, and in particular 1.680 to 1.720.

The Abbe number (ν) of the polycarbonate resin is preferably 24 or lower, more preferably 20 or lower, and still more preferably 18 or lower. The Abbe number may be calculated by use of the following equation based on the refractive index at wavelengths of 487 nm, 589 nm and 656 nm at 23° C.

$$v = ( {n}_{D} - 1 ) / ( {n}_{F} - {n}_{C} )$$

n_{D}: refractive index at a wavelength of 589 nm
n_{C}: refractive index at a wavelength of 656 nm
n_{F}: refractive index at a wavelength of 486 nm

The glass transition temperature (Tg) of the polycarbonate resin as an example of the thermoplastic resin according to the present invention is, in consideration of that the polycarbonate is usable for injection molding, preferably 90 to 185° C., more preferably 125 to 175° C., and still more preferably 140 to 165° C. With regard to the molding fluidity and the molding heat resistance, the lower limit of Tg is preferably 130° C. and more preferably 135° C., and the upper limit of Tg is preferably 185° C. and more preferably 175° C. A glass transition temperature (Tg) in the above given ranges provides a significant range of usable temperature and avoids the risk that the melting temperature of the resin may be too high, and thus the resin may be undesirably decomposed or colored. What is more, it allows for preparing molds having have a high surface accuracy.

In the preferred group (10) of embodiments the absolute value of the orientation birefringence of the thermoplastic resin is preferably in the range of 0 to 1×10⁻², more preferable in the range of 0 to 5×10⁻³, even more preferablein the range of 0 to 2×10⁻³, in particular in the range of 0 to 1×10⁻³, and specifically in the range of 0 to 0.4×10⁻³.

An optical molded body such as an optical element produced by using a polycarbonate resin of the present invention has a total light transmittance of preferably 85% or higher, more preferably 87% or higher, and especially preferably 88% or higher. A total light transmittance of preferably 85% or higher is as good as that provided by bisphenol A type polycarbonate resin or the like.

The thermoplastic resin according to the present invention has high moisture and heat resistance. The moisture and heat resistance may be evaluated by performing a “PCT test” (pressure cooker test) on a molded body such as an optical element produced by use of the thermoplastic resin and then measuring the total light transmittance of the molded body after the PCT test. In the PCT test, first, an injection molded body having a diameter of 50 mm and a thickness of 3 mm is kept for 20 hours with PC305S III made by HIRAYAMA Corporation under the conditions of 120° C., 0.2 MPa, 100% RH for 20 hours. Then, the sample of the injection molded body is removed from the device and the total light transmittance is measured using the SE2000 type spectroscopic parallax measuring instrument made by Nippon Denshoku Industries Co., Ltd in accordance with the method of JIS-K-7361-1.

The thermoplastic resin according to the present invention has a post-PCT test total light transmittance of 60% or higher, preferably 70% or higher, more preferably 75% or higher, still more preferably 80% or higher, and especially preferably 85% or higher. As long as the total light transmittance is 60% or higher, the thermoplastic resin is considered to have a higher moisture and heat resistance than that of the conventional thermoplastic resin.

The thermoplastic resin according to the present invention has a b value, which represents the hue, of preferably 5 or lower. As the b value is smaller, the color is less yellowish, which is good as a hue.

According to the invention, the diol component, which is used in the preparation of the polycarbonates or polyesters, may additionally comprise one or more diol monomers, which are different from the monomer compound of the formula (I), such as one or more monomers of the formula (IV).

Suitable diol monomers, which are different from the monomer compound of the formula (I), are those, which are conventionally used in the preparation of polycarbonates, e.g.
aliphatic diols such as ethylene glycol, propanediol, butanediol, pentanediol and hexanediol;
alicyclic diols such as tricyclo[5.2.1.02,6]decane dimethanol, cyclohexane-1,4-dimethanol, decalin-2,6-dimethanol, norbornane dimethanol, pentacyclopentadecane dimethanol, cyclopentane-1,3-dimethanol, spiroglycol, 1,4:3,6-dianhydro-D-sorbitol, 1,4:3,6-dianhydro-D-mannitol and 1,4:3,6-dianhydro-L-iditol are also included in examples of the diol; and
aromatic diols, in particular aromatic diols of the formula (IV) such as bis(4-hydroxyphenyl)methane, 1,1-bis(4-hydroxyphenyl)ethane, bis(4-hydroxyphenyl)ether, bis(4-hydroxyphenyl)sulfoxide, bis(4-hydroxyphenyl)sulfide, bis(4-hydroxyphenyl)sulfone, bis(4-hydroxyphenyl)ketone, 2,2-bis(4-hydroxyphenyl)propane, 2,2-bis(4-hydroxy-3-t-butylphenyl)propane, 2,2-bis(4-hydroxy-3-methylphenyl)propane, 1,1-bis(4-hydroxyphenyl)cyclopentane, 1,1-bis(4-hydroxyphenyl)cyclohexane, 2,2-bis(4-hydroxyphenyl)hexafluoropropane, bis(4-hydroxyphenyl)diphenylmethane, 1,1-bis(4-hydroxyphenyl)-1-phenylethane, α,ω-bis[2-(phydroxyphenyl)ethyl]polydimethylsiloxane, α,ω-bis[3-(o-hydroxyphenyl)propyl]polydimethylsiloxane, 4,4′-[1,3-phenylenebis(1-methylethylidene)hydroxyphenyl]-1-phenylethane, 9,9-bis(4-hydroxyphenyl)fluorene, 9,9-bis[4-(2-hydroxyethoxy)-3-methylphenyl]fluorene, 9,9-bis[4-(2-hydroxyethoxy)-3-tert-butylphenyl]fluorene, 9,9-bis[4-(2-hydroxyethoxy)-3-isopropylphenyl]fluorene, 9,9-bis[4-(2-hydroxyethoxy)-3-cyclohexylphenyl]fluorene, 9,9-bis(4-hydroxy-3-phenylphenyl)fluorene, 9,9-bis(4-(2-hydroxyethyl)phenyl)fluorene, 9,9-bis(4-(2-hydroxyethyl)-3-phenylphenyl)fluorene, 9,9-bis(6-hydroxy-2-naphthyl)fluorene, 9,9-bis(6-(2-hydroxyethyl)-2-naphthyl)fluorene, 10,10-bis(4-hydroxyphenyl)anthracen-9-on, 10,10-bis(4-(2-hydroxyethyl)phenyl)anthracen-9-on and 2,2′-[1,1′-binaphthalene-2,2′-diylbis(oxy)]diethanol, also termed 2,2′-bis(2-hydroxyethoxy)-1,1′-binaphthyl or 2,2′-bis(2-hydroxyethoxy)-1,1′-binaphthalene (BNE).

Preferably, the diol component comprises at least one monomer of the formula (IV) in addition to the monomer of formula (I). In particular, the total amount of monomers of formulae (I) and (IV) contribute to the diol component by at least 90% by weight, based on the total weight of the diol component or by at least 90 mol-%, based on the total molar amount of the diol monomers of the diol component. In particular, the diol component comprises at least one monomer selected from the monomers of formulae (IV-11) to (IV-20) in addition to the monomer of formula (I). More particularly, the diol component comprises at least one monomer selected from the monomers of formulae (IV-11), (IV-12), (IV-14), (IV-19) and (IV-20) in addition to the monomer of formula (I). Especially, the diol component comprises at least one monomer selected from 2,2′-bis(2-hydroxyethoxy)-1,1′-binaphtyl, 2,2′-bis(2-hydroxyethoxy)-6,6′-diphenyl-1,1′-binaphtyl, 9,9-bis(6-(2-hydroxyethoxy)-2-naphthyl)fluorene, 9,9-bis(4-(2-hydroxyethoxy)phenyl)fluorene and 9,9-bis(4-(2-hydroxyethoxy)-3-phenylphenyl)fluorene and combinations thereof in addition to the monomer of formula (I).

Frequently, the relative amount of monomer compound of formula (I), based on the total weight of the diol component, is at least 1% by weight, preferably at least 2% or at least 5% by weight, in particular at least 8% by weight or at least 10% by weight and especially at least 12% by weight or at least 15% by weight, preferably in the range of 1 to 90% by weight or in the range of 5 to 90% by weight, in particular in the range of 2 to 80% by weight or in the range of 5 to 80% by weight or in the range of 8 to 80% by weight or in the range 10 to 80% by weight, especially in the range of of 5 to 70% by weight or in the range of 8 to 70% by weight or in the range 10 to 70% by weight or in the range of 15 to 70% by weight, but may also be as high as 100% by weight.

Frequently, the relative molar amount of monomer compound of formula (I), based on the total molar of the diol component, is at least 1 mol-%, preferably at least 2 mol-% or at least 5 mol-%, in particular at least 8 mol-% or at least 10 mol-% and especially at least 12 mol-% or at least 15 mol-%, preferably in the range of 1 to 80 mol-% or in the range of 2 to 80 mol-% or in the range of 5 to 80 mol-% or in the range of 8 to 80 mol-%, in particular in the range of 2 to 70 mol-% or in the range of 5 to 70 mol-% or in the range of 8 to 70 mol-% or in the range of 10 to 70 mol-%, especially in the range of 5 to 60 mol-% or in the range of 8 to 60 mol-% or in the range of 10 to 60 mol-% or in the range of 12 to 60 mol-% or in the range of 15 to 60 mol-%, but may also be as high as 100 mol-%.

Consequently, the relative molar amount of monomer compound of formula (IV), based on the total molar of the diol component, will not exceed 99 mol-% or 98 mol-% or 95 mol-%, in particular not exceed 92 mol-% or 90 mol-% and especially not exceed 88 mol-% or 85 mol-%, and is preferably in the range of 20 to 99 mol-% or in the range of 20 to 98 mol-% or in the range of 20 to 95 mol-% or in the range of 20 to 92 mol-%, in particular in the range of 30 to 98 mol-% or in the range of 30 to 95 mol-% or in the range of 30 to 92 mol-% or in the range of 30 to 90 mol-%, especially in the range of 40 to 95 mol-% or in the range of 40 to 92 mol-% or in the range of 40 to 90 mol-% or in the range of 40 to 88 mol-% or in the range of 40 to 85 mol-%, but may also be as high as 99.9 mol-%.

Frequently, the total molar amount of monomers of formula (I) and monomers of formula (IV) is at least 80 mol-%, in particular at least 90 mol-%, especially at least 95 mol-% or up to 100 mol-%, based on the total molar amount of the diol monomers in the diol component.

Examples of further preferred aromatic dihydroxy compound, which can be used in addition to the monomers of formula (I) and optionally monomers of formula (IV) include, but are not limited to bisphenol A, bisphenol AP, bisphenol AF, bisphenol B, bisphenol BP, bisphenol C, bisphenol E, bisphenol F, bisphenol G, bisphenol M, bisphenol S, bisphenol P, bisphenol PH, bisphenol TMC, bisphenol Z and the like.

In order to adjust the molecular weight and the melt viscosity, the monomers forming the thermoplastic polymer may also include a monofunctional compound, in case of polycarbonates a monofunctional alcohol and in case of polyesters a monofunctional alcohol or a monofunctional carboxylic acid. Suitable monoalcohols are butanol, hexanol and octanol. Suitable monocarboxylic acids include e.g. benzoic acid, propionic acid and butyric acid. In order to increase the molecular weight and the melt viscosity, the monomers forming the thermoplastic polymer may also include a polyfunctional compound, in case of polycarbonates a polyfunctional alcohol having three or more hydroxyl groups and in case of polyesters a polyfunctional alcohol having three or more hydroxyl groups or a polyfunctional carboxylic acid having three or more carboxyl groups. Suitable polyfunctional alcohols are e.g. glycerine, trimethylol propane, pentaerythrit and 1,3,5-trihydroxy pentane. Suitable polyfunctional carboxylic acids having three or more carboxyl groups are e.g. trimellitic acid and pyromellitic acid. The total amount of these compounds, will frequently not exceed 10 mol-%, based on the molar amount of the diol component.

Suitable carbonate forming monomers, are those, which are conventionally used as carbonate forming monomers in the preparation of polycarbonates, include, but are not limited to phosgene, diphosgene and diester carbonates such as diethyl carbonate, diphenyl carbonate, di-p-tolyl carbonate, phenyl-p-tolyl carbonate, di-p-chlorophenyl carbonate and dinaphthyl carbonate. Out of these, diphenyl carbonate is particularly preferred. The carbonate forming monomer is frequently used at a ratio of 0.97 to 1.20 mol, and more preferably 0.98 to 1.10 mol, with respect to 1 mol of the dihydroxy compound(s) in total.

Suitable dicarboxylic acids include, but are not limited to
aliphatic dicarboxylic acids such as oxalic acid, malonic acid, succinic acid, glutaric acid, adipic acid, pimelic acid, suberic acid, azelaic acid;
alicyclic dicarboxylic acids such as tricyclo[5.2.1.02,6]decane dicarboxylic acid, cyclohexane-1,4-dicarboxylic acid, decalin-2,6-dicarboxylic acid, and norbornandicarboxylic acid; and
aromatic dicarboxylic acids, such as benzene dicarboxylic acids, specifically phthalic acid, isophthalic acid, 2-methylterephthalic acid or terephthalic acid, and naphthalene dicarboxylic acids, specifically naphthalene-1,3-dicarboxylic acid, naphthalene-1,4-dicarboxylic acid, naphthalene-1,5-dicarboxylic acid, naphthalene-1,6-dicarboxylic acid, naphthalene-1,7-dicarboxylic acid, naphthalene-2,5-dicarboxylic acid, naphthalene-2,6-dicarboxylic acid, 2-[9-(carboxymethyl)fluoren-9-yl]acetic acid (formula DC1), 2-[9-(carboxymethyl)fluoren-9-yl]propionic acid (formula DC2), 2,2′-bis(carboxymethyloxy)-1,1′-binaphthyl (formula DC3) and naphthalene-2,7-dicarboxylic acid.

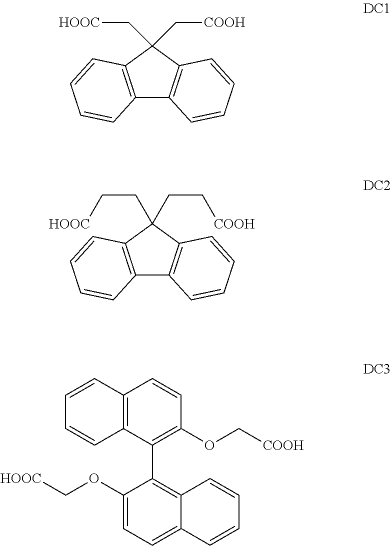 [SMILES: O=C(O)CC1(CC(=O)O)c2ccccc2-c2ccccc21 ; O=C(O)CCC1(CCC(=O)O)c2ccccc2-c2ccccc21 ; O=C(O)COc1ccc2ccccc2c1-c1c(OCC(=O)O)ccc2ccccc12]

Suitable ester forming derivatives of dicarboxylic acids include, but are not limited to the dialkyl esters, the diphenyl esters and the ditolyl esters.

In case of polyesters, the ester forming monomer is frequently used at a ratio of 0.97 to 1.20 mol, and more preferably 0.98 to 1.10 mol, with respect to 1 mol of the dihydroxy compound(s) in total.

The polycarbonates of the present invention can be prepared by reacting a diol component comprising a monomer of formula (I) and optionally a further diol monomer such as a monomer of the formula (IV) and a carbonate forming monomer by analogy to the well known preparation of polycarbonates as described e.g. in U.S. Pat. No. 9,360,593, US 2016/0319069 and US 2017/0276837, to which full reference is made.

The polyesters of the present invention can be prepared by reacting a diol component comprising a monomer of formula (I) and optionally a further diol monomer such as a monomer of the formula (IV) and a dicarboxylic acid or its ester forming derivative by analogy to the well known preparation of polyesters as described e.g. in US 2017/044311 and the references cited therein, to which full reference is made.

The polyestercarbonates of the present invention can be prepared by reacting a diol component comprising a monomer of formula (I) and optionally a further diol monomer such as a monomer of the formula (IV), a carbonate forming monomer and a dicarboxylic acid or its ester forming derivative by analogy to the well known preparation of polyestercarbonates as described in the art.

The polycarbonates, polyesters and polyestercarbonates are usually prepared by reacting the monomers of the diol component with the carbonate forming monomers and/or the ester forming monomers, i.e. the dicarboxylic acids or the ester forming derivatives thereof, in the presence of an esterification catalyst, in particular a transesterification catalyst, in case a carbonate forming monomer or an ester forming derivative of a polycarboxylic acid is used.

Suitable transesterification catalysts are basic compounds, which specifically include but are not limited to alkaline metal compounds, alkaline earth metal compound, nitrogen-containing compounds, and the like. Likewise, suitable transesterification catalysts are acidic compounds, which specifically include but are not limited to Lewis acid compounds of polyvalent metals, including compounds such as zinc, tin, titanium, zirconium, lead, and the like.

Examples of suitable alkaline metal compound include alkaline metal salts of an organic acid such as acetic acid, stearic acid, benzoic acid, or phenylphorsphoric acid, alkaline metal phenolates, alkaline metal oxides, alkaline metal carbonates, alkaline metal borohydrides, alkaline metal hydrogen carbonates, alkaline metal phosphate, alkaline metal hydrogenphosphate, alkaline metal hydroxides, alkaline metal hydrides, alkaline metal alkoxides, and the like. Specific examples thereof include sodium hydroxide, potassium hydroxide, cesium hydroxide, lithium hydroxide, sodium hydrogen carbonate, sodium carbonate, potassium carbonate, cesium carbonate, lithium carbonate, sodium acetate, potassium acetate, cesium acetate, lithium acetate, sodium stearate, potassium stearate, cesium stearate, lithium stearate, sodium borohydride, sodium borophenoxide, sodium benzoate, potassium benzoate, cesium benzoate, lithium benzoate, disodium hydrogen phosphate, dipotassium hydrogen phosphate, dilithium hydrogen phosphate, and disodium phenylphosphate; and also include disodium salt, dipotassium salt, dicesium salt, dilithium salt of bisphenol A, sodium salt, potassium salt, cesium salt and lithium salt of phenol; and the like.

Examples of the alkaline earth metal compound include alkaline earth metal salts of an organic acid such as acetic acid, stearic acid, benzoic acid, or phenylphorsphoric acid, alkaline earth metal phenolates, alkaline earth metal earth oxides, alkaline earth metal carbonates, alkaline metal borohydrides, alkaline earth metal hydrogen carbonates, alkaline earth metal hydroxides, alkaline earth metal hydrides, alkaline earth metal alkoxides, and the like. Specific examples thereof include magnesium hydroxide, calcium hydroxide, strontium hydroxide, barium hydroxide, magnesium hydrogen carbonate, calcium hydrogen carbonate, strontium hydrogen carbonate, barium hydrogen carbonate, magnesium carbonate, calcium carbonate, strontium carbonate, barium carbonate, magnesium acetate, calcium acetate, strontium acetate, barium acetate, magnesium stearate, calcium stearate, calcium benzoate, magnesium phenylphosphate, and the like.

Examples of the nitrogen-containing compound include quaternary ammoniumhydroxide, salt thereof, amines, and the like. Specific examples thereof include quaternary ammoniumhydroxides including an alkyl group, an aryl group or the like, such as tetramethylammoniumhydroxide, tetraethylammoniumhydroxide, tetrapropylammoniumhydroxide, tetrabutylammoniumhydroxide, trimethylbenzylammoniumhydroxide, and the like; tertiary amines such as triphenylamine, dimethylbenzylamine, triphenylamine, and the like; secondary amines such as diethylamine, dibutylamine, and the like; primary amines such as propylamine, butylamine, and the like; imidazoles such as 2-methylimidazole, 2-phenylimidazole, benzoimidazole, and the like; bases or basic salts such as ammonia, tetramethylammoniumborohydride, tetrabutylammoniumborohydride, tetrabutylammoniumtetraphenylborate, tetraphenylammoniumtetraphenylborate, and the like.

Preferred examples of the transesterification catalyst include salts of polyvalent metals such as zinc, tin, titanium, zirconium, lead, and the like, in particular the chlorides, alkoxyides, alkanoates, benzoates, acetylacetonates and the like. They may be used independently or in a combination of two or more. Specific examples of such transesterification catalyst include zinc acetate, zinc benzoate, zinc 2-ethylhexanoate, tin chloride (II), tin chloride (IV), tin acetate (II), tin acetate (IV), dibutyltinlaurate, dibutyltinoxide, dibutyltinmethoxide, zirconiumacetylacetonate, zirconium oxyacetate, zirconiumtetrabutoxide, lead acetate (II), lead acetate (IV), and the like.

The transesterification catalyst are frequently used at a ratio of 10⁻⁹ to 10⁻³ mol, preferably 10⁻⁷ to 10⁻⁴ mol, with respect to 1 mol of the dihydroxy compound(s) in total.

Frequently, the polycarbonates, polyesters and polyestercarbonates are prepared by a melt polycondensation method. In the melt polycondensation the monomers are reacted in the absence of an additional inert solvent. While the reaction is performed any byproduct formed in the transesterification reaction is removed by heating the reaction mixture at ambient pressure or reduced pressure.

The melt polycondensation reaction preferably comprises charging the monomers and catalyst into a reactor and subjecting the reaction mixture to conditions, where the reaction between the monomers and the formation of the byproduct takes place. It has been found advantages, if the byproduct resides for at least a while in the polycondensation reaction. However, in order to drive the polycondensation reaction to the product side, it is beneficial to remove at least a portion of the formed byproduct during or preferably at the end of the polycondensation reaction. In order to allow the byproduct in the reaction mixture, the pressure may be controlled by closing the reactor, or by increasing or decreasing the pressure. The reaction time for this step is 20 minutes or longer and 240 minutes or shorter, preferably 40 minutes or longer and 180 minutes or shorter, and especially preferably 60 minutes or longer and 150 minutes or shorter. In this step, in the case where the byproduct is removed by distillation soon after being generated, the finally obtained thermoplastic resin has a low content of high molecular-weight resin molecules. By contrast, in the case where the byproduct is allowed to reside in the reactor for a certain time, the finally obtained thermoplastic resin has a high content of high molecular-weight resin molecules.

The melt polycondensation reaction may be performed in a continuous system or in a batch system. The reactor usable for the reaction may be of a vertical type including an anchor-type stirring blade, a Maxblend® stirring blade, a helical ribbon-type stirring blade or the like; of a horizontal type including a paddle blade, a lattice blade, an eye glass-type blade or the like; or an extruder type including a screw. A reactor including a combination of such reactors is preferably usable in consideration of the viscosity of the polymerization product.

According to the method for producing the thermoplastic resin, such as a polycarbonate resin, after the polymerization reaction is finished, the catalyst may be removed or deactivated in order to maintain the thermal stability and the hydrolysis stability. A preferred method for deactivating the catalyst is the addition of an acidic substance. Specific examples of the acidic substance include esters such as butyl benzoate and the like; aromatic sulfonates such as p-toluenesulfonic acid and the like; aromatic sulfonic acid esters such as butyl p-toluenesulfonate, hexyl p-toluenesulfonate, and the like; phosphoric acids such as phosphorous acid, phosphoric acid, phosphonic acid, and the like; phosphorous acid esters such as triphenyl phosphite, monophenyl phosphite, diphenyl phosphite, diethyl phosphite, di-n-propyl phosphite, di-n-butyl phosphite, di-n-hexyl phosphite, dioctyl phosphite, monooctyl phosphite, and the like; phosphoric acid esters such as triphenyl phosphate, diphenyl phosphate, monophenyl phosphate, dibutyl phosphate, dioctyl phosphate, monooctyl phosphate, and the like; phosphonic acids such as diphenyl phosphonic acid, dioctyl phosphonic acid, dibutyl phosphonic acid, and the like; phosphonic acid esters such as diethyl phenylphosphonate, and the like; phosphines such as triphenylphosphine, bis(diphenylphosphino)ethane, and the like; boric acids such as boric acid, phenylboric acid, and the like; aromatic sulfonic acid salts such as tetarabutylphosphonium dodecylbenzensulfonate salt, and the like; organic halides such as chloride stearate, benzoyl chloride, chloride p-toluenesulfonate, and the like; alkylsulfonic acids such as dimethylsulfonic acid, and the like; organic halides such as benzyl chloride, and the like. These deactivators are frequently used at 0.01 to 50 mol, preferably 0.3 to 20 mol, with respect to the catalyst. After the catalyst has been deactivated, there may be a step of removing low boiling point compounds from the polymer by distillation. The distillation is preferably performed at reduced pressure, e.g. at a pressure of 0.1 to 1 mm Hg at a temperature of 200 to 350° C. For this step, a horizontal device including a stirring blade having a high surface renewal capability such as a paddle blade, a lattice blade, an eye glass-type blade or the like, or a thin film evaporator is preferably used.

It is desirable that the thermoplastic resin such as a polycarbonate resin has a very small amount of foreign objects. Therefore, the molten product is preferably filtered to remove any solids from the melt. The mesh of the filter is preferably 5 μm or less, and more preferably 1 μm or less. It is preferred that the generated polymer is filtrated by a polymer filter. The mesh of the polymer filter is preferably 100 μm or less, and more preferably 30 μm or less. A step of sampling a resin pellet needs to be performed in a low dust environment, needless to say. The dust environment is preferably of class 6 or lower, and more preferably of class 5 or lower.

The thermoplastic resin may be molded by any conventional molding procedure for producing optical elements. Suitable molding procedures include but are not limited to injection molding, compression molding, casting, roll processing, extrusion molding, extension and the like.

While it is possible to mold the thermoplastic resin of the invention as such, it is also possible to mold a resin composition, which contains at least one thermoplastic resin of the invention and which further contains at least one additive and/or further resin. Suitable additives include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like. Suitable further resins are e.g. another polycarbonate resin, polyester carbonate resin, polyester resin, polyamide, polyacetal and the like, which does not contain repeating units of the formula (I).

Examples of the antioxidant include but are not limited to triethyleneglycol-bis[3-(3-tertbutyl-5-methyl-4-hydroxyphenyl)propionate], 1,6-hexanediol-bis[3-(3,5-di-tert-butyl-4-hydroxyphenyl)propionate], pentaerythritol-tetrakis[3-(3,5-di-tert-butyl-4-hydroxyphenyl)propionate], octadecyl-3-(3,5-di-tert-butyl-4-hydroxyphenyl)propionate, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2 (3H)-one, 5,7-Di-tert-butyl-3-(1,2dimethylphenyl)benzofuran-2 (3H)-one, 1,3,5-trimethyl-2,4,6-tris(3,5-di-tert-butyl-4-hydroxybenzyl)benzene, N,N-hexamethylenebis(3,5-di-tert-butyl-4-hydroxy-hydrocinnamide, 3,5-di-tert-butyl-4-hydroxy-benzylphosphonate-diethylester, tris(3,5-di-tert-butyl-4-hydroxybenzyl)isocyanurate, and 3,9-bis{1,1-dimethyl-2-[β-(3-tert-butyl-4-hydroxy-5-methylphenyl)propionyloxy]ethyl}-2,4,8,10-tetraoxaspiro(5,5)undecane, and the like. Among these examples, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2 (3H)-one, and 5,7-Di-tert-butyl-3-(1,2dimethylphenyl)benzofuran-2 (3H)-one are more preferred. The content of the antioxidant in the thermoplastic resin is preferably 0.001 to 0.3 parts by weight with respect to 100 parts by weight of the thermoplastic resin.

Examples of the processing stabilizer include but are not limited to phosphorus-based processing stabilizers, sulfur-based processing stabilizers, and the like. Examples of the phosphorus-based processing stabilizer include phosphorous acid, phosphoric acid, phosphonous acid, phosphonic acid, esters thereof, and the like. Specific examples thereof include triphenylphosphite, tris(nonylphenyl)phosphite, tris(2,4-di-tert-butylphenyl)phosphite, tris(2,6-di-tert-butylphenyl)phosphite, tridecylphosphite, trioctylphosphite, trioctadecylphosphite, didecylmonophenylphosphite, dioctylmonophenylphosphite, diisopropylmonophenylphosphite, monobutyldiphenylphosphite, monodecyldiphenylphosphite, monooctyldiphenylphosphite, bis(2,6-di-tert-butyl-4-methylphenyl)pentaerythritoldiphosphite, 2,2-methylenebis(4,6-di-tertbutylphenyl)octylphosphite, bis(nonylphenyl)pentaerythritoldiphosphite, bis(2,4-dicumylphenyl)pentaerythritoldiphosphite, bis(2,4-di-tert-butylphenyl)pentaerythritoldiphosphite, distearylpentaerythritoldiphosphite, tributylphosphate, triethylphosphate, trimethylphosphate, triphenylphosphate, diphenylmono-orthoxenylphosphate, dibutylphosphate, dioctylphosphate, diisopropylphosphate, dimethyl benzenephosphonate, diethyl benzenephosphonate, dipropyl benzenephosphonate, tetrakis(2,4-di-tbutylphenyl)-4,4′-biphenylenediphosphonite, tetrakis(2,4-di-t-butylphenyl)-4,3′-biphenylenediphosphonite, tetrakis(2,4-di-t-butylphenyl)-3,3′-biphenylenediphosphonite, bis(2,4-di-tert-butylphenyl)-4-phenyl-phenylphosphonite, bis(2,4-di-tert-butylphenyl)-3-phenyl-phenylphosphonite, and the like. The content of the phosphorus-based processing stabilizer in the thermoplastic resin composition is preferably 0.001 to 0.2 parts by weight with respect to 100 parts by weight of the thermoplastic resin.

Examples of the sulfur-based processing stabilizer include but are not limited to pentaerythritol-tetrakis(3-laurylthiopropionate), pentaerythritol-tetrakis(3-myristylthiopropionate), pentaerythritol-tetrakis(3-stearylthiopropionate), dilauryl-3,3′-thiodipropionate, dimyristyl-3,3′-thiodipropionate, distearyl-3,3′-thiodipropionate, and the like. The content of the sulfur-based processing stabilizer in the thermoplastic resin compositon is preferably 0.001 to 0.2 parts by weight with respect to 100 parts by weight of the thermoplastic resin.

Preferred releasing agents contain at least 90% by weight of an ester of an alcohol and a fatty acid. Specific examples of the ester of an alcohol and a fatty acid include an ester of a monovalent alcohol and a fatty acid, and a partial ester or a total ester of a polyvalent alcohol and a fatty acid. Preferred examples of the above-described ester of an alcohol and a fatty acid include the esters of a monovalent alcohol having a carbon number of 1 to 20 and a saturated fatty acid having a carbon number of 10 to 30. Preferred examples of partial or total esters of a polyvalent alcohol and a fatty acid include the partial or total ester of a polyvalent alcohol having a carbon number of 2 to 25 and a saturated fatty acid having a carbon number of 10 to 30. Specific examples of the ester of a monovalent alcohol and a fatty acid include stearyl stearate, palmityl palmitate, butyl stearate, methyl laurate, isopropyl palmitate, and the like. Specific examples of the partial or total ester of a polyvalent alcohol and a fatty acid include monoglyceride stearate, monoglyceride stearate, diglyceride stearate, triglyceride stearate, monosorbitate stearate, monoglyceride behenate, monoglyceride caprylate, monoglyceride laurate, pentaerythritol monostearate, pentaerythritol tetrastearate, pentaerythritol tetrapelargonate, propyleneglycol monostearate, biphenyl biphenate, sorbitan monostearate, 2-ethylhexylstearate, total or partial esters of dipentaerythritol such as dipentaerythritol hexastearate and the like, etc. The content of the releasing agent in the resin composition is preferably 0.005 to 2.0 parts by weight, more preferably 0.01 to 0.6 parts by weight, and still more preferably 0.02 to 0.5 parts by weight, with respect to 100 parts by weight of the thermoplastic resin.

Preferred ultraviolet absorbers are selected from the group consisting of benzotriazole-based ultraviolet absorbers, benzophenone-based ultraviolet absorbers, triazine-based ultraviolet absorbers, cyclic iminoester-based ultraviolet absorbers, and cyanoacrylate-based ultraviolet absorbers. Namely, the following ultraviolet absorbers may be used independently or in a combination of two or more.

Examples of benzotriazole-based ultraviolet absorbers include 2-(2-hydroxy-5-methylphenyl)benzotriazole, 2-(2-hydroxy-5-tert-octylphenyl)benzotriazole, 2-(2-hydroxy-3,5-dicumylphenyl)phenylbenzotriazole, 2-(2-hydroxy-3-tert-butyl-5-methylphenyl)-5-chlorobenzotriazole, 2,2′-methylenebis[4-(1,1,3,3-tetramethylbutyl)-6-(2N-benzotriazole-2-yl)phenol)], 2-(2-hydroxy-3,5-di-tert-butylphenyl)benzotriazole, 2-(2-hydroxy-3,5-di-tert-butylphenyl)-5-chlorobenzotriazole, 2-(2-hydroxy-3,5-di-tertamylphenyl)benzotriazole, 2-(2-hydroxy-5-tert-octylphenyl)benzotriazole, 2-(2-hydroxy-5-tert-butylphenyl)benzotriazole, 2-(2-hydroxy-4-octoxyphenyl)benzotriazole, 2,2′-methylenebis(4-cumyl-6-benzotriazolephenyl), 2,2′-p-phenylenebis(1,3-benzoxazine-4-one), 2-[2-hydroxy-3-(3,4,5,6-tetrahydrophthalimidemethyl)-5-methylphenyl]benzotriazole, and the like.

Examples of benzophenone-based ultraviolet absorbers include 2,4-dihydroxybenzophenone, 2-hydroxy-4-methoxybenzophenone, 2-hydroxy-4-octoxybenzophenone, 2-hydroxy-4-benzyloxybenzophenone, 2-hydroxy-4-methoxy-5-sulfoxybenzophenone, 2-hydroxy-4-methoxybenzophenone-5-sulfonic acid hydrate, 2,2′-dihydroxy-4-methoxybenzophenone, 2,2′,4,4′-tetrahydroxybenzophenone, 2,2′-dihydroxy-4,4′-dimethoxybenzophenone, 2,2′-dihydroxy-4,4′-dimethoxy-5-sodiumsulfoxybenzophenone, bis(5-benzoyl-4-hydroxy-2-methoxyphenyl)methane, 2-hydroxy-4-n-dodecyloxybenzophenone, 2-hydroxy-4-methoxy-2′-carboxybenzophenone, and the like.

Examples of triazine-based ultraviolet absorbers include 2-(4,6-diphenyl-1,3,5-triazine-2-yl)-5-([(hexyl)oxy]-phenol, 2-(4,6-bis(2,4-dimethylphenyl)-1,3,5-triazine-2-yl)-5-([(octyl)oxy]-phenol, and the like.

Examples of cyclic iminoester-based ultraviolet absorbers include 2,2′-bis(3,1-benzoxazine-4-one), 2,2′-p-phenylenebis(3,1-benzoxazine-4-one), 2,2′-m-phenylenebis(3,1-benzoxazine-4-one), 2,2′-(4,4′diphenylene)bis(3,1-benzoxazine-4-one), 2,2′-(2,6-naphthalene)bis(3,1-benzoxazine-4-one), 2,2′-(1,5-naphthalene)bis(3,1-benzoxazine-4-one), 2,2′-(2-methyl-p-phenylene)bis(3,1-benzoxazine-4-one), 2,2′-(2-nitro-p-phenylene)bis(3,1-benzoxazine-4-one), 2,2′-(2-chloro-p-phenylene)bis(3,1-benzoxazine-4-one), and the like.

Examples of cyanoacrylate-based ultraviolet absorbers include 1,3-bis-[(2′-cyano-3′,3′-diphenylacryloyl)oxy]-2,2-bis(((2-cyano-3,3-diphenylacryloyl)oxy)methyl)propane, 1,3-bis-[(2-cyano-3,3-diphenylacryloyl)oxy]benzene, and the like.

The content of the ultraviolet absorber in the resin composition is preferably 0.01 to 3.0 parts by weight, more preferably 0.02 to 1.0 parts by weight, and still more preferably 0.05 to 0.8 parts by weight, with respect to 100 parts by weight of the thermoplastic resin. The ultraviolet absorber contained in such a range of content in accordance with the use may provide a sufficient climate resistance to the thermoplastic resin.

As mentioned above, the thermoplastic polymer resins, in particular the polycarbonate resins, comprising repeating units of formulae (II), (IIa), (IIa-1) and (IIa-2), respectively, as described herein, provide high transparency and high refractive index to thermoplastic resins, which therefore are suitable for preparing optical devices, where high transparency and high refractive index is required. More precisely, the thermoplastic polycarbonates having structural units of formulae (II), (IIa), (IIa-1) and (IIa-2), respectively, are characterized by having a high refractive index, which is preferably at least 1.660, more preferably at least 1.680, in particular at least 1.690.

The contribution of the monomer of the formulae (I), (Ia), (Ia-1) and (Ia-2), respectively, to the refractive index of the thermoplastic resin, in particular a polycarbonate resin, will depend from the refractive index of said monomer and the relative amount of said monomer in the thermoplastic resin. In general, a higher refractive index of the monomer contained in the thermoplastic resin will result in a higher refractive index of the resulting thermoplastic resin. Apart from that, the refractive index of a thermoplastic resin comprising structural units of the formula (II) can be calculated from the refractive indices of the monomers used for preparing the thermoplastic resin, either from the refractive index of the monomers or ab initio, e.g. by using the computer software ACD/ChemSketch 2012 (Advanced Chemistry Development, Inc.).

In case of thermoplastic copolymer resins, the refractive index of the thermoplastic resin, in particular a polycarbonate resin, can be calculated from the refractive indices of the homopolymers of the respective monomers, which form the copolymer resin, by the following so called “Fox equation”:

$$1 / {n}_{D} = {x}_{1} / {n}_{D 1} + {x}_{2} / {n}_{D 2} + … + {x}_{n} / {n}_{Dn} ,$$

where n_{D} is the refractive index of the copolymer, x₁, x₂, . . . xₙ are the mass fractions of the monomers 1, 2, . . . n in the copolymer and n_{D1}, n_{D2}, . . . n_{Dn} are the refractive indices of the homopolymers synthesized from only one of the monomers 1, 2, . . . n at a time. In case of polycarbonates, x₁, x₂, . . . xₙ are the mass fractions of the OH monomers 1, 2, . . . n, based on the total amount of OH monomer. It is apparent that a higher refractive index of a homopolymer will result in a higher refractive index of the copolymer.

The refractive indices of the thermoplastic resins can be determined directly or indirectly. For direct determination, the refractive indices n_{D} of the thermoplastic resins are measured at wavelength of 589 nm in accordance with the protocol JIS-K-7142 using an Abbe refractometer and applying a 0.1 mm film of the thermoplastic resin. In case of the refractive indices of the homopolycarbonates of the compounds of formula (I), the refractive indices can also be determined indirectly. For this, a co-polycarbonate of the respective monomer of formula (I) with 9,9-bis(4-(2-hydroxyethoxy)phenyl)fluorene and diphenyl carbonate is prepared according to the protocol of example 1 in column 48 of U.S. Pat. No. 9,360,593 and the refractive indices n_{D} of the co-polycarbonate is measured at wavelength of 589 nm in accordance with the protocol JIS-K-7142 using an Abbe refractometer and applying a 0.1 mm film of the co-polycarbonate. From the thus measured refractive indices n_{D}, the refractive index of the homopolycarbonate of the respective monomer can be calculated by applying the Fox equation and the known refractive index of 9,9-bis(4-(2-hydroxyethoxy)phenyl)fluorene (n_{D}(589 nm)=1.639).

As mentioned before, compounds of formula (I), which do not bear color-imparting radicals, such as some of the radicals R¹, R², R³, R⁴ and Ar¹, can also be obtained in a purity, which provides for a low yellowness index Y.I., as determined in accordance with ASTM E313, which may also be important for the use in the preparation of optical resins.

More precisely, the yellowness index Y.I., as determined in accordance with ASTM E313, of the compounds of formula (I) preferably does not exceed 200, more preferably 100, even more preferably 50, in particular 20 or 10.

The thermoplastic resin according to the present invention has a high refractive index and a low Abbe number. The thermoplastic resin of the present invention can be used for producing a transparent conductive substrate usable for a liquid crystal display, an organic EL display, a solar cell and the like. Also, the thermoplastic resin of the present invention can be used as a structural material for optical parts, such as, optical disks, liquid crystal panels, optical cards, optical sheets, optical fibers, connectors, evaporated plastic reflecting mirrors, displays, and the like; or used as optical devices suitable for functional material purpose.

Accordingly, molded articles, such as optical devices can be formed using the thermoplastic resins of the present invention. The optical devices include optical lenses, and optical films. The specific examples of the optical devices include lenses, films, mirrors, filters, prisms, and so on. These optical devices can be formed by arbitrary production process, for example, by injection molding, compression molding, injection compression molding, extrusion molding, or solution casting.

Because of an excellent moldability and a high heat resistance, the thermoplastic resins of the present invention are very suitable for production of optical lenses which requires injection molding. For molding, the thermoplastic resins of the present invention, such as the polycarbonate resin, can be used with other thermoplastic resins, for example, different polycarbonate resin, polyestercarbonate resin, polyester resin, and other resins, as a mixture.

In addition, the thermoplastic resins of the present invention can be mixed with additives for forming the optical devices. As the additives for forming the optical devices, above-mentioned ones can be used. The additives may include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like.

As is clear from the above, another aspect of the present invention relates to an optical device made of a thermoplastic resin as defined above, where the thermoplastic resin comprises a structural unit represented by the formula (II) and optionally of formula (V). As regards to the preferred meanings and preferred embodiments of the structural units of the formulae (II) and (V), reference is made to the statements given above.

An optical device made of an optical resin comprising the repeating units of the formula (II) and optionally repeating units of the formula (V) as defined herein are usually optical molded articles such as optical lenses, for example car head lamp lenses, Fresnel lenses, fθ lenses for laser printers, camera lenses, lenses for glasses and projection lenses for rear projection TV's, CD-ROM pick-up lenses, but also optical disks, optical elements for image display media, optical films, film substrates, optical filters or prisms, liquid crystal panels, optical cards, optical sheets, optical fibers, optical connectors, eposition plastic reflective mirrors, and the like. Here particular preference is given to optical lenses and optical films. Optical resins comprising repeating units of the formula (II) and optionally repeating units of the formula (V) are also useful for producing a transparent conductive substrate usable for an optical device suitable as a structural member or a functional member of a transparent conductive substrate for a liquid crystal display, an organic EL display, a solar cell and the like.

The optical lens produced from the thermoplastic resin according to the present invention has a high refractive index, a low Abbe number and a low degree of birefringence, and is highly moisture and heat resistant. Therefore, the optical lens can be used in the field in which a costly glass lens having a high refractive index is conventionally used, such as for a telescope, binoculars, a TV projector and the like. It is preferred that the optical lens is used in the form of an aspherical lens. Merely one aspherical lens may make the spherical aberration substantially zero. Therefore, it is not necessary to use a plurality of spherical lenses to remove the spherical aberration. Thereby the weight and the production cost of a device including the spherical aberration is decreased. An aspherical lens is useful especially as a camera lens among various types of optical lenses. The present invention easily provides an aspherical lens having a high refractive index and a low level of birefringence, which is technologically difficult to produce by processing glass.

An optical lens of the present invention may be formed, for example, by injection molding, compression molding, injection compression molding or casting the resin the repeating units of the formula (II) and optionally repeating units of the formula (V) as defined herein.

The optical lens of the present invention is characterized by a small optical distortion. An optical lens comprising a conventional optical resin has a large optical distortion. Although it is not impossible to reduce the value of an optical distortion by molding conditions, the condition widths are very small, thereby making molding extremely difficult. Since the resin having repeating units of the formula (II) and optionally repeating units of the formula (V) as defined herein has an extremely small optical distortion caused by the orientation of the resin and a small molding distortion, an excellent optical element can be obtained without setting molding conditions strictly.

To manufacture the optical lens of the present invention by injection molding, it is preferred that the lens should be molded at a cylinder temperature of 260° C. to 320° C. and a mold temperature of 100° C. to 140° C.

The optical lens of the present invention is advantageously used as an aspherical lens as required. Since spherical aberration can be substantially nullified with a single aspherical lens, spherical aberration does not need to be removed with a combination of spherical lenses, thereby making it possible to reduce the weight and the production cost. Therefore, out of optical lenses, the aspherical lens is particularly useful as a camera lens.

Since resins having repeating units of the formula (II) and optionally repeating units of the formula (V) as defined herein have a high moldability, they are particularly useful as the material of an optical lens, which is thin and small in size and has a complex shape. As a lens size, the thickness of the center part of the lens is 0.05 to 3.0 mm, preferably 0.05 to 2.0 mm, more preferably 0.1 to 2.0 mm. The diameter of the lens is 1.0 to 20.0 mm, preferably 1.0 to 10.0 mm, more preferably 3.0 to 10.0 mm. It is preferably a meniscus lens, which is convex on one side and concave on the other side.

The surface of the optical lens of the present invention may have a coating layer such as an antireflection layer or a hard coat layer as required. The antireflection layer may be a single layer or a multi-layer and composed of an organic material or inorganic material but preferably an inorganic material. Examples of the inorganic material include oxides and fluorides such as silicon oxide, aluminum oxide, zirconium oxide, titanium oxide, cerium oxide, magnesium oxide and magnesium fluoride.

The optical lens of the present invention may be formed by an arbitrary method such as metal molding, cutting, polishing, laser machining, discharge machining or edging. Metal molding is preferred.

An optical film produced by the use of the thermoplastic resin according to the present invention is high in transparency and heat resistance, and therefore is preferably usable for a liquid crystal substrate film, an optical memory card or the like. In order to avoid foreign objects from being incorporated into the optical film as much as possible, the molding needs to be performed in a low dust environment, needless to say. The dust environment is preferably of class 6 or lower, and more preferably of class 5 or lower.

The following examples serve as further illustration of the invention.

### 1. Abbreviations

m.p.: melting point
RT: room temperature
THF: tetrahydrofuran
TLC: thin layer chromatography

### 2. Preparation of Monomers of Formula (I)

### 2.1 Analytics Relating to Monomers of Formula (I):

¹H-NMR spectra were determined at 23° C. using a 80 MHz NMR-spectrometer (Magritek Spinsolve 80). If not stated otherwise the solvent was CDCl₃.

IR spectra were recorded by ATR FT-IR, using a Shimadzu FTIR-8400S spectrometer (no. of scans: 45, resolution: 4 cm⁻¹; apodization: Happ-Genzel).

DSC (differential scanning calorimetry) measurements were performed using a Linseis Chip-DSC 10.

Melting points of the compounds were determined by Buchi Melting Point B-545.

UPLC (Ultra Performance Liquid Chromatography) analyses were carried out using the following system and conditions:

Waters Acquity UPLC H-Class Systems; column: Acquity UPLC BEH C18, 1.7 μm, 2.1×100 mm; column temperature: 25° C., gradient: acetonitrile/water: with acetonitrile at 0 min 80%, at 4.0 min 100%; at 6.0 min 100%; at 6.1 min 80%; at 8.0 min 80%); injection volume: 2.0 μl; run time: 8 min; detection at 210 nm.

The yellowness index YI of the compounds of formula (I) can be determined by analogy with ASTM E313 using the following protocol: 1 g of the compound of formula (I) is dissolved in 19 g of a solvent, e.g. methanol or methylene chloride. The solution is transferred into a 50 mm cuvette and transmission is determined in the range of 300 to 800 nm by a Shimadzu UV-Visible spectrophotometer UV-1900. The solvent itself, e.g. methanol, is used as a reference. From the spectra the yellowness index can be calculated by using the Software “RCA-software UV2DAT” in accordance with ASTM E308 (Standard practice for computing the colors of objects by using the CIE System) and ASTM E 313 (Standard practice for calculating yellowness and whiteness indices from instrumentally measured color coordinates).

### 2.2 Preparation Examples

### Example 1: 2-[4-[1-[4-(2-hydroxyethoxy)-3,5-diphenyl-phenyl]-1-methyl-ethyl]-2,6-diphenyl-phenoxy]ethanol (compound of formula (Ia-1), with X=C(CH₃)₂, R¹=R²=R³=R⁴=phenyl and Z¹=Z²=2-hydroxyethyl; compound 33 of table A)

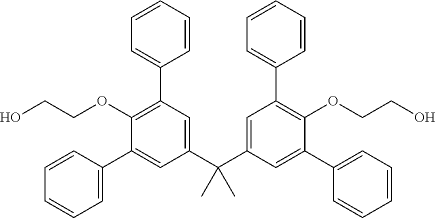 [SMILES: CC(C)(c1cc(-c2ccccc2)c(OCCO)c(-c2ccccc2)c1)c1cc(-c2ccccc2)c(OCCO)c(-c2ccccc2)c1]

To 2-[2,6-dibromo-4-[1-[3,5-dibromo-4-(2-hydroxyethoxy)phenyl]-1-methylethyl]phenoxy]ethanol (135.91 g; 200 mmol; purity=93%) were added phenylboronic acid (102.42 g; 840 mmol; 4.2 equivalents), tris(o-tolyl)phosphane (243.5 mg; 0.8 mmol) and anisole (800 mL). To this mixture were added K₃PO₄ (178.3 g) dissolved in water (396 g). The mixture was stirred at 60-70° C. until two clear phases were formed and was purged with nitrogen. To this mixture was added Pd(OCOCH₃)₂ (45 mg; 0.2 mmol) under argon and the mixture was stirred under reflux until the TLC showed a complete conversion. The reaction mixture was then cooled to 70° C. and the organic layer was separated at this temperature and washed subsequently with an aqueous solution of NaOH (10% by weight), an aqueous solution of HCl (2 M) and brine. The organic layer was treated with activated charcoal (Norit® DX Ultra, Cabot Corp.) and the mixture was stirred for 2.5 hours. Then, the mixture was filtered over celite and the solution was concentrated under reduced pressure. The obtained mixture was cooled to RT and stirred overnight. The formed crystals were collected by filtration to yield the crude title compound as off-white solid (97.8 g; 79% yield). The crude material was recrystallized from acetone or ethanol mixture to give the title compound as a white solid with chemical purity of >99% and a yellowness index of 1.3 (APHA 5).

m.p. (DSC): 108.3° C.;

¹H NMR (80 MHz, CDCl₃): δ=7.93-7.31 (m, 24H, 3.44 (m, 8H), 1.72 (s, 6H), 1.2 (s, 2H, OH) ppm.

IR (ATR): 725.3 (84.84); 746.5 (60.86); 842.9 (88.30); 883.4 (72.19); 1008.8 (67.89); 1022.3 (74.21); 1072.5 (80.22); 1182.4 88.48); 1211.3 (73.28); 1361.8 (85.89); 1421.6 (72.15); 1467.9 (75.76); 1597.1 (90.24); 2357.1 (92.49); 2883.7 (89.41); 2935.8 (89.02); 2958.9 (86.06); 3030.3 (90.52); 3057.3 (90.63); 3184.6 (91.52); 3352.4 (90.54) cm⁻¹.

### Example 2: 2-[4-[1-[4-(2-hydroxyethoxy)-3,5-di(napthalen-1-yl)phenyl]-1-methyl-ethyl]-2,6-di(naphthalen-1-yl)phenoxy]ethanol (compound of formula (Ia-1), with X=C(CH₃)₂, R¹=R²=R³=R⁴=naphthalen-1-yl and Z¹=Z²=2-hydroxyethyl; compound 34 of Table A)

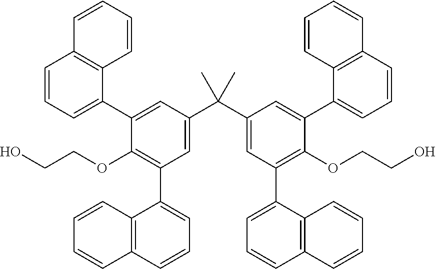 [SMILES: CC(C)(c1cc(-c2cccc3ccccc23)c(OCCO)c(-c2cccc3ccccc23)c1)c1cc(-c2cccc3ccccc23)c(OCCO)c(-c2cccc3ccccc23)c1]

To 2-[2,6-dibromo-4-[1-[3,5-dibromo-4-(2-hydroxyethoxy)phenyl]-1-methylethyl]phenoxy]ethanol (90.4 g; 133 mmol; purity=93%) were added naphthalen-1-ylboronic acid (114.4 g; 665 mmol; 5 equivalents) and tris(o-tolyl)phosphane (1.62 g; 5.32 mmol). To this mixture were added anisole (800 mL) and 148.2 g K₃PO₄ (148.2 g) dissolved in water (331 g). The mixture was stirred at 60-70° C. until two clear phases were formed and was purged with nitrogen. To this mixture was added Pd(OCOCH₃)₂ (299 mg; 1.33 mmol) under argon and the mixture was stirred under reflux until the TLC showed a complete conversion. The reaction mixture was then cooled to 70° C. and the organic layer was separated at this temperature and washed subsequently with an aqueous solution of NaOH (10% by weight), an aqueous solution of HCl (2 M) and brine. The organic layer was treated with activated charcoal (Norit® DX Ultra, Cabot Corp.) and the mixture was stirred for 2.5 hours. Then, the mixture was filtered over celite and the solution was concentrated under reduced pressure. The obtained mixture was cooled to RT and stirred overnight. The formed crystals were collected by filtration to yield the crude title compound as off-white solid (86.3 g; 83% yield) with chemical purity of 96.7%. The crude material can be recrystallized from anisole or toluene/MeOH mixture.

m.p.=235.6-236.9° C.;

m.p. (DSC): 232.9° C.;

¹H NMR (80 MHz, CDCl₃): δ=8.07-7.12 (m, 32H, 3.24-2.76 (m, 4H), 3.07-2.59 (m, 4H), 1.81 (s, 6H), −0.72 (s, 2H, OH) ppm.

IR (ATR): 731.1 (78.73); 777.3 (46.43); 798.6 (65.68); 889.2 (80.73); 1012.7 (70.29); 1068.6 (73.00); 1114.9 (85.15); 1221.0 (74.61); 1334.8 (86.63); 1386.9 (76.67); 1456.3 (82.21); 2870.2 (90.63); 2935.8 (90.42); 2964.7 (90.34); 3057.3 (90.78); 3556.9 (88.32) cm⁻¹.

### Example 3: 2-[4-[1-[4-(2-hydroxyethoxy)-3,5-di(napthalen-2-yl)phenyl]-1-methyl-ethyl]-2,6-di(naphthalen-2-yl)phenoxy]ethanol (compound of formula (Ia-1), with X=C(CH₃)₂, R¹=R²=R³=R⁴=naphthalen-2-yl and Z¹=Z²=2-hydroxyethyl; compound 35 of table A)

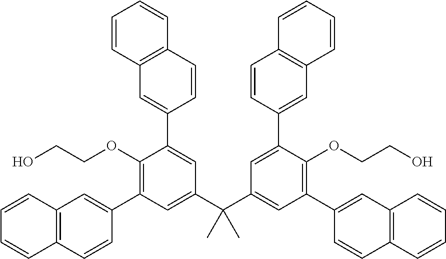 [SMILES: CC(C)(c1cc(-c2ccc3ccccc3c2)c(OCCO)c(-c2ccc3ccccc3c2)c1)c1cc(-c2ccc3ccccc3c2)c(OCCO)c(-c2ccc3ccccc3c2)c1]

To 2-[2,6-dibromo-4-[1-[3,5-dibromo-4-(2-hydroxyethoxy)phenyl]-1-methylethyl]phenoxy]ethanol (90.4 g; 133 mmol; purity=93%) were added naphthalen-2-ylboronic acid (138 g; 800 mmol; 6 equivalents) and tris(o-tolyl)phosphane (1.62 g; 5.32 mmol). To this mixture were added anisole (800 mL) and K₃PO₄ (178 g) dissolved in water (396 g). The mixture was stirred at 60-70° C. until two clear phases were formed and was purged with nitrogen. To this mixture was added Pd(OCOCH₃)₂ (299 mg; 1.33 mmol) under argon and the mixture was stirred under reflux (about 5 to 6 hours) until the TLC showed a complete conversion. The reaction mixture was then cooled to 70° C. and the organic layer was separated at this temperature and washed subsequently with an aqueous solution of NaOH (10% by weight), an aqueous solution of HCl (2 M) and brine. The organic layer was treated with activated charcoal (Norit® DX Ultra, Cabot Corp.) and the mixture was stirred for 2.5 hours. Then, the mixture was filtered over celite and the solution was concentrated under reduced pressure. The obtained mixture was cooled to RT and stirred overnight. The formed crystals were collected by filtration to yield the crude title compound as off-white solid, which was crystallized from toluene/anisole mixture to give 92.3 g of the title compound as off-white solid with chemical purity of 92%. After recrystallization from methanol title compound was obtained as white solid with chemical purity of >95%.

m.p.=210.1-211.7° C.;

m.p. (DSC): 207.1° C.;

¹H NMR (80 MHz, CDCl₃): δ=8.19-7.75 & 7.74-7.35 (m, 32H), 3.51-3.13 (m, 8H), 1.82 (s, 6H), 1.28 (s, 2H, OH) ppm.

IR (ATR): 744.6 (55.26); 819.8 (64.42); 854.5 (69.17); 885.4 (71.58); 1003.0 (78.82); 1037.7 (79.19); 1072.5 (81.63); 1190.1 (80.56); 1207.5 (73.83); 1446.7 (81.33); 1462.1 (81.60); 1504.5 (84.20); 2870.2 (90.17); 2930.0 (89.00); 2964.7 (86.99); 3051.5 (88.37); 3281.0 (90.10) cm⁻¹.

### Example 4a: 2,6-di(phenanthren-9-yl)-4-[1-(3,5-di(phenanthren-9-yl)-4-hydroxyphenyl)-1-methyl-ethyl]phenol (compound of formula (Ia-1), with X=C(CH₃)₂, R¹=R²=R³=R=phenanthren-9-yl and Z¹=Z²=hydrogen; compound 4 of table A)

To 2,6-dibromo-4-[1-(3,5-dibromo-4-hydroxy-phenyl)-1-methyl-ethyl]phenol (=3,3′,5,5′-tetrabromobisphenol A) (56.1 g; 100 mmol; purity=97%) were added phenanthren-9-ylboronic acid (133.3 g; 600 mmol; 6 equivalents) and tris(o-tolyl)phosphane (1.22 g; 4 mmol). To this mixture were added anisole (600 mL) and K₃PO₄ (133 g) dissolved in water (198 g). The mixture was stirred at 60-70° C. until two clear phases were formed and was purged with nitrogen. To this mixture was added Pd(OCOCH₃)₂ (225 mg; 1 mmol) under argon and the mixture was stirred under reflux until the TLC showed a complete conversion. The reaction mixture was then cooled to 70° C. and the organic layer was separated at this temperature and washed subsequently with an aqueous solution of NaOH (10% by weight), an aqueous solution of HCl (2 M) and brine. The organic layer was treated with activated charcoal (Norit® DX Ultra, Cabot Corp.) and the mixture was stirred for 2.5 hours. Then, the mixture was filtered over celite and the solvent was removed under reduced pressure. The crude title compound thus obtained as off-white solid was dissolved in a toluene/methanol mixture (400 g) at elevated temperature. The mixture was cooled to RT and stirred overnight. The formed crystals were collected by filtration to yield the title compound as off-white solid (90.9 g) with a chemical purity of 95%, which was used in the next step without further purification.

### Example 4b: 2-[4-[1-[4-(2-hydroxyethoxy)-3,5-di(phenanthren-9-yl)-phenyl]-1-methylethyl]-2,6-di(phenanthren-9-yl)-phenoxy]ethanol (compound of formula (Ia-1), with X=C(CH₃)₂, R¹=R²=R³=R⁴=phenanthren-9-yl and Z¹=Z²=2-hydroxyethyl; compound 36 of table A)

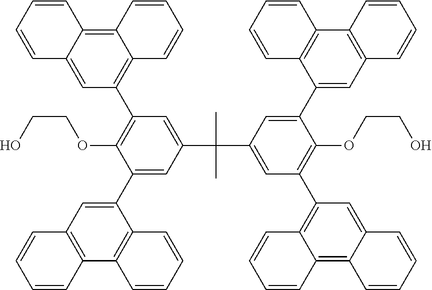 [SMILES: CC(C)(c1cc(-c2cc3ccccc3c3ccccc23)c(OCCO)c(-c2cc3ccccc3c3ccccc23)c1)c1cc(-c2cc3ccccc3c3ccccc23)c(OCCO)c(-c2cc3ccccc3c3ccccc23)c1]

To 2,6-di(phenanthren-9-yl)-4-[1-(3,5-di(phenanthren-9-yl)-4-hydroxy-phenyl)-1-methylethyl]phenol (111.7 g; 110 mmol; purity=95%) were added ethylene carbonate (77.5 g; 880 mmol; 8 equivalents), anisole (315 g) and K₂CO₃ (12.2 g) as a solid. The mixture was stirred at 135° C. until TLC showed complete conversion. The reaction mixture was then cooled to 70-75° C. and the organic layer was separated at this temperature and washed subsequently with an aqueous solution of NaOH (10% by weight) and brine. The organic layer was treated with activated charcoal (Norit® DX Ultra, Cabot Corp.) and the mixture was stirred for 2 to 3 hours. Then, the mixture was filtered over celite and the solvent was removed under reduced pressure. The crude title compound thus obtained as off-white solid was dissolved in methyl ethyl ketone (250 g) at reflux. The mixture was afterwards cooled to RT and stirred overnight. The formed crystals were collected by filtration to yield the title compound as white solid (90.5 g; 79% yield) with chemical purity of >97% and a yellowness index of 2.9.

m.p. (DSC): 299.7° C. (302.5° C.);

¹H NMR (80 MHz, CDCl₃): δ=8.99-8.72 (m, 8H), 8.13-7.69 (m, 32H), 3.33-3.11 (m, 4H), 3.10-2.87 (m, 4H), 2.19 (s, 6H), 1.16 (s, 2H, OH) ppm.

IR (ATR): 725.3 (71.39); 750.3 (69.02); 763.8 (83.89); 792.8 (90.23); 856.4 (92.27); 891.1 (85.61); 1010.7 (87.29); 1066.7 (89.17); 1215.2 (89.68); 1278.9 (93.54); 1361.8 (92.15); 1448.6 (89.01); 1462.1 (91.02); 2283.8 (95.22); 2868.2 (95.44); 2918.4 (95.24); 3063.1 (95.44); 3551.1 (93.88) cm⁻¹.

### Example 5: 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)-phenoxy]ethanol (compound of formula (Ia-1), with X=C(CH₃)₂, R¹=R²=R³=R⁴=dibenzo[b,d]thiophen-4-yl and Z¹=Z²=2-hydroxyethyl; compound 51 of table A)

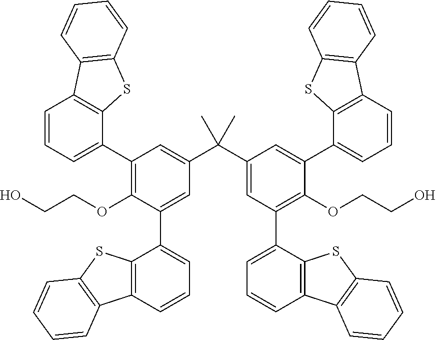 [SMILES: CC(C)(c1cc(-c2cccc3c2sc2ccccc23)c(OCCO)c(-c2cccc3c2sc2ccccc23)c1)c1cc(-c2cccc3c2sc2ccccc23)c(OCCO)c(-c2cccc3c2sc2ccccc23)c1]

To 2-[2,6-dibromo-4-[1-[3,5-dibromo-4-(2-hydroxyethoxy)phenyl]-1-methylethyl]phenoxy]ethanol (74.75 g; 110 mmol; purity=93%) were added (dibenzo[b,d]thiophen-4-yl)boronic acid (125.4 g; 550 mmol; 5 equivalents) and tris(o-tolyl)phosphane (4.02 g; 13.2 mmol). To this mixture were added anisole (660 mL) and K₃PO₄ (122.6 g) dissolved in water (285 g). The mixture was stirred at 60-70° C. until two clear phases were formed and was purged with nitrogen. To this mixture was added Pd(OCOCH₃)₂ (741 mg; 3.3 mmol) under argon and the mixture was stirred under reflux (about 5 to 6 hours) until TLC showed complete conversion. The reaction mixture was then cooled to RT and the formed grey solid product was collected by filtration, washed with water and methanol, and was dried. The crude product was dissolved in THE (790 mL) at 50° C., activated charcoal (Norit® DX Ultra, Cabot Corp.) was then added and mixture was stirred for 2 to 2.5 hours at 50° C. The hot mixture was filtered over celite and the obtained solution was concentrated under reduced pressure. The resulting off-white solid was suspended in methanol (500 mL) and stirred at reflux for 2 to 3 hours. The product was filtered off, washed with methanol and dried in vacuo to give 87 g of the title compound as a white powder. Purification by treatment with activated charcoal (Norit® DX Ultra, Cabot Corp.) was repeated once again and recrystallization from a THE/toluene mixture gave the title compound as a white crystalline powder in 87% yield and a chemical purity of >98%.

m.p. (DSC): 287.5° C.;

¹H NMR (80 MHz, CDCl₃): δ=8.39-8.05 (m, 8H), 7.89-7.13 (m, 24H), 3.35-3.12 (m, 4H), 3.12-2.89 (m, 4H), 1.95 (s, 6H), 1.2 (s, 2H, OH) ppm.

IR (ATR): 727.2 (64.11); 752.3 (28.21); 804.3 (76.60); 866.1 (80.90); 893.1 (71.52); 1010.7 (62.08); 1045.5 (65.61); 1070.5 (69.28); 1107.2 (77.73); 1219.1 (67.42); 1248.0 (78.54); 1363.7 (77.84); 1379.2 (66.51); 1440.9 (67.41); 1466.0 (76.10); 2868.2 (87.64); 3061.1 (88.60); 3554.9 (82.08) cm⁻¹.

### Example 6: 2-[4-[4-(2-hydroxyethoxy)-3,5-di(naphthalen-1-yl)phenyl]sulfonyl-2,6-di(naphthalen-1-yl)phenoxy]ethanol (compound of formula (Ia-1), with X=SO₂, R¹=R²=R³=R⁴=naphthalen-1-yl and Z¹=Z²=2-hydroxyethyl; compound 55 of table A)

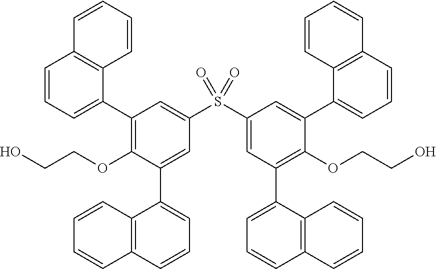 [SMILES: O=S(=O)(c1cc(-c2cccc3ccccc23)c(OCCO)c(-c2cccc3ccccc23)c1)c1cc(-c2cccc3ccccc23)c(OCCO)c(-c2cccc3ccccc23)c1]

To 2-[4-[4-(2-hydroxyethoxy)-3,5-dibromophenyl]sulfonyl-2,6-dibromophenoxy]ethanol (170.3 g; 250 mmol; purity=96%) were added naphthalen-1-ylboronic acid (219.37 g; 1.25 mol; 5 eq.; purity=98%) and tris(o-tolyl)phosphan (1.52 g; 5.0 mmol). To this mixture were added anisole (750 mL) and 276 g K₃PO₄ dissolved in 613 g of water. The mixture was stirred at 60-70° C. until two clear phases are formed and was purged with nitrogen. To this mixture was added Pd(OCOCH₃)₂ (281 mg; 1.25 mmol) under argon and the mixture was stirred under reflux until the TLC showed complete conversion. The mixture was cooled to 70° C. and the organic layer was separated at 70° C. and washed subsequently with an aqueous solution of NaOH (10% by weight), an aqueous HCl solution (2 M) and brine. To the organic layer was added activated charcoal (Norit DX Ultra) and the mixture was stirred for 2.5 hours. Then, the mixture was filtered over celite and the solution was concentrated under reduced pressure. The mixture was cooled to RT and stirred overnight. The formed crystals were collected by filtration to yield the crude product as a white solid (149.33 g; 65%) with chemical purity of 92.14%. The crude material can be recrystallized from 1.6 L toluene/MeOH (1:1 (v/v)) mixture to obtain 79.6 g of the title compound as a white solid with chemical purity of approximately 97%.

m.p. (DSC): 213.4° C.;

¹H NMR (80 MHz, CDCl₃): δ=8.07 (s, 4H), 7.83-7.12 (m, 28H), 3.31-3.06 (m, 4H), 3.04-2.72 (m, 4H), 0.79 (s, 2H, OH) ppm.

IR (ATR): 439.78 (50.37); 509.22 (45.63); 609.53 (59.16); 621.1 (69.13); 651.96 (63.19); 723.33 (73.09); 734.9 (72.97); 775.41 (41.64); 798.56 (64.26); 895.0 (76.4); 1018.45 (71.93); 1068.6 (72.88); 1091.75 (66.93); 1118.75 (70.24); 1147.68 (56.44); 1226.77 (75.63); 1323.21 (70.4); 1423.51 (78.3); 1506.46 (86.42); 1573.97 (89.54); 1593.25 (91.18); 2947.33 (90.31); 3057.27 (90.11); 3570.36 (89.31) cm⁻¹.

### Example 7: 2-[4-[4-(2-hydroxyethoxy)-3,5-di(naphthalen-1-yl)phenyl]sulfanyl-2,6-di(naphthalen-1-yl)phenoxy]ethanol (compound of formula (Ia-1), with X=S, R¹=R²=R³=R⁴=naphthalen-1-yl and Z¹=Z²=2-hydroxyethyl; compound 76 of table A)

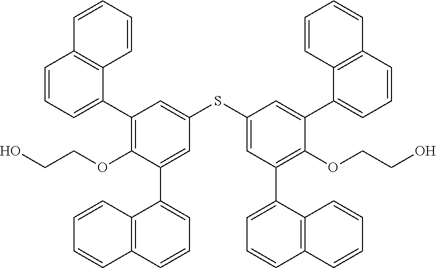 [SMILES: OCCOc1c(-c2cccc3ccccc23)cc(Sc2cc(-c3cccc4ccccc34)c(OCCO)c(-c3cccc4ccccc34)c2)cc1-c1cccc2ccccc12]

To a cooled (−18° C.) solution of lithium aluminium hydride (19 g; 500 mmol) in THE (600 mL) was slowly added a solution of TiCl₄ (47.5 g; 250 mmol) in 2-methyl-THF (1 L) and the mixture was stirred under inert gas for 30 min at 0° C. To this mixture 2-[4-[4-(2-hydroxyethoxy)-3,5-di(naphthalene-1-yl)phenyl]sulfonyl-2,6-di(naphthalene-1-yl)phenoxy]ethanol obtained in Example 6 (43.9 g; 50 mmol) was slowly added portionwise at 0° C. After complete addition the mixture was stirred for further 30 min at 0° C. under inert gas. The mixture was slowly warmed to RT and was stirred at RT for 1 hour. TLC (eluent: cyclohexane:ethyl acetate 2:1) showed complete conversion. To this mixture a mixture water (19 g) and THE (30 mL) was slowly added to destroy the excess of lithium aluminium hydride. The solvent was removed under reduced pressure and the resulting residue was purified by column chromatography (silica gel; cyclohexane: ethyl acetate 5:1) to yield 24.6 g of the title compound as a white solid with chemical purity of approximately 99.7%.

m.p. (DSC): 97° C.;

¹H NMR (80 MHz, CDCl₃): δ=8.1-7.2 (m, 32H), 3.2-3.0 (m, 4H), 2.8-2.5 (m, 4H), 0.2-0.5 (m, 2H, O) ppm.

### Example 8: 2-[4-[4-(2-hydroxyethoxy)-3,5-di(phenanthren-9-yl)phenyl]sulfonyl-2,6-di(phenanthren-9-yl)phenoxy]ethanol (compound of formula (Ia-1), with X=SO₂, R¹=R²=R³=R⁴=phenanthren-9-yl and Z¹=Z²=2-hydroxyethyl; compound 57 of table A)

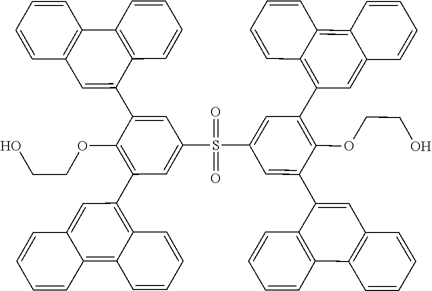 [SMILES: O=S(=O)(c1cc(-c2cc3ccccc3c3ccccc23)c(OCCO)c(-c2cc3ccccc3c3ccccc23)c1)c1cc(-c2cc3ccccc3c3ccccc23)c(OCCO)c(-c2cc3ccccc3c3ccccc23)c1]

To 2-[4-[4-(2-hydroxyethoxy)-3,5-dibromophenyl]sulfonyl-2,6-dibromophenoxy]ethanol (148.0 g; 217.26 mmol; purity=96%) were added phenanthren-9-ylboronic acid (246.13 g; 1,0863 mol; 5 eq.; purity=98%) and tris(o-tolyl)phosphane (1.33 g; 4.35 mmol). To this mixture were added anisole (700 g) and 240 g K₃PO₄ dissolved in 532 g of water. The mixture was stirred at 60-70° C. until two clear phases were formed and was purged with nitrogen. To this mixture was added Pd(OCOCH₃)₂ (244 mg; 1.086 mmol) under argon and the mixture was stirred under reflux for 9 hours. Because TLC (eluent: e.g. cyclohexane:ethyl acetate 1:2) afterwards showed complete reaction, another portion of phenanthren-9-ylboronic acid (24.613 g; 108.63 mmol), and 24 g K₃PO₄ dissolved in 53.2 g of water as well as Pd-catalyst [Pd(OCOCH₃)₂ (24.4 mg; 0.1086 mmol) and tris(o-tolyl)phosphane (133 mg; 0.435 mmol)] were added. The reaction mixture was stirred under reflux for another 90 minutes until the TLC (eluent: e.g. cyclohexane:ethyl acetate 1:2) showed an almost complete conversion. The mixture was cooled to RT and stirred for 1 hour.

The crude product was filtered off, washed with anisole and 2-methyltetrahydrofuran and dried at 60° C. The crude product was dissolved in 3 L THE and 10 g of activated charcoal (Norit DX Ultra) were added. The mixture was stirred at 40° C. for 2 hours and after filtration of activated charcoal over celite, the solvent was completely removed under reduced pressure. The product was crystallized from toluene to obtain 157.7 g of the title compound as a white solid with chemical purity of >94%. The product was recrystallized from toluene to afford the title compound with chemical purity of >97%.

m.p. (DSC): 233.8° C. (toluene-solvate); 242.2° C.

¹H NMR (80 MHz, CDCl₃): δ=8.43-9.03 (m, 8H), 8.22 (dd, J=3.1, 1.4 Hz, 4H), 7.55-8.01 (m, 28H), 7.20-7.27 (m, 4H), 3.01-3.49 (m, 4H, —CH₂—), 2.50-2.92 (m, 4H, —CH₂—), 0.32-0.56 (m, 2H) ppm.

IR (ATR): 405.06 (29.54); 561.3 (78.99); 615.31 (63.9); 632.67 (63.18); 723.33 (48.06); 744.55 (52.77); 763.84 (75.45); 887.28 (76.3); 964.44 (83.77); 1012.66 (79.76); 1066.67 (82.66); 1082.1 (76.5); 1091.75 (75.17); 1132.25 (63.8); 1145.75 (76.3); 1178.55 (85.26); 1230.63 (81.87); 1261.49 (82.73); 1319.35 (80.0); 1419.66 (82.14); 1450.52 (81.1) cm⁻¹.

n_{D} calc.: 1.76 (calculated using the software ACD/ChemSketch 2012 from Advanced Chemistry Development, Inc.)

### Example 9: 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol (compound of formula (Ia-1), with X=SO₂, R¹=R²=R³=R⁴=dibenzo[b,d]thien-4-yl and Z¹=Z²=2-hydroxyethyl; compound 72 of table A)

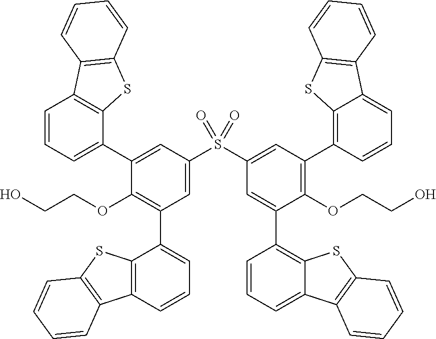 [SMILES: O=S(=O)(c1cc(-c2cccc3c2sc2ccccc23)c(OCCO)c(-c2cccc3c2sc2ccccc23)c1)c1cc(-c2cccc3c2sc2ccccc23)c(OCCO)c(-c2cccc3c2sc2ccccc23)c1]

To 2-[4-[4-(2-hydroxyethoxy)-3,5-dibromophenyl]sulfonyl-2,6-dibromophenoxy]ethanol (143.05 g; 210 mmol; purity=96%) were added dibenzo[b,d]thiophen-4-ylboronic acid (244.37 g; 1.05 mol; 5 eq.; purity=98%) and tris(o-tolyl)phosphan (1.28 g; 4.2 mmol). To this mixture were added anisole (700 g) and 232 g K₃PO₄ dissolved in 515 g of water. The mixture was stirred at 60-70° C. until two clear phases were formed and was purged with nitrogen. To this mixture was added Pd(OCOCH₃)₂ (236 mg; 1.05 mmol) under argon and the mixture was stirred under reflux for 3 hour until TLC (eluent: e.g. cyclohexane:ethyl acetate 1:2) showed complete conversion (remark: If according to TLC the reaction after 9 hours at reflux is not complete, another portion of dibenzo[b,d]thiophen-4-ylboronic acid (24.44 g; 105 mmol) and 23.2 g K₃PO₄ dissolved in 51.5 g of water as well as Pd-catalyst [Pd(OCOCH₃)₂ (23.6 mg; 0.105 mmol) and tris(o-tolyl)phosphan (128 mg; 0.42 mmol)] should be added and the reaction mixture should be stirred under reflux until the TLC shows at least almost complete conversion). The mixture was cooled to 70° C. and the organic layer was separated at 70° C. and the aqueous phase was extracted with 250 mL of 2-methyltetrahydrofuran. To the combined organic layers a 10% aqueous solution of NaOH (375 mL) was added at 70° C. Since the product crystallized, the suspension was cooled to RT and the crude product was filtered off, washed subsequently with water and THF and dried at 60° C. to give 207.5 g of crude product.

The obtained solid was dissolved in 3 L anisole at reflux and then cooled to 120° C. Then 10 g of activated charcoal (Norit DX Ultra) were added and the mixture was stirred at 120° C. for 1 hour. The activated charcoal was filtered off at 120° C. using celite and the solution was concentrated under reduced pressure to ca. 1000 g. The clear solution was cooled to RT and THF (500 mL) was added. The formed crystals were collected by filtration and dried at 60° C. to yield the crude title compound as a white solid (174.4 g; 77.8%). The crude product can be recrystallized from an anisole/THF (1/1 v/v) or an anisole/2-propanol (1/1 v/v) mixture to obtain the title compound with a chemical purity of >95% (by NMR). (Remark: If the anisole/THF mixture was used a THF-solvate of the title compound was obtained).

m.p. (DSC): 204.1° C. (TH F-solvate); 294.1° C.

¹H NMR (80 MHz, CDCl₃): δ=8.52 (s, 4H), 8.32-8.51 (m, 8H), 7.09-7.88 (m, 20H), 3.40-3.55 (m, 4H), 3.13-3.29 (m, 4H), 1.00 (t, J=6.5 Hz, 2H, OH) ppm.

IR (ATR): 495.72 (37.35); 551.66 (81.77); 565.16 (83.67); 607.6 (62.68); 630.74 (69.8); 704.04 (76.01); 723.33 (77.99); 748.41 (49.59); 885.36 (81.46); 906.57 (81.63); 1003.02 (77.16); 1045.45 (68.95); 1068.6 (80.44); 1105.25 (78.89); 1120.68 (80.61); 1145.75 (67.34); 1234.48 (80.97); 1246.06 (79.06); 1303.92 (81.87); 1319.35 (80.33); 1375.29 (80.49); 1383.01 (81.94); 1429.3 (77.66); 1442.8 (82.59) cm⁻¹.

no calc.: 1.79 (calculated using the software ACD/ChemSketch 2012 from Advanced Chemistry Development, Inc.)

### Example 10: 2-[4-[4-(2-hydroxyethoxy)-3,5-di(thianthrene-1-yl)phenyl]sulfonyl-2,6-di(thianthrene-1-yl)phenoxy]ethanol (compound of formula (Ia-1), with X=SO₂, R¹=R²=R³=R⁴=thianthrene-1-yl and Z¹=Z²=2-hydroxyethyl; compound 73 of table A)

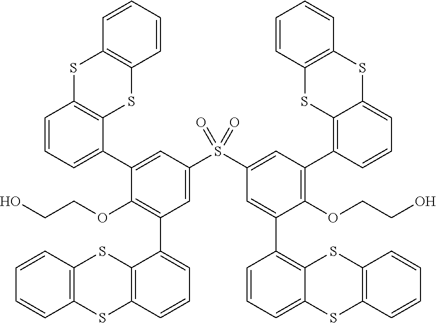 [SMILES: O=S(=O)(c1cc(-c2cccc3c2Sc2ccccc2S3)c(OCCO)c(-c2cccc3c2Sc2ccccc2S3)c1)c1cc(-c2cccc3c2Sc2ccccc2S3)c(OCCO)c(-c2cccc3c2Sc2ccccc2S3)c1]

To 2-[4-[4-(2-hydroxyethoxy)-3,5-dibromophenyl]sulfonyl-2,6-dibromophenoxy]ethanol (143.05 g; 210 mmol; purity=96%) were added thianthren-1-ylboronic acid (278.72 g; 1.05 mol; 5 eq.; purity=98%) and tris(o-tolyl)phosphan (1.28 g; 4.2 mmol). To this mixture were added anisole (700 g) and 232 g K₃PO₄ dissolved in 515 g of water. The mixture was stirred at 60-70° C. until two clear phases were formed and was purged with nitrogen. To this mixture was added Pd(OCOCH₃)₂ (236 mg; 1.05 mmol) under argon and the mixture was stirred under reflux until the TLC (eluent: e.g. cyclohexane:ethyl acetate 1:2) showed complete conversion. (Note: If according TLC the reaction after 9 hours at reflux is not complete, another portion of thianthren-1-ylboronic acid (27.87 g; 105 mmol) and 23.2 g K₃PO₄ dissolved in 51.5 g of water as well as Pd-catalyst [Pd(OCOCH₃)₂ (23.6 mg; 0.105 mmol) and tris(o-tolyl)phosphan (128 mg; 0.42 mmol)]should be added. The reaction mixture should then be stirred under reflux until TLC showed at least almost complete conversion.)

The mixture was cooled to 70° C. and the organic layer was separated at 70° C. and washed subsequently with an aqueous solution of NaOH (10% by weight), an aqueous HCl solution (2 M) and brine. To the organic layer 100 g of Na₂SO₄ and 10 g of activated charcoal (Norit DX Ultra) were added and the mixture was stirred for 1 hour. Then, the mixture was filtered over celite and the solvent was completely removed under reduced pressure. The crude product was dissolved in 860-880 mL of a toluene/methanol (1/1 v/v) mixture at 55° C. and then the clear solution was cooled to RT and stirred overnight. The formed crystals were collected by filtration to yield after drying at 60° C. the crude title compound as a white solid (210.6 g; 83.9%) with chemical purity of 97.61%. The product may be further purified by additional recrystallization from toluene/methanol mixtures.

m.p. (DSC): 224.0° C.;

¹H NMR (80 MHz, CDCl₃): δ=8.25 (s, 4H), 7.97-7.23 (m, 28H), 3.55-3.35 (m, 4H), 3.43-3.05 (m, 4H), 1.41-1.30 (m, 2H, OH), ppm.

IR (ATR): 572.88 (85.64); 615.31 (56.0); 663.53 (83.11); 700.18 (68.44); 721.4 (79.65); 746.48 (51.87); 783.13 (72.49); 794.7 (79.81); 904.64 (81.82); 1010.73 (76.44); 1057.03 (84.15); 1074.39 (80.9); 1099.46 (73.27); 1118.75 (83.89); 1145.75 (63.26); 1190.12 (86.98); 1226.77 (80.11); 1249.91 (83.27); 1319.35 (71.87); 1361.79 (85.74); 1394.58 (74.99); 1431.23 (74.95); 1448.59 (80.7); 1558.54 (87.15) cm⁻¹.

no calc.: 1.77 (calculated using the software ACD/ChemSketch 2012 from Advanced Chemistry Development, Inc.)

### Example 11: 2,2′-(sulfonylbis{[2,6-di(naphthalen-1-yl)-4,1-phenylene]oxy})di(ethan-1-ol) (compound of formula (Ia-1), with X=SO₂, R¹=R²=R³=R⁴=naphthalen-2-yl and Z¹=Z²=2-hydroxyethyl; compound 56 of table A)

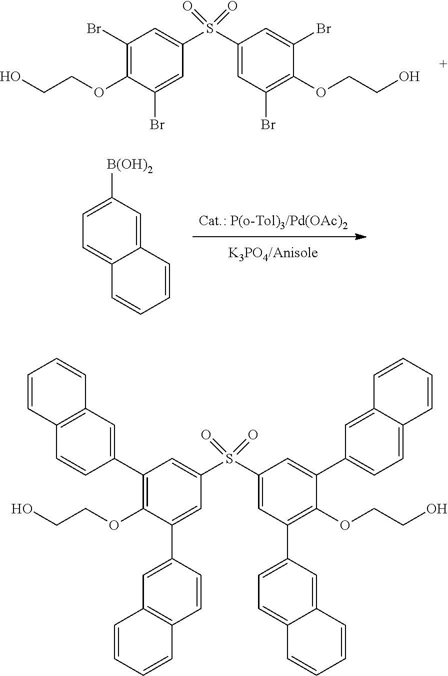 [SMILES: O=S(=O)(c1cc(Br)c(OCCO)c(Br)c1)c1cc(Br)c(OCCO)c(Br)c1 ; OB(O)c1ccc2ccccc2c1 ; O=S(=O)(c1cc(-c2ccc3ccccc3c2)c(OCCO)c(-c2ccc3ccccc3c2)c1)c1cc(-c2ccc3ccccc3c2)c(OCCO)c(-c2ccc3ccccc3c2)c1]

To 2,2′-{sulfonylbis[(2,6-dibromo-4,1-phenylene)oxy]}di(ethan-1-ol) (170.3 g; 250 mmol; purity=96%) were added naphthalen-2-ylboronic acid (219.37 g; 1.25 mol; 5 eq.; purity=98%) and tris(o-tolyl)phosphan (1.52 g; 5.0 mmol). To this mixture were added anisole (750 mL) and 276 g K₃PO₄ dissolved in 613 g of water. The mixture was stirred at 60-70° C. until two clear phases were formed and was purged with argon. To this mixture was added Pd(OCOCH₃)₂ (281 mg; 1.25 mmol) under argon and the mixture was stirred under reflux until TLC showed complete conversion.

The mixture was cooled to 70° C., the organic layer was separated at 70° C. and washed subsequently with an aqueous solution of NaOH (10% by weight), an aqueous HCl solution (2 M) and brine. To the organic layer was added activated charcoal (Norit DX Ultra) and the mixture was stirred at 70° C. for 2.5 hours. Then, the mixture was filtered over celite and the solution was concentrated under reduced pressure. The mixture was cooled to ambient temperature and stirred overnight. The formed crystals were collected by filtration to yield the crude title compound as a white solid (149.33 g; 65%) with chemical purity of 92.2%. The crude material was recrystallized subsequently from toluene/MeOH (1/1 v/v) and then from methyl ethyl ketone to obtain 78.71 g of the title compound as a white solid with chemical purity of ca. 94%.

m.p. (DSC): 252.2° C.;

¹H NMR (80 MHz, CDCl₃): δ=8.21 (s, 4H, CAr—H), 8.10-7.42 (m, 28H, CAr—H), 3.44 3.34 (m, 4H, CH₂), 3.22-3.11 (m, 4H, CH₂), 1.11 (bs, 2H) ppm.

IR (ATR): 740.69 (60.15); 812.06 (67.64); 866.07 (73.57); 895 (74.44); 949.01 (80.71); 1016.52 (74.98); 1101.39 (65.76); 1143.83 (55.87); 1219.05 (75.88); 1319.35 (64.85); 1421.58 (79.99); 1442.8 (83.05) cm⁻¹.

### Example 12: 4,4′-(propane-2,2-diyl)bis[2,6-di(thianthren-1-yl)phenol] (compound of formula (Ia-1), with X=C(CH₃)₂, R¹=R²=R³=R⁴=thianthren-1-yl and Z¹=Z²=hydrogen; compound 7 of table A)

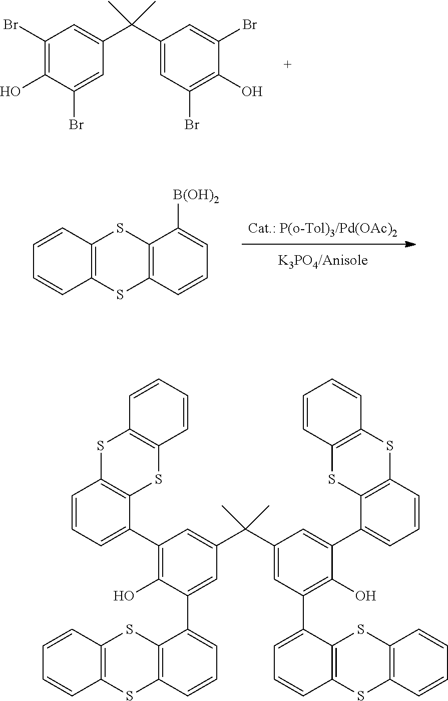 [SMILES: CC(C)(c1cc(Br)c(O)c(Br)c1)c1cc(Br)c(O)c(Br)c1 ; OB(O)c1cccc2c1Sc1ccccc1S2 ; CC(C)(c1cc(-c2cccc3c2Sc2ccccc2S3)c(O)c(-c2cccc3c2Sc2ccccc2S3)c1)c1cc(-c2cccc3c2Sc2ccccc2S3)c(O)c(-c2cccc3c2Sc2ccccc2S3)c1]

To 4,4′-(propane-2,2-diyl)bis(2,6-dibromophenol) (70.1 g; 125 mmol; purity: 97%) were added thianthren-1-ylboronic acid (166 g; 625 mmol; 5 eq.; purity; 98%) and tris(o-tolyl)phosphan (1.52 g; 5.0 mmol). To this mixture were added anisole (500 g) and 11.4 g of K₃PO₄ dissolved in 248 g of water. The mixture was stirred at 60-70° C. until two clear phases were formed and was purged with argon. To this mixture was added Pd(OCOCH₃)₂ (281 mg; 1.25 mmol) under argon and the mixture was stirred under reflux until TLC (eluent: e.g. cyclohexane/ethyl acetate 3:1) showed complete conversion.

The mixture was cooled to 70° C. and the organic layer was separated at 70° C. and washed subsequently with an aqueous solution of NaHCO₃ (10% by weight), an aqueous HCl solution (2 M) and brine. To the organic layer 6.75 g of activated charcoal (Norit DX Ultra) and 13.5 g of Na₂SO₄ were added and the mixture was stirred for 1 hour. Then, the mixture was filtered over celite and the solvent was completely removed under reduced pressure. The crude product can be used for the next step without additional purification or recrystallized from a toluene/MeOH (1/1 v/v) mixture.

### Example 13: 2,2′-(propane-2,2-diylbis{[2,6-di(thianthren-1-yl)-4,1-phenylene]oxy})di(ethan-1-ol) (compound of formula (Ia-1), with X=C(CH₃)₂, R¹=R²=R³=R⁴=thianthren-1-yl and Z¹=Z²=2-hydroxyethyl; compound 52 of table A)

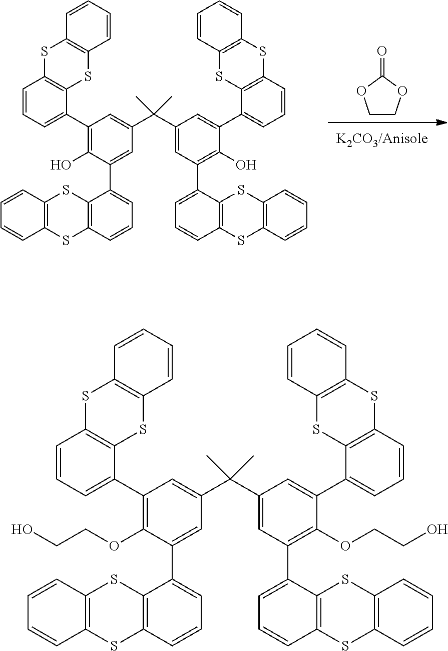 [SMILES: CC(C)(c1cc(-c2cccc3c2Sc2ccccc2S3)c(O)c(-c2cccc3c2Sc2ccccc2S3)c1)c1cc(-c2cccc3c2Sc2ccccc2S3)c(O)c(-c2cccc3c2Sc2ccccc2S3)c1 ; O=C1OCCO1 ; CC(C)(c1cc(-c2cccc3c2Sc2ccccc2S3)c(OCCO)c(-c2cccc3c2Sc2ccccc2S3)c1)c1cc(-c2cccc3c2Sc2ccccc2S3)c(OCCO)c(-c2cccc3c2Sc2ccccc2S3)c1]

To 4,4′-(propane-2,2-diyl)bis[2,6-di(thianthren-1-yl)phenol] obtained in Example 12 (125 mmol) in anisole (360 g) were added 5.2 g of K₂CO₃ and 33 g of ethylene carbonate. The reaction mixture was stirred at reflux until TLC (eluent: e.g. cyclohexane/ethyl acetate 3:1) showed complete conversion.

The mixture was cooled to 70-80° C. and the organic layer was separated at 70° C. and washed subsequently with brine, an aqueous solution of NaOH (10% by weight), an aqueous HCl solution (2 M) and brine. The organic layer was dried with Na₂SO₄ and after filtration through celite the solvent was completely removed under reduced pressure. The crude product was purified with column chromatography to obtain 70.8 g of the title compound as a white solid with chemical purity of ca. 97-98%.

m.p. (DSC): 110.6° C.;

¹H NMR (80 MHz, CDCl₃): δ=7.92-7.25 (m, 32H, C_{Ar}—H), 3.5-3.0 (m, 8H, CH₂), 2.1 (bs, 6H, CH₃), ppm.

IR (ATR): 723.33 (68.04); 744.55 (47.89); 779.27 (73.45); 792.77 (77.93); 885.36 (77.60); 1026.16 (74.34); 1070.53 (78.20); 1111.03 (80.06); 1219.05 (76.24); 1247.99 (79.74); 1386.86 (70.11); 1448.59 (64.40); 1552.75 (85.03); 2848.96 (83.56); 2924.18 (78.87); 3053.42 (87.35) cm⁻¹.

### Example 14a: 3,3′,5,5′-tetrabromo[1,1′-biphenyl]-4,4′-diol

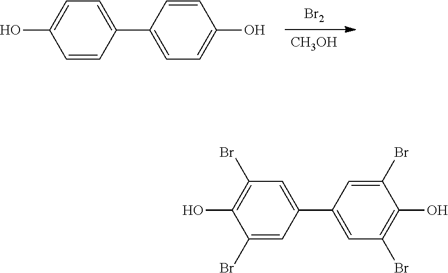 [SMILES: Oc1ccc(-c2ccc(O)cc2)cc1 ; "Br2" ; "CH3OH" ; Oc1c(Br)cc(-c2cc(Br)c(O)c(Br)c2)cc1Br]

To a solution of 82.7 mL (ca. 258 g) of bromine in 750 g methanol was slowly added a solution of 60 g of [1,1′-biphenyl]-4,4′-diol in 750 g of methanol at 0° C. The reaction mixture was stirred for an additional hour at 0° C. and then overnight at ambient temperature. The precipitated product was filtered off, washed subsequently with cold methanol, then with an aqueous solution of ascorbic acid (20% by weight) and finally twice with water. The crude product (149 g) was crystallized from a THE/toluene mixture to give 130.3 g of the title product as off-white powder with chemical purity of >97.5%.

¹H NMR (80 MHz, DMSO-d₆): δ=9.91 (s, 2H, OH), 7.72 (s, 4H, C_{Ar}—H) ppm.

### Example 14b: 2,2′-[(3,3′,5,5′-tetrabromo[1,1′-biphenyl]-4,4′-diyl)bis(oxy)]di(ethan-1-ol)

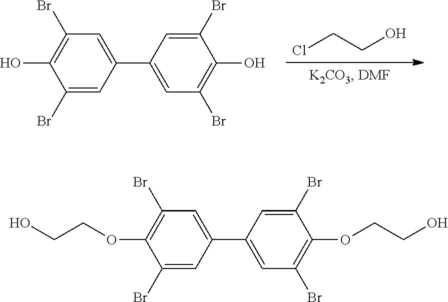

To a solution of 161.3 g of 3,3′,5,5′-tetrabromo[1,1′-biphenyl]-4,4′-diol obtained in Examples 14a in 483 g (ca. 512 mL) of DMF (homogenized at 60° C.) 172.4 g of K₂CO₃ were added. The mixture was stirred for further 10-20 minutes at 60° C. and then 201.24 g of 2-chloroethan-1-ol were added. The reaction mixture was stirred 3 hours at 120° C. After cooling to ambient temperature, the reaction mixture was poured into 1.5 L of water to form a white precipitate. After slow neutralization with conc. HCl, the crude product was filtered off and washed subsequently with water (3×500 mL) and ethanol (500 mL). The crude product was dissolved in 1450 g of THE at reflux, 4.0 g of activated charcoal (Norit DX Ultra) were added and the mixture was stirred at reflux for 1 hour. Then, the mixture was filtered over celite at 60° C., and solvent was removed under reduced pressure. After crystallization from a THE/toluene mixture, the obtained crystals were filtered off and washed with 500 g of toluene to give 157.3 g (yield approx. 85.5%) of title compound with chemical purity of >97.7%).

m.p. (DSC): 232.1° C.;

¹H NMR (80 MHz, DMSO-d₆): δ=7.8 (s, 4H, C_{Ar}—H), 4.74 (t, J=5.4 Hz, 2H, OH), 4.2 3.5 (m, 8H, CH₂) ppm.

### Example 14c: 2,2′-{[3,3′,5,5′-tetra(naphthalen-1-yl)[1,1′-biphenyl]-4,4′-diyl]bis(oxy)}di(ethan-1-ol) (compound of formula (Ia-1), with X=a single bond, R¹=R²=R³=R⁴=naphthalen-1-yl and Z¹=Z²=2-hydroxyethyl; compound 244 of table A)

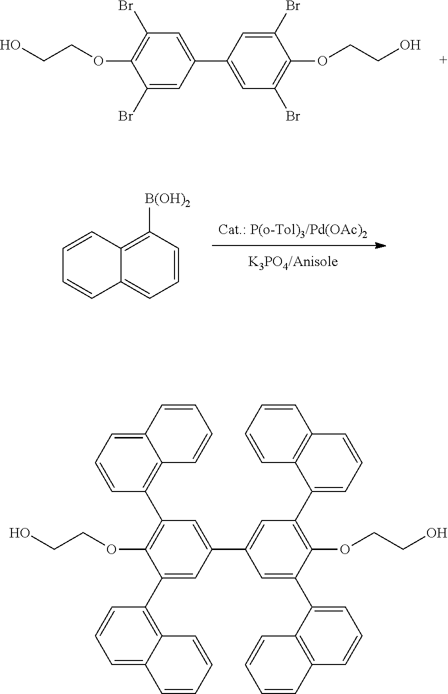 [SMILES: OCCOc1c(Br)cc(-c2cc(Br)c(OCCO)c(Br)c2)cc1Br ; OB(O)c1cccc2ccccc12 ; OCCOc1c(-c2cccc3ccccc23)cc(-c2cc(-c3cccc4ccccc34)c(OCCO)c(-c3cccc4ccccc34)c2)cc1-c1cccc2ccccc12]

To 2,2′-[(3,3′,5,5′-tetrabromo[1,1′-biphenyl]-4,4′-diyl)bis(oxy)]di(ethan-1-ol) obtained in Example 14b (74.5 g; 123.77 mmol) were added naphthalen-1-ylboronic acid (110 g; 626.8 mmol; 5.06 eq.; purity=98%) and anisole (390 mL). To this mixture were added 138 g of K₃PO₄ dissolved in 306 g of water. The mixture was stirred at 60-70° C. until two clear phases were formed and then purged with argon. To this mixture was added tris(o-tolyl)phosphan (1.5 g; 4.95 mmol) and Pd(OCOCH₃)₂ (281 mg; 1.25 mmol) in 5 mL of anisole under argon and the mixture was stirred under reflux until TLC showed complete conversion.

The mixture was cooled to 70° C. and the organic layer was separated at 70° C. and washed subsequently with an aqueous solution of NaOH (10% by weight), an aqueous HCl solution (2 M) and brine. To the organic layer were added 10 g of activated charcoal (Norit DX Ultra) and 100 g of Na₂SO₄ and the mixture was stirred at 70° C. for 1 hour. Then, the mixture was filtered over celite and the solvent was removed under reduced pressure. The crude product can be re-crystallized from a mixture of toluene/i-propanol or toluene/MeOH (1/1 v/v) and/or purified via column chromatography (eluent: cyclohexane/ethyl acetate) to obtain the title compound as a white solid with chemical purity of ca. 93%.

m.p. (DSC): 249.6° C.;

¹H NMR (80 MHz, CDCl₃): δ=8.20-7.73 (m, 16H, C_{Ar}—H), 7.73-7.33 (m, 16H, C_{Ar}—H), 3.3-2.96 (m, 4H, CH₂), 3.0-2.53 (m, 4H, CH₂), 0.45 (bs, 2H, O) ppm.

IR (ATR): 607.6 (76.1); 734.9 (75.7); 773.48 (34.8); 788.91 (56.9); 800.49 (68.7); 887.28 (68.9); 1018.45 (66.0); 1066.67 (66.2); 1087.89 (74.9); 1222.91 (65.5); 1361.79 (78.5); 1396.51 (70.4); 1427.37 (72.5); 1440.87 (75.9); 1506.46 (75.0) cm⁻¹.

### Example 15: 2,2′-{[3,3′,5,5′-tetra(naphthalen-2-yl)[1,1′-biphenyl]-4,4′-diyl]bis(oxy)}di(ethan-1-ol) (compound of formula (Ia-1), with X=a single bond, R¹=R²=R³=R⁴=naphthalen-1-yl and Z¹=Z²=2-hydroxyethyl; compound 245 of table A)

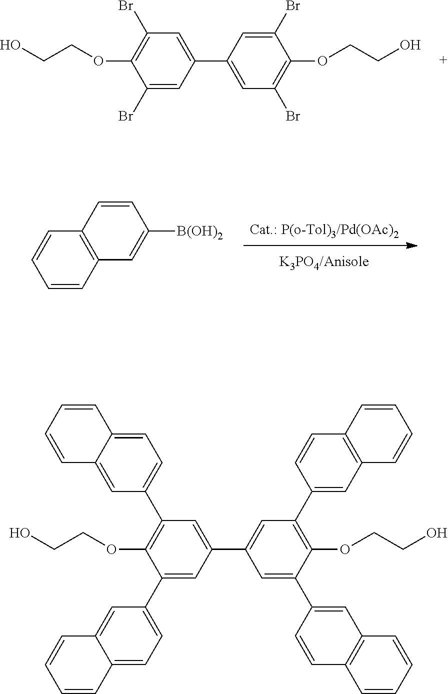 [SMILES: OCCOc1c(Br)cc(-c2cc(Br)c(OCCO)c(Br)c2)cc1Br ; OB(O)c1ccc2ccccc2c1 ; OCCOc1c(-c2ccc3ccccc3c2)cc(-c2cc(-c3ccc4ccccc4c3)c(OCCO)c(-c3ccc4ccccc4c3)c2)cc1-c1ccc2ccccc2c1]

To 2,2′-[(3,3′,5,5′-tetrabromo[1,1′-biphenyl]-4,4′-diyl)bis(oxy)]di(ethan-1-ol) (74.5 g; 123.77 mmol) were added naphthalen-2-ylboronic acid (165 g; 937.5 mmol; 7.5 eq.; purity=98%) and anisole (440 mL). To this mixture were added 207 g K₃PO₄ dissolved in 460 g of water. The mixture was stirred at 60-70° C. until two clear phases were formed and was purged with argon. To this mixture was added tris(o-tolyl)phosphan (3.05 g; 4.95 mmol) and Pd(OCOCH₃)₂ (561 mg; 2.5 mmol) in 10 mL of anisole under argon and the mixture was stirred under reflux until TLC showed complete conversion. The mixture was cooled to 70° C. and the organic layer was separated at 70° C. and washed subsequently with an aqueous solution of NaOH (10% by weight), an aqueous HCl solution (2 M) and brine. To the organic layer were added 10 g of activated charcoal (Norit DX Ultra) and 100 g of Na₂SO₄ and the mixture was stirred at 70° C. for 1 hour. Then, the mixture was filtered over celite and the solvent was removed under reduced pressure. The crude product was re-crystallized from a mixture of toluene/MeOH (7/3 w/w) and/or purified via column chromatography (eluent: cyclohexane/ethyl acetate) to obtain the title compound as a white solid with chemical purity of about 94%.

m.p. (DSC): 256.4° C.;

¹H NMR (80 MHz, CDCl₃): δ=8.2 (s, 4H, C_{Ar}—H), 8.12-7.77 (m, 20H, C_{Ar}—H), 7.71 7.42 (m, 8H, C_{Ar}—H), 3.57-3.38 (m, 4H, CH₂), 3.37-3.13 (m, 4H, CH₂), 1.13 (m, 2H, OH) ppm.

IR (ATR): 515.01 (67.1); 651.96 (78.1); 744.55 (37.5); 775.41 (77.9); 800.49 (77.7); 823.63 (53.4); 860.28 (51.3); 893.07 (71.0); 906.57 (74.4); 995.3 (77.1); 1014.59 (70.5); 1045.45 (74.2); 1072.46 (72.0); 1080.17 (68.6); 1217.12 (67,0); 1236.41 (76.2); 1336.71 (76.6); 1419.66 (67.7); 1442.8 (69.8); 1504.53 (76.0) cm⁻¹.

### Example 16: 2,2′-{[3,3′,5,5′-tetra(phenanthren-9-yl)[1,1′-biphenyl]-4,4′-diyl]bis(oxy)}di(ethan-1-ol) (compound of formula (Ia-1), with X=a single bond, R¹=R²=R³=R⁴=phenanthren-9-yl and Z¹=Z²=2-hydroxyethyl; compound 246 of table A)

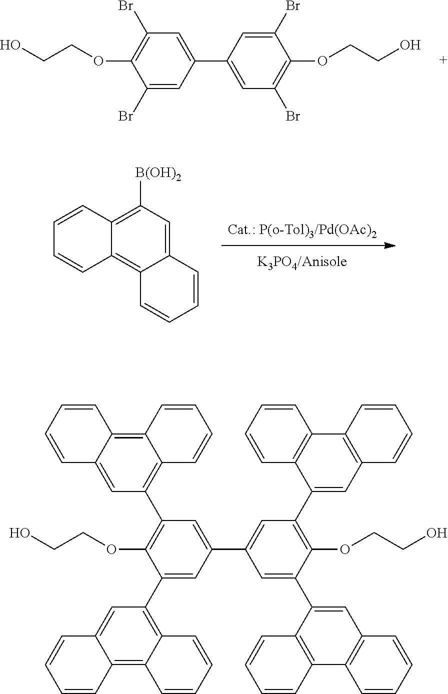 [SMILES: OCCOc1c(Br)cc(-c2cc(Br)c(OCCO)c(Br)c2)cc1Br ; OB(O)c1cc2ccccc2c2ccccc12 ; OCCOc1c(-c2cc3ccccc3c3ccccc23)cc(-c2cc(-c3cc4ccccc4c4ccccc34)c(OCCO)c(-c3cc4ccccc4c4ccccc34)c2)cc1-c1cc2ccccc2c2ccccc12]

To 2,2′-[(3,3′,5,5′-tetrabromo[1,1′-biphenyl]-4,4′-diyl)bis(oxy)]di(ethan-1-ol) (75.2 g; 125 mmol) were added phenanthren-9-ylboronic acid (141.61 g; 625 mmol; 7.5 eq.; purity: 98%) and anisole (390 mL). To this mixture were added 138 g K₃PO₄ dissolved in 306 g of water. The mixture was stirred at 60-70° C. until two clear phases were formed and was purged with argon. To this mixture was added tris(o-tolyl)phosphan (1.52 g; 5.0 mmol) and Pd(OCOCH₃)₂ (281 mg; 1.25 mmol) in 5 mL of anisole under argon and the mixture was stirred under reflux until TLC showed complete conversion.

The mixture was cooled to 70° C. and the organic layer was separated at 70° C. and washed subsequently with an aqueous solution of NaOH (10% by weight), an aqueous HCl solution (2 M) and brine. To the organic layer were added 10 g of activated charcoal (Norit DX Ultra) and 100 g of Na₂SO₄ and the mixture was stirred at 70° C. for 1 hour. Then, the mixture was filtered over celite and the solvent was removed under reduced pressure. The crude product was re-crystallized from a mixture of toluene/MeOH (7/3 w/w) and/or purified via column chromatography (eluent: cyclohexane/ethyl acetate) to obtain the title compound as a white solid with chemical purity of >98%.

m.p. (DSC): 347.2° C. and 392.1° C.;

¹H NMR (80 MHz, CDCl₃): δ=8.79-8.41 (m, 8H, C_{Ar}—H), 8.09-7.32 (m, 32H, C_{Ar}—H), 3.34-3.18 (m, 4H, CH₂), 2.84-2.68 (m, 4H, CH₂), 0.57-0.45 (m, 2H, OH) ppm.

IR (ATR): 488.01 (37.8); 567.09 (72.2); 617.24 (70.9); 692.47 (78.9); 725.26 (43.6); 744.55 (47.9); 765.77 (66.8); 885.36 (71.0); 1014.59 (74.9); 1068.6 (76.0); 1220.98 (71.6); 1361.79 (79.7); 1429.3 (67.4); 1448.59 (72.5) cm⁻¹.

### Example 17: 2,2′-{[3,3′,5,5′-tetrakis(dibenzo[b,d]thiophen-4-yl)[1,1′-biphenyl]-4,4′-diyl]bis(oxy)}di(ethan-1-ol) (compound of formula (Ia-1), with X=a single bond, R¹=R²=R³=R⁴=dibenzo[b,d]thien-4-yl and Z¹=Z²=2-hydroxyethyl; compound 248 of table A)

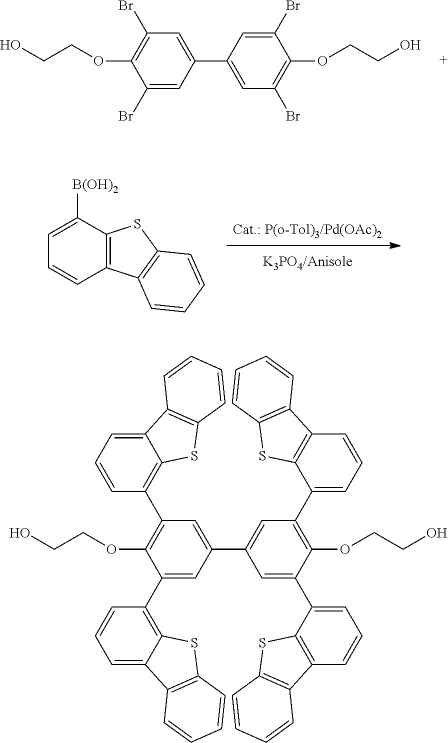

To 2,2′-[(3,3′,5,5′-tetrabromo[1,1′-biphenyl]-4,4′-diyl)bis(oxy)]di(ethan-1-ol) (10 g; 16.3 mmol) were added dibenzo[b,d]thiophen-4-ylboronic acid (19.1 g; 81.37 mmol; 5 eq.; purity=98%) and anisole (51 mL). To this mixture were added 18 g of K₃PO₄ dissolved in 40 g of water. The mixture was stirred at 60-70° C. until two clear phases were formed and was purged with argon. To this mixture was added tris(o-tolyl)phosphan (198 mg; 0.651 mmol) and Pd(OCOCH₃)₂ (37 mg; 0.163 mmol) in 5 mL of anisole under argon and the mixture was stirred under reflux until TLC showed complete conversion.

The mixture was cooled to 70° C. and the organic layer was separated at 70° C. and washed subsequently with an aqueous solution of NaOH (10% by weight), an aqueous HCl solution (2 M) and brine. To the organic layer were added 1.0 g of activated charcoal (Norit DX Ultra) and 10.0 g of Na₂SO₄ and the mixture was stirred at 70° C. for 1 hour. Then, the mixture was filtered over celite and the solvent was removed under reduced pressure. The crude product was re-crystallized from methyl ethyl ketone and/or purified via column chromatography (eluent: cyclohexane/ethyl acetate) to obtain the title compound as a white solid with chemical purity of >94%.

m.p. (DSC): 390.9° C.;

¹H NMR (80 MHz, CDCl₃): δ=8.21-6.98 (m, 32H, C_{Ar}—H), 3.34-3.18 (m, 4H, CH₂), 3.05-2.79 (m, 4H, CH₂), 1.05-0.85 (m, 2H, OH) ppm.

IR (ATR): 607.6 (74.1); 617.24 (65.8); 650.03 (74.3); 686.68 (70.2); 704.04 (71.5); 721.4 (64.4); 744.55 (29.7); 887.28 (61.2); 1031.95 (63.0); 1047.38 (60.8); 1078.24 (64.8); 1232.55 (64.9); 1249.91 (74.9); 1303.92 (78.5); 1357.93 (75.7); 1379.15 (68.1); 1442.8 (65.3) cm⁻¹.

### Example 18: 2,2′-{[3,3′,5,5′-tetra(thianthren-1-yl)[1,1′-biphenyl]-4,4′-diyl]bis(oxy)}di(ethan-1-ol) (compound of formula (Ia-1), with X=a single bond, R¹=R²=R³=R⁴=thianthren-1-yl and Z¹=Z²=2-hydroxyethyl; compound 249 of table A)

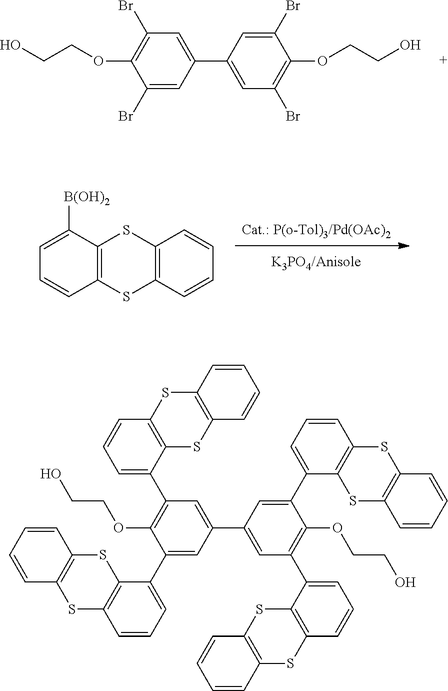 [SMILES: OCCOc1c(Br)cc(-c2cc(Br)c(OCCO)c(Br)c2)cc1Br ; OB(O)c1cccc2c1Sc1ccccc1S2 ; OCCOc1c(-c2cccc3c2Sc2ccccc2S3)cc(-c2cc(-c3cccc4c3Sc3ccccc3S4)c(OCCO)c(-c3cccc4c3Sc3ccccc3S4)c2)cc1-c1cccc2c1Sc1ccccc1S2]

To 2,2′-[(3,3′,5,5′-tetrabromo[1,1′-biphenyl]-4,4′-diyl)bis(oxy)]di(ethan-1-ol) (12.04 g; 20 mmol) were added thianthren-1-ylboronic acid (26.5 g; 100 mmol; 5 eq.; purity: 98%) and anisole (62 mL). To this mixture were added 22.1 g K₃PO₄ dissolved in 49 g of water. The mixture was stirred at 60-70° C. until two clear phases were formed and was purged with argon. To this mixture was added tris(o-tolyl)phosphan (244 mg; 0.8 mmol) and Pd(OCOCH₃)₂ (45 mg; 0.2 mmol) in 5 mL of anisole under argon and the mixture was stirred under reflux until TLC showed complete conversion.

The mixture was cooled to 70° C. and the organic layer was separated at 70° C. and washed subsequently with an aqueous solution of NaOH (10% by weight), an aqueous HCl solution (2 M) and brine. To the organic layer were added 1.0 g of activated charcoal (Norit DX Ultra) and 10.0 g of Na₂SO₄ and the mixture was stirred at 70° C. for 1 hour. Then, the mixture was filtered over celite and the solvent was removed under reduced pressure. The crude product was purified via column chromatography (eluent: cyclohexane/ethyl acetate) to obtain the title compound as a white solid with chemical purity of >94%.

m.p. (DSC): 276.5° C.;

¹H NMR (80 MHz, DMSO-d₆): δ=7.85 (s, 4H, C_{Ar}—H), 7.74-7.16 (m, 28H, C_{Ar}—H), 4.15-4.00 (m, 2H, OH), 3.27-3.05 (m, 4H, CH₂), 2.90-2.70 (m, 4H, CH₂) ppm.

IR (ATR): 410.85 (5.61); 432.07 (43.16); 461.00 (48.69); 476.43 (49.07); 663.53 (75.92); 725.26 (68.4); 746.48 (45.2); 790.84 (73.4); 875.71 (75.99); 1018.45 (75.63); 1070.53 (77.53); 1224.84 (73.71); 1390.72 (71.63); 1431.23 (64.16); 1446.66 (65.87) cm⁻¹.

### Example 19a: 3,3′,5,5′-tetrabromo[1,1′-biphenyl]-2,2′-diol

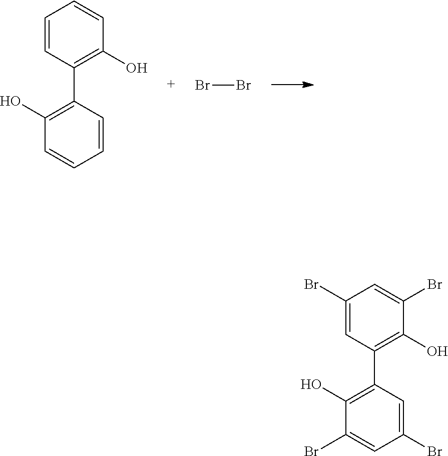

To a solution of 1,1′-biphenyl-2,2′-diol (25.0 g; 134 mmol) in methanol (1000 mL) was added bromine (107 g, 671 mmol, 5.0 eq.) dropwise at 0° C. The reaction was warmed to room temperature and stirred until TLC (heptane/ethyl acetate 2:1) showed complete conversion. The precipitate was filtered off and washed with cold methanol to give the crude product as a yellow solid (49.8 g, 99.2 mmol; yield: 74%). The crude product was recrystallized from acetone to obtain 35.9 g of the titlte compound as an off-white solid.

m.p. (DSC): decomposition at 300° C.

¹H NMR (80 MHz, DMSO-d6): δ=8.67 (br s, 2H), 7.74 (d, J=2.4 Hz, 2H), 7.29 (d, J=2.4 Hz, 2H ppm.

### Example 19b: 2,2′-[(3,3′,5,5′-tetrabromo[1,1′-biphenyl]-2,2′-diyl)bis(oxy)]di(ethan-1-ol)

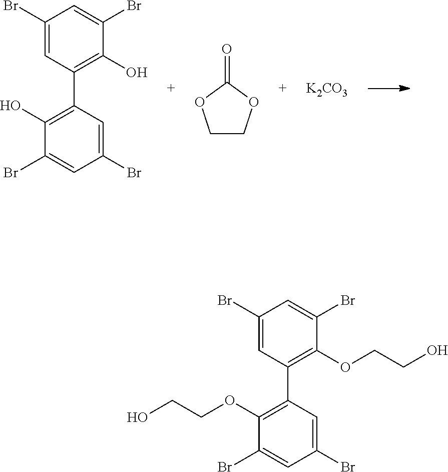

To a suspension of 3,3′,5,5′-tetrabromo[1,1′-biphenyl]-2,2′-diol obtained in Example 19a (29.0 g; 57.8 mmol) in anisole (450 mL) was added K₂CO₃ (31.9 g, 231 mmol, 4.0 eq) and the mixture was stirred for 30 min at 50° C. Ethylene carbonate (50.9 g, 578 mmol, 10 eq.) was added portionwise and the reaction was heated to reflux until TLC (cyclohexane/ethyl acetate (2:1) with roughly 1% of acetic acid) showed complete conversion. The mixture was cooled to room temperature, ethyl acetate (200 mL) and water (100 mL) were added and the phases were separated. The aqueous phase was extracted with ethyl acetate (100 mL). The combined org. phases were washed with water (2×100 mL), dried over Na₂SO₄ and the solvent was removed under reduced pressure to give the crude product as an off-white solid (35.2 g, 57.8 mmol, 100%).

The crude product was recrystallized from toluene to yield 20 g of the title compound as a slightly off-white solid.

¹H NMR (80 MHz, CDCl₃): δ=7.76 (d, J=2.4 Hz, 2H), 7.45 (d, J=2.4 Hz, 2H), 3.85-3.49 (m, 8H), 2.08 (t, J=5.6 Hz, 2H) ppm.

### Example 19c: 2,2′-{[3,3′,5,5′-tetra(phenanthren-9-yl)[1,1′-biphenyl]-2,2′-diyl]bis(oxy)}di(ethan-1-ol) (compound of formula (Ia-1), with X=a single bond, R¹=R²=R³=R⁴=thianthren-1-yl and Z¹=Z²=2-hydroxyethyl; compound 12 of table B)

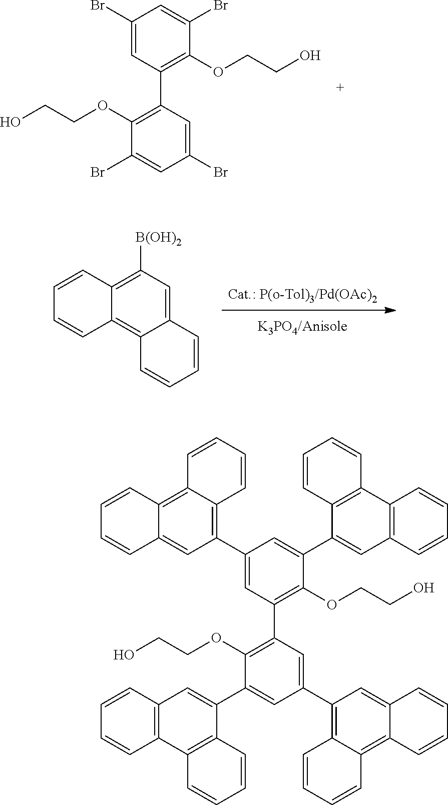 [SMILES: OCCOc1c(Br)cc(Br)cc1-c1cc(Br)cc(Br)c1OCCO ; OB(O)c1cc2ccccc2c2ccccc12 ; OCCOc1c(-c2cc(-c3cc4ccccc4c4ccccc34)cc(-c3cc4ccccc4c4ccccc34)c2OCCO)cc(-c2cc3ccccc3c3ccccc23)cc1-c1cc2ccccc2c2ccccc12]

To a mixture of 2,2′-[(3,3′,5,5′-tetrabromo[1,1′-biphenyl]-2,2′-diyl)bis(oxy)]di(ethan-1-ol) obtained in Example 19b (17.0 g; 28.8 mmol) and 9-phenanthrylboronic acid (32.0 g; 144 mmol; 5.0 eq.) in anisole (100 g) was added a solution of K₃PO₄ (31.8 g, 150 mmol, 5.2 eq.) in water (70 g) and the mixture heated to 70° C. Tris(o-tolyl)phosphane (0.175 g, 0.576 mmol, 2.0 mol %) and Pd(OCOCH₃)₂ (32.4 mg; 0.144 mmol, 0.5 mol %) were added and the reaction mixture was heated to reflux until TLC (cyclohexane/ethyl acetate 3:1) showed no further progress.

The reaction mixture was cooled to room temperature, ethyl acetate (100 g) was added and the layers separated. The aqueous phase was extracted with ethyl acetate (50 g). The combined organic phases were washed successively with water (100 g), then brine (100 g), dried over Na₂SO₄ and the solvent removed under reduced pressure to give the crude product. Purification via column chromatography (cyclohexane/ethyl acetate 3:1) gave 21.5 g of the title compound as a white solid with a chemical purity of >95%.

m.p.=196° C.-205° C.

¹H NMR (80 MHz, CDCl₃): δ=9.04-8.54 (m, 8H), 8.43-7.42 (m, 32H), 3.95-3.05 (m, 8H), 1.71 (t, J=6.2 Hz, 2H) ppm.

### 2.3 Refractive Indices No of Monomers of Formula (I):

The following table C lists refractive indices of some monomers of formula (I) that were calculated using the software ACD/ChemSketch 2012 (Advanced Chemistry Development, Inc.). The individual monomers are identified in table C by their entry numbers in tables A and B, respectively. In addition, it has been verified by quantum chemical calculations for all monomers included in table C that they do not, or only to a negligible extent, absorb in the visible light range and are therefore basically colorless.

TABLE C

| | from | n_{D} (calc.) |
|---|---|---|
| Entry # | Table | monomer |
| 1 | A | 1.64 |
| 2 | A | 1.73 |
| 3 | A | 1.73 |
| 4 | A | 1.785 |
| 5 | A | 1.83 |
| 6 | A | 1.83 |
| 7 | A | 1.79 |
| 8 | A | 1.79 |
| 9 | A | 1.67 |
| 10 | A | 1.75 |
| 11 | A | 1.75 |
| 12 | A | 1.80 |
| 13 | A | 1.84 |
| 14 | A | 1.84 |
| 15 | A | 1.80 |
| 16 | A | 1.80 |
| 17 | A | 1.76 |
| 18 | A | 1.84 |
| 19 | A | 1.84 |
| 20 | A | 1.90 |
| 21 | A | 1.94 |
| 22 | A | 1.94 |
| 23 | A | 1.955 |
| 24 | A | 1.955 |
| 25 | A | 1.66 |
| 26 | A | 1.75 |
| 27 | A | 1.75 |
| 28 | A | 1.81 |
| 29 | A | 1.86 |
| 30 | A | 1.86 |
| 31 | A | 1.815 |
| 32 | A | 1.815 |
| 33 | A | 1.615 |
| 34 | A | 1.69 |
| 35 | A | 1.69 |
| 36 | A | 1.75 |
| 50 | A | 1.78 |
| 51 | A | 1.78 |
| 52 | A | 1.76 |
| 53 | A | 1.76 |
| 54 | A | 1.63 |
| 55 | A | 1.71 |
| 56 | A | 1.71 |
| 57 | A | 1.76 |
| 71 | A | 1.79 |
| 72 | A | 1.79 |
| 73 | A | 1.77 |
| 74 | A | 1.77 |
| 75 | A | 1.71 |
| 76 | A | 1.79 |
| 77 | A | 1.79 |
| 78 | A | 1.85 |
| 92 | A | 1.885 |
| 93 | A | 1.885 |
| 94 | A | 1.90 |
| 95 | A | 1.90 |
| 243 | A | 1.63 |
| 244 | A | 1.71 |
| 245 | A | 1.71 |
| 246 | A | 1.77 |
| 247 | A | 1.81 |
| 248 | A | 1.81 |
| 249 | A | 1.775 |
| 250 | A | 1.775 |
| 251 | A | 1.60 |
| 252 | A | 1.67 |
| 253 | A | 1.67 |
| 254 | A | 1.73 |
| 255 | A | 1.76 |
| 256 | A | 1.76 |
| 257 | A | 1.73 |
| 258 | A | 1.73 |
| 259 | A | 1.61 |
| 260 | A | 1.68 |
| 261 | A | 1.68 |
| 262 | A | 1.74 |
| 263 | A | 1.765 |
| 264 | A | 1.765 |
| 265 | A | 1.74 |
| 266 | A | 1.74 |
| 267 | A | 1.68 |
| 268 | A | 1.76 |
| 269 | A | 1.76 |
| 270 | A | 1.82 |
| 271 | A | 1.85 |
| 272 | A | 1.85 |
| 273 | A | 1.86 |
| 274 | A | 1.86 |
| 275 | A | 1.60 |
| 276 | A | 1.685 |
| 277 | A | 1.685 |
| 278 | A | 1.74 |
| 279 | A | 1.78 |
| 280 | A | 1.78 |
| 281 | A | 1.75 |
| 282 | A | 1.75 |
| 283 | A | 1.64 |
| 284 | A | 1.70 |
| 285 | A | 1.70 |
| 286 | A | 1.75 |
| 287 | A | 1.78 |
| 288 | A | 1.78 |
| 289 | A | 1.76 |
| 290 | A | 1.76 |
| 291 | A | 1.655 |
| 292 | A | 1.72 |
| 293 | A | 1.72 |
| 294 | A | 1.76 |
| 295 | A | 1.79 |
| 296 | A | 1.79 |
| 297 | A | 1.765 |
| 298 | A | 1.765 |
| 299 | A | 1.73 |
| 300 | A | 1.795 |
| 301 | A | 1.795 |
| 302 | A | 1.84 |
| 303 | A | 1.875 |
| 304 | A | 1.875 |
| 305 | A | 1.89 |
| 306 | A | 1.89 |
| 307 | A | 1.65 |
| 308 | A | 1.72 |
| 309 | A | 1.72 |
| 310 | A | 1.77 |
| 311 | A | 1.80 |
| 312 | A | 1.80 |
| 313 | A | 1.77 |
| 314 | A | 1.77 |
| 315 | A | 1.64 |
| 316 | A | 1.70 |
| 317 | A | 1.70 |
| 318 | A | 1.75 |
| 319 | A | 1.78 |
| 320 | A | 1.78 |
| 321 | A | 1.76 |
| 322 | A | 1.76 |
| 323 | A | 1.655 |
| 324 | A | 1.72 |
| 325 | A | 1.72 |
| 326 | A | 1.76 |
| 327 | A | 1.79 |
| 328 | A | 1.79 |
| 329 | A | 1.765 |
| 330 | A | 1.765 |
| 331 | A | 1.73 |
| 332 | A | 1.795 |
| 333 | A | 1.795 |
| 334 | A | 1.84 |
| 335 | A | 1.875 |
| 336 | A | 1.875 |
| 337 | A | 1.89 |
| 338 | A | 1.89 |
| 339 | A | 1.65 |
| 340 | A | 1.72 |
| 341 | A | 1.72 |
| 342 | A | 1.77 |
| 343 | A | 1.80 |
| 344 | A | 1.80 |
| 345 | A | 1.77 |
| 346 | A | 1.77 |
| 347 | A | 1.67 |
| 348 | A | 1.73 |
| 349 | A | 1.73 |
| 350 | A | 1.77 |
| 351 | A | 1.805 |
| 352 | A | 1.805 |
| 353 | A | 1.775 |
| 354 | A | 1.775 |
| 355 | A | 1.69 |
| 356 | A | 1.74 |
| 357 | A | 1.74 |
| 358 | A | 1.78 |
| 359 | A | 1.805 |
| 360 | A | 1.805 |
| 361 | A | 1.78 |
| 362 | A | 1.78 |
| 363 | A | 1.765 |
| 364 | A | 1.82 |
| 365 | A | 1.82 |
| 366 | A | 1.86 |
| 367 | A | 1.89 |
| 368 | A | 1.89 |
| 369 | A | 1.905 |
| 370 | A | 1.905 |
| 371 | A | 1.69 |
| 372 | A | 1.745 |
| 373 | A | 1.745 |
| 374 | A | 1.79 |
| 375 | A | 1.82 |
| 376 | A | 1.82 |
| 377 | A | 1.79 |
| 378 | A | 1.79 |
| 379 | A | 1.67 |
| 380 | A | 1.73 |
| 381 | A | 1.73 |
| 382 | A | 1.77 |
| 383 | A | 1.80 |
| 384 | A | 1.80 |
| 385 | A | 1.775 |
| 386 | A | 1.775 |
| 387 | A | 1.69 |
| 388 | A | 1.74 |
| 389 | A | 1.74 |
| 390 | A | 1.78 |
| 391 | A | 1.805 |
| 392 | A | 1.805 |
| 393 | A | 1.78 |
| 394 | A | 1.78 |
| 395 | A | 1.765 |
| 396 | A | 1.82 |
| 397 | A | 1.82 |
| 398 | A | 1.86 |
| 399 | A | 1.89 |
| 400 | A | 1.89 |
| 401 | A | 1.905 |
| 402 | A | 1.905 |
| 403 | A | 1.69 |
| 404 | A | 1.745 |
| 405 | A | 1.745 |
| 406 | A | 1.79 |
| 407 | A | 1.82 |
| 408 | A | 1.82 |
| 409 | A | 1.79 |
| 410 | A | 1.79 |
| 411 | A | 1.67 |
| 412 | A | 1.73 |
| 413 | A | 1.73 |
| 414 | A | 1.77 |
| 415 | A | 1.80 |
| 416 | A | 1.80 |
| 417 | A | 1.775 |
| 418 | A | 1.775 |
| 419 | A | 1.69 |
| 420 | A | 1.74 |
| 421 | A | 1.74 |
| 422 | A | 1.78 |
| 423 | A | 1.805 |
| 424 | A | 1.805 |
| 425 | A | 1.78 |
| 426 | A | 1.78 |
| 427 | A | 1.765 |
| 428 | A | 1.82 |
| 429 | A | 1.82 |
| 430 | A | 1.86 |
| 431 | A | 1.89 |
| 432 | A | 1.89 |
| 433 | A | 1.905 |
| 434 | A | 1.905 |
| 435 | A | 1.69 |
| 436 | A | 1.745 |
| 437 | A | 1.745 |
| 438 | A | 1.79 |
| 439 | A | 1.82 |
| 440 | A | 1.82 |
| 441 | A | 1.79 |
| 442 | A | 1.79 |
| 1 | B | 1.66 |
| 2 | B | 1.75 |
| 3 | B | 1.75 |
| 4 | B | 1.81 |
| 5 | B | 1.86 |
| 6 | B | 1.86 |
| 7 | B | 1.815 |
| 8 | B | 1.815 |
| 9 | B | 1.63 |
| 10 | B | 1.71 |
| 11 | B | 1.71 |
| 12 | B | 1.77 |
| 13 | B | 1.81 |
| 14 | B | 1.81 |
| 15 | B | 1.775 |
| 16 | B | 1.775 |
| 17 | B | 1.60 |
| 18 | B | 1.685 |
| 19 | B | 1.685 |
| 20 | B | 1.74 |
| 21 | B | 1.78 |
| 22 | B | 1.78 |
| 23 | B | 1.75 |
| 24 | B | 1.75 |
| 25 | B | 1.65 |
| 26 | B | 1.72 |
| 27 | B | 1.72 |
| 28 | B | 1.77 |
| 29 | B | 1.80 |
| 30 | B | 1.80 |
| 31 | B | 1.77 |
| 32 | B | 1.77 |
| 33 | B | 1.65 |
| 34 | B | 1.72 |
| 35 | B | 1.72 |
| 36 | B | 1.77 |
| 37 | B | 1.80 |
| 38 | B | 1.80 |
| 39 | B | 1.77 |
| 40 | B | 1.77 |
| 41 | B | 1.69 |
| 42 | B | 1.745 |
| 43 | B | 1.745 |
| 44 | B | 1.79 |
| 45 | B | 1.82 |
| 46 | B | 1.82 |
| 47 | B | 1.79 |
| 48 | B | 1.79 |
| 49 | B | 1.69 |
| 50 | B | 1.745 |
| 51 | B | 1.745 |
| 52 | B | 1.79 |
| 53 | B | 1.82 |
| 54 | B | 1.82 |
| 55 | B | 1.79 |
| 56 | B | 1.79 |
| 57 | B | 1.69 |
| 58 | B | 1.745 |
| 59 | B | 1.745 |
| 60 | B | 1.79 |
| 61 | B | 1.82 |
| 62 | B | 1.82 |
| 63 | B | 1.79 |
| 64 | B | 1.79 |

3. Preparation of Polycarbonate Resins from Monomers of Formula (I)
3.1 Analytics Relating to Resins Prepared from Monomers of Formula (I):

### Refractive Index (n_{D}):

The refractive index was measured using a disk shaped test piece with a thickness of 3 mm made by polycarbonate resin as a test piece according to JIS B 7071-2:2018. The measurement was conducted at 23° C. using the refractive index measurement device below.
Refractive index measurement device:
KPR-3000 manufactured by Shimadzu Corporation
   ABBE number (ν):

A disk shaped test piece with a thickness of 3 mm which is same as the test piece used in the refractive index measurement was used. The refractive index values were measured using the refractive index measurement device below at 23° C. and at wavelengths of 486 nm, 589 nm and 656 nm. Then, the Abbe number was calculated using the below-described formula.
Refractive index measurement device:
KPR-3000 manufactured by Shimadzu Corporation

$$v = ( nD - 1 ) / ( nF - nC )$$

n_{D}: refractive index at a wavelength of 589 nm
n_{C}: refractive index at a wavelength of 656 nm
n_{F}: refractive index at a wavelength of 486 nm

### Glass Transition Temperature (Tg):

The glass transition temperature was measured by differential scanning calorimetry (DSC) using a 10° C./minute heating program according to JIS K7121-1987.
Differential scanning calorimetry device:
X-DSC7000 manufactured by Hitachi High-Tech Science Corporation

### Molecular Weight

The values of the weight average molecular weight (Mw) of the resins were measured in accordance with the gel permeation chromatography (GPC) method and calculated by the standard polystyrene conversion approach. The following devices, columns and measurement conditions were used:

GPC device: HLC-8420GPC (from Tosoh Corporation);
Columns: three TSKgel SuperHM-M (from Tosoh Corporation),
one guard column SuperHM-M (from Tosoh Corporation),
one TSKgel SuperH-RC (from Tosoh Corporation);
   Detection Device: RI detection
   Standard polystyrene: PstQuick C as standard polystyrene kit (from Tosoh Corporation);
   Eluent: tetrahydrofuran;
   Flow rate of eluent: 0.6 ml/min;
   Column temperature: 40° C.

The number average molecular weight (Mn) values can be calculated using similar methods to those used for measuring the Mw values described above. The polystyrene converted weight average molecular weights (Mw) and number average molecular weights (Mn) were calculated using a previously prepared standard curve of polystyrene. Specifically, the standard curve was prepared using a standard polystyrene for which the molecular weight was known (“PStQuick C” from Tosoh Corporation). Further, a calibration curve was obtained by plotting the elution time and molecular weight value of each of the peaks based on the measured data of the standard polystyrene, and conducting three-dimensional approximation. The values for Mw and Mn were calculated based on the following calculation formulae:

$$Mw = Σ ( Wi \times Mi ) \div Σ ( Wi ) \\ Mn = Σ ( Ni \times Mi ) \div Σ ( Wi )$$

In the calculation formulae, “i” represents the “i”th dividing point, “Wi” represents the molecular weight (g) of the polymer at the “i”th dividing point, “Ni” represents the number of the molecules of the polymer at the “i”th dividing point, and “Mi” represents the molecular mass at the “i”th dividing point. The molecular mass (M) represents the value of the molecular mass of polystyrene at the corresponding elution time in the calibration curve.

### Contents of Low Molecular Weight Compounds (CLWC)

The content of low molecular weight compounds (CLWC) represents to the ratio of the combined peak areas of compounds with Mw values below 1000 to the total area of all peaks, where the peak areas are determined according the GPC analysis described above. Therefore, CLWC values were determined using the following formula:

$$CLWC ( \% ) = \frac{\begin{matrix}the total area of peaks of compounds with \\ Mw lower than 1. on GPC analysis\end{matrix}}{( the total area of all peaks of compounds on GPC analysis )} \times 100$$

The GPC analyses used in this context were performed in analogy to those described above for measuring the molecular weight of the thermoplastic resins.

### Birefringence (Δn):

Each resin example to be analyzed was dissolved in methylene chloride (solvent) to form a solution with the concentration of 10 weight-%. The obtained solution was casted on an SUS plate whose surface had been treated with electroplating and a cast film was made followed by evaporating the solvent at 25° C. A square film piece of 50 mm per side having a thickness of 100 μm was cut out from the cast film. The film piece was stretched 1.5-fold below at a temperature 20° C. higher than the Tg of the resin. Streching was carried out using the stretching machine SS-70 manufactured by Shibayama Scientific Co., Ltd. The obtained stretched film was subjected to retardation measurement using the ellipsometer M-220 manufactured by JASCO Corporation.

From the retardation/phase difference Re the birefringence values Δn are calculated by the following equation:

$$Δ n = \vert Re / d \vert$$

Δn: orientation birefringence
Re: phase difference [nm]
d: thickness [nm]

The algebraic sign of the birefringence is represented by the following equation with the use of the refractive index (n_{∥}) in the stretching direction of the film and the refractive index (n_{⊥}) in the direction perpendicular to the stretching direction:

$$Δ n = {n}_{\vert} - {n}_{⊥}$$

If Δn is positive, it is called positive birefringence, while if Δn is negative, it is called negative birefringence.

### 3.2 Examples for the Preparation of Homopolycarbonates:

### Examples 20 to 30

The following Table 1 lists physical properties, namely refractive indices (n_{D}), Abbe numbers (ν), glass transistion temperatures (Tg) and birefringences (Δn), of the homopolycarbonates of Examples 20 to 30 that are obtainable by reacting one of the monomers of formula (Ia-1) prepared in Examples 1, 2, 3, 4, 5, 6, 7, 8, 9, 10 or 13 as diol component with a carbonate forming monomer, such as diphenylcarbonate, by analogy methods for preparing polyestercarbonates well known in the art. Table 1 also lists the n_{D}- and Tg-values of two comparative homopolycarbonates prepared from 9,9-bis(4-(2-hydroxyethoxy)phenyl)fluorene and Biphenol A as diol component, respectively. Accordingly, the homopolycarbonates of Examples 20 to 30 each consist of the respective structural units of the formula (IIa-1) and structural units of the formula (III-1), while the comparative homopolycarbonates consist of the structural unites derived from the monomers 9,9-bis(4-(2-hydroxyethoxy)phenyl)fluorene and bisphenol A, respectively, and structural units of formula (III-1).

The n_{D}-, ν-, Tg- and Δn-values of the homopolycarbonates of Examples 20 to 30 given in Table 1 were calculated from the respective values of the corresponding copolymers derived from the monomer of Example 1, 2, 3, 4, 5, 6, 7, 8, 9, 10 or 13 by using the above-mentioned Fox equation. The preparations of these copolymers and their physical data are described in the Examples 31 to 40 below. The n_{D}- and Tg-values of the comparative homopolycarbonates are taken from U.S. Pat. No. 9,360,593.

TABLE 1
Properties of homopolycarbonates of Examples 20 to 30

| | | | | Tg | |
|---|---|---|---|---|---|
| Example | Monomer | n_{D} | ν | [° C.] | Δn |
| 20 | 2-[4-[1-[4-(2-hydroxyethoxy)- | 1.626 | 23.3 | 131 | Very weakly |
| | 3,5-diphenyl-phenyl]-1-methyl- | | | | positive Δn |
| | ethyl]-2,6-diphenyl- | | | | |
| | phenoxy]ethanol (see Ex. 1) | | | | |
| 21 | 2-[4-[1-[4-(2-hydroxyethoxy)- | 1.669 | 18.5 | 145 | |
| | 3,5-di(naphthalen-1-yl)- | | | | |
| | phenyl]-1-methyl-ethyl]-2,6- | | | | |
| | di(naphthalen-1-yl)- | | | | |
| | phenoxy]ethanol (see Ex. 2) | | | | |
| 22 | 2-[4-[1-[4-(2-hydroxyethoxy)- | 1.669 | 16.6 | 154 | Very weakly |
| | 3,5-di(naphthalen-2-yl)- | | | | positive Δn |
| | phenyl]-1-methyl-ethyl]-2,6- | | | | |
| | di(naphthalen-2-yl)- | | | | |
| | phenoxyethanol (see Ex. 3) | | | | |
| 23 | 2-[4-[1-[4-(2-hydroxyethoxy)- | 1.712 | 16 | 235 | Strongly |
| | 3,5-di(phenanthren-9-yl)- | | | | negative Δn |
| | phenyl]-1-methyl-ethyl]-2,6- | | | | |
| | di(phenanthren-9-yl)- | | | | |
| | phenoxy]ethanol (see Ex. 4b) | | | | |
| 24 | 2-[4-[1-[4-(2-hydroxyethoxy)- | 1.712 | 17.9 | 195 | |
| | 3,5-di(1,2-dibenzo[b,d]thien-4- | | | | |
| | yl)-phenyl]-1-methyl-ethyl]-2,6- | | | | |
| | di(1,2-dibenzo[b,d]thien-4-yl)- | | | | |
| | phenoxy]ethanol (see Ex. 5) | | | | |
| 25 | 2-[4-[4-(2-hydroxyethoxy)-3,5- | 1.679 | 18.6 | 155 | |
| | di(naphthalen-1- | | | | |
| | yl)phenyl]sulfonyl-2,6- | | | | |
| | di(naphthalen-1- | | | | |
| | yl)phenoxy]ethanol (see Ex. 6) | | | | |
| 26 | 2-[4-[4-(2-hydroxyethoxy)-3,5- | 1.684 | 18.9 | 74 | |
| | di(naphthalen-1- | | | | |
| | yl)phenyl]sulfanyl-2,6- | | | | |
| | di(naphthalen-1- | | | | |
| | yl)phenoxy]ethanol (see Ex. 7) | | | | |
| 27 | 2-[4-[4-(2-hydroxyethoxy)-3,5- | 1.716 | 16.7 | 197 | |
| | di(phenanthren-9- | | | | |
| | yl)phenyl]sulfonyl-2,6- | | | | |
| | di(phenanthren-9- | | | | |
| | yl)phenoxy]ethanol (see Ex. 8) | | | | |
| 28 | 2-[4-[4-(2-hydroxyethoxy)-3,5- | 1.716 | 16.9 | 214 | |
| | di(dibenzo[b,d]thien-4- | | | | |
| | yl)phenyl]sulfonyl-2,6- | | | | |
| | dibenzo[b,d]thien-4- | | | | |
| | yl)phenoxy]ethanol (see Ex. 9) | | | | |
| 29 | 2-[4-[4-(2-hydroxyethoxy)-3,5- | 1.709 | 20.1 | 166 | |
| | di(thianthrene-1- | | | | |
| | yl)phenyl]sulfonyl-2,6- | | | | |
| | di(thianthrene-1- | | | | |
| | yl)phenoxy]ethanol (see Ex. | | | | |
| | 10) | | | | |
| 30 | 2,2′-(propane-2,2-diylbis{[2,6- | 1.702 | 20.6 | 197 | |
| | di(thianthren-1-yl)-4,1- | | | | |
| | phenylene]oxy})di(ethan-1-ol) | | | | |
| | (see Ex. 13) | | | | |
| — | 9,9-bis(4-(2-hydroxyethoxy)- | 1.639* | | 149* | |
| | phenyl)fluorene* | | | | |
| — | Bisphenol A* | 1.589* | | 148* | |

*Comparative compounds; np values and Tg values as reported in U.S. Pat. No. 9,360,593

### 3.3 Examples for the Preparation of Copolycarbonates:

### Example 31: Copolymer Prepared from Monomer of Example 1 and BPEF

As materials, 3.00 kg (4.83 mol) of 2-[4-[1-[4-(2-hydroxyethoxy)-3,5-diphenyl-phenyl]-1-methyl-ethyl]-2,6-diphenyl-phenoxy]ethanol (see Example 1, hereinafter also designated as TPBHBPA), 12.01 kg (27.39 mol) of 9,9-bis[4-(2-hydroxyethoxy)phenyl)fluorene (BPEF), 7.11 kg (33.19 mol) of diphenylcarbonate (DPC) and 15 ml of a 2.5×10² mol/l (7.8×10⁻⁴ mol) aqueous solution of sodium hydrogen carbonate were put into a 50 liter reactor with a stirrer and a distillation device.

After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. Then, the pressure was reduced to 120 Torr in 10 minutes, and the reaction conditions of 240° C. and 120 Torr were maintained for 70 minutes. Then, the pressure was reduced to 100 Torr in 10 minutes, and the reaction conditions of 240° C. and 100 Torr were maintained for 10 minutes. Further, the pressure was reduced to 1 Torr or lower in 40 minutes, and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 2 below.

TABLE 2

| | |
|---|---|
| Monomers | Properties |

| | (mol-%) | | | | Tg |
|---|---|---|---|---|---|
| Resin | TPBHBPA | BPEF | n_{D} | ν | [° C.] |
| Example 31 | 15 | 85 | 1.637 | 23.5 | 142 |
| Example 20* | 100 | 0 | 1.626 | 23.3 | 131 |

*the values given for the homopolymer of Example 20 correspond to those already listed in Table 1.

### Example 32: Copolymer Prepared from Monomer of Example 2 and BPEF

As materials, 4.70 kg (5.72 mol) of 2-[4-[1-[4-(2-hydroxyethoxy)-3,5-di(naphthalen-1-yl)phenyl]-1-methyl-ethyl]-2,6-di(naphthalen-1-yl)phenoxy]ethanol (see Example 2, hereinafter also designated as T1NBHBPA), 10.04 kg (22.90 mol) of 9,9-bis[4-(2-hydroxyethoxy)phenyl)fluorene (BPEF), 6.41 kg (29.90 mol) of diphenylcarbonate (DPC) and 11 ml of a 2.5×10⁻² mol/l (2.8×10−4 mol) aqueous solution of sodium hydrogen carbonate were put into a 50 liter reactor with a stirrer and a distillation device. After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. Then, the pressure was reduced to 120 Torr in 10 minutes and the reaction conditions of 240° C. and 120 Torr were maintained for 70 minutes. Afterwards, the pressure was reduced to 100 Torr in 10 minutes, and the reaction conditions of 240° C. and 100 Torr were maintained for 10 minutes. Further, the pressure was reduced to 1 Torr or lower in 40 minutes, and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 3.

TABLE 3

| | |
|---|---|
| Monomers | Properties |

| | (mol-%) | | | | Tg |
|---|---|---|---|---|---|
| Resin | T1NBHBPA | BPEF | n_{D} | ν | [° C.] |
| Example 32 | 20 | 80 | 1.649 | 21.7 | 145 |
| Example 21* | 100 | 0 | 1.669 | 18.5 | 145 |

*the values given for the homopolymer of Example 21 correspond to those already listed in Table 1.

### Example 33: Copolymer Prepared from Monomer of Example 3 and BPEF

As materials, 6.51 kg (7.93 mol) of 2-[4-[1-[4-(2-hydroxyethoxy)-3,5-di(naphthalen-2-yl)phenyl]-1-methyl-ethyl]-2,6-di(naphthalen-2-yl)phenoxy]ethanol (see Example 3, hereinafter also designated as T2NBHBPA), 8.12 kg (18.51 mol) of 9,9-bis[4-(2-hydroxyethoxy)phenyl)fluorene (BPEF), 5.83 kg (27.22 mol) of diphenylcarbonate (DPC) and 31 ml of a 2.5×10⁻² mol/l (7.8×10−4 mol) aqueous solution of sodium hydrogen carbonate were put into a 50 liter reactor with a stirrer and a distillation device. After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction had been conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. Then, the pressure was reduced to 120 Torr in 10 minutes, and the reaction conditions of 240° C. and 120 Torr were maintained for 70 minutes. Afterwards, the pressure was reduced to 100 Torr in 10 minutes, and the reaction conditions of 240° C. and 100 Torr were maintained for 10 minutes. Further, the pressure was reduced to 1 Torr or lower in 40 minutes, and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 4.

TABLE 4

| | |
|---|---|
| Monomers | Properties |

| | (mol-%) | | | | Tg |
|---|---|---|---|---|---|
| Resin | T2NBHBPA | BPEF | n_{D} | ν | [° C.] |
| Example 33 | 30 | 70 | 1.665 | 19.85 | 149 |
| Example 22* | 100 | 0 | 1.669 | 16.61 | 154 |

*the values given for the homopolymer of Example 22 correspond to those already listed in Table 1.

### Example 34: Copolymer Prepared from Monomer of Example 4b and BPEF

As materials, 8.32 kg (8.16 mol) of 2-[4-[1-[4-(2-hydroxyethoxy)-3,5-di(phenanthren-9-yl)-phenyl]-1-methyl-ethyl]-2,6-di(phenanthren-9-yl)-phenoxy]ethanol (see Example 4b, hereinafter also designated as T9PNBHBPA), 8.35 kg (19.04 mol) of 9,9-bis[4-(2-hydroxyethoxy)phenyl)fluorene (BPEF), 6.00 kg (28.01 mol) of diphenylcarbonate (DPC) and 32 ml of a 2.5×10−2 mol/l (7.8×10−4 mol) aqueous solution of sodium hydrogen carbonate were put into a 50 liter reactor with a stirrer and a distillation device. After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction had been conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. Then, the pressure was reduced to 120 Torr in 10 minutes, and the reaction conditions of 240° C. and 120 Torr were maintained for 70 minutes. Afterwards, the pressure was reduced to 100 Torr in 10 minutes, and the reaction conditions of 240° C. and 100 Torr were maintained for 10 minutes. Further, the pressure was reduced to 1 Torr or lower in 40 minutes, and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 5.

TABLE 5

| | Monomers | | Properties | | |
|---|---|---|---|---|---|
| | (mol-%) | | | | Tg |
| Resin | T9PNBHBPA | BPEF | n_{D} | ν | [° C.] |
| Example 34 | 30 | 70 | 1.675 | 19.1 | 179 |
| Example 23* | 100 | 0 | 1.712 | 16 | 235 |

*the values given for the homopolymer of Example 23 correspond to those already listed in Table 1.

### Example 35: Copolymer Prepared from Monomer of Example 5 and BPEF

As materials, 9.00 kg (8.61 mol) of 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)phenoxy]ethanol (or 2,2′-((propane-2,2-diylbis(2,6-bis(dibenzo[b,d]thiophen-4-yl)-4,1-phenylene))bis(oxy))bis(ethan-1-ol), see Example 5, hereinafter also designated as T4DBTBHBPA), 8.81 kg (20.09 mol) of 9,9-bis[4-(2-hydroxyethoxy)phenyl)fluorene (BPEF), 6.33 kg (29.56 mol) of diphenylcarbonate (DPC) and 11 ml of a 2.5×10⁻² mol/l (2.8×10⁻⁴ mol) aqueous solution of sodium hydrogen carbonate were put into a 50 liter reactor with a stirrer and a distillation device.

After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. Afterwards, the pressure was reduced to 120 Torr in 10 minutes, and the reaction conditions of 240° C. and 120 Torr were maintained for 70 minutes. The pressure was then reduced to 100 Torr in 10 minutes and the reaction conditions of 240° C. and 100 Torr were maintained for 10 minutes. Further, the pressure was reduced to 1 Torr or lower in 40 minutes, and the polymerization reaction was conducted at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 6 below.

TABLE 6

| | Monomers | | Properties | | | | |
|---|---|---|---|---|---|---|---|
| | (mol-%) | | | | Tg | | |
| Resin | T4DBTBHBPA | BPEF | n_{D} | ν | [° C.] | Mw | Mn |
| Example | 30 | 70 | 1.675 | 20.3 | — | 26459 | 6524 |
| 35 | | | | | | | |
| Example | 100 | 0 | 1.712 | 17.9 | 195 | — | — |
| 24* | | | | | | | |

*the values given for the homopolymer of Example 24 correspond to those already listed in Table 1.

### Example 36: Copolymer Prepared from Monomer of Example 6 and BPEF

As materials, 7.00 kg (8.30 mol) of 2-[4-[4-(2-hydroxyethoxy)-3,5-di(naphthalen-1-yl)phenyl]sulfonyl-2,6-di(naphthalen-1-yl)phenoxy]ethanol (see Example 6, hereinafter also designated as T1NBHBPS), 8.50 kg (19.38 mol) of 9,9-bis[4-(2-hydroxyethoxy)phenyl)fluorene (BPEF), 6.11 kg (28.51 mol) of diphenylcarbonate (DPC) and 11 ml of a 2.5×10⁻² mol/l (2.8×10⁻⁴ mol) aqueous solution of sodium hydrogen carbonate were put into a 50 liter reactor with a stirrer and a distillation device. After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. Afterwards, the pressure was reduced to 120 Torr in 10 minutes, and the reaction conditions of 240° C. and 120 Torr were maintained for 70 minutes. The pressure was then reduced to 100 Torr in 10 minutes, and the reaction conditions of 240° C. and 100 Torr were maintained for 10 minutes. Further, the pressure was reduced to 1 Torr or lower in 40 minutes, and the polymerization reaction was conducted at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 7 below.

TABLE 7

| | |
|---|---|
| Monomers | Properties |

| | (mol-%) | | | | Tg |
|---|---|---|---|---|---|
| Resin | T1NBHBPS | BPEF | n_{D} | ν | [° C.] |
| Example 37 | 30 | 70 | 1.658 | 20.9 | 150 |
| Example 25* | 100 | 0 | 1.679 | 18.6 | 155 |

*the values given for the homopolymer of Example 25 correspond to those already listed in Table 1.

### Example 37: Copolymer Prepared from Monomer of Example 7 and BPEF

As materials, 4.50 kg (5.55 mol) of 2-[4-[4-(2-hydroxyethoxy)-3,5-di(naphthalen-1-yl)phenyl]sulfanyl-2,6-di(naphthalen-1-yl)phenoxy]ethanol (or 2,2′-((thiobis(2,6-di(naphthalen-1-yl)-4,1-phenylene))bis(oxy))bis(ethan-1-ol see Example 7, hereinafter also designated as T1NBHTDP), 9.73 kg (22.19 mol) of 9,9-bis[4-(2-hydroxyethoxy)phenyl)fluorene (BPEF), 6.64 kg (28.58 mol) of diphenylcarbonate (DPC) and 11 ml of a 2.5×10⁻² mol/l (2.8×10⁻⁴ mol) aqueous solution of sodium hydrogen carbonate were put into a 50 liter reactor with a stirrer and a distillation device. After the reactor had been flushed with nitrogen, the reaction mixture was heated for 1 hour to 205° C. and stirred at a pressure of 760 Torr. After the reaction mixture was completely dissolved, the pressure was reduced to 150 Torr in 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. And then, the pressure was reduced to 120 Torr in 10 minutes and the reaction conditions of 240° C. and 120 Torr were maintained for 70 minutes. Afterwards, the pressure was reduced to 100 Torr in 10 minutes and the reaction conditions of 240° C. and 100 Torr were maintained for 10 minutes. Further, the pressure was reduced to 1 Torr or lower in 40 minutes and the polymerization reaction was conducted at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 8.

TABLE 8

| | Monomers | | Properties | | | | |
|---|---|---|---|---|---|---|---|
| | (mol-%) | | | | Tg | | |
| Resin | T1NBHTDP | BPEF | n_{D} | ν | [° C.] | Mw | Mn |
| Example | 20 | 80 | 1.653 | 21.8 | 112 | 6846 | 6206 |
| 37 | | | | | | | |
| Example | 100 | | 1.684 | 18.9 | 74 | — | — |
| 26* | | | | | | | |

*the values given for the homopolymer of Example 26 correspond to those already listed in Table 1.

### Example 38: Copolymer Prepared from Monomer of Example 8 and BPEF

As materials, 4.24 kg (4.06 mol) of 2-[4-[4-(2-hydroxyethoxy)-3,5-di(phenanthren-9-yl)phenyl]sulfonyl-2,6-di(phenanthren-9-yl)phenoxy]ethanol (see Example 8, hereinafter also designated as T9PNBHBPS), 10.09 kg (23.02 mol) of 9,9-bis[4-(2-hydroxyethoxy)phenyl)fluorene (also designated as BPEF), 5.92 kg (27.62 mol) of diphenylcarbonate (also designated as DPC) and 11 ml of a 2.5×10⁻² mol/l (2.7×10⁻⁴ mol (10×10⁻⁶ mol to 1 mol of the total amount of the dihydroxy compounds)) aqueous solution of sodium hydrogen carbonate were put into a 50 liter reactor with a stirrer and a distillation device.

After the reactor had been flushed with nitrogen, the reaction mixture was heated for 1 hour to 205° C. and stirred at a pressure of 760 Torr. After the reaction mixture was completely dissolved, the pressure was reduced to 150 Torr within 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. And then, the pressure was reduced to 120 Torr in 10 minutes and the reaction conditions of 240° C. and 120 Torr were maintained for 70 minutes. Afterwards, the pressure was reduced to 100 Torr in 10 minutes and the reaction conditions of 240° C. and 100 Torr were maintained for 10 minutes. Further, the pressure was reduced to 1 Torr or lower in 40 minutes and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 9.

TABLE 9

| | Monomers | | Properties | | |
|---|---|---|---|---|---|
| | (mol-%) | | | | Tg |
| Resin | T9PNBHBPS | BPEF | n_{D} | ν | [° C.] |
| Example 38 | 15 | 85 | 1.661 | 21.0 | 157 |

### Example 39: Copolymer Prepared from Monomer of Example 10 and BPEF

As materials, 5.06 kg (4.23 mol) of 2-[4-[4-(2-hydroxyethoxy)-3,5-di(thianthrene-1-yl)phenyl]sulfonyl-2,6-di(thianthrene-1-yl)phenoxy]ethanol (or 2,2′-((sulfonylbis(2,6-di(thianthren-1-yl)-4,1-phenylene))bis(oxy))bis(ethan-1-ol), see Example 10, hereinafter also designated as T1TNTBHBPS), 10.51 kg (23.96 mol) of 9,9-bis[4-(2-hydroxyethoxy)phenyl)fluorene (also designated as BPEF), 6.19 kg (28.89 mol) of diphenylcarbonate (also designated as DPC) and 11 ml of a 2.5×10⁻² mol/l (2.8×10⁻⁴ mol (10×10⁻⁶ mol to 1 mol of the total amount of the dihydroxy compounds)) aqueous solution of sodium hydrogen carbonate were put into a 50 liter reactor with a stirrer and a distillation device.

After the reactor had been flushed with nitrogen, the reaction mixture was heated for 1 hour to 205° C. and stirred at a pressure of 760 Torr. After the reaction mixture was completely dissolved, the pressure was reduced to 150 Torr within 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. And then, the pressure was reduced to 120 Torr in 10 minutes and the reaction conditions of 240° C. and 120 Torr were maintained for 70 minutes. Afterwards, the pressure was reduced to 100 Torr in 10 minutes and the reaction conditions of 240° C. and 100 Torr were maintained for 10 minutes. Further, the pressure was reduced to 1 Torr or lower in 40 minutes and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 10.

TABLE 10

| | Monomers | | Properties | | | | |
|---|---|---|---|---|---|---|---|
| | (mol-%) | | | | Tg | | |
| Resin | T1TNTBHBPS | BPEF | n_{D} | ν | [° C.] | Mw | Mn |
| Example 39 | 15 | 85 | 1.661 | 22.3 | 151 | 21766 | 13021 |
| Example 29* | 100 | | 1.709 | 21.0 | 166 | — | — |

*the values given for the homopolymer of Example 29 correspond to those already listed in Table 1.

### Example 40: Copolymer Prepared from Monomer of Example 13 and BPEF

As materials, 9.91 kg (8.44 mol) of 2,2′-(propane-2,2-diylbis{[2,6-di(thianthren-1-yl)-4,1-phenylene]oxy})di(ethan-1-ol) (or 2,2′-((propane-2,2-diylbis(2,6-di(thianthren-1-yl)-4,1-phenylene))bis(oxy))bis(ethan-1-ol)), see Example 13, hereinafter also designated as T1TNTBHBPA), 8.64 kg (19.70 mol) of 9,9-bis[4-(2-hydroxyethoxy)phenyl)fluorene (also designated as BPEF), 6.21 kg (28.99 mol) of diphenylcarbonate (also designated as DPC) and 11 ml of a 2.5×10⁻² mol/l (2.8×10⁻⁴ mol (10×10⁻⁶ mol to 1 mol of the total amount of the dihydroxy compounds)) aqueous solution of sodium hydrogen carbonate were put into a 50 liter reactor with a stirrer and a distillation device.

After the reactor had been flushed with nitrogen, the reaction mixture was heated for 1 hour to 205° C. and stirred at a pressure of 760 Torr. After the reaction mixture was completely dissolved, the pressure was reduced to 150 Torr within 15 minutes, and then an ester exchange reaction was conducted for 20 minutes at 205° C. and 150 Torr. Further, the reaction mixture was heated to 240° C. at a heating ratio of 37.5° C./h and the reaction conditions of 240° C. and 150 Torr were maintained for 10 minutes. And then, the pressure was reduced to 120 Torr in 10 minutes and the reaction conditions of 240° C. and 120 Torr were maintained for 70 minutes. Afterwards, the pressure was reduced to 100 Torr in 10 minutes and the reaction conditions of 240° C. and 100 Torr were maintained for 10 minutes. Further, the pressure was reduced to 1 Torr or lower in 40 minutes and the polymerization reaction was conducted with stirring at 240° C. and 1 Torr for 10 minutes. After the reaction was completed, the pressure was increased by introducing nitrogen into the reactor and the generated polycarbonate resin was pelletized and removed from the reactor. The characteristics of the obtained polycarbonate resin are summarized in Table 11.

TABLE 11

| | Monomers | | Properties | | | | |
|---|---|---|---|---|---|---|---|
| | (mol-%) | | | | Tg | | |
| Resin | T1TNTBHBPA | BPEF | n_{D} | ν | [° C.] | Mw | Mn |
| Example | 30 | 70 | 1.671 | 21.9 | 169 | 18336 | 11051 |
| 40 | | | | | | | |
| Example | 100 | | 1.702 | 20.6 | 197 | | |
| 30* | | | | | | | |

*the values given for the homopolymer of Example 30 correspond to those already listed in Table 1.

### Example 41: Copolymer Prepared from Monomer of Example 15 and BPEF

As materials, 14.7047 g (0.0335 mol) of 9,9-bis[4-(2-hydroxyethoxy)phenyl)fluorene (BPEF), 11.1975 g (0.0144 mol) of 2,2′-{[3,3′,5,5′-tetra(naphthalen-2-yl)[1,1′-biphenyl]-4,4′-diyl]bis(oxy)}di(ethan-1-ol) (T2NBHB4P) obtained in Example 15, 10.5222 g (0.0491 mol) of diphenylcarbonate (DPC) and 0.4025×104 g (0.4771×10⁻⁶ mol) of sodium hydrogen carbonate were put into a 300 milliliter reactor with a stirrer and a distillation device.

After the reactor had been flushed with nitrogen, the inside pressure was set to 101.3 kPa. The reactor was immersed in an oil bath heated to 200° C. to initiate the ester exchange reaction. Stirring of the reaction mixture was started 5 minutes after the start of the reaction. After 20 minutes the pressure was reduced from 101.3 kPa to 26.66 kPa over a period of 10 minutes, during which time the reaction mixture was heated to 210° C. The reaction mixture was further heated to reach 220° within 60 minutes after the start of the reaction. The pressure was reduced to 20.00 kPa over a period of 10 minutes from the 80-minute point after the start of the reaction, and the reaction mixture was then heated to 240° C. while the pressure was reduced to 0.1 kPa or below. The conditions of 240° C. and 0.1 kPa or below were then maintained for 30 minutes. Afterwards the pressure was returned to 101.3 kPa by introducing nitrogen gas to obtain the desired polycarbonate resin. The characteristics of the obtained resin are summarized in Table 12.

### Example 42: Copolymer Prepared from Monomer of Example 14c and BPEF

As materials, 16.0001 g (0.0365 mol) of 9,9-bis[4-(2-hydroxyethoxy)phenyl)fluorene (BPEF), 12.1815 g (0.0156 mol) of 2,2′-{[3,3′,5,5′-tetra(naphthalen-1-yl)[1,1′-biphenyl]-4,4′-diyl]bis(oxy)}di(ethan-1-ol) (T1NBHB4P) obtained in Example 14c, 11.4484 g (0.0534 mol) of diphenylcarbonate (DPC) and 0.4379×10⁻⁴ g (0.5213×10⁻⁶ mol) of sodium hydrogen carbonate were put into a 300 milliliter reactor with a stirrer and a distillation device.

After the reactor had been flushed with nitrogen, the inside pressure was set to 101.3 kPa. The reactor was immersed in an oil bath heated to 200° C. to initiate the ester exchange reaction. Stirring of the reaction mixture was started 5 minutes after the start of the reaction. After 20 minutes the pressure was reduced from 101.3 kPa to 26.66 kPa over a period of 10 minutes, during which time and the reaction mixture was heated to 210° C. The reaction mixture was further heated to reach 220° C. within 60 minutes after the start of the reaction. The pressure was reduced to 20.00 kPa over a period of 10 minutes from the 80-minute point after the start of the reaction, and the reaction mixture was then heated to 240° C. while the pressure was reduced to 0.1 kPa or below. The conditions of 240° C. and 0.1 kPa or below were then maintained for 30 minutes. Afterwards the pressure was returned to 101.3 kPa by introducing nitrogen gas to obtain the desired polycarbonate resin. The characteristics of the obtained resin are summarized in Table 12.

### Comparative Example: Copolycarbonate Prepared from BNE and BPEF

The copolycarbonate resin of this Comparative Example was prepared in analogy to the process described for Example 41 above, with the exception that instead of T2NBHB4P 2,2′-bis(2-hydroxyethoxy)-1,1′-binaphtyl (BNE) was used as comonomer. The characteristics of the obtained resin are summarized in Table 12.

TABLE 12

| | Monomers | | | | Properties | | | | |
|---|---|---|---|---|---|---|---|---|---|
| | (mol-%) | | | | | | Tg | | CLWC* |
| Resin | T2NBHB4P | T1NBHB4P | BNE | BPEF | n_{D} | ν | [° C.] | Mw | [%] |
| Example 41 | 30 | 0 | 0 | 70 | 1.6741 | 18.76 | 157 | 30000 | 1.6 |
| Example 42 | 0 | 30 | 0 | 70 | 1.6637 | 20.25 | 159 | 23000 | 1.2 |
| Comparative | 0 | 0 | 30 | 70 | 1.6470 | 22.05 | 137 | 34000 | 1.8 |
| Example | | | | | | | | | |

*the CLWC (content of low molecular weight compounds) values were determined as described below.

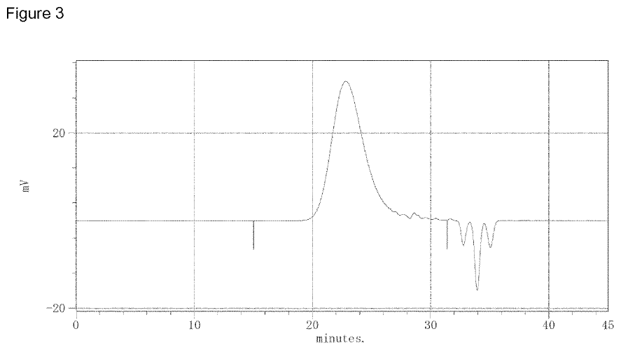

The contents of low molecular weight compounds (CLWC) listed in Table 12 were calculated using the procedure detailed above which is based on GPC data calibrated with polystyrene standards. For example, the CLWC value reported in Table 12 for the resin of Example 41 was calculated from the areas of the individual peaks obtained from the GPC diagram of the resin shown in FIG. 3, using the above described formula. The relevant peak data are summarized in Table 13 below. Specifically, the sum of the peak areas of compounds with Mw values below 1000 was calculated (66697+23135+12863=102695) and related to the total area of all peaks (6318321). Accordingly, the content of low molecular weight compounds in the resin of Example 41was calculated to be 1.6% (102695/6318321×100).

TABLE 13
Data on the peaks of the GPC diagram for the resin of Example 41

| | Retention time of | Mw at | Peak | Peak area |
|---|---|---|---|---|
| No. of peak | peak top [min] | peak top | area | proportions [%] |
| 1 | 22.810 | 29962 | 6105782 | 96.6361 |
| 2 | 27.007 | 2477 | 53104 | 0.8405 |
| 3 | 27.640 | 1664 | 56740 | 0.8980 |
| 4 | 28.598 | 903 | 66697 | 1.0556 |
| 5 | 29.553 | 485 | 23135 | 0.3662 |
| 6 | 30.430 | 271 | 12863 | 0.2036 |
| total | — | — | 6318321 | 100 |

### Birefringence Measurements:

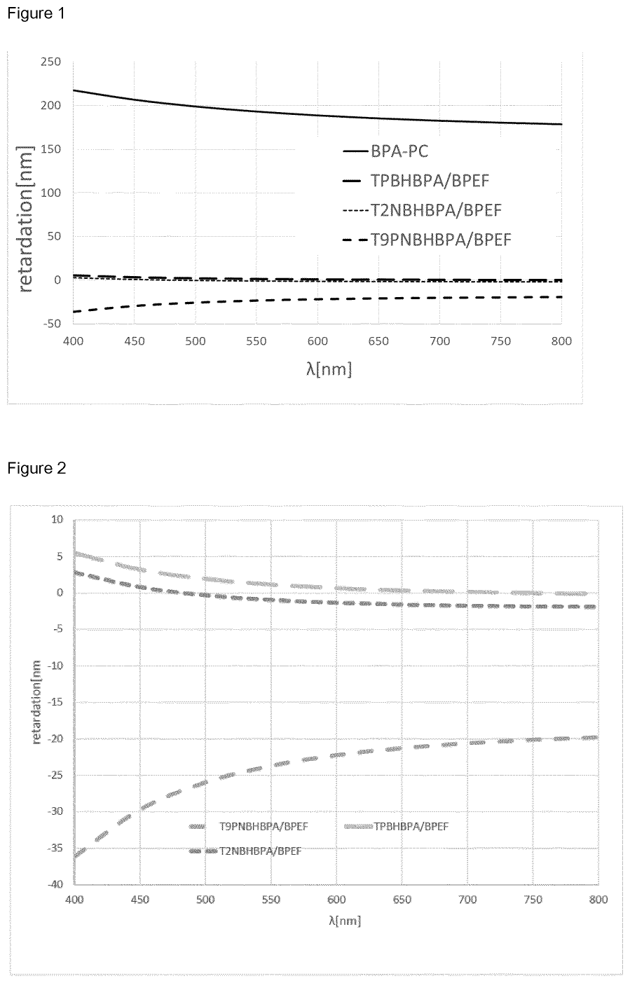

FIG. 1 shows the results of the measurement of the retardation or the birefringence of the resins prepared in examples 31, 33 and 34 and and polycarbonate resin from bisphenol A.

FIG. 2 is a partially enlarged portion of FIG. 1 for the retardation or birefringence of the polymers of the Examples 31, 33 and 34.

In FIG. 1, “TPBHBPA/BPEF” stands for the copolycarbonate of Example 31 (prepared from monomers TPBHBPA and BPEF in a molar ratio of 15:85), “T2NBHBPA/BPEF” stands for the copolycarbonate of Example 33 (prepared from monomers T2NBHBPA and BPEF in a molar ratio of 30:70), “T9PNBHBPA/BPEF” stands for the copolycarbonate of Example 34 (prepared from monomers T9PNBHBPA and BPEF in a molar ratio of 30:70), and “BPA-PC” stands for the homopolycarbonate prepared from bisphenol A (BPA) as diol monomer (compare last entry of Table 1).

It is confirmed by FIG. 1 that TPBHBPA, T2NBHBPA and T9PNBHBPA provide for polycarbonates having very low absolute values of positive or negative birefringence. These characteristics have not been found in conventional materials and TPBHBPA, T2NBHBPA and T9PNBHBPA are particularly useful as optical materials.

We claim:

1. A compound of the formula (I) 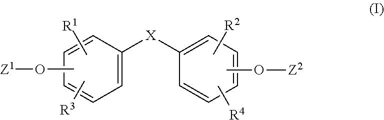
where
X is selected from the group consisting of a single bond, O, N—(C₁-C₄)-alkyl, N—Ar¹, CR⁵R⁶, S, S(O) and SO₂;
Z¹ and Z² are independently selected from hydrogen, -Alk-OH, —CH₂—Ar²—CH₂—OH, -Alk′-C(O)OR^{x}, —CH₂—Ar²—C(O)OR^{x} and —C(O)—Ar²—C(O)OR^{x},
where R^{x} is selected from the group consisting of hydrogen, phenyl, benzyl and C₁-C₄-alkyl;
R¹ and R² are independently selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms,
where mono- or polycyclic aryl and mono- or polycyclic hetaryl are unsubstituted or carry 1, 2, 3 or 4 radicals R^{Ar};
R³ and R⁴ are independently selected from the group consisting of hydrogen, mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms,
where mono- or polycyclic aryl and mono- or polycyclic hetaryl are unsubstituted or carry 1, 2, 3 or 4 radicals R^{Ar};
R⁵ is selected from the group consisting of hydrogen and C₁-C₄-alkyl;
R⁶ is selected from the group consisting of hydrogen and C₁-C₄-alkyl;
Ar¹ is selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these atoms of hetaryl are carbon atoms,
where mono- or polycyclic aryl and mono- or polycyclic hetaryl are unsubstituted or carry 1, 2, 3 or 4 radicals R^{Ar};
Ar² is selected from the group consisting of phenylene, naphthylene and biphenylylene;
Alk is C₂-C₄-alkandiyl;
Alk′ is C₁-C₄-alkandiyl;
R^{Ar} is selected from the group consisting of R, OR, CH, R₃₋ₙ, NR₂ and CH═CHR′, where R^{Ar} may identical or different if more than one is present on the same (het)aryl group;
R is selected from the group consisting of methyl, ethyl, phenyl, naphthyl, phenanthrenyl and triphenylenyl, where phenyl, naphthyl, phenanthrenyl and triphenylenyl are unsubstituted or substituted by 1, 2, 3 or 4 identical or different radicals R″;
R′ is selected from hydrogen, methyl, phenyl and naphthyl, where phenyl and naphthyl are unsubstituted or substituted by 1, 2, 3 or 4 identical or different radicals R″;
R″ is selected from the group consisting of phenyl, OCH₃, CH₃, N(CH₃)₂ and C(O)CH₃; and
n is 0, 1 or 2;
provided that R¹ and R² are not both phenyl, if R³ and R⁴ are both hydrogen.

2. The compound of claim 1, where formula (I) is represented by formula (Ia):

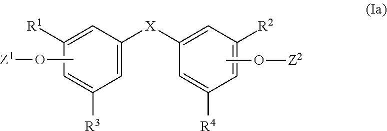

3. The compound of claim 1 or claim 2, where X is selected from the group consisting of a single bond, O, N-methyl, N-ethyl, N-isopropyl, N-phenyl, N-naphthyl, N-phenanthryl, CH₂, C(CH₃)₂, CH(CH₃), S, and SO₂, and in particular selected from the group consisting of a single bond, O, CH₂, C(CH₃)₂, CH(CH₃), S, and SO₂.

4. The compound of any one of the preceding claims, where Z¹ and Z² are selected from hydrogen, 2-hydroxyethyl, methoxycarbonyl-methyl, hydroxymethyl-phenylmethyl, hydroxymethyl-naphthyl-methyl, hydroxymethyl-biphenylyl-methyl, methoxycarbonyl-phenyl-methyl and methoxycarbonyl-naphthyl-methyl, in particular from hydrogen, 2-hydroxyethyl, methoxycarbonyl-methyl, (4-(hydroxylmethyl)phenyl)methyl, (3-(hydroxymethyl)phenyl)methyl, (4-(hydroxymethyl)-1-naphthyl)methyl, (5-(hydroxymethyl)-1-naphthyl)methyl, (6-(hydroxymethyl)-2-naphthyl)methyl, 4′-(hydroxymethyl)-1,1′-biphenylyl-4-methyl, (4-(methoxycarbonyl)phenyl)methyl, (3-(methoxycarbonyl)phenyl)methyl, (4-(methoxycarbonyl)-1-naphthyl)methyl and (6-(methoxycarbonyl)-2-naphthyl)methyl.

5. The compound of any one of the preceding claims, where R¹ and R² are independently selected from the group consisting of phenyl, naphthyl, 1,2-dihydroacenaphthylenyl, biphenylyl, fluorenyl, 11H-benzo[a]fluorenyl, 11H-benzo[b]fluorenyl, 7H-benzo[c]fluorenyl, phenanthrenyl, benzo[c]phenanthrenyl, pyrenyl, chrysenyl, picenyl, triphenylenyl, benzo[b]furanyl, dibenzo[b,d]furanyl, naphtho[1,2-b]furanyl, naphtho[2,3-b]furanyl, naphtho[2,1-b]furanyl, benzo[b]naphtho[1,2-d]furanyl, benzo[b]naphtho[2,3-d]furanyl, benzo[b]naphtho[2,1-d]furanyl, benzo[1,2-b:4,3-b′]difuranyl, benzo[1,2-b:6,5-b′]difuranyl, benzo[1,2-b:5,4-b′]difuranyl, benzo[1,2-b:4,5-b′]difuranyl, tribenzo[b,d,f]oxepinyl, 2H-naphtho[1,8-d,e][1,3]dioxinyl, dinaphtho[2,3-b:2′,3′-d]furanyl, oxanthrenyl, benzo[a]oxanthrenyl, benzo[b]oxanthrenyl, benzo[b]thienyl, dibenzo[b,d]thienyl, naphtho[1,2-b]thienyl, naphtho[2,3-b]thienyl, naphtho[2,1-b]thienyl, benzo[b]naphtho[1,2-d]thienyl, benzo[b]naphtho[2,3-d]thienyl, benzo[b]naphtho[2,1-d]thienyl, benzo[1,2-b:4,3-b′]dithienyl, benzo[1,2-b:6,5-b′]dithienyl, benzo[1,2-b:5,4-b′]dithienyl, benzo[1,2-b:4,5-b′]dithienyl, 9H-thioxanthenyl, 6H-dibenzo[b,d]thiopyranyl, 1,4-benzodithiinyl, naphtho[1,2-b][1,4]dithiinyl, naphtho[2,3-b][1,4]dithiinyl, thianthrenyl, benzo[a]thianthrenyl, benzo[b]thianthrenyl, dibenzo[a,c]thianthrenyl, dibenzo[a,h]thianthrenyl, dibenzo[a,i]thianthrenyl, dibenzo[a,j]thianthrenyl, dibenzo[b,i]thianthrenyl, 2H-naphtho[1,8-b,c]thienyl, dibenzo[b,d]thiepinyl, dibenzo[b,f]thiepinyl, 5H-phenanthro[4,5-b,c,d]thiopyranyl, tribenzo[b,d,f]thiepinyl, 2,5-dihydronaphtho[1,8-b,c:4,5-b′c′]dithienyl, 2,6-dihydronaphtho[1,8-b,c:5,4-b′c′]dithienyl, tribenzo[a,c,i]thianthrenyl, benzo[b]naphtho[1,8-e,f][1,4]dithiepinyl, dinaphtho[2,3-b:2′,3′-d]thienyl, 5H-phenanthro[1,10-b,c]thienyl, 7H-phenanthro[1,10-c,b]thienyl, dibenzo[d,d′]benzo[1,2-b:4,5-b′]dithienyl and dibenzo[d,d′]benzo[1,2-b:5,4-b′]dithienyl.

6. The compound of claim 5, where R¹ and R² are independently selected from the group consisting of phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl and thianthrenyl.

7. The compound of any one of the preceding claims, where R¹ and R² have the same meaning.

8. The compound of any one of the preceding claims, where R³ and R⁴ are selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms,
where mono- or polycyclic aryl and mono- or polycyclic hetaryl are unsubstituted or carry 1, 2, 3 or 4 radicals R^{Ar}.

9. The compound of claim 8, where R³ and R⁴ are independently selected from the group consisting of phenyl, naphthyl, 1,2-dihydroacenaphthylenyl, biphenylyl, fluorenyl, 11H-benzo[a]fluorenyl, 11H-benzo[b]fluorenyl, 7H-benzo[c]fluorenyl, phenanthrenyl, benzo[c]phenanthrenyl, pyrenyl, chrysenyl, picenyl, triphenylenyl, benzo[b]furanyl, dibenzo[b,d]furanyl, naphtho[1,2-b]furanyl, naphtho[2,3-b]furanyl, naphtho[2,1-b]furanyl, benzo[b]naphtho[1,2-d]furanyl, benzo[b]naphtho[2,3-d]furanyl, benzo[b]naphtho[2,1-d]furanyl, benzo[1,2-b:4,3-b′]difuranyl, benzo[1,2-b:6,5-b′]difuranyl, benzo[1,2-b:5,4-b′]difuranyl, benzo[1,2-b:4,5-b′]difuranyl, tribenzo[b,d,f]oxepinyl, 2H-naphtho[1,8-d,e][1,3]dioxinyl, dinaphtho[2,3-b:2′,3′-d]furanyl, oxanthrenyl, benzo[a]oxanthrenyl, benzo[b]oxanthrenyl, benzo[b]thienyl, dibenzo[b,d]thienyl, naphtho[1,2-b]thienyl, naphtho[2,3-b]thienyl, naphtho[2,1-b]thienyl, benzo[b]naphtho[1,2-d]thienyl, benzo[b]naphtho[2,3-d]thienyl, benzo[b]naphtho[2,1-d]thienyl, benzo[1,2-b:4,3-b′]dithienyl, benzo[1,2-b:6,5-b′]dithienyl, benzo[1,2-b:5,4-b′]dithienyl, benzo[1,2-b:4,5-b′]dithienyl, 9H-thioxanthenyl, 6H-dibenzo[b,d]thiopyranyl, 1,4-benzodithiinyl, naphtho[1,2-b][1,4]dithiinyl, naphtho[2,3-b][1,4]dithiinyl, thianthrenyl, benzo[a]thianthrenyl, benzo[b]thianthrenyl, dibenzo[a,c]thianthrenyl, dibenzo[a,h]thianthrenyl, dibenzo[a,i]thianthrenyl, dibenzo[a,j]thianthrenyl, dibenzo[b,i]thianthrenyl, 2H-naphtho[1,8-b,c]thienyl, dibenzo[b,d]thiepinyl, dibenzo[b,f]thiepinyl, 5H-phenanthro[4,5-b,c,d]thiopyranyl, tribenzo[b,d,f]thiepinyl, 2,5-dihydronaphtho[1,8-b,c:4,5-b′c′]dithienyl, 2,6-dihydronaphtho[1,8-b,c:5,4-b′c′]dithienyl, tribenzo[a,c,i]thianthrenyl, benzo[b]naphtho[1,8-e,f][1,4]dithiepinyl, dinaphtho[2,3-b:2′,3′-d]thienyl, 5H-phenanthro[1,10-b,c]thienyl, 7H-phenanthro[1,10-c,b]thienyl, dibenzo[d,d′]benzo[1,2-b:4,5-b′]dithienyl and dibenzo[d,d′]benzo[1,2-b:5,4-b′]dithienyl.

10. The compound of claim 9, where R³ and R⁴ are independently selected from the group consisting of phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl and thianthrenyl.

11. The compound of any one of the preceding claims, where R¹, R², R³ and R⁴ have the same meaning.

12. The compound of any one of the preceding claims, where formula (I) is represented by formula (Ia-1), wherein Z is defined as Z¹ and Z² in claim 1 or claim 4:

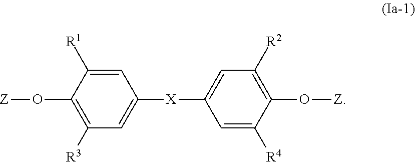

13. The compound of claim 12, where R^{y}, X and Z are as defined in one row of table A′, wherein R^{y} represents the identical meaning of the substituents R¹, R², R³ and R⁴:
TABLE A′

| | X | R^{y} (R¹ = R² = R³ = R⁴) | Z |
|---|---|---|---|
| 1 | C(CH₃)₂ | phenyl | hydrogen |
| 2 | C(CH₃)₂ | naphth-1-yl | hydrogen |
| 3 | C(CH₃)₂ | naphth-2-yl | hydrogen |
| 4 | C(CH₃)₂ | phenanthren-9-yl | hydrogen |
| 5 | C(CH₃)₂ | dibenzo[b,d]thien-2-yl | hydrogen |
| 6 | C(CH₃)₂ | dibenzo[b,d]thien-4-yl | hydrogen |
| 7 | C(CH₃)₂ | thianthren-1-yl | hydrogen |
| 8 | C(CH₃)₂ | thianthren-2-yl | hydrogen |
| 9 | SO₂ | phenyl | hydrogen |
| 10 | SO₂ | naphth-1-yl | hydrogen |
| 11 | SO₂ | naphth-2-yl | hydrogen |
| 12 | SO₂ | phenanthren-9-yl | hydrogen |
| 13 | SO₂ | dibenzo[b,d]thien-2-yl | hydrogen |
| 14 | SO₂ | dibenzo[b,d]thien-4-yl | hydrogen |
| 15 | SO₂ | thianthren-1-yl | hydrogen |
| 16 | SO₂ | thianthren-2-yl | hydrogen |
| 17 | S | phenyl | hydrogen |
| 18 | S | naphth-1-yl | hydrogen |
| 19 | S | naphth-2-yl | hydrogen |
| 20 | S | phenanthren-9-yl | hydrogen |
| 21 | S | dibenzo[b,d]thien-2-yl | hydrogen |
| 22 | S | dibenzo[b,d]thien-4-yl | hydrogen |
| 23 | S | thianthren-1-yl | hydrogen |
| 24 | S | thianthren-2-yl | hydrogen |
| 25 | single bond | phenyl | hydrogen |
| 26 | single bond | naphth-1-yl | hydrogen |
| 27 | single bond | naphth-2-yl | hydrogen |
| 28 | single bond | phenanthren-9-yl | hydrogen |
| 29 | single bond | dibenzo[b,d]thien-2-yl | hydrogen |
| 30 | single bond | dibenzo[b,d]thien-4-yl | hydrogen |
| 31 | single bond | thianthren-1-yl | hydrogen |
| 32 | single bond | thianthren-2-yl | hydrogen |
| 33 | C(CH₃)₂ | phenyl | 2-hydroxyethyl |
| 34 | C(CH₃)₂ | naphth-1-yl | 2-hydroxyethyl |
| 35 | C(CH₃)₂ | naphth-2-yl | 2-hydroxyethyl |
| 36 | C(CH₃)₂ | phenanthren-9-yl | 2-hydroxyethyl |
| 37 | C(CH₃)₂ | triphenylen-1-yl | 2-hydroxyethyl |
| 38 | C(CH₃)₂ | triphenylen-2-yl | 2-hydroxyethyl |
| 39 | C(CH₃)₂ | pyren-1-yl | 2-hydroxyethyl |
| 40 | C(CH₃)₂ | pyren-2-yl | 2-hydroxyethyl |
| 41 | C(CH₃)₂ | pyren-4-yl | 2-hydroxyethyl |
| 42 | C(CH₃)₂ | 1,2-dihydro- | 2-hydroxyethyl |
| | | acenaphthylen-5-yl | |
| 43 | C(CH₃)₂ | dibenzo[b,d]furan-2-yl | 2-hydroxyethyl |
| 44 | C(CH₃)₂ | dibenzo[b,d]furan-4-yl | 2-hydroxyethyl |
| 45 | C(CH₃)₂ | benzo[b]thien-3-yl | 2-hydroxyethyl |
| 46 | C(CH₃)₂ | benzo[b]thien-4-yl | 2-hydroxyethyl |
| 47 | C(CH₃)₂ | benzo[b]thien-5-yl | 2-hydroxyethyl |
| 48 | C(CH₃)₂ | benzo[b]thien-6-yl | 2-hydroxyethyl |
| 49 | C(CH₃)₂ | benzo[b]thien-7-yl | 2-hydroxyethyl |
| 50 | C(CH₃)₂ | dibenzo[b,d]thien-2-yl | 2-hydroxyethyl |
| 51 | C(CH₃)₂ | dibenzo[b,d]thien-4-yl | 2-hydroxyethyl |
| 52 | C(CH₃)₂ | thianthren-1-yl | 2-hydroxyethyl |
| 53 | C(CH₃)₂ | thianthren-2-yl | 2-hydroxyethyl |
| 54 | SO₂ | phenyl | 2-hydroxyethyl |
| 55 | SO₂ | naphth-1-yl | 2-hydroxyethyl |
| 56 | SO₂ | naphth-2-yl | 2-hydroxyethyl |
| 57 | SO₂ | phenanthren-9-yl | 2-hydroxyethyl |
| 58 | SO₂ | triphenylen-1-yl | 2-hydroxyethyl |
| 59 | SO₂ | triphenylen-2-yl | 2-hydroxyethyl |
| 60 | SO₂ | pyren-1-yl | 2-hydroxyethyl |
| 61 | SO₂ | pyren-2-yl | 2-hydroxyethyl |
| 62 | SO₂ | pyren-4-yl | 2-hydroxyethyl |
| 63 | SO₂ | 1,2-dihydro- | 2-hydroxyethyl |
| | | acenaphthylen-5-yl | |
| 64 | SO₂ | dibenzo[b,d]furan-2-yl | 2-hydroxyethyl |
| 65 | SO₂ | dibenzo[b,d]furan-4-yl | 2-hydroxyethyl |
| 66 | SO₂ | benzo[b]thien-3-yl | 2-hydroxyethyl |
| 67 | SO₂ | benzo[b]thien-4-yl | 2-hydroxyethyl |
| 68 | SO₂ | benzo[b]thien-5-yl | 2-hydroxyethyl |
| 69 | SO₂ | benzo[b]thien-6-yl | 2-hydroxyethyl |
| 70 | SO₂ | benzo[b]thien-7-yl | 2-hydroxyethyl |
| 71 | SO₂ | dibenzo[b,d]thien-2-yl | 2-hydroxyethyl |
| 72 | SO₂ | dibenzo[b,d]thien-4-yl | 2-hydroxyethyl |
| 73 | SO₂ | thianthren-1-yl | 2-hydroxyethyl |
| 74 | SO₂ | thianthren-2-yl | 2-hydroxyethyl |
| 75 | S | phenyl | 2-hydroxyethyl |
| 76 | S | naphth-1-yl | 2-hydroxyethyl |
| 77 | S | naphth-2-yl | 2-hydroxyethyl |
| 78 | S | phenanthren-9-yl | 2-hydroxyethyl |
| 79 | S | triphenylen-1-yl | 2-hydroxyethyl |
| 80 | S | triphenylen-2-yl | 2-hydroxyethyl |
| 81 | S | pyren-1-yl | 2-hydroxyethyl |
| 82 | S | pyren-2-yl | 2-hydroxyethyl |
| 83 | S | pyren-4-yl | 2-hydroxyethyl |
| 84 | S | 1,2-dihydro- | 2-hydroxyethyl |
| | | acenaphthylen-5-yl | |
| 85 | S | dibenzo[b,d]furan-2-yl | 2-hydroxyethyl |
| 86 | S | dibenzo[b,d]furan-4-yl | 2-hydroxyethyl |
| 87 | S | benzo[b]thien-3-yl | 2-hydroxyethyl |
| 88 | S | benzo[b]thien-4-yl | 2-hydroxyethyl |
| 89 | S | benzo[b]thien-5-yl | 2-hydroxyethyl |
| 90 | S | benzo[b]thien-6-yl | 2-hydroxyethyl |
| 91 | S | benzo[b]thien-7-yl | 2-hydroxyethyl |
| 92 | S | dibenzo[b,d]thien-2-yl | 2-hydroxyethyl |
| 93 | S | dibenzo[b,d]thien-4-yl | 2-hydroxyethyl |
| 94 | S | thianthren-1-yl | 2-hydroxyethyl |
| 95 | S | thianthren-2-yl | 2-hydroxyethyl |
| 96 | single bond | phenyl | 2-hydroxyethyl |
| 97 | single bond | naphth-1-yl | 2-hydroxyethyl |
| 98 | single bond | naphth-2-yl | 2-hydroxyethyl |
| 99 | single bond | phenanthren-9-yl | 2-hydroxyethyl |
| 100 | single bond | dibenzo[b,d]thien-2-yl | 2-hydroxyethyl |
| 101 | single bond | dibenzo[b,d]thien-4-yl | 2-hydroxyethyl |
| 102 | single bond | thianthren-1-yl | 2-hydroxyethyl |
| 103 | single bond | thianthren-2-yl | 2-hydroxyethyl |
| 104 | C(CH₃)₂ | phenyl | methoxycarbonyl-methyl |
| 105 | C(CH₃)₂ | naphth-1-yl | methoxycarbonyl-methyl |
| 106 | C(CH₃)₂ | naphth-2-yl | methoxycarbonyl-methyl |
| 107 | C(CH₃)₂ | phenanthren-9-yl | methoxycarbonyl-methyl |
| 108 | C(CH₃)₂ | dibenzo[b,d]thien-2-yl | methoxycarbonyl-methyl |
| 109 | C(CH₃)₂ | dibenzo[b,d]thien-4-yl | methoxycarbonyl-methyl |
| 110 | C(CH₃)₂ | thianthren-1-yl | methoxycarbonyl-methyl |
| 111 | C(CH₃)₂ | thianthren-2-yl | methoxycarbonyl-methyl |
| 112 | SO₂ | phenyl | methoxycarbonyl-methyl |
| 113 | SO₂ | naphth-1-yl | methoxycarbonyl-methyl |
| 114 | SO₂ | naphth-2-yl | methoxycarbonyl-methyl |
| 115 | SO₂ | phenanthren-9-yl | methoxycarbonyl-methyl |
| 116 | SO₂ | dibenzo[b,d]thien-2-yl | methoxycarbonyl-methyl |
| 117 | SO₂ | dibenzo[b,d]thien-4-yl | methoxycarbonyl-methyl |
| 118 | SO₂ | thianthren-1-yl | methoxycarbonyl-methyl |
| 119 | SO₂ | thianthren-2-yl | methoxycarbonyl-methyl |
| 120 | S | phenyl | methoxycarbonyl-methyl |
| 121 | S | naphth-1-yl | methoxycarbonyl-methyl |
| 122 | S | naphth-2-yl | methoxycarbonyl-methyl |
| 123 | S | phenanthren-9-yl | methoxycarbonyl-methyl |
| 124 | S | dibenzo[b,d]thien-2-yl | methoxycarbonyl-methyl |
| 125 | S | dibenzo[b,d]thien-4-yl | methoxycarbonyl-methyl |
| 126 | S | thianthren-1-yl | methoxycarbonyl-methyl |
| 127 | S | thianthren-2-yl | methoxycarbonyl-methyl |
| 128 | single bond | phenyl | methoxycarbonyl-methyl |
| 129 | single bond | naphth-1-yl | methoxycarbonyl-methyl |
| 130 | single bond | naphth-2-yl | methoxycarbonyl-methyl |
| 131 | single bond | phenanthren-9-yl | methoxycarbonyl-methyl |
| 132 | single bond | dibenzo[b,d]thien-2-yl | methoxycarbonyl-methyl |
| 133 | single bond | dibenzo[b,d]thien-4-yl | methoxycarbonyl-methyl |
| 134 | single bond | thianthren-1-yl | methoxycarbonyl-methyl |
| 135 | single bond | thianthren-2-yl | methoxycarbonyl-methyl |
| 136 | C(CH₃)₂ | phenyl | (4-(hydroxymethyl)phenyl)methyl |
| 137 | C(CH₃)₂ | naphth-1-yl | (4-(hydroxymethyl)phenyl)methyl |
| 138 | C(CH₃)₂ | naphth-2-yl | (4-(hydroxymethyl)phenyl)methyl |
| 139 | C(CH₃)₂ | phenanthren-9-yl | (4-(hydroxymethyl)phenyl)methyl |
| 140 | C(CH₃)₂ | dibenzo[b,d]thien-2-yl | (4-(hydroxymethyl)phenyl)methyl |
| 141 | C(CH₃)₂ | dibenzo[b,d]thien-4-yl | (4-(hydroxymethyl)phenyl)methyl |
| 142 | C(CH₃)₂ | thianthren-1-yl | (4-(hydroxymethyl)phenyl)methyl |
| 143 | C(CH₃)₂ | thianthren-2-yl | (4-(hydroxymethyl)phenyl)methyl |
| 144 | SO₂ | phenyl | (4-(hydroxymethyl)phenyl)methyl |
| 145 | SO₂ | naphth-1-yl | (4-(hydroxymethyl)phenyl)methyl |
| 146 | SO₂ | naphth-2-yl | (4-(hydroxymethyl)phenyl)methyl |
| 147 | SO₂ | phenanthren-9-yl | (4-(hydroxymethyl)phenyl)methyl |
| 148 | SO₂ | dibenzo[b,d]thien-2-yl | (4-(hydroxymethyl)phenyl)methyl |
| 149 | SO₂ | dibenzo[b,d]thien-4-yl | (4-(hydroxymethyl)phenyl)methyl |
| 150 | SO₂ | thianthren-1-yl | (4-(hydroxymethyl)phenyl)methyl |
| 151 | SO₂ | thianthren-2-yl | (4-(hydroxymethyl)phenyl)methyl |
| 152 | S | phenyl | (4-(hydroxymethyl)phenyl)methyl |
| 153 | S | naphth-1-yl | (4-(hydroxymethyl)phenyl)methyl |
| 154 | S | naphth-2-yl | (4-(hydroxymethyl)phenyl)methyl |
| 155 | S | phenanthren-9-yl | (4-(hydroxymethyl)phenyl)methyl |
| 156 | S | dibenzo[b,d]thien-2-yl | (4-(hydroxymethyl)phenyl)methyl |
| 157 | S | dibenzo[b,d]thien-4-yl | (4-(hydroxymethyl)phenyl)methyl |
| 158 | S | thianthren-1-yl | (4-(hydroxymethyl)phenyl)methyl |
| 159 | S | thianthren-2-yl | (4-(hydroxymethyl)phenyl)methyl |
| 160 | single bond | phenyl | (4-(hydroxymethyl)phenyl)methyl |
| 161 | single bond | naphth-1-yl | (4-(hydroxymethyl)phenyl)methyl |
| 162 | single bond | naphth-2-yl | (4-(hydroxymethyl)phenyl)methyl |
| 163 | single bond | phenanthren-9-yl | (4-(hydroxymethyl)phenyl)methyl |
| 164 | single bond | dibenzo[b,d]thien-2-yl | (4-(hydroxymethyl)phenyl)methyl |
| 165 | single bond | dibenzo[b,d]thien-4-yl | (4-(hydroxymethyl)phenyl)methyl |
| 166 | single bond | thianthren-1-yl | (4-(hydroxymethyl)phenyl)methyl |
| 167 | single bond | thianthren-2-yl | (4-(hydroxymethyl)phenyl)methyl |
| 168 | C(CH₃)₂ | phenyl | (3-(hydroxymethyl)phenyl)methyl |
| 169 | C(CH₃)₂ | naphth-1-yl | (3-(hydroxymethyl)phenyl)methyl |
| 170 | C(CH₃)₂ | naphth-2-yl | (3-(hydroxymethyl)phenyl)methyl |
| 171 | C(CH₃)₂ | phenanthren-9-yl | (3-(hydroxymethyl)phenyl)methyl |
| 172 | C(CH₃)₂ | dibenzo[b,d]thien-2-yl | (3-(hydroxymethyl)phenyl)methyl |
| 173 | C(CH₃)₂ | dibenzo[b,a]thien-4-yl | (3-(hydroxymethyl)phenyl)methyl |
| 174 | C(CH₃)₂ | thianthren-1-yl | (3-(hydroxymethyl)phenyl)methyl |
| 175 | C(CH₃)₂ | thianthren-2-yl | (3-(hydroxymethyl)phenyl)methyl |
| 176 | SO₂ | phenyl | (3-(hydroxymethyl)phenyl)methyl |
| 177 | SO₂ | naphth-1-yl | (3-(hydroxymethyl)phenyl)methyl |
| 178 | SO₂ | naphth-2-yl | (3-(hydroxymethyl)phenyl)methyl |
| 179 | SO₂ | phenanthren-9-yl | (3-(hydroxymethyl)phenyl)methyl |
| 180 | SO₂ | dibenzo[b,d]thien-2-yl | (3-(hydroxymethyl)phenyl)methyl |
| 181 | SO₂ | dibenzo[b,d]thien-4-yl | (3-(hydroxymethyl)phenyl)methyl |
| 182 | SO₂ | thianthren-1-yl | (3-(hydroxymethyl)phenyl)methyl |
| 183 | SO₂ | thianthren-2-yl | (3-(hydroxymethyl)phenyl)methyl |
| 184 | S | phenyl | (3-(hydroxymethyl)phenyl)methyl |
| 185 | S | naphth-1-yl | (3-(hydroxymethyl)phenyl)methyl |
| 186 | S | naphth-2-yl | (3-(hydroxymethyl)phenyl)methyl |
| 187 | S | phenanthren-9-yl | (3-(hydroxymethyl)phenyl)methyl |
| 188 | S | dibenzo[b,d]thien-2-yl | (3-(hydroxymethyl)phenyl)methyl |
| 189 | S | dibenzo[b,d]thien-4-yl | (3-(hydroxymethyl)phenyl)methyl |
| 190 | S | thianthren-1-yl | (3-(hydroxymethyl)phenyl)methyl |
| 191 | S | thianthren-2-yl | (3-(hydroxymethyl)phenyl)methyl |
| 192 | single bond | phenyl | (3-(hydroxymethyl)phenyl)methyl |
| 193 | single bond | naphth-1-yl | (3-(hydroxymethyl)phenyl)methyl |
| 194 | single bond | naphth-2-yl | (3-(hydroxymethyl)phenyl)methyl |
| 195 | single bond | phenanthren-9-yl | (3-(hydroxymethyl)phenyl)methyl |
| 196 | single bond | dibenzo[b,d]thien-2-yl | (3-(hydroxymethyl)phenyl)methyl |
| 197 | single bond | dibenzo[b,d]thien-4-yl | (3-(hydroxymethyl)phenyl)methyl |
| 198 | single bond | thianthren-1-yl | (3-(hydroxymethyl)phenyl)methyl |
| 199 | single bond | thianthren-2-yl | (3-(hydroxymethyl)phenyl)methyl |
| 200 | C(CH₃)₂ | phenyl | (4-(hydroxymethyl)-1-naphthyl)methyl |
| 201 | C(CH₃)₂ | naphth-1-yl | (4-(hydroxymethyl)-1-naphthyl)methyl |
| 202 | C(CH₃)₂ | naphth-2-yl | (4-(hydroxymethyl)-1-naphthyl)methyl |
| 203 | C(CH₃)₂ | phenanthren-9-yl | (4-(hydroxymethyl)-1-naphthyl)methyl |
| 204 | C(CH₃)₂ | dibenzo[b,d]thien-2-yl | (4-(hydroxymethyl)-1-naphthyl)methyl |
| 205 | C(CH₃)₂ | dibenzo[b,d]thien-4-yl | (4-(hydroxymethyl)-1-naphthyl)methyl |
| 206 | C(CH₃)₂ | thianthren-1-yl | (4-(hydroxymethyl)-1-naphthyl)methyl |
| 207 | C(CH₃)₂ | thianthren-2-yl | (4-(hydroxymethyl)-1-naphthyl)methyl |
| 208 | SO₂ | phenyl | (4-(hydroxymethyl)-1-naphthyl)methyl |
| 209 | SO₂ | naphth-1-yl | (4-(hydroxymethyl)-1-naphthyl)methyl |
| 210 | SO₂ | naphth-2-yl | (4-(hydroxymethyl)-1-naphthyl)methyl |
| 211 | SO₂ | phenanthren-9-yl | (4-(hydroxymethyl)-1-naphthyl)methyl |
| 212 | SO₂ | dibenzo[b,d]thien-2-yl | (4-(hydroxymethyl)-1-naphthyl)methyl |
| 213 | SO₂ | dibenzo[b,d]thien-4-yl | (4-(hydroxymethyl)-1-naphthyl)methyl |
| 214 | SO₂ | thianthren-1-yl | (4-(hydroxymethyl)-1-naphthyl)methyl |
| 215 | SO₂ | thianthren-2-yl | (4-(hydroxymethyl)-1-naphthyl)methyl |
| 216 | S | phenyl | (4-(hydroxymethyl)-1-naphthyl)methyl |
| 217 | S | naphth-1-yl | (4-(hydroxymethyl)-1-naphthyl)methyl |
| 218 | S | naphth-2-yl | (4-(hydroxymethyl)-1-naphthyl)methyl |
| 219 | S | phenanthren-9-yl | (4-(hydroxymethyl)-1-naphthyl)methyl |
| 220 | S | dibenzo[b,d]thien-2-yl | (4-(hydroxymethyl)-1-naphthyl)methyl |
| 221 | S | dibenzo[b,d]thien-4-yl | (4-(hydroxymethyl)-1-naphthyl)methyl |
| 222 | S | thianthren-1-yl | (4-(hydroxymethyl)-1-naphthyl)methyl |
| 223 | S | thianthren-2-yl | (4-(hydroxymethyl)-1-naphthyl)methyl |
| 224 | single bond | phenyl | (4-(hydroxymethyl)-1-naphthyl)methyl |
| 225 | single bond | naphth-1-yl | (4-(hydroxymethyl)-1-naphthyl)methyl |
| 226 | single bond | naphth-2-yl | (4-(hydroxymethyl)-1-naphthyl)methyl |
| 227 | single bond | phenanthren-9-yl | (4-(hydroxymethyl)-1-naphthyl)methyl |
| 228 | single bond | dibenzo[b,d]thien-2-yl | (4-(hydroxymethyl)-1-naphthyl)methyl |
| 229 | single bond | dibenzo[b,d]thien-4-yl | (4-(hydroxymethyl)-1-naphthyl)methyl |
| 230 | single bond | thianthren-1-yl | (4-(hydroxymethyl)-1-naphthyl)methyl |
| 231 | single bond | thianthren-2-yl | (4-(hydroxymethyl)-1-naphthyl)methyl |
| 232 | C(CH₃)₂ | phenyl | (5-(hydroxymethyl)-1-naphthyl)methyl |
| 233 | C(CH₃)₂ | naphth-1-yl | (5-(hydroxymethyl)-1-naphthyl)methyl |
| 234 | C(CH₃)₂ | naphth-2-yl | (5-(hydroxymethyl)-1-naphthyl)methyl |
| 235 | C(CH₃)₂ | phenanthren-9-yl | (5-(hydroxymethyl)-1-naphthyl)methyl |
| 236 | C(CH₃)₂ | dibenzo[b,d]thien-2-yl | (5-(hydroxymethyl)-1-naphthyl)methyl |
| 237 | C(CH₃)₂ | dibenzo[b,d]thien-4-yl | (5-(hydroxymethyl)-1-naphthyl)methyl |
| 238 | C(CH₃)₂ | thianthren-1-yl | (5-(hydroxymethyl)-1-naphthyl)methyl |
| 239 | C(CH₃)₂ | thianthren-2-yl | (5-(hydroxymethyl)-1-naphthyl)methyl |
| 240 | SO₂ | phenyl | (5-(hydroxymethyl)-1-naphthyl)methyl |
| 241 | SO₂ | naphth-1-yl | (5-(hydroxymethyl)-1-naphthyl)methyl |
| 242 | SO₂ | naphth-2-yl | (5-(hydroxymethyl)-1-naphthyl)methyl |
| 243 | SO₂ | phenanthren-9-yl | (5-(hydroxymethyl)-1-naphthyl)methyl |
| 244 | SO₂ | dibenzo[b,d]thien-2-yl | (5-(hydroxymethyl)-1-naphthyl)methyl |
| 245 | SO₂ | dibenzo[b,d]thien-4-yl | (5-(hydroxymethyl)-1-naphthyl)methyl |
| 246 | SO₂ | thianthren-1-yl | (5-(hydroxymethyl)-1-naphthyl)methyl |
| 247 | SO₂ | thianthren-2-yl | (5-(hydroxymethyl)-1-naphthyl)methyl |
| 248 | S | phenyl | (5-(hydroxymethyl)-1-naphthyl)methyl |
| 249 | S | naphth-1-yl | (5-(hydroxymethyl)-1-naphthyl)methyl |
| 250 | S | naphth-2-yl | (5-(hydroxymethyl)-1-naphthyl)methyl |
| 251 | S | phenanthren-9-yl | (5-(hydroxymethyl)-1-naphthyl)methyl |
| 252 | S | dibenzo[b,d]thien-2-yl | (5-(hydroxymethyl)-1-naphthyl)methyl |
| 253 | S | dibenzo[b,a]thien-4-yl | (5-(hydroxymethyl)-1-naphthyl)methyl |
| 254 | S | thianthren-1-yl | (5-(hydroxymethyl)-1-naphthyl)methyl |
| 255 | S | thianthren-2-yl | (5-(hydroxymethyl)-1-naphthyl)methyl |
| 256 | single bond | phenyl | (5-(hydroxymethyl)-1-naphthyl)methyl |
| 257 | single bond | naphth-1-yl | (5-(hydroxymethyl)-1-naphthyl)methyl |
| 258 | single bond | naphth-2-yl | (5-(hydroxymethyl)-1-naphthyl)methyl |
| 259 | single bond | phenanthren-9-yl | (5-(hydroxymethyl)-1-naphthyl)methyl |
| 260 | single bond | dibenzo[b,d]thien-2-yl | (5-(hydroxymethyl)-1-naphthyl)methyl |
| 261 | single bond | dibenzo[b,d]thien-4-yl | (5-(hydroxymethyl)-1-naphthyl)methyl |
| 262 | single bond | thianthren-1-yl | (5-(hydroxymethyl)-1-naphthyl)methyl |
| 263 | single bond | thianthren-2-yl | (5-(hydroxymethyl)-1-naphthyl)methyl |
| 264 | C(CH₃)₂ | phenyl | (6-(hydroxymethyl)-2-naphthyl)methyl |
| 265 | C(CH₃)₂ | naphth-1-yl | (6-(hydroxymethyl)-2-naphthyl)methyl |
| 266 | C(CH₃)₂ | naphth-2-yl | (6-(hydroxymethyl)-2-naphthyl)methyl |
| 267 | C(CH₃)₂ | phenanthren-9-yl | (6-(hydroxymethyl)-2-naphthyl)methyl |
| 268 | C(CH₃)₂ | dibenzo[b,d]thien-2-yl | (6-(hydroxymethyl)-2-naphthyl)methyl |
| 269 | C(CH₃)₂ | dibenzo[b,d]thien-4-yl | (6-(hydroxymethyl)-2-naphthyl)methyl |
| 270 | C(CH₃)₂ | thianthren-1-yl | (6-(hydroxymethyl)-2-naphthyl)methyl |
| 271 | C(CH₃)₂ | thianthren-2-yl | (6-(hydroxymethyl)-2-naphthyl)methyl |
| 272 | SO₂ | phenyl | (6-(hydroxymethyl)-2-naphthyl)methyl |
| 273 | SO₂ | naphth-1-yl | (6-(hydroxymethyl)-2-naphthyl)methyl |
| 274 | SO₂ | naphth-2-yl | (6-(hydroxymethyl)-2-naphthyl)methyl |
| 275 | SO₂ | phenanthren-9-yl | (6-(hydroxymethyl)-2-naphthyl)methyl |
| 276 | SO₂ | dibenzo[b,d]thien-2-yl | (6-(hydroxymethyl)-2-naphthyl)methyl |
| 277 | SO₂ | dibenzo[b,d]thien-4-yl | (6-(hydroxymethyl)-2-naphthyl)methyl |
| 278 | SO₂ | thianthren-1-yl | (6-(hydroxymethyl)-2-naphthyl)methyl |
| 279 | SO₂ | thianthren-2-yl | (6-(hydroxymethyl)-2-naphthyl)methyl |
| 280 | S | phenyl | (6-(hydroxymethyl)-2-naphthyl)methyl |
| 281 | S | naphth-1-yl | (6-(hydroxymethyl)-2-naphthyl)methyl |
| 282 | S | naphth-2-yl | (6-(hydroxymethyl)-2-naphthyl)methyl |
| 283 | S | phenanthren-9-yl | (6-(hydroxymethyl)-2-naphthyl)methyl |
| 284 | S | dibenzo[b,d]thien-2-yl | (6-(hydroxymethyl)-2-naphthyl)methyl |
| 285 | S | dibenzo[b,d]thien-4-yl | (6-(hydroxymethyl)-2-naphthyl)methyl |
| 286 | S | thianthren-1-yl | (6-(hydroxymethyl)-2-naphthyl)methyl |
| 287 | S | thianthren-2-yl | (6-(hydroxymethyl)-2-naphthyl)methyl |
| 288 | single bond | phenyl | (6-(hydroxymethyl)-2-naphthyl)methyl |
| 289 | single bond | naphth-1-yl | (6-(hydroxymethyl)-2-naphthyl)methyl |
| 290 | single bond | naphth-2-yl | (6-(hydroxymethyl)-2-naphthyl)methyl |
| 291 | single bond | phenanthren-9-yl | (6-(hydroxymethyl)-2-naphthyl)methyl |
| 292 | single bond | dibenzo[b,d]thien-2-yl | (6-(hydroxymethyl)-2-naphthyl)methyl |
| 293 | single bond | dibenzo[b,d]thien-4-yl | (6-(hydroxymethyl)-2-naphthyl)methyl |
| 294 | single bond | thianthren-1-yl | (6-(hydroxymethyl)-2-naphthyl)methyl |
| 295 | single bond | thianthren-2-yl | (6-(hydroxymethyl)-2-naphthyl)methyl |

14. The compound of any one of the preceding claims, where formula (I) is represented by formula (Ia-2), where Z is defined as Z¹ and Z² in claim 1 or claim 4:

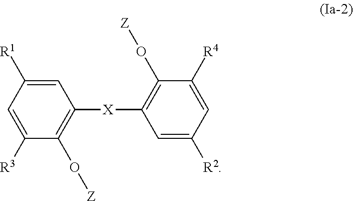

15. The compound of claim 12, where R^{y}, X and Z are as defined in one row of table B, wherein R^{y} represents the identical meaning of the substituents R¹, R², R³ and R⁴:
TABLE B

| | X | R^{y} (R¹ = R² = R³ = R⁴) | Z |
|---|---|---|---|
| 1 | single bond | phenyl | hydrogen |
| 2 | single bond | naphth-1-yl | hydrogen |
| 3 | single bond | naphth-2-yl | hydrogen |
| 4 | single bond | phenanthren-9-yl | hydrogen |
| 5 | single bond | dibenzo[b,d]thien-2-yl | hydrogen |
| 6 | single bond | dibenzo[b,d]thien-4-yl | hydrogen |
| 7 | single bond | thianthren-1-yl | hydrogen |
| 8 | single bond | thianthren-2-yl | hydrogen |
| 9 | single bond | phenyl | 2-hydroxyethyl |
| 10 | single bond | naphth-1-yl | 2-hydroxyethyl |
| 11 | single bond | naphth-2-yl | 2-hydroxyethyl |
| 12 | single bond | phenanthren-9-yl | 2-hydroxyethyl |
| 13 | single bond | dibenzo[b,d]thien-2-yl | 2-hydroxyethyl |
| 14 | single bond | dibenzo[b,d]thien-4-yl | 2-hydroxyethyl |
| 15 | single bond | thianthren-1-yl | 2-hydroxyethyl |
| 16 | single bond | thianthren-2-yl | 2-hydroxyethyl |
| 17 | single bond | phenyl | methoxycarbony-methyl |
| 18 | single bond | naphth-1-yl | methoxycarbony-methyl |
| 19 | single bond | naphth-2-yl | methoxycarbony-methyl |
| 20 | single bond | phenanthren-9-yl | methoxycarbony-methyl |
| 21 | single bond | dibenzo[b,d]thien-2-yl | methoxycarbony-methyl |
| 22 | single bond | dibenzo[b,d]thien-4-yl | methoxycarbony-methyl |
| 23 | single bond | thianthren-1-yl | methoxycarbony-methyl |
| 24 | single bond | thianthren-2-yl | methoxycarbony-methyl |
| 25 | single bond | phenyl | (4-(hydroxymethyl)phenyl)methyl |
| 26 | single bond | naphth-1-yl | (4-(hydroxymethyl)phenyl)methyl |
| 27 | single bond | naphth-2-yl | (4-(hydroxymethyl)phenyl)methyl |
| 28 | single bond | phenanthren-9-yl | (4-(hydroxymethyl)phenyl)methyl |
| 29 | single bond | dibenzo[b,d]thien-2-yl | (4-(hydroxymethyl)phenyl)methyl |
| 30 | single bond | dibenzo[b,d]thien-4-yl | (4-(hydroxymethyl)phenyl)methyl |
| 31 | single bond | thianthren-1-yl | (4-(hydroxymethyl)phenyl)methyl |
| 32 | single bond | thianthren-2-yl | (4-(hydroxymethyl)phenyl)methyl |
| 33 | single bond | phenyl | (3-(hydroxymethyl)phenyl)methyl |
| 34 | single bond | naphth-1-yl | (3-(hydroxymethyl)phenyl)methyl |
| 35 | single bond | naphth-2-yl | (3-(hydroxymethyl)phenyl)methyl |
| 36 | single bond | phenanthren-9-yl | (3-(hydroxymethyl)phenyl)methyl |
| 37 | single bond | dibenzo[b,d]thien-2-yl | (3-(hydroxymethyl)phenyl)methyl |
| 38 | single bond | dibenzo[b,d]thien-4-yl | (3-(hydroxymethyl)phenyl)methyl |
| 39 | single bond | thianthren-1-yl | (3-(hydroxymethyl)phenyl)methyl |
| 40 | single bond | thianthren-2-yl | (3-(hydroxymethyl)phenyl)methyl |
| 41 | single bond | phenyl | (4-(hydroxymethyl)-1-naphthyl)methyl |
| 42 | single bond | naphth-1-yl | (4-(hydroxymethyl)-1-naphthyl)methyl |
| 43 | single bond | naphth-2-yl | (4-(hydroxymethyl)-1-naphthyl)methyl |
| 44 | single bond | phenanthren-9-yl | (4-(hydroxymethyl)-1-naphthyl)methyl |
| 45 | single bond | dibenzo[b,d]thien-2-yl | (4-(hydroxymethyl)-1-naphthyl)methyl |
| 46 | single bond | dibenzo[b,d]thien-4-yl | (4-(hydroxymethyl)-1-naphthyl)methyl |
| 47 | single bond | thianthren-1-yl | (4-(hydroxymethyl)-1-naphthyl)methyl |
| 48 | single bond | thianthren-2-yl | (4-(hydroxymethyl)-1-naphthyl)methyl |
| 49 | single bond | phenyl | (5-(hydroxymethyl)-1-naphthyl)methyl |
| 50 | single bond | naphth-1-yl | (5-(hydroxymethyl)-1-naphthyl)methyl |
| 51 | single bond | naphth-2-yl | (5-(hydroxymethyl)-1-naphthyl)methyl |
| 52 | single bond | phenanthren-9-yl | (5-(hydroxymethyl)-1-naphthyl)methyl |
| 53 | single bond | dibenzo[b,d]thien-2-yl | (5-(hydroxymethyl)-1-naphthyl)methyl |
| 54 | single bond | dibenzo[b,d]thien-4-yl | (5-(hydroxymethyl)-1-naphthyl)methyl |
| 55 | single bond | thianthren-1-yl | (5-(hydroxymethyl)-1-naphthyl)methyl |
| 56 | single bond | thianthren-2-yl | (5-(hydroxymethyl)-1-naphthyl)methyl |
| 57 | single bond | phenyl | (6-(hydroxymethyl)-2-naphthyl)methyl |
| 58 | single bond | naphth-1-yl | (6-(hydroxymethyl)-2-naphthyl)methyl |
| 59 | single bond | naphth-2-yl | (6-(hydroxymethyl)-2-naphthyl)methyl |
| 60 | single bond | phenanthren-9-yl | (6-(hydroxymethyl)-2-naphthyl)methyl |
| 61 | single bond | dibenzo[b,d]thien-2-yl | (6-(hydroxymethyl)-2-naphthyl)methyl |
| 62 | single bond | dibenzo[b,d]thien-4-yl | (6-(hydroxymethyl)-2-naphthyl)methyl |
| 63 | single bond | thianthren-1-yl | (6-(hydroxymethyl)-2-naphthyl)methyl |
| 64 | single bond | thianthren-2-yl | (6-(hydroxymethyl)-2-naphthyl)methyl |

16. A thermoplastic resin comprising a structural unit represented by formulae (II) below 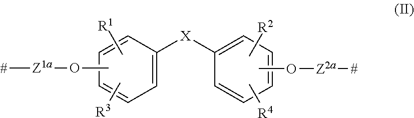
where
# represents a connection point to a neighboring structural unit;
and where Z^{1a} and Z^{2a}, respectively, is derived from Z¹ or Z² in formula (I), if Z¹ or Z² is hydrogen, by replacing hydrogen with a single bond, or, if Z¹ or Z² is not hydrogen, by replacing the —OH or —OR^{x} group of Z¹ or Z² with an oxo (—O—) unit, and where Z¹, Z², X, R¹, R², R³ and R⁴ are as defined in any one of claims 1 to 15.

17. The thermoplastic resin of claim 16, where formula (II) is represented by formula (IIa):

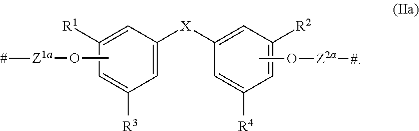

18. The thermoplastic resin of claim 17, where formula (IIa) is represented by formula (IIa-1), wherein Z^{a} is defined as Z^{1a} and Z^{2a} in claim 16:

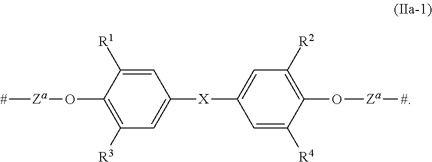

19. The thermoplastic resin of claim 17, where formula (IIa) is represented by formula (IIa-2), wherein Z^{a} is defined as Z^{1a} and Z^{2a} in claim 16:

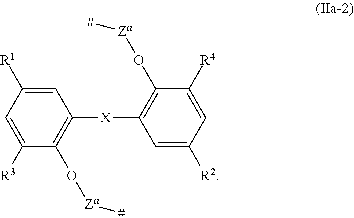

20. The thermoplastic resin of any one of claims 16 to 19, where the structural unit of the formula (II), where Z¹ and Z² is selected from hydrogen,
Alk-OH and —CH₂—Ar²—CH₂—OH, is connected to one of the structures represented by formulae (III-1) to (III-5) below, 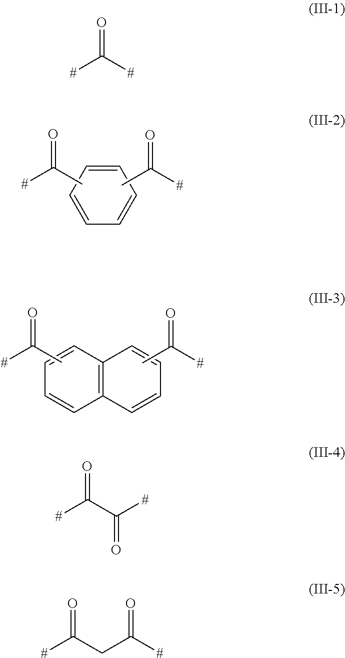
where
# represents a connection point to a neighboring structural unit.

21. The thermoplastic resin of any one of claims 16 to 20, which is selected from copolycarbonate resins, copolyestercarbonate resins and copolyester resins, where the thermoplastic resin in addition to structural units represented by formula (II) comprises a structural unit of the formula (V),
#-O—R^{z}-A¹-R^{z}—O-#- (V)
where
# represents a connection point to a neighboring structural unit;
A¹ is a polycyclic radical bearing at least 2 benzene rings, wherein the benzene rings may be connected by A and/or directly fused to each other and/or fused by a non-benzene carbocycle, where A¹ is unsubstituted or substituted by 1, 2 or 3 radicals R^{aa}, which are selected from the group consisting of halogen, C₁-C₆-alkyl, C₅-C₆-cycloalkyl and phenyl;
A is selected from the group consisting of a single bond, O, C═O, S, SO₂, CH₂, CH—Ar, CAr₂, CH(CH₃), C(CH₃)₂ and a radical of the formula (A′) 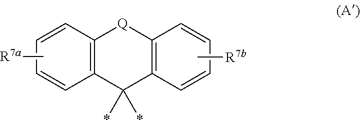
where
Q represents a single bond, O, NH, C═O, CH₂ or CH═CH;
R^{7a}, R^{7b}, independently of each other are selected from the group consisting of hydrogen, fluorine, CN, R, OR, CHₖR₃₋ₖ, NR₂, C(O)R and C(O)NH₂, where R is as defined in claim 1 and k is 0, 1, 2 or 3; and
* represents the connection point to a benzene ring;
Ar is selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulphur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk¹, O-Alk²-, O-Alk²-[O-Alk²-]ₚ- or O-Alk³-C(O)— where O is bound to A¹, and where
p is an integer from 1 to 10;
Alk¹ is C₁-C₄-alkandiyl;
Alk² is C₂-C₄-alkandiyl; and
Alk³ is C₁-C₄-alkandiyl.

22. The thermoplastic resin of claim 21, where the structural unit of the formula V is represented by one of the following formulae V-1 to V-6: 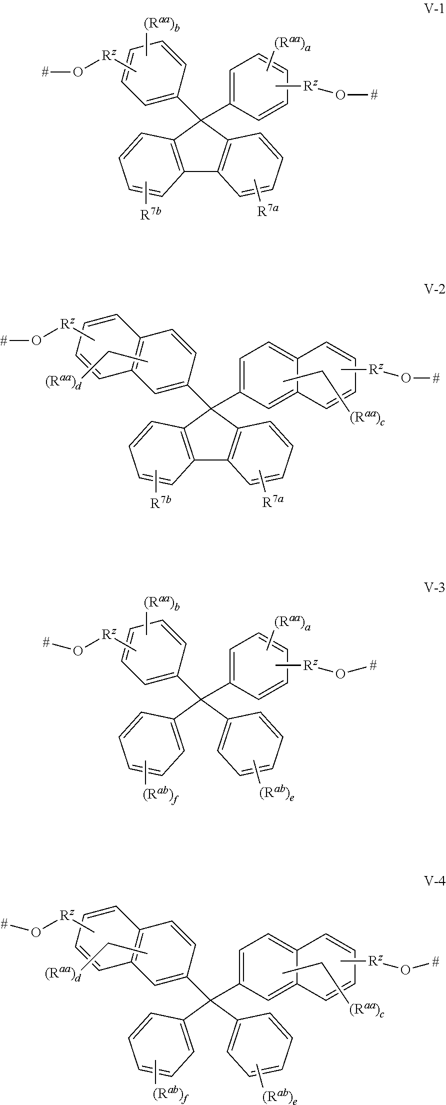 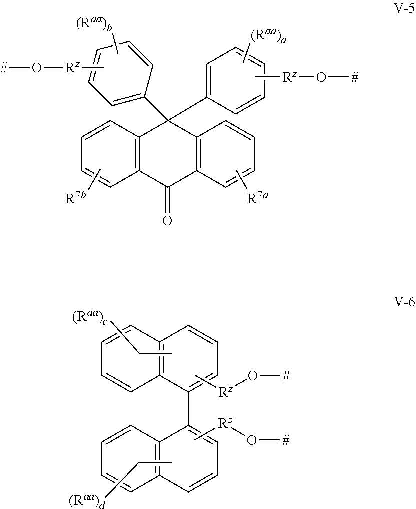
where
a and b are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
and where R^{z}, R^{aa}, R^{ab}, R^{7a} and R^{7b} are as defined for formula (V).

23. The thermoplastic resin of any one of claims 21 or 22, where the molar ratio of the structural units of the formula (II) is from 1 to 70 mol-% based on the total molar amount of structural units of the formulae (II) and (V) and where the molar ratio of the structural units of the formula (V) is from 30 to 99 mol-% based on the total molar amount of structural units of the formulae (II) and (V).

24. The thermoplastic resin of any one of claims 16 to 23, which has a refractive index of 1.640 or higher.

25. The thermoplastic resin of any one of claims 16 to 24, which has an Abbe number of 24 or lower.

26. The thermoplastic resin of any one of claims 16 to 25, which has a glass transition temperature (Tg) of 90 to 185° C.

27. The thermoplastic resin of any one of claims 16 to 26, which has a weight-average molecular weight of 10000 to 50000 as determined by gel permeation chromatography against a polystyrene standard.

28. The thermoplastic resin of any one of claims 16 to 27 comprising low molecular weight compounds with molecular weight (Mw) of below 1000, wherein the content of the low molecular weight compounds (CLWC) in the thermoplastic resin is in the range of 0.3% by weight to 7.0% by weight, based on the total weight of the thermoplastic resin, and wherein the CLWC is represented by following formula:
$$CLWC ( \% ) = \frac{\begin{matrix}the total area of peaks of compounds with \\ Mw lower than 1. on GPC analysis\end{matrix}}{( the total area of all peaks of compounds on GPC analysis )} \times 100$$

29. An optical device made of a thermoplastic resin as defined in any one of claims 16 to 28.

30. Use of the compound of the formula (I) as defined in any one of claims 1 to 15, as a monomer of the thermoplastic resin as defined in any one of claims 16 to 29.
